(19) United States
(12) Patent Application Publication
(10) Pub. No.: US 2021/0177916 A1
(43) Pub. Date: Jun. 17, 2021

(54) LONG CHAIN DICARBOXYLIC FATTY ACID (LCDFA) PRODUCING MICROBES AND USES THEREOF

(71) Applicant: MED-LIFE DISCOVERIES LP, Saskatoon (CA)

(72) Inventor: Shawn RITCHIE, Saskatoon (CA)

(73) Assignee: Med-Life Discoveries LP, Saskatoon, SK (CA)

(21) Appl. No.: 17/046,903

(22) PCT Filed: Apr. 12, 2019
(86) PCT No.: PCT/CA2019/050449

§ 371 Date: Oct. 12, 2020

Related U.S. Application Data
(60) Provisional application No. 62/657,500, filed on Apr. 13, 2018.

(51) Int. Cl. A61K 35/74; G01N 33/92

(52) U.S. Cl. CPC A61K 35/74; G01N 33/92

(57) ABSTRACT

A method for increasing gastric tract acid (GTA) production in a mammalian subject. The method comprises administering a therapeutically-effective amount of a composition comprising at least one live or attenuated culture of a microbial species selected from the genus Blautia, species Faecalibacterium prausnitzii, genus Bacteroides, family Ruminococcaceae, family Lachnospiraceae, genus Coprococcus, genus Roseburia, genus Oscillospira, species Ruminococcus bromii, genus Ruminococcus, family Costridiaceae, species Dorea formicigenerans, species Bacteroides uniformis, genus Dorea, genus Streptococcus, order Clostridiales, genus Anaerostipes, genus Dialister, species Bifidobacterium adolescentis, family Coriobacteriaceae, genus Faecalibacterium, genus Sutterella, species Bacteroides ovatus, genus Parabacteroides, genus Ruminococcus, species Bacteroides faecis, species Eubacterium biforme, genus Phascolartobacterium, and family Enterobacteriaceae; or a prebiotic composition which increases growth and/or viability of said microbial species in the gut. Administering the composition increases the synthesis of at least one GTA dicarboxylic fatty acid metabolite in said subject. Also described are method for determining gastrointestinal inflammation status and kits for detecting and treating a gastric tract acid (GTA) insufficiency.

### FIELD OF INVENTION

The present invention relates to the treatment of gastrointestinal inflammation and gastric tract acid (GTA) long-chain fatty acid deficiency through the manipulation of the gut microbiome. The invention also relates to compositions and methods of increasing gastric tract acid (GTA) production in a mammalian subject.

### BACKGROUND OF THE INVENTION

Chronic inflammation is widely accepted as the primary underlying cause of gastrointestinal (GI) cancers, including colorectal cancer, pancreatic cancer, gastric cancer, esophageal cancer, ovarian cancer, and others (Marusawa and Jenkins 2014, Hussain and Harris 2007, Chapkin, McMurray and Lupton 2007, Demaria et al. 2010, Itzkowitz and Yio 2004, Maccio and Madeddu 2012, Schwartsburd 2004, Terzic et al. 2010, Wu et al. 2014). Chronic inflammation can lead to oxidative stress, which can subsequently result in carcinogenic events and genetic mutations that drive the malignant transformation of cells. (Mannick et al and Zhang et al). Cancer growth is subsequently driven by the proinflammatory milieu of cytokines and angiogenic factors in the microenvironment.

Despite the preponderance of evidence linking GI cancers to chronic inflammation, all of the emphasis on the early detection of cancer (take for example colorectal cancer), has focused exclusively on the improved detection of tumor-derived markers or precancerous lesions, and not underlying metabolic or inflammatory risk factors. In the case of colorectal cancer, the primary screening modalities are either direct visualization of cancer growth or precancerous lesions by endoscopy, the detection of occult blood in the stool, or more recently methylated tumor DNA in either feces or blood. For each of these modalities to be effective, there has to be a minimal tumor burden present that is of sufficient magnitude to either physically view or biopsy a lesion, or sufficient tumor load to produce detectable levels of tumor-derived biomarkers in either the feces or blood. Therefore, such approaches offer no hope for preventing the occurrence of the disease to begin with; but rather only offer hope in the form of early-stage detection where treatment is generally more effective.

A key component of the inflammatory status of the gut was identified by Ritchie et al. through a non-targeted metabolomic analysis of small molecules that differentiated colorectal cancer, pancreatic cancer, and ovarian cancer from disease-free subjects (Ritchie et al. 2010a, Ritchie et al. 2010c, Ritchie et al. 2010b, Ritchie et al. 2011, Ritchie et al. 2013b, Ritchie et al. 2013a, Ritchie et al. 2015). A novel family of metabolites initially proposed to be vitamin E metabolites, but subsequently shown to be novel long-chain polyunsaturated dicarboxylic fatty acids (called GTAs for gastric tract acids), ranging between 28 and 36 carbons in size, and with molecular weights between approximately 446 and 596 Da, were shown to be consistently reduced in the serum of subjects with these cancers compared to controls.

GTAs were shown to possess anti-inflammatory as well as anti-proliferative activity in vitro (Ritchie et al. 2011) though the co-administration of semi-purified GTA-containing and GTA-deficient extracts with LPS in various cell systems. The anti-inflammatory activity was shown to be mediated through NF-κB, a transcription factor involved in the activation of several pro-inflammatory cytokines, including TNF-alpha and Interleukin-1β. Specifically, GTAs significantly upregulated IκBα, an inhibitory protein that inactivates NF-κB. NF-κB overexpression has been linked to multiple aspects of chronic inflammation, and has been the target of therapeutic downregulation by synthetic and natural products (Ben-Neriah and Karin 2011, Spehlmann and Eckmann 2009, Surh et al. 2001, Xu et al. 2005, Freitas and Fraga 2018).

These GTAs continue to be a source of interest and research to better understand their mechanism of action and potential role in treating inflammation and disease, such as cancer.

### SUMMARY OF THE INVENTION

It is an object of the invention to provide new methods for detecting and reducing gastrointestinal (GI) inflammation in a subject.

An approach is thus provided herein for identifying subjects with GI inflammation by measuring levels of long-chain dicarboxylic fatty acids (LCDFAs), or GTAs, in the blood, followed by treatment with chemical agents or micro-organisms to restore or augment these GTA levels.

In certain embodiments, the micro-organisms are long-chain fatty acid-producing bacteria, especially those from the genus Blautia and species Faecalibacterium prausnitzii.

Particular microbial species are also shown to be associated with these GTA levels, and thus strategies are provided to augment these microbial species for the purpose of reducing GI inflammation.

Also provided is an approach for identifying an underlying metabolic inflammatory condition associated with the development of various GI-related cancers, including but not limited to colorectal cancer, pancreatic cancer, and ovarian cancer, followed by an approach to reduce risk through therapeutic treatment of the underlying inflammation.

Accordingly, there is provided a method for increasing gastric tract acid (GTA) production in a mammalian subject. The method comprises administering a therapeutically-effective amount of a composition comprising at least one live or attenuated culture of a microbial species selected from the genus Blautia, species Faecalibacterium prausnitzii, genus Bacteroides, family Ruminococcaceae, family Lachnospiraceae, genus Coprococcus, genus Roseburia, genus Oscillospira, species Ruminococcus bromii, genus Ruminococcus, family Costridiaceae, species Dorea formicigenerans, species Bacteroides uniformis, genus Dorea, genus Streptococcus, order Clostridiales, genus Anaerostipes, genus Dialister, species Bifidobacterium adolescentis, family Coriobacteriaceae, genus Faecalibacterium, genus Sutterella, species Bacteroides ovatus, genus Parabacteroides, genus Ruminococcus, species Bacteroides faecis, species Eubacterium biforme, genus Phascolartobacterium, and family Enterobacteriaceae; or a prebiotic composition which increases growth and/or viability of said microbial species in the gut; wherein the composition increases the synthesis of at least one GTA dicarboxylic fatty acid metabolite in said subject.

In certain embodiments, the method further comprises a step of measuring circulating levels of one or more GTA dicarboxylic fatty acid metabolite in the subject. The composition may thus be administered if the levels of the one or more GTA dicarboxylic fatty acid metabolite are found to be lower in the subject than a predetermined control level, an earlier test value obtained for the subject, or a normal level for healthy subjects. For example, yet without wishing to be limiting, the control may include a predetermined threshold value for the at least one GTA dicarboxylic fatty acid metabolite that is typical of a healthy individual.

In further embodiments, the composition may comprise a live or attenuated culture of a microbial species from the genus Blautia, a live or attenuated culture of Faecalibacterium prausnitzii, or a combination thereof. Typically such cultures will be formulated within a pharmaceutically-acceptable excipient or carrier suitable for administration to the gastrointestinal tract of a subject.

In yet further embodiments, the GTA dicarboxylic fatty acid metabolite is a dicarboxylic fatty acid between 28 and 36 carbons comprised of a dimeric fatty acid structure of two shorter chains ranging between 14 and 18 carbons in length joined by a single or double bond. In further embodiments, the GTA dicarboxylic fatty acid metabolite may be one of the following: GTA-446, GTA-448, GTA-450, GTA-452, GTA-464, GTA-466, GTA-468, GTA-474, GTA-476, GTA-478, GTA-484, GTA-490, GTA-492, GTA-494, GTA-502, GTA-504, GTA-512, GTA-518, GTA-520, GTA-522, GTA-524, GTA-530, GTA-532, GTA-536, GTA-538, GTA-540, GTA-550, GTA-574, GTA-576, GTA-580, GTA-590, GTA-592, GTA-594, and GTA-596.

In non-limiting embodiments, the GTA dicarboxylic fatty acid metabolite may have an accurate neutral mass within 1 PPM Dalton mass accuracy of 446.3396 (GTA-446), 448.3553 (GTA-448), 450.3709 (GTA-450), 452.3866 (GTA-452), 464.3522 (GTA-464), 466.3661 (GTA-466), 468.3814 (GTA-468), 474.3736 (GTA-474), 476.3866 GTA-476, 478.4022 (GTA-478), 484.3764 (GTA-484), 490.3658 (GTA-490), 492.3815 (GTA-492), 494.3971 (GTA-494), 502.4022 (GTA-502), 504.4195 (GTA-504), 512.4077 (GTA-512), 518.3974 (GTA-518), 520.4128 (GTA-520), 522.4284 (GTA-522), 524.4441 (GTA-524), 530.4335 (GTA-530), 532.4492 (GTA-532), 536.4077 (GTA-536), 538.4233 (GTA-538), 540.4389 (GTA-540), 550.4597 (GTA-550), 574.4597 (GTA-574), 576.4754 (GTA-576), 580.5067 (GTA-580), 590.4546 (GTA-590), 592.4703 (GTA-592), 594.4859 (GTA-594), or 596.5016 (GTA-596).

In further non-limiting embodiments, the GTA dicarboxylic fatty acid metabolite may have a molecular formula of C₂₈H₄₆O₄ (GTA-446), C₂₈H₄₈O₄ (GTA-448), C₂₈H₅₀O₄ (GTA-450), C₂₈H₅₂O₄ (GTA-452), 464.3522 (GTA-464), 466.3661 (GTA-466), C₂₈H₅₂O₅ (GTA-468), C₃₀H₅₀O₄ (GTA-474), C₃₀H₅₂O₄ GTA-476, C₃₄H₅₄O₄ (GTA-478), C₂₈₅₂O₆ (GTA-484), C₃₀H₅₀O₅ (GTA-490), C₃₀H₅₂O₅ (GTA-492), C₃₄H₅₄O₅ (GTA-494), C₃₂H₅₄O₄ (GTA-502), C₃₂H₅₆O₄ (GTA-504), C₃₀H₅₆O₆ (GTA-512), C₃₂H₅₄O₅ (GTA-518), C₃₂H₅₆O₅ (GTA-520), C₃₂H₅₈O₅ (GTA-522), C₃₂H₆₀O₅ GTA-524, C₃₄H₅₈O₄ (GTA-530), C₃₄H₆₀O₄ (GTA-532), C₃₂H₅₆O₆ (GTA-536), C₃₂H₅₈O₆ (GTA-538), C₃₂H₆₀O₆ (GTA-540), C₃₄H₆₂O₅ (GTA-550), C₃₆H₆₂O₅ (GTA-574), C₃₆H₆₄O₅ (GTA-576), C₃₆H₆₈O₅ (GTA-580), C₃₆H₆₂O₆ (GTA-590), C₃₆H₆₄O₆ (GTA-592), C₃₆H₆₆O₆ (GTA-594), or C₃₆H₆₈O₆ (GTA-596).

In further non-limiting embodiments, the GTA dicarboxylic fatty acid metabolite may be measured using collision induced dissociation (CID) tandem mass spectrometry. The GTAs may be one or more of the GTA dicarboxylic fatty acid metabolites listed below:

GTA-446, having an accurate neutral mass within 1 PPM Dalton mass accuracy of 446.3396, the molecular formula of C₂₈H₄₆O₄, and characterized by a OD MS/MS fragmentation pattern using N₂ as collision gas and analyzed in atmospheric pressure chemical ionization (APCI) under negative ionization comprising daughter ions of parent [M−H] mass 445: 427, 409, 401, and 383,

GTA-448, having an accurate neutral mass within 1 PPM Dalton mass accuracy of 448.3553, the molecular formula of C₂₈H₄₈O₄, and characterized by a OD MS/MS fragmentation pattern using N₂ as collision gas and analyzed in atmospheric pressure chemical ionization (APCI) under negative ionization comprising daughter ions of parent [M−H] mass 447: 429, 411, 403, and 385,

GTA-450, having an accurate neutral mass within 1 PPM Dalton mass accuracy of 450.3709, the molecular formula of C₂₈H₅₀O₄, and characterized by a OD MS/MS fragmentation pattern using N₂ as collision gas and analyzed in atmospheric pressure chemical ionization (APCI) under negative ionization comprising daughter ions of parent [M−H] mass 449: 431, 413, 405, and 387,

GTA-452, having an accurate neutral mass within 1 PPM Dalton mass accuracy of 452.3866, the molecular formula of C₂₈H₅₂O₄, and characterized by a OD MS/MS fragmentation pattern using N₂ as collision gas and analyzed in atmospheric pressure chemical ionization (APCI) under negative ionization comprising daughter ions of parent [M−H] mass 451: 433, 407, and 389,

GTA-464, having an accurate neutral mass within 1 PPM Dalton mass accuracy of 464.3522, the molecular formula of C₂₈H₄₈O₅, and characterized by a OD MS/MS fragmentation pattern using N₂ as collision gas and analyzed in atmospheric pressure chemical ionization (APCI) under negative ionization comprising daughter ions of parent [M−H] mass 463: 445, 419, 401, and 383,

GTA-466, having an accurate neutral mass within 1 PPM Dalton mass accuracy of 466.3661, the molecular formula of C₂₈H₅₀O₅, and characterized by a OD MS/MS fragmentation pattern using N₂ as collision gas and analyzed in atmospheric pressure chemical ionization (APCI) under negative ionization comprising daughter ions of parent [M−H] mass 465: 447, 421, and 403,

GTA-468, having an accurate neutral mass within 1 PPM Dalton mass accuracy of 468.3814, having the molecular formula of C₂₈H₅₂O₅, and characterized by a OD MS/MS fragmentation pattern using N₂ as collision gas and analyzed in atmospheric pressure chemical ionization (APCI) under negative ionization comprising daughter ions of parent [M−H] mass 467: 449, 423, and 405,

GTA-474, having an accurate neutral mass within 1 PPM Dalton mass accuracy of 474.3736, having the molecular formula of C₃₀H₅₀O₄, and characterized by a OD MS/MS fragmentation pattern using N₂ as collision gas and analyzed in atmospheric pressure chemical ionization (APCI) under negative ionization comprising daughter ions of parent [M−H] mass 473: 455, 429, and 411,

GTA-476, having an accurate neutral mass within 1 PPM Dalton mass accuracy of 476.3866, having the molecular formula of C₃₀H₅₂O₄, and characterized by a OD MS/MS fragmentation pattern using N₂ as collision gas and analyzed in atmospheric pressure chemical ionization (APCI) under negative ionization comprising daughter ions of parent [M−H] mass 475: 457, 431, 439 and 413,

GTA-478, having an accurate neutral mass within 1 PPM Dalton mass accuracy of 478.4022, having the molecular formula of C₃₀H₅₄O₄, and characterized by a OD MS/MS fragmentation pattern using N₂ as collision gas and analyzed in atmospheric pressure chemical ionization (APCI) under negative ionization comprising daughter ions of parent [M−H] mass 477: 459, 433, 441 and 415,

GTA-484, having an accurate neutral mass within 1 PPM Dalton mass accuracy of 484.3764, having the molecular formula of C₂₈H₅₂O₆, and characterized by a OD MS/MS fragmentation pattern using N₂ as collision gas and analyzed in atmospheric pressure chemical ionization (APCI) under negative ionization comprising daughter ions of parent [M−H] mass 483: 465, 315, 439 483, 421, and 447,

GTA-490, having an accurate neutral mass within 1 PPM Dalton mass accuracy of 490.3658, having the molecular formula of C₃₀H₅₀O₅, and characterized by a OD MS/MS fragmentation pattern using N₂ as collision gas and analyzed in atmospheric pressure chemical ionization (APCI) under negative ionization comprising daughter ions of parent [M−H] mass 489: 445, 471, 427 and 319,

GTA-492, having an accurate neutral mass within 1 PPM Dalton mass accuracy of 492.3815, having the molecular formula of C₃₀H₅₂O₅, and characterized by a OD MS/MS fragmentation pattern using N₂ as collision gas and analyzed in atmospheric pressure chemical ionization (APCI) under negative ionization comprising daughter ions of parent [M−H] mass 491: 241, 249, 267, 473, and 447,

GTA-494, having an accurate neutral mass within 1 PPM Dalton mass accuracy of 494.3971, having the molecular formula of C₃₀H₅₄O₅, and characterized by a OD MS/MS fragmentation pattern using N₂ as collision gas and analyzed in atmospheric pressure chemical ionization (APCI) under negative ionization comprising daughter ions of parent [M−H] mass 493: 475, 215, and 449,

GTA-502, having an accurate neutral mass within 1 PPM Dalton mass accuracy of 502.4022, having the molecular formula of C₃₂H₅₄O₄, and characterized by a OD MS/MS fragmentation pattern using N₂ as collision gas and analyzed in atmospheric pressure chemical ionization (APCI) under negative ionization comprising daughter ions of parent [M−H] mass 501: 483, 457, 465 and 439,

GTA-504, having an accurate neutral mass within 1 PPM Dalton mass accuracy of 504.4195, having the molecular formula of C₃₂H₅₆O₄, and characterized by a OD MS/MS fragmentation pattern using N₂ as collision gas and analyzed in atmospheric pressure chemical ionization (APCI) under negative ionization comprising daughter ions of parent [M−H] mass 503: 485, 459, 467 and 441,

GTA-512, having an accurate neutral mass within 1 PPM Dalton mass accuracy of 512.4077, having the molecular formula of C₃₀H₅₆O₆, and characterized by a OD MS/MS fragmentation pattern using N₂ as collision gas and analyzed in atmospheric pressure chemical ionization (APCI) under negative ionization comprising daughter ions of parent [M−H] mass 511: 493, 315, and 467,

GTA-518, having an accurate neutral mass within 1 PPM Dalton mass accuracy of 518.3974, having the molecular formula of C₃₂H₅₄O₅, and characterized by a OD MS/MS fragmentation pattern using N₂ as collision gas and analyzed in atmospheric pressure chemical ionization (APCI) under negative ionization comprising daughter ions of parent [M−H] mass 517: 499, 473, 499, 481 and 445,

GTA-520, having an accurate neutral mass within 1 PPM Dalton mass accuracy of 520.4128, having the molecular formula of C₃₂H₅₆O₅, and characterized by a OD MS/MS fragmentation pattern using N₂ as collision gas and analyzed in atmospheric pressure chemical ionization (APCI) under negative ionization comprising daughter ions of parent [M−H] mass 519: 501, 457, 475, 459, 447 and 483,

GTA-522, having an accurate neutral mass within 1 PPM Dalton mass accuracy of 522.4284, having the molecular formula of C₃₂H₅₈O₅, and characterized by a OD MS/MS fragmentation pattern using N₂ as collision gas and analyzed in atmospheric pressure chemical ionization (APCI) under negative ionization comprising daughter ions of parent [M−H] mass 521: 503, 459, 477, 504, 441 and 485,

GTA-524, having an accurate neutral mass within 1 PPM Dalton mass accuracy of 524.4441, having the molecular formula of C₃₂H₆₀O₅, and characterized by a OD MS/MS fragmentation pattern using N₂ as collision gas and analyzed in atmospheric pressure chemical ionization (APCI) under negative ionization comprising daughter ions of parent [M−H] mass 523: 505, 461, 479, 506, 443 and 487,

GTA-530, having an accurate neutral mass within 1 PPM Dalton mass accuracy of 530.4335, having the molecular formula of C₃₄H₅₈O₄, and characterized by a OD MS/MS fragmentation pattern using N₂ as collision gas and analyzed in atmospheric pressure chemical ionization (APCI) under negative ionization comprising daughter ions of parent [M−H] mass 529: 467, 511 and 485,

GTA-532, having an accurate neutral mass within 1 PPM Dalton mass accuracy of 532.4492, having the molecular formula of C₃₄H₆₀O₄, and characterized by a OD MS/MS fragmentation pattern using N₂ as collision gas and analyzed in atmospheric pressure chemical ionization (APCI) under negative ionization comprising daughter ions of parent [M−H] mass 531: 513, 469, 487 and 495,

GTA-536, having an accurate neutral mass within 1 PPM Dalton mass accuracy of 536.4077, having the molecular formula of C₃₂H56O₆, and characterized by a OD MS/MS fragmentation pattern using N₂ as collision gas and analyzed in atmospheric pressure chemical ionization (APCI) under negative ionization comprising daughter ion of parent [M−H] mass 535: 473,

GTA-538, having an accurate neutral mass within 1 PPM Dalton mass accuracy of 538.4233, having the molecular formula of C₃₂H₅₈O₆, and characterized by a OD MS/MS fragmentation pattern using N₂ as collision gas and analyzed in atmospheric pressure chemical ionization (APCI) under negative ionization comprising daughter ions of parent [M−H] mass 537: 519, 475, 493, 501 and 457,

GTA-540, having an accurate neutral mass within 1 PPM Dalton mass accuracy of 540.4389, having the molecular formula of C₃₂H₆₀O₆, and characterized by a OD MS/MS fragmentation pattern using N₂ as collision gas and analyzed in atmospheric pressure chemical ionization (APCI) under negative ionization comprising daughter ions of parent [M−H] mass 539: 315, 521, 495 and 477,

GTA-550, having an accurate neutral mass within 1 PPM Dalton mass accuracy of 550.4597, having the molecular formula of C₃₄H₆₂O₅, and characterized by a OD MS/MS fragmentation pattern using N₂ as collision gas and analyzed in atmospheric pressure chemical ionization (APCI) under negative ionization comprising daughter ions of parent [M−H] mass 549: 487, 531, 251, 253, 513, 469 and 506,

GTA-574, having an accurate neutral mass within 1 PPM Dalton mass accuracy of 574.4597, having the molecular formula of C₃₆H₆₂O₅, and characterized by a OD MS/MS fragmentation pattern using N₂ as collision gas and analyzed in atmospheric pressure chemical ionization (APCI) under negative ionization comprising daughter ions of parent [M−H] mass 573: 295, 223, 555 and 511,

GTA-576, having an accurate neutral mass within 1 PPM Dalton mass accuracy of 576.4754, having the molecular formula of C₃₆H₆₄O₅, and characterized by a OD MS/MS fragmentation pattern using N₂ as collision gas and analyzed in atmospheric pressure chemical ionization (APCI) under negative ionization comprising daughter ions of parent [M−H] mass 575: 277, 297, 557, 513 and 495,

GTA-580, having an accurate neutral mass within 1 PPM Dalton mass accuracy of 580.5067, having the molecular formula of C₃₆H₆₈O₅, and characterized by a OD MS/MS fragmentation pattern using N₂ as collision gas and analyzed in atmospheric pressure chemical ionization (APCI) under negative ionization comprising daughter ions of parent [M−H] mass 579: 561, 543, 535, 517 and 499,

GTA-590, having an accurate neutral mass within 1 PPM Dalton mass accuracy of 590.4546, having the molecular formula of C₃₆H₆₂O₆, and characterized by a OD MS/MS fragmentation pattern using N₂ as collision gas and analyzed in atmospheric pressure chemical ionization (APCI) under negative ionization comprising daughter ion of parent [M−H] mass 589: 545,

GTA-592, having an accurate neutral mass within 1 PPM Dalton mass accuracy of 592.4703, having the molecular formula of C₃₆H₆₄O₆, and characterized by a OD MS/MS fragmentation pattern using N₂ as collision gas and analyzed in atmospheric pressure chemical ionization (APCI) under negative ionization comprising daughter ions of parent [M−H] mass 591: 555 and 113,

GTA-594, having an accurate neutral mass within 1 PPM Dalton mass accuracy of 594.4859, having the molecular formula of C₃₆H₆₆O₆, and characterized by a OD MS/MS fragmentation pattern using N₂ as collision gas and analyzed in atmospheric pressure chemical ionization (APCI) under negative ionization comprising daughter ions of parent [M−H] mass 593: 557 371, 315 and 277, and

GTA-596, having an accurate neutral mass within 1 PPM Dalton mass accuracy of 596.5016, having the molecular formula of C₃₆H₆₈O₆, and characterized by a OD MS/MS fragmentation pattern using N₂ as collision gas and analyzed in atmospheric pressure chemical ionization (APCI) under negative ionization comprising daughter ions of parent [M−H] mass 595: 279, 315, 297, 577 and 559.

For example, the GTA dicarboxylic fatty acid metabolite may be GTA-446, which has the formula C₂₈H₄₆O₄ and the structure:

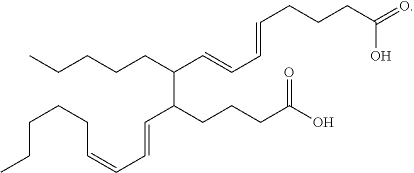 [SMILES: CCCCC/C=C\C=C\C(CCCC(=O)O)C(/C=C/C=C/CCCC(=O)O)CCCCC]

There is also provided herein a method for determining gastrointestinal inflammation status within the body of a mammalian subject. The method comprises measuring circulating levels of one or more GTA dicarboxylic fatty acid metabolite, wherein the GTA dicarboxylic fatty acid metabolite is a dicarboxylic fatty acid between 28 and 36 carbons comprised of a dimeric fatty acid structure of two shorter chains ranging between 14 and 18 carbons in length joined by a single or double bond, and if a level one or more of these GTA dicarboxylic fatty acid metabolites is detected to be lower than a predetermined control level, an earlier test value for the subject, or a normal level for healthy subjects, the subject is assessed as having or being at risk for gastrointestinal inflammation.

In further embodiments of the method, the GTA dicarboxylic fatty acid metabolite may be one of the following: GTA-446, GTA-448, GTA-450, GTA-452, GTA-464, GTA-466, GTA-468, GTA-474, GTA-476, GTA-478, GTA-484, GTA-490, GTA-492, GTA-494, GTA-502, GTA-504, GTA-512, GTA-518, GTA-520, GTA-522, GTA-524, GTA-530, GTA-532, GTA-536, GTA-538, GTA-540, GTA-550, GTA-574, GTA-576, GTA-580, GTA-590, GTA-592, GTA-594, and GTA-596.

In non-limiting embodiments, the GTA dicarboxylic fatty acid metabolite may have an accurate neutral mass within 1 PPM Dalton mass accuracy of 446.3396 (GTA-446), 448.3553 (GTA-448), 450.3709 (GTA-450), 452.3866 (GTA-452), 464.3522 (GTA-464), 466.3661 (GTA-466), 468.3814 (GTA-468), 474.3736 (GTA-474), 476.3866 GTA-476, 478.4022 (GTA-478), 484.3764 (GTA-484), 490.3658 (GTA-490), 492.3815 (GTA-492), 494.3971 (GTA-494), 502.4022 (GTA-502), 504.4195 (GTA-504), 512.4077 (GTA-512), 518.3974 (GTA-518), 520.4128 (GTA-520), 522.4284 (GTA-522), 524.4441 (GTA-524), 530.4335 (GTA-530), 532.4492 (GTA-532), 536.4077 (GTA-536), 538.4233 (GTA-538), 540.4389 (GTA-540), 550.4597 (GTA-550), 574.4597 (GTA-574), 576.4754 (GTA-576), 580.5067 (GTA-580), 590.4546 (GTA-590), 592.4703 (GTA-592), 594.4859 (GTA-594), or 596.5016 (GTA-596).

In further non-limiting embodiments, the GTA dicarboxylic fatty acid metabolite may have a molecular formula of C₂₈H₄₆O₄ (GTA-446), C₂₈H₄₈O₄ (GTA-448), C₂₈H₅₀O₄ (GTA-450), C₂₈H₅₂O₄ (GTA-452), 464.3522 (GTA-464), 466.3661 (GTA-466), C₂₈H₅₂O(GTA-468), C₃₀H₅₀O₄ (GTA-474), C₃₀H₅₂O₄ GTA-476, C₃₀H₅₄O₄ (GTA-478), C₂₈H₅₂O₆ (GTA-484), C₃₀H₅₀O₅ (GTA-490), C₃₀H₅₂O(GTA-492), C₃₀H₅₄O₅ (GTA-494), C₃₂H₅₄O₄ (GTA-502), C₃₂H₅₆O₄ (GTA-504), C₃₀H₅₆O₆ (GTA-512), C₃₂H₅₄O₅ (GTA-518), C₃₂H₅₆O₅ (GTA-520), C₃₂H₅₈O₅ (GTA-522), C₃₂H₆₀O₅ GTA-524, C₃₄H₅₈O₄ (GTA-530), C₃₄H₆₀O₄ (GTA-532), C₃₂H₅₆O₆ (GTA-536), C₃₂H₅₈O₆ (GTA-538), C₃₂H₆₀O₆ (GTA-540), C₃₄H₆₂O₅ (GTA-550), C₃₆H₆₂O₅ (GTA-574), C₃₆H₆₄O₅ (GTA-576), C₃₆H₆₈O₅ (GTA-580), C₃₆H₆₂O₆ (GTA-590), C₃₆H₆₄O₆ (GTA-592), C₃₆H₆₆O₆ (GTA-594), or C₃₆H₆₈O₆ (GTA-596).

In further non-limiting embodiments, the GTA dicarboxylic fatty acid metabolite may be measured using collision induced dissociation (CID) tandem mass spectrometry. The GTAs may be one or more of the GTA dicarboxylic fatty acid metabolites listed below:

GTA-446, having an accurate neutral mass within 1 PPM Dalton mass accuracy of 446.3396, the molecular formula of C₂₈H₄₆O₄, and characterized by a OD MS/MS fragmentation pattern using N₂ as collision gas and analyzed in atmospheric pressure chemical ionization (APCI) under negative ionization comprising daughter ions of parent [M−H] mass 445: 427, 409, 401, and 383,

GTA-448, having an accurate neutral mass within 1 PPM Dalton mass accuracy of 448.3553, the molecular formula of C₂₈H₄₈O₄, and characterized by a OD MS/MS fragmentation pattern using N₂ as collision gas and analyzed in atmospheric pressure chemical ionization (APCI) under negative ionization comprising daughter ions of parent [M−H] mass 447: 429, 411, 403, and 385,

GTA-450, having an accurate neutral mass within 1 PPM Dalton mass accuracy of 450.3709, the molecular formula of C₂₈H₅₀O₄, and characterized by a OD MS/MS fragmentation pattern using N₂ as collision gas and analyzed in atmospheric pressure chemical ionization (APCI) under negative ionization comprising daughter ions of parent [M−H] mass 449: 431, 413, 405, and 387,

GTA-452, having an accurate neutral mass within 1 PPM Dalton mass accuracy of 452.3866, the molecular formula of C₂₈H₅₂O₄, and characterized by a OD MS/MS fragmentation pattern using N₂ as collision gas and analyzed in atmospheric pressure chemical ionization (APCI) under negative ionization comprising daughter ions of parent [M−H] mass 451: 433, 407, and 389,

GTA-464, having an accurate neutral mass within 1 PPM Dalton mass accuracy of 464.3522, the molecular formula of C₂₈H₄₈O₅, and characterized by a OD MS/MS fragmentation pattern using N₂ as collision gas and analyzed in atmospheric pressure chemical ionization (APCI) under negative ionization comprising daughter ions of parent [M−H] mass 463: 445, 419, 401, and 383,

GTA-466, having an accurate neutral mass within 1 PPM Dalton mass accuracy of 466.3661, the molecular formula of C₂₈H₅₀O₅, and characterized by a OD MS/MS fragmentation pattern using N₂ as collision gas and analyzed in atmospheric pressure chemical ionization (APCI) under negative ionization comprising daughter ions of parent [M−H] mass 465: 447, 421, and 403,

GTA-468, having an accurate neutral mass within 1 PPM Dalton mass accuracy of 468.3814, having the molecular formula of C₂₈H₅₂O, and characterized by a OD MS/MS fragmentation pattern using N₂ as collision gas and analyzed in atmospheric pressure chemical ionization (APCI) under negative ionization comprising daughter ions of parent [M−H] mass 467: 449, 423, and 405,

GTA-474, having an accurate neutral mass within 1 PPM Dalton mass accuracy of 474.3736, having the molecular formula of C₃₀H₅₀O₄, and characterized by a OD MS/MS fragmentation pattern using N₂ as collision gas and analyzed in atmospheric pressure chemical ionization (APCI) under negative ionization comprising daughter ions of parent [M−H] mass 473: 455, 429, and 411,

GTA-476, having an accurate neutral mass within 1 PPM Dalton mass accuracy of 476.3866, having the molecular formula of C₃₀H₅₂O₄, and characterized by a OD MS/MS fragmentation pattern using N₂ as collision gas and analyzed in atmospheric pressure chemical ionization (APCI) under negative ionization comprising daughter ions of parent [M−H] mass 475: 457, 431, 439 and 413,

GTA-478, having an accurate neutral mass within 1 PPM Dalton mass accuracy of 478.4022, having the molecular formula of C₃₀H₅₄O₄, and characterized by a OD MS/MS fragmentation pattern using N₂ as collision gas and analyzed in atmospheric pressure chemical ionization (APCI) under negative ionization comprising daughter ions of parent [M−H] mass 477: 459, 433, 441 and 415,

GTA-484, having an accurate neutral mass within 1 PPM Dalton mass accuracy of 484.3764, having the molecular formula of C₂₈H₅₂O₆, and characterized by a OD MS/MS fragmentation pattern using N₂ as collision gas and analyzed in atmospheric pressure chemical ionization (APCI) under negative ionization comprising daughter ions of parent [M−H] mass 483: 465, 315, 439 483, 421, and 447,

GTA-490, having an accurate neutral mass within 1 PPM Dalton mass accuracy of 490.3658, having the molecular formula of C₃₀H₅₀O₅, and characterized by a OD MS/MS fragmentation pattern using N₂ as collision gas and analyzed in atmospheric pressure chemical ionization (APCI) under negative ionization comprising daughter ions of parent [M−H] mass 489: 445, 471, 427 and 319,

GTA-492, having an accurate neutral mass within 1 PPM Dalton mass accuracy of 492.3815, having the molecular formula of C₃₀H₅₂O₅, and characterized by a OD MS/MS fragmentation pattern using N₂ as collision gas and analyzed in atmospheric pressure chemical ionization (APCI) under negative ionization comprising daughter ions of parent [M−H] mass 491: 241, 249, 267, 473, and 447,

GTA-494, having an accurate neutral mass within 1 PPM Dalton mass accuracy of 494.3971, having the molecular formula of C₃₀H₅₄O₅, and characterized by a OD MS/MS fragmentation pattern using N₂ as collision gas and analyzed in atmospheric pressure chemical ionization (APCI) under negative ionization comprising daughter ions of parent [M−H] mass 493: 475, 215, and 449,

GTA-502, having an accurate neutral mass within 1 PPM Dalton mass accuracy of 502.4022, having the molecular formula of C₃₂H₅₄O₄, and characterized by a OD MS/MS fragmentation pattern using N₂ as collision gas and analyzed in atmospheric pressure chemical ionization (APCI) under negative ionization comprising daughter ions of parent [M−H] mass 501: 483, 457, 465 and 439,

GTA-504, having an accurate neutral mass within 1 PPM Dalton mass accuracy of 504.4195, having the molecular formula of C₃₂H₅₆O₄, and characterized by a OD MS/MS fragmentation pattern using N₂ as collision gas and analyzed in atmospheric pressure chemical ionization (APCI) under negative ionization comprising daughter ions of parent [M−H] mass 503: 485, 459, 467 and 441,

GTA-512, having an accurate neutral mass within 1 PPM Dalton mass accuracy of 512.4077, having the molecular formula of C₃₀H₅₆O₆, and characterized by a OD MS/MS fragmentation pattern using N₂ as collision gas and analyzed in atmospheric pressure chemical ionization (APCI) under negative ionization comprising daughter ions of parent [M−H] mass 511: 493, 315, and 467,

GTA-518, having an accurate neutral mass within 1 PPM Dalton mass accuracy of 518.3974, having the molecular formula of C₃₂H₅₄O₅, and characterized by a OD MS/MS fragmentation pattern using N₂ as collision gas and analyzed in atmospheric pressure chemical ionization (APCI) under negative ionization comprising daughter ions of parent [M−H] mass 517: 499, 473, 499, 481 and 445,

GTA-520, having an accurate neutral mass within 1 PPM Dalton mass accuracy of 520.4128, having the molecular formula of C₃₂H₅₆O₅, and characterized by a OD MS/MS fragmentation pattern using N₂ as collision gas and analyzed in atmospheric pressure chemical ionization (APCI) under negative ionization comprising daughter ions of parent [M−H] mass 519: 501, 457, 475, 459, 447 and 483,

GTA-522, having an accurate neutral mass within 1 PPM Dalton mass accuracy of 522.4284, having the molecular formula of C₃₂H₅₈O₅, and characterized by a OD MS/MS fragmentation pattern using N₂ as collision gas and analyzed in atmospheric pressure chemical ionization (APCI) under negative ionization comprising daughter ions of parent [M−H] mass 521: 503, 459, 477, 504, 441 and 485,

GTA-524, having an accurate neutral mass within 1 PPM Dalton mass accuracy of 524.4441, having the molecular formula of C₃₂H₆₀O₅, and characterized by a OD MS/MS fragmentation pattern using N₂ as collision gas and analyzed in atmospheric pressure chemical ionization (APCI) under negative ionization comprising daughter ions of parent [M−H] mass 523: 505, 461, 479, 506, 443 and 487,

GTA-530, having an accurate neutral mass within 1 PPM Dalton mass accuracy of 530.4335, having the molecular formula of C₃₄H₅₈O₄, and characterized by a OD MS/MS fragmentation pattern using N₂ as collision gas and analyzed in atmospheric pressure chemical ionization (APCI) under negative ionization comprising daughter ions of parent [M−H] mass 529: 467, 511 and 485,

GTA-532, having an accurate neutral mass within 1 PPM Dalton mass accuracy of 532.4492, having the molecular formula of C₃₄H₆₀O₄, and characterized by a OD MS/MS fragmentation pattern using N₂ as collision gas and analyzed in atmospheric pressure chemical ionization (APCI) under negative ionization comprising daughter ions of parent [M−H] mass 531: 513, 469, 487 and 495,

GTA-536, having an accurate neutral mass within 1 PPM Dalton mass accuracy of 536.4077, having the molecular formula of C₃₂H₅₆O₆, and characterized by a OD MS/MS fragmentation pattern using N₂ as collision gas and analyzed in atmospheric pressure chemical ionization (APCI) under negative ionization comprising daughter ion of parent [M−H] mass 535: 473,

GTA-538, having an accurate neutral mass within 1 PPM Dalton mass accuracy of 538.4233, having the molecular formula of C₃₂H₅₈O₆, and characterized by a OD MS/MS fragmentation pattern using N₂ as collision gas and analyzed in atmospheric pressure chemical ionization (APCI) under negative ionization comprising daughter ions of parent [M−H] mass 537: 519, 475, 493, 501 and 457,

GTA-540, having an accurate neutral mass within 1 PPM Dalton mass accuracy of 540.4389, having the molecular formula of C₃₂H₆₀O₆, and characterized by a OD MS/MS fragmentation pattern using N₂ as collision gas and analyzed in atmospheric pressure chemical ionization (APCI) under negative ionization comprising daughter ions of parent [M−H] mass 539: 315, 521, 495 and 477,

GTA-550, having an accurate neutral mass within 1 PPM Dalton mass accuracy of 550.4597, having the molecular formula of C₃₄H₆₂O₅, and characterized by a OD MS/MS fragmentation pattern using N₂ as collision gas and analyzed in atmospheric pressure chemical ionization (APCI) under negative ionization comprising daughter ions of parent [M−H] mass 549: 487, 531, 251, 253, 513, 469 and 506,

GTA-574, having an accurate neutral mass within 1 PPM Dalton mass accuracy of 574.4597, having the molecular formula of C₃₆H₆₂O₅, and characterized by a OD MS/MS fragmentation pattern using N₂ as collision gas and analyzed in atmospheric pressure chemical ionization (APCI) under negative ionization comprising daughter ions of parent [M−H] mass 573: 295, 223, 555 and 511,

GTA-576, having an accurate neutral mass within 1 PPM Dalton mass accuracy of 576.4754, having the molecular formula of C₃₆H₆₄O₅, and characterized by a OD MS/MS fragmentation pattern using N₂ as collision gas and analyzed in atmospheric pressure chemical ionization (APCI) under negative ionization comprising daughter ions of parent [M−H] mass 575: 277, 297, 557, 513 and 495,

GTA-580, having an accurate neutral mass within 1 PPM Dalton mass accuracy of 580.5067, having the molecular formula of C₃₆H₆₈O₅, and characterized by a OD MS/MS fragmentation pattern using N₂ as collision gas and analyzed in atmospheric pressure chemical ionization (APCI) under negative ionization comprising daughter ions of parent [M−H] mass 579: 561, 543, 535, 517 and 499,

GTA-590, having an accurate neutral mass within 1 PPM Dalton mass accuracy of 590.4546, having the molecular formula of C₃₆H₆₂O₆, and characterized by a OD MS/MS fragmentation pattern using N₂ as collision gas and analyzed in atmospheric pressure chemical ionization (APCI) under negative ionization comprising daughter ion of parent [M−H] mass 589: 545,

GTA-592, having an accurate neutral mass within 1 PPM Dalton mass accuracy of 592.4703, having the molecular formula of C₃₆H₆₄O₆, and characterized by a OD MS/MS fragmentation pattern using N₂ as collision gas and analyzed in atmospheric pressure chemical ionization (APCI) under negative ionization comprising daughter ions of parent [M−H] mass 591: 555 and 113,

GTA-594, having an accurate neutral mass within 1 PPM Dalton mass accuracy of 594.4859, having the molecular formula of C₃₆H₆₆O₆, and characterized by a OD MS/MS fragmentation pattern using N₂ as collision gas and analyzed in atmospheric pressure chemical ionization (APCI) under negative ionization comprising daughter ions of parent [M−H] mass 593: 557 371, 315 and 277, and

GTA-596, having an accurate neutral mass within 1 PPM Dalton mass accuracy of 596.5016, having the molecular formula of C₃₆H₆₈O₆, and characterized by a OD MS/MS fragmentation pattern using N₂ as collision gas and analyzed in atmospheric pressure chemical ionization (APCI) under negative ionization comprising daughter ions of parent [M−H] mass 595: 279, 315, 297, 577 and 559.

In one specific embodiment, the GTA dicarboxylic fatty acid metabolite may be GTA-446, which has the formula C₂₈H₄₆O₄ and the structure:

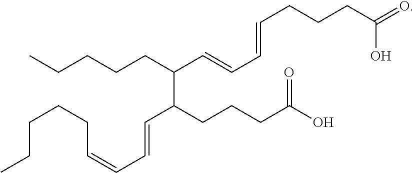 [SMILES: CCCCC/C=C\C=C\C(CCCC(=O)O)C(/C=C/C=C/CCCC(=O)O)CCCCC]

Also provided herein is a kit for detecting and treating a gastric tract acid (GTA) insufficiency in a mammalian subject. The kit comprises:

a blood specimen collection device for collecting a blood sample from the mammalian subject,

packaging and instructions for submitting the blood sample to a central processing facility to test levels in the blood sample of one or more GTA dicarboxylic fatty acid metabolite, wherein the GTA dicarboxylic fatty acid metabolite is a dicarboxylic fatty acid between 28 and 36 carbons comprised of a dimeric fatty acid structure of two shorter chains ranging between 14 and 18 carbons in length joined by a single or double bond; and

instructions for obtaining the results of testing the blood sample from the central processing facility, wherein in the case of a positive test result comprising a detected low GTA level, a GTA-augmenting anti-inflammatory prebiotic, probiotic, or synthetic GTA product is provided.

In certain embodiments of the kit, the GTA-augmenting anti-inflammatory prebiotic, probiotic, or synthetic GTA product is a composition comprising at least one live or attenuated culture containing a microbial species selected from the genus Blautia, species Faecalibacterium prausnitzii, genus Bacteroides, family Ruminococcaceae, family Lachnospiraceae, genus Coprococcus, genus Roseburia, genus Oscillospira, species Ruminococcus bromii, genus Ruminococcus, family Costridiaceae, species Dorea formicigenerans, species Bacteroides uniformis, genus Dorea, genus Streptococcus, order Clostridiales, genus Anaerostipes, genus Dialister, species Bifidobacterium adolescentis, family Coriobacteriaceae, genus Faecalibacterium, genus Sutterella, species Bacteroides ovatus, genus Parabacteroides, genus Ruminococcus, species Bacteroides faecis, species Eubacterium biforme, genus Phascolartobacterium, and family Enterobacteriaceae; or a prebiotic composition which increases growth and/or viability of the microbial species in the gut; and when administered to the subject the composition increases the synthesis of at least one GTA dicarboxylic fatty acid metabolite in the subject.

In further embodiments, the composition is provided if the levels of the one or more GTA dicarboxylic fatty acid metabolites in the subject are determined to be lower than a predetermined control level, an earlier test value for said subject, or a normal level for healthy subjects. In one particular example, the control may be a predetermined threshold value for the at least one GTA dicarboxylic fatty acid metabolite.

In yet further embodiments, the composition may comprise a live or attenuated culture of a microbial species from the genus Blautia, a live or attenuated culture of Faecalibacterium prausnitzii, or a combination thereof, within a pharmaceutically-acceptable carrier suitable for administration to the gastrointestinal tract of the subject.

In further embodiments of the kit, the GTA dicarboxylic fatty acid metabolite may be one of the following: GTA-446, GTA-448, GTA-450, GTA-452, GTA-464, GTA-466, GTA-468, GTA-474, GTA-476, GTA-478, GTA-484, GTA-490, GTA-492, GTA-494, GTA-502, GTA-504, GTA-512, GTA-518, GTA-520, GTA-522, GTA-524, GTA-530, GTA-532, GTA-536, GTA-538, GTA-540, GTA-550, GTA-574, GTA-576, GTA-580, GTA-590, GTA-592, GTA-594, and GTA-596.

In non-limiting embodiments, the GTA dicarboxylic fatty acid metabolite may have an accurate neutral mass within 1 PPM Dalton mass accuracy of 446.3396 (GTA-446), 448.3553 (GTA-448), 450.3709 (GTA-450), 452.3866 (GTA-452), 464.3522 (GTA-464), 466.3661 (GTA-466), 468.3814 (GTA-468), 474.3736 (GTA-474), 476.3866 GTA-476, 478.4022 (GTA-478), 484.3764 (GTA-484), 490.3658 (GTA-490), 492.3815 (GTA-492), 494.3971 (GTA-494), 502.4022 (GTA-502), 504.4195 (GTA-504), 512.4077 (GTA-512), 518.3974 (GTA-518), 520.4128 (GTA-520), 522.4284 (GTA-522), 524.4441 (GTA-524), 530.4335 (GTA-530), 532.4492 (GTA-532), 536.4077 (GTA-536), 538.4233 (GTA-538), 540.4389 (GTA-540), 550.4597 (GTA-550), 574.4597 (GTA-574), 576.4754 (GTA-576), 580.5067 (GTA-580), 590.4546 (GTA-590), 592.4703 (GTA-592), 594.4859 (GTA-594), or 596.5016 (GTA-596).

In further non-limiting embodiments, the GTA dicarboxylic fatty acid metabolite may have a molecular formula of C₂₈H₄₆O₄ (GTA-446), C₂₈H₄₈O₄ (GTA-448), C₂₈H₅₀O₄ (GTA-450), C₂₈H₅₂O₄ (GTA-452), 464.3522 (GTA-464), 466.3661 (GTA-466), C₂₈H₅₂O(GTA-468), C₃₀H₅₀O₄ (GTA-474), C₃₀H₅₂O₄ GTA-476, C₃₄H₅₄O₄ (GTA-478), C₂₈H₅₂O₆ (GTA-484), C₃₀H₅₀O₅ (GTA-490), C₃₀H₅₂O₅ (GTA-492), C₃₄H₅₄O₅ (GTA-494), C₃₂H₅₄O₄ (GTA-502), C₃₂H₅₆O₄ (GTA-504), C₃₀H₅₆O₆ (GTA-512), C₃₂H₅₄O₅ (GTA-518), C₃₂H₅₆O₅ (GTA-520), C₃₂H₅₈O₅ (GTA-522), C₃₂H₆₀O₅ GTA-524, C₃₄H₅₈O₄ (GTA-530), C₃₄H₆₀O₄ (GTA-532), C₃₂H₅₆O₆ (GTA-536), C₃₂H₅₈O₆ (GTA-538), C₃₂H₆₀O₆ (GTA-540), C₃₄H₆₂O₅ (GTA-550), C₃₆H₆₂O₅ (GTA-574), C₃₆H₆₄O₅ (GTA-576), C₃₆H₆₈O₅ (GTA-580), C₃₆H₆₂O₆ (GTA-590), C₃₆H₆₄O₆ (GTA-592), C₃₆H₆₆O₆ (GTA-594), or C₃₆H₆₈O₆ (GTA-596).

In further non-limiting embodiments, the GTA dicarboxylic fatty acid metabolite may be measured using collision induced dissociation (CID) tandem mass spectrometry. The GTAs may be one or more of the GTA dicarboxylic fatty acid metabolites listed below:

GTA-446, having an accurate neutral mass within 1 PPM Dalton mass accuracy of 446.3396, the molecular formula of C₂₈H₄₆O₄, and characterized by a OD MS/MS fragmentation pattern using N₂ as collision gas and analyzed in atmospheric pressure chemical ionization (APCI) under negative ionization comprising daughter ions of parent [M−H] mass 445: 427, 409, 401, and 383,

GTA-448, having an accurate neutral mass within 1 PPM Dalton mass accuracy of 448.3553, the molecular formula of C₂₈H₄₈O₄, and characterized by a OD MS/MS fragmentation pattern using N₂ as collision gas and analyzed in atmospheric pressure chemical ionization (APCI) under negative ionization comprising daughter ions of parent [M−H] mass 447: 429, 411, 403, and 385,

GTA-450, having an accurate neutral mass within 1 PPM Dalton mass accuracy of 450.3709, the molecular formula of C₂₈H₅₀O₄, and characterized by a OD MS/MS fragmentation pattern using N₂ as collision gas and analyzed in atmospheric pressure chemical ionization (APCI) under negative ionization comprising daughter ions of parent [M−H] mass 449: 431, 413, 405, and 387,

GTA-452, having an accurate neutral mass within 1 PPM Dalton mass accuracy of 452.3866, the molecular formula of C₂₈H₅₂O₄, and characterized by a OD MS/MS fragmentation pattern using N₂ as collision gas and analyzed in atmospheric pressure chemical ionization (APCI) under negative ionization comprising daughter ions of parent [M−H] mass 451: 433, 407, and 389,

GTA-464, having an accurate neutral mass within 1 PPM Dalton mass accuracy of 464.3522, the molecular formula of C₂₈H₄₈O₅, and characterized by a OD MS/MS fragmentation pattern using N₂ as collision gas and analyzed in atmospheric pressure chemical ionization (APCI) under negative ionization comprising daughter ions of parent [M−H] mass 463: 445, 419, 401, and 383,

GTA-466, having an accurate neutral mass within 1 PPM Dalton mass accuracy of 466.3661, the molecular formula of C₂₈H₅₀O₅, and characterized by a OD MS/MS fragmentation pattern using N₂ as collision gas and analyzed in atmospheric pressure chemical ionization (APCI) under negative ionization comprising daughter ions of parent [M−H] mass 465: 447, 421, and 403,

GTA-468, having an accurate neutral mass within 1 PPM Dalton mass accuracy of 468.3814, having the molecular formula of C₂₈H₅₂O, and characterized by a OD MS/MS fragmentation pattern using N₂ as collision gas and analyzed in atmospheric pressure chemical ionization (APCI) under negative ionization comprising daughter ions of parent [M−H] mass 467: 449, 423, and 405,

GTA-474, having an accurate neutral mass within 1 PPM Dalton mass accuracy of 474.3736, having the molecular formula of C₃₀H₅₀O₄, and characterized by a OD MS/MS fragmentation pattern using N₂ as collision gas and analyzed in atmospheric pressure chemical ionization (APCI) under negative ionization comprising daughter ions of parent [M−H] mass 473: 455, 429, and 411,

GTA-476, having an accurate neutral mass within 1 PPM Dalton mass accuracy of 476.3866, having the molecular formula of C₃₀H₅₂O₄, and characterized by a OD MS/MS fragmentation pattern using N₂ as collision gas and analyzed in atmospheric pressure chemical ionization (APCI) under negative ionization comprising daughter ions of parent [M−H] mass 475: 457, 431, 439 and 413,

GTA-478, having an accurate neutral mass within 1 PPM Dalton mass accuracy of 478.4022, having the molecular formula of C₃₀H₅₄O₄, and characterized by a OD MS/MS fragmentation pattern using N₂ as collision gas and analyzed in atmospheric pressure chemical ionization (APCI) under negative ionization comprising daughter ions of parent [M−H] mass 477: 459, 433, 441 and 415,

GTA-484, having an accurate neutral mass within 1 PPM Dalton mass accuracy of 484.3764, having the molecular formula of C₂₈H₅₂O₆, and characterized by a OD MS/MS fragmentation pattern using N₂ as collision gas and analyzed in atmospheric pressure chemical ionization (APCI) under negative ionization comprising daughter ions of parent [M−H] mass 483: 465, 315, 439 483, 421, and 447,

GTA-490, having an accurate neutral mass within 1 PPM Dalton mass accuracy of 490.3658, having the molecular formula of C₃₀H₅₀O₅, and characterized by a OD MS/MS fragmentation pattern using N₂ as collision gas and analyzed in atmospheric pressure chemical ionization (APCI) under negative ionization comprising daughter ions of parent [M−H] mass 489: 445, 471, 427 and 319,

GTA-492, having an accurate neutral mass within 1 PPM Dalton mass accuracy of 492.3815, having the molecular formula of C₃₀H₅₂O₅, and characterized by a OD MS/MS fragmentation pattern using N₂ as collision gas and analyzed in atmospheric pressure chemical ionization (APCI) under negative ionization comprising daughter ions of parent [M−H] mass 491: 241, 249, 267, 473, and 447,

GTA-494, having an accurate neutral mass within 1 PPM Dalton mass accuracy of 494.3971, having the molecular formula of C₃₀H₅₄O₅, and characterized by a OD MS/MS fragmentation pattern using N₂ as collision gas and analyzed in atmospheric pressure chemical ionization (APCI) under negative ionization comprising daughter ions of parent [M−H] mass 493: 475, 215, and 449,

GTA-502, having an accurate neutral mass within 1 PPM Dalton mass accuracy of 502.4022, having the molecular formula of C₃₂H₅₄O₄, and characterized by a OD MS/MS fragmentation pattern using N₂ as collision gas and analyzed in atmospheric pressure chemical ionization (APCI) under negative ionization comprising daughter ions of parent [M−H] mass 501: 483, 457, 465 and 439,

GTA-504, having an accurate neutral mass within 1 PPM Dalton mass accuracy of 504.4195, having the molecular formula of C₃₂H₅₆O₄, and characterized by a OD MS/MS fragmentation pattern using N₂ as collision gas and analyzed in atmospheric pressure chemical ionization (APCI) under negative ionization comprising daughter ions of parent [M−H] mass 503: 485, 459, 467 and 441,

GTA-512, having an accurate neutral mass within 1 PPM Dalton mass accuracy of 512.4077, having the molecular formula of C₃₀H₅₆O₆, and characterized by a OD MS/MS fragmentation pattern using N₂ as collision gas and analyzed in atmospheric pressure chemical ionization (APCI) under negative ionization comprising daughter ions of parent [M−H] mass 511: 493, 315, and 467,

GTA-518, having an accurate neutral mass within 1 PPM Dalton mass accuracy of 518.3974, having the molecular formula of C₃₂H₅₄O₅, and characterized by a OD MS/MS fragmentation pattern using N₂ as collision gas and analyzed in atmospheric pressure chemical ionization (APCI) under negative ionization comprising daughter ions of parent [M−H] mass 517: 499, 473, 499, 481 and 445,

GTA-520, having an accurate neutral mass within 1 PPM Dalton mass accuracy of 520.4128, having the molecular formula of C₃₂H₅₆O₅, and characterized by a OD MS/MS fragmentation pattern using N₂ as collision gas and analyzed in atmospheric pressure chemical ionization (APCI) under negative ionization comprising daughter ions of parent [M−H] mass 519: 501, 457, 475, 459, 447 and 483,

GTA-522, having an accurate neutral mass within 1 PPM Dalton mass accuracy of 522.4284, having the molecular formula of C₃₂H₅₈O₅, and characterized by a OD MS/MS fragmentation pattern using N₂ as collision gas and analyzed in atmospheric pressure chemical ionization (APCI) under negative ionization comprising daughter ions of parent [M−H] mass 521: 503, 459, 477, 504, 441 and 485,

GTA-524, having an accurate neutral mass within 1 PPM Dalton mass accuracy of 524.4441, having the molecular formula of C₃₂H₆₀O₅, and characterized by a OD MS/MS fragmentation pattern using N₂ as collision gas and analyzed in atmospheric pressure chemical ionization (APCI) under negative ionization comprising daughter ions of parent [M−H] mass 523: 505, 461, 479, 506, 443 and 487,

GTA-530, having an accurate neutral mass within 1 PPM Dalton mass accuracy of 530.4335, having the molecular formula of C₃₄H₅₈O₄, and characterized by a OD MS/MS fragmentation pattern using N₂ as collision gas and analyzed in atmospheric pressure chemical ionization (APCI) under negative ionization comprising daughter ions of parent [M−H] mass 529: 467, 511 and 485,

GTA-532, having an accurate neutral mass within 1 PPM Dalton mass accuracy of 532.4492, having the molecular formula of C₃₄H₆₀O₄, and characterized by a OD MS/MS fragmentation pattern using N₂ as collision gas and analyzed in atmospheric pressure chemical ionization (APCI) under negative ionization comprising daughter ions of parent [M−H] mass 531: 513, 469, 487 and 495,

GTA-536, having an accurate neutral mass within 1 PPM Dalton mass accuracy of 536.4077, having the molecular formula of C₃₂H₅₆O₆, and characterized by a OD MS/MS fragmentation pattern using N₂ as collision gas and analyzed in atmospheric pressure chemical ionization (APCI) under negative ionization comprising daughter ion of parent [M−H] mass 535: 473,

GTA-538, having an accurate neutral mass within 1 PPM Dalton mass accuracy of 538.4233, having the molecular formula of C₃₂H₅₈O₆, and characterized by a OD MS/MS fragmentation pattern using N₂ as collision gas and analyzed in atmospheric pressure chemical ionization (APCI) under negative ionization comprising daughter ions of parent [M−H] mass 537: 519, 475, 493, 501 and 457,

GTA-540, having an accurate neutral mass within 1 PPM Dalton mass accuracy of 540.4389, having the molecular formula of C₃₂H₆₀O₆, and characterized by a OD MS/MS fragmentation pattern using N₂ as collision gas and analyzed in atmospheric pressure chemical ionization (APCI) under negative ionization comprising daughter ions of parent [M−H] mass 539: 315, 521, 495 and 477,

GTA-550, having an accurate neutral mass within 1 PPM Dalton mass accuracy of 550.4597, having the molecular formula of C₃₄H₆₂O₅, and characterized by a OD MS/MS fragmentation pattern using N₂ as collision gas and analyzed in atmospheric pressure chemical ionization (APCI) under negative ionization comprising daughter ions of parent [M−H] mass 549: 487, 531, 251, 253, 513, 469 and 506,

GTA-574, having an accurate neutral mass within 1 PPM Dalton mass accuracy of 574.4597, having the molecular formula of C₃₆H₆₂O₅, and characterized by a OD MS/MS fragmentation pattern using N₂ as collision gas and analyzed in atmospheric pressure chemical ionization (APCI) under negative ionization comprising daughter ions of parent [M−H] mass 573: 295, 223, 555 and 511,

GTA-576, having an accurate neutral mass within 1 PPM Dalton mass accuracy of 576.4754, having the molecular formula of C₃₆H₆₄O₅, and characterized by a OD MS/MS fragmentation pattern using N₂ as collision gas and analyzed in atmospheric pressure chemical ionization (APCI) under negative ionization comprising daughter ions of parent [M−H] mass 575: 277, 297, 557, 513 and 495,

GTA-580, having an accurate neutral mass within 1 PPM Dalton mass accuracy of 580.5067, having the molecular formula of C₃₆H₆₈O₅, and characterized by a OD MS/MS fragmentation pattern using N₂ as collision gas and analyzed in atmospheric pressure chemical ionization (APCI) under negative ionization comprising daughter ions of parent [M−H] mass 579: 561, 543, 535, 517 and 499,

GTA-590, having an accurate neutral mass within 1 PPM Dalton mass accuracy of 590.4546, having the molecular formula of C₃₆H₆₂O₆, and characterized by a OD MS/MS fragmentation pattern using N₂ as collision gas and analyzed in atmospheric pressure chemical ionization (APCI) under negative ionization comprising daughter ion of parent [M−H] mass 589: 545,

GTA-592, having an accurate neutral mass within 1 PPM Dalton mass accuracy of 592.4703, having the molecular formula of C₃₆H₆₄O₆, and characterized by a OD MS/MS fragmentation pattern using N₂ as collision gas and analyzed in atmospheric pressure chemical ionization (APCI) under negative ionization comprising daughter ions of parent [M−H] mass 591: 555 and 113,

GTA-594, having an accurate neutral mass within 1 PPM Dalton mass accuracy of 594.4859, having the molecular formula of C₃₆H₆₆O₆, and characterized by a OD MS/MS fragmentation pattern using N₂ as collision gas and analyzed in atmospheric pressure chemical ionization (APCI) under negative ionization comprising daughter ions of parent [M−H] mass 593: 557 371, 315 and 277, and

GTA-596, having an accurate neutral mass within 1 PPM Dalton mass accuracy of 596.5016, having the molecular formula of C₃₆H₆₈O₆, and characterized by a OD MS/MS fragmentation pattern using N₂ as collision gas and analyzed in atmospheric pressure chemical ionization (APCI) under negative ionization comprising daughter ions of parent [M−H] mass 595: 279, 315, 297, 577 and 559.

In one particular example of this kit, the GTA dicarboxylic fatty acid metabolite is GTA-446, having the formula C₂₈H₄₆O₄ and the structure:

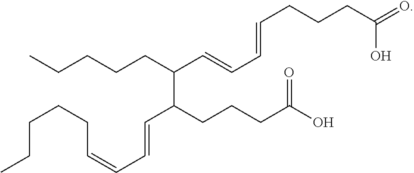 [SMILES: CCCCC/C=C\C=C\C(CCCC(=O)O)C(/C=C/C=C/CCCC(=O)O)CCCCC]

In accordance with further embodiments of the above described methods and kit, the gastric tract acid (GTA) insufficiency may be an indicator of a gastrointestinal (GI) inflammatory state.

### BRIEF DESCRIPTION OF THE DRAWINGS

These and other features of the invention will become more apparent from the following description in which reference is made to the appended drawings wherein:

FIG. 1 illustrates the mechanism provided herein by which compromised GTA levels as a results of altered microbiome composition can lead to inflammation and cancer development.

FIG. 2 illustrates a schematic diagram of a personalized GTA testing and treatment method described herein; and

FIG. 3 shows a graph of the operational taxonomic units (OTUs) representing particular genus and species-level microbes associated with low or high GTA levels.

FIG. 4 shows a graph illustrating production of GTA 445.4/383.4 (also described herein as GTA 446) by gut microbes in humans and animals (dog and pig).

FIG. 5 shows a graph illustrating production of GTA 447.4/385.4 (also described herein as GTA 448) by gut microbes in humans and animals (dog and pig).

FIG. 6 shows a graph illustrating production of GTA 449.4/405.4 (also described herein as GTA 450) by gut microbes in humans and animals (dog and pig).

FIG. 7 shows a graph illustrating production of GTA 463.4/419.4 (also described herein as GTA 464) by gut microbes in humans and animals (dog and pig).

FIG. 8 shows a graph illustrating production of GTA 465.4/403.4 (also described herein as GTA 466) by gut microbes in humans and animals (dog and pig).

FIG. 9 shows a graph illustrating production of GTA 467.4/423.4 (also described herein as GTA 468) by gut microbes in humans and animals (dog and pig).

### DETAILED DESCRIPTION

Gastric tract acids (GTAs) are shown herein to be produced by specific gut microbes. Changes in the microbiome over time may thus result in compromised ability to produce GTAs.

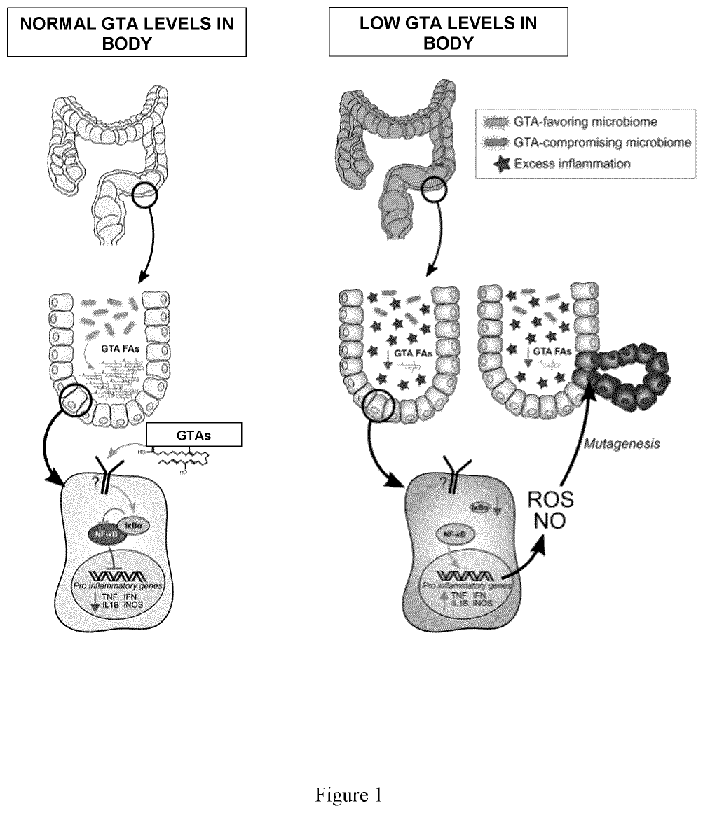

As illustrated in FIG. 1, GTA metabolites are involved in protecting against chronic inflammation through the downregulation of NFκB, as shown in the left-hand panel. Under this state, a relatively low level of NF-κB expression is maintained by adequate GTA levels in the body as a result of optimal microbiome composition. When levels of GTAs become deficient (right panel) due to altered microbiome composition, changes in the relative abundances of particular microbial species, or changes in diversity, NFκB expression is no longer suppressed resulting in the induction of multiple proinflammatory proteins. This creates an oxidative environment in the gastrointestinal tract that can lead to DNA mutations in cells, and ultimately increased cancer risk. GTA deficiency is therefore not a tumor marker like occult blood or methylated DNA; but rather pre-a disease metabolic deficiency that results in a pro-cancer environment within the body.

Based on this new understanding of the role of gut microbes in GTA production, a method is provided herein to increase GTA levels in the body through either augmentation of particular strains with a pre or probiotic approach, or administration of purely synthetic GTAs.

Using the described method to increase GTA levels also represents a novel approach for reducing inflammation within the gastrointestinal tract.

In particular, it has been found that microbial species from the genus Blautia, species Faecalibacterium prausnitzii, genus Bacteroides, family Ruminococcaceae, family Lachnospiraceae, genus Coprococcus, genus Roseburia, genus Oscillospira, species Ruminococcus bromii, genus Ruminococcus, family Costridiaceae, species Dorea formicigenerans, species Bacteroides uniformis, genus Dorea, genus Streptococcus, order Clostridiales, genus Anaerostipes, genus Dialister, species Bifidobacterium adolescentis, family Coriobacteriaceae, genus Faecalibacterium, genus Sutterella, species Bacteroides ovatus, genus Parabacteroides, genus Ruminococcus, species Bacteroides faecis, species Eubacterium biforme, genus Phascolartobacterium, and family Enterobacteriaceae are important for GTA biosynthesis in the gut. Administering a therapeutically-effective amount of a composition comprising at least one live or attenuated culture of at least one of these microbial species, or a prebiotic composition which increases growth and/or viability of the microbial species in the gut, therefore provides a means to increase GTA synthesis in an individual. The subject may be a mammal, in particular a human subject.

In most embodiments, the GTA will be a dicarboxylic fatty acid between 28 and 36 carbons comprised of a dimeric fatty acid structure of two shorter chains ranging between 14 and 18 carbons in length joined by a single or double bond. For example, the GTA dicarboxylic fatty acid metabolite may be GTA-446, which has the formula C₂₈H₄₆O₄ and the structure:

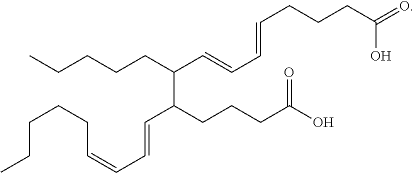 [SMILES: CCCCC/C=C\C=C\C(CCCC(=O)O)C(/C=C/C=C/CCCC(=O)O)CCCCC]

Other examples of these GTAs include GTA-448, GTA-450, GTA-452, GTA-464, GTA-466, GTA-468, GTA-474, GTA-476, GTA-478, GTA-484, GTA-490, GTA-492, GTA-494, GTA-502, GTA-504, GTA-512, GTA-518, GTA-520, GTA-522, GTA-524, GTA-530, GTA-532, GTA-536, GTA-538, GTA-540, GTA-550, GTA-574, GTA-576, GTA-580, GTA-590, GTA-592, GTA-594, and GTA-596.

Levels of these GTAs can be measured in a variety of ways, including mass spectrometric methods. For example, they may each be identified or measured based on the detection of one or more daughter ion fragment resulting from collision induced dissociation (OD) tandem mass spectrometry. The fragments for each GTA listed above are listed in the following tables.

Although the complete OD fragmentation patterns described below represent unique fingerprints of these target analytes, one will appreciate that not every daughter fragment ion needs to be detected to practice the described methods. In fact, one will appreciate that any number or combination of daughter ion masses could be selected for the purpose of specifically detecting and measuring levels of the parent analyte in a sample. One will further appreciate that the selection of appropriate daughter ions is dependent on multiple criteria such as signal-to-noise ratio, specificity of the transition for the selected analyte, reproducibility of signal, interferences across various matrices, complexity and anticipated specificity of the neutral loss to the parent structure, and more. In many cases, a single daughter fragment ion can be selected based on these criteria and used to quantify the corresponding parent analyte.

### GTA Dicarboxylic Fatty Acid Metabolites:

### 446.3396 (GTA-446):

446.4
CE: −35 V

| m/z (amu) | intensity (counts) | % intensity |
|---|---|---|
| 401 | 10.3333 | 100 |
| 445 | 8.1667 | 79.0323 |
| 427 | 4.5 | 43.5484 |
| 83 | 2.8333 | 27.4194 |
| 223 | 2.5 | 24.1935 |
| 222 | 2.1667 | 20.9677 |
| 205 | 1.8333 | 17.7419 |
| 383 | 1.8333 | 17.7419 |
| 59 | 1.6667 | 16.129 |
| 97 | 1 | 9.6774 |
| 81 | 0.6667 | 6.4516 |
| 109 | 0.6667 | 6.4516 |
| 203 | 0.6667 | 6.4516 |
| 221 | 0.6667 | 6.4516 |
| 409 | 0.6667 | 6.4516 |
| 123 | 0.5 | 4.8387 |
| 177 | 0.5 | 4.8387 |
| 233 | 0.5 | 4.8387 |
| 259 | 0.5 | 4.8387 |
| 428 | 0.5 | 4.8387 |

The metabolite 446.3396 (GTA-446) has the molecular formula C₂₈H₄₆O₄ and can be characterized by the full CID MS/MS fragmentation pattern shown in the above table. For detection and measurement purposes, however, a subset or even one of these fragments will be far more practical. Thus, in certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 445: 427, 409, 401, and 383. In certain preferred embodiments, nominal parent/daughter mass 445/401 or 445/383 may be used for measuring GTA-446 levels.

### 448.3553 (GTA-448):

448.4
CE: −35 V

| m/z (amu) | intensity (counts) | % intensity |
|---|---|---|
| 403 | 3.75 | 100 |
| 429 | 1.75 | 46.6667 |
| 447 | 1.5 | 40 |
| 385 | 1 | 26.6667 |
| 83 | 0.75 | 20 |
| 447 | 0.75 | 20 |
| 111 | 0.5 | 13.3333 |
| 151 | 0.5 | 13.3333 |
| 402 | 0.5 | 13.3333 |
| 411 | 0.5 | 13.3333 |
| 429 | 0.5 | 13.3333 |
| 59 | 0.25 | 6.6667 |
| 69 | 0.25 | 6.6667 |
| 74 | 0.25 | 6.6667 |
| 81 | 0.25 | 6.6667 |
| 187 | 0.25 | 6.6667 |
| 223 | 0.25 | 6.6667 |
| 279 | 0.25 | 6.6667 |
| 386 | 0.25 | 6.6667 |
| 404 | 0.25 | 6.6667 |

The metabolite 448.3553 (GTA-448) has the molecular formula C₂₈H₄₈O₄ and can be characterized by the full CID MS/MS fragmentation pattern shown in the above table. For detection and measurement purposes, however, a subset or even one of these fragments will be far more practical. Thus, in certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 447: 429, 411, 403, and 385. In certain preferred embodiments, nominal parent/daughter mass 447/385 may be used for measuring GTA-448 levels.

### 450.3709 (GTA-450):

450.4
CE: −35 V

| m/z (amu) | intensity (counts) | % intensity |
|---|---|---|
| 431 | 19 | 100 |
| 449 | 15.25 | 80.2632 |
| 405 | 10 | 52.6316 |
| 387 | 4.5 | 23.6842 |
| 405 | 1.5 | 7.8947 |
| 111 | 1.25 | 6.5789 |
| 413 | 1.25 | 6.5789 |
| 432 | 1 | 5.2632 |
| 59 | 0.75 | 3.9474 |
| 71 | 0.75 | 3.9474 |
| 97 | 0.75 | 3.9474 |
| 281 | 0.75 | 3.9474 |
| 406 | 0.75 | 3.9474 |
| 450 | 0.75 | 3.9474 |
| 57 | 0.5 | 2.6316 |
| 83 | 0.5 | 2.6316 |
| 123 | 0.5 | 2.6316 |
| 125 | 0.5 | 2.6316 |
| 181 | 0.5 | 2.6316 |
| 233 | 0.5 | 2.6316 |

The metabolite 450.3709 (GTA-450) has the molecular formula C₂₈H₅₀O₄ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 449: 431, 413, 405, and 387. In certain preferred embodiments, nominal parent/daughter mass 449/405 may be used for measuring GTA-450 levels.

### 452.3866 (GTA-452):

452.4
CE: −35 V
m/z (amu)
451(M − H+)
433
407
389
281
279
183
169
153
139
125
111
97

The metabolite 452.3866 (GTA-452) has the molecular formula C₂₈H₅₂O₄ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 451: 433, 407, and 389. In certain preferred embodiments, nominal parent/daughter mass 451/407 may be used for measuring GTA-452 levels.

### 464.3522 (GTA-464):

464.4
CE: −35 V
m/z (amu)
463(M − H+)
445
419
401
383
315
297
241

The metabolite 464.3522 (GTA-464) has the molecular formula C₂₈H₄₈O₅ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 463: 445, 419, 401, and 383. In certain preferred embodiments, nominal parent/daughter mass 463/419 may be used for measuring GTA-464 levels.

### 466.3661 (GTA-466):

466.4
CE: −35 V
m/z (amu)
465(M − H+)
447
433
421
405
403
349
297
279
241
223
185

The metabolite 466.3661 (GTA-466) has the molecular formula C₂₈H₅₀O₅ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 465: 447, 421, and 403. In certain preferred embodiments, nominal parent/daughter mass 465/403 may be used for measuring GTA-466 levels.

### 468.3814 (GTA-468):

468.4
CE: −35 V

| m/z (amu) | intensity (counts) | % intensity |
|---|---|---|
| 449 | 10.5 | 100 |
| 467 | 7.5 | 71.4286 |
| 187 | 4 | 38.0952 |
| 449 | 2 | 19.0476 |
| 263 | 1.5 | 14.2857 |
| 423 | 1.5 | 14.2857 |
| 141 | 1.25 | 11.9048 |
| 279 | 1.25 | 11.9048 |
| 169 | 1 | 9.5238 |
| 450 | 1 | 9.5238 |
| 215 | 0.75 | 7.1429 |
| 297 | 0.75 | 7.1429 |
| 405 | 0.75 | 7.1429 |
| 468 | 0.75 | 7.1429 |
| 185 | 0.5 | 4.7619 |
| 188 | 0.5 | 4.7619 |
| 213 | 0.5 | 4.7619 |
| 251 | 0.5 | 4.7619 |
| 281 | 0.5 | 4.7619 |
| 113 | 0.25 | 2.381 |

The metabolite 468.3814 (GTA-468) has the molecular formula C₂₈H₅₂Oand can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 467: 449, 423, and 405. In certain preferred embodiments, nominal parent/daughter mass 467/423 may be used for measuring GTA-468 levels.

### 474.3736 (GTA-474):

474.4
CE: −35 V

| m/z (amu) | intensity (counts) | % intensity |
|---|---|---|
| 473 | 1.8 | 100 |
| 455 | 1.05 | 58.3333 |
| 85 | 0.45 | 25 |
| 113 | 0.45 | 25 |
| 455 | 0.35 | 19.4444 |
| 57 | 0.15 | 8.3333 |
| 71 | 0.15 | 8.3333 |
| 97 | 0.15 | 8.3333 |
| 117 | 0.15 | 8.3333 |
| 222 | 0.15 | 8.3333 |
| 456 | 0.15 | 8.3333 |
| 474 | 0.15 | 8.3333 |
| 411 | 0.7 | 38.8889 |
| 429 | 0.6 | 33.3333 |
| 75 | 0.5 | 27.7778 |
| 474 | 0.3 | 16.6667 |
| 474 | 0.3 | 16.6667 |
| 223 | 0.2 | 11.1111 |
| 429 | 0.2 | 11.1111 |
| 59 | 0.1 | 5.5556 |

The metabolite 474.3736 (GTA-474) has the molecular formula C₃₀H₅₀O₄ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 473: 455, 429, and 411. In certain preferred embodiments, nominal parent/daughter mass 473/429 may be used for measuring GTA-474 levels.

### 476.3866 (GTA-476):

476.5
CE: −35 V

| m/z (amu) | intensity (counts) | % intensity |
|---|---|---|
| 475 | 4.1818 | 100 |
| 457 | 2.9091 | 69.5652 |
| 431 | 1.5455 | 36.9565 |
| 413 | 0.8182 | 19.5652 |
| 279 | 0.4545 | 10.8696 |
| 439 | 0.3636 | 8.6957 |
| 458 | 0.3636 | 8.6957 |
| 458 | 0.3636 | 8.6957 |
| 476 | 0.2727 | 6.5217 |
| 57 | 0.1818 | 4.3478 |
| 59 | 0.1818 | 4.3478 |
| 83 | 0.1818 | 4.3478 |
| 97 | 0.1818 | 4.3478 |
| 111 | 0.1818 | 4.3478 |
| 123 | 0.1818 | 4.3478 |
| 235 | 0.1818 | 4.3478 |
| 251 | 0.1818 | 4.3478 |
| 414 | 0.1818 | 4.3478 |
| 432 | 0.1818 | 4.3478 |
| 71 | 0.0909 | 2.1739 |

The metabolite 476.3866 (GTA-476) has the molecular formula C₃₀H₅₂O₄ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 475: 457, 431, 439 and 413. In certain preferred embodiments, nominal parent/daughter mass 475/431 may be used for measuring GTA-476 levels.

### 478.4022 (GTA-478):

478.4
CE: −35 V

| m/z (amu) | intensity (counts) | % intensity |
|---|---|---|
| 477 | 7.4286 | 100 |
| 459 | 5.2857 | 71.1538 |
| 433 | 2 | 26.9231 |
| 415 | 1.6429 | 22.1154 |
| 478 | 0.7857 | 10.5769 |
| 434 | 0.5 | 6.7308 |
| 460 | 0.5 | 6.7308 |
| 125 | 0.3571 | 4.8077 |
| 281 | 0.3571 | 4.8077 |
| 97 | 0.2857 | 3.8462 |
| 111 | 0.2857 | 3.8462 |
| 435 | 0.2857 | 3.8462 |
| 59 | 0.2143 | 2.8846 |
| 123 | 0.2143 | 2.8846 |
| 223 | 0.2143 | 2.8846 |
| 416 | 0.2143 | 2.8846 |
| 434 | 0.2143 | 2.8846 |
| 435 | 0.2143 | 2.8846 |
| 441 | 0.2143 | 2.8846 |
| 477 | 0.2143 | 2.8846 |

The metabolite 478.4022 (GTA-478) has the molecular formula C₃₀H₅₄O₄ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 477: 459, 433, 441 and 415. In certain preferred embodiments, nominal parent/daughter mass 477/433 may be used for measuring GTA-478 levels.

### 484.3764 (GTA-484):

484.4
CE: −40 V

| m/z (amu) | intensity (counts) | % intensity |
|---|---|---|
| 315 | 1.8333 | 100 |
| 123 | 0.8333 | 45.4545 |
| 297 | 0.8333 | 45.4545 |
| 185 | 0.6667 | 36.3636 |
| 465 | 0.6667 | 36.3636 |
| 279 | 0.5 | 27.2727 |
| 439 | 0.5 | 27.2727 |
| 483 | 0.5 | 27.2727 |
| 171 | 0.3333 | 18.1818 |
| 187 | 0.3333 | 18.1818 |
| 201 | 0.3333 | 18.1818 |
| 223 | 0.3333 | 18.1818 |
| 241 | 0.3333 | 18.1818 |
| 295 | 0.3333 | 18.1818 |
| 313 | 0.3333 | 18.1818 |
| 315 | 0.3333 | 18.1818 |
| 421 | 0.3333 | 18.1818 |
| 447 | 0.3333 | 18.1818 |
| 101 | 0.1667 | 9.0909 |
| 111 | 0.1667 | 9.0909 |

The metabolite 484.3764 (GTA-484) has the molecular formula C₂₈H₅₂O₆ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 483: 465, 315, 439, 483, 421, and 447. In certain preferred embodiments, nominal parent/daughter mass 483/315 may be used for measuring GTA-484 levels.

### 490.3658 (GTA-490):

490.4
CE: −35 V

| m/z (amu) | intensity (counts) | % intensity |
|---|---|---|
| 489 | 1.1739 | 100 |
| 319 | 0.413 | 35.1852 |
| 445 | 0.3696 | 31.4815 |
| 241 | 0.3478 | 29.6296 |
| 471 | 0.3478 | 29.6296 |
| 427 | 0.1957 | 16.6667 |
| 113 | 0.1739 | 14.8148 |
| 195 | 0.1739 | 14.8148 |
| 223 | 0.1739 | 14.8148 |
| 249 | 0.1739 | 14.8148 |
| 490 | 0.1739 | 14.8148 |
| 97 | 0.1522 | 12.963 |
| 267 | 0.1522 | 12.963 |
| 345 | 0.1304 | 11.1111 |
| 57 | 0.1087 | 9.2593 |
| 101 | 0.1087 | 9.2593 |
| 143 | 0.1087 | 9.2593 |
| 265 | 0.1087 | 9.2593 |
| 373 | 0.1087 | 9.2593 |
| 472 | 0.1087 | 9.2593 |

The metabolite 490.3658 (GTA-490) has the molecular formula C₃₀H₅₀O₅ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 489: 445, 471, 427 and 319. In certain preferred embodiments, nominal parent/daughter mass 489/445 may be used for measuring GTA-490 levels.

### 492.3815 (GTA-492):

492.4
CE: −40 V

| m/z (amu) | intensity (counts) | % intensity |
|---|---|---|
| 241 | 4.3077 | 100 |
| 249 | 2.6923 | 62.5 |
| 267 | 2.4615 | 57.1429 |
| 97 | 1.8462 | 42.8571 |
| 473 | 1.3846 | 32.1429 |
| 223 | 1.1538 | 26.7857 |
| 195 | 1 | 23.2143 |
| 143 | 0.9231 | 21.4286 |
| 447 | 0.9231 | 21.4286 |
| 101 | 0.8462 | 19.6429 |
| 491 | 0.8462 | 19.6429 |
| 113 | 0.7692 | 17.8571 |
| 319 | 0.6923 | 16.0714 |
| 57 | 0.5385 | 12.5 |
| 59 | 0.4615 | 10.7143 |
| 213 | 0.4615 | 10.7143 |
| 167 | 0.3846 | 8.9286 |
| 171 | 0.3846 | 8.9286 |
| 179 | 0.3846 | 8.9286 |
| 193 | 0.3846 | 8.9286 |

The metabolite 492.3815 (GTA-492) has the molecular formula C₃₀H₅₂Oand can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 491: 241, 249, 267, 473, and 447. In certain preferred embodiments, nominal parent/daughter mass 491/241 may be used for measuring GTA-492 levels.

### 494.3971 (GTA-494):

494.4
CE: −35 V

| m/z (amu) | intensity (counts) | % intensity |
|---|---|---|
| 493 | 3 | 100 |
| 475 | 2.6667 | 88.8889 |
| 215 | 1.6667 | 55.5556 |
| 195 | 1.3333 | 44.4444 |
| 213 | 1.3333 | 44.4444 |
| 449 | 1 | 33.3333 |
| 167 | 0.6667 | 22.2222 |
| 171 | 0.6667 | 22.2222 |
| 241 | 0.6667 | 22.2222 |
| 267 | 0.6667 | 22.2222 |
| 279 | 0.6667 | 22.2222 |
| 297 | 0.6667 | 22.2222 |
| 307 | 0.6667 | 22.2222 |
| 431 | 0.6667 | 22.2222 |
| 494 | 0.6667 | 22.2222 |
| 494 | 0.6667 | 22.2222 |
| 113 | 0.3333 | 11.1111 |
| 141 | 0.3333 | 11.1111 |
| 151 | 0.3333 | 11.1111 |
| 197 | 0.3333 | 11.1111 |

The metabolite 494.3971 (GTA-494) has the molecular formula C₃₀H₅₄O₅ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 493: 475, 215, and 449. In certain preferred embodiments, nominal parent/daughter mass 493/449 may be used for measuring GTA-494 levels.

### 502.4022 (GTA-502):

502.4
CE: −35 V

| m/z (amu) | intensity (counts) | % intensity |
|---|---|---|
| 483 | 1.0435 | 100 |
| 501 | 0.913 | 87.5 |
| 439 | 0.7391 | 70.8333 |
| 457 | 0.5217 | 50 |
| 502 | 0.2609 | 25 |
| 279 | 0.1739 | 16.6667 |
| 458 | 0.1739 | 16.6667 |
| 484 | 0.1739 | 16.6667 |
| 502 | 0.1739 | 16.6667 |
| 59 | 0.1304 | 12.5 |
| 109 | 0.1304 | 12.5 |
| 111 | 0.1304 | 12.5 |
| 123 | 0.1304 | 12.5 |
| 196 | 0.1304 | 12.5 |
| 221 | 0.1304 | 12.5 |
| 222 | 0.1304 | 12.5 |
| 277 | 0.1304 | 12.5 |
| 317 | 0.1304 | 12.5 |
| 440 | 0.1304 | 12.5 |
| 465 | 0.1304 | 12.5 |

The metabolite 502.4022 (GTA-502) has the molecular formula C₃₂H₅₄O₄ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 501: 483, 457, 465 and 439. In certain preferred embodiments, nominal parent/daughter mass 501/457 may be used for measuring GTA-502 levels.

### 504.4195 (GTA-504):

504.4
CE: −40 V

| m/z (amu) | intensity (counts) | % intensity |
|---|---|---|
| 485 | 5.8947 | 100 |
| 503 | 4.0526 | 68.75 |
| 441 | 2.5789 | 43.75 |
| 459 | 1.2105 | 20.5357 |
| 486 | 0.6842 | 11.6071 |
| 97 | 0.4211 | 7.1429 |
| 111 | 0.3684 | 6.25 |
| 467 | 0.3158 | 5.3571 |
| 504 | 0.3158 | 5.3571 |
| 57 | 0.2632 | 4.4643 |
| 223 | 0.2632 | 4.4643 |
| 263 | 0.2632 | 4.4643 |
| 377 | 0.2632 | 4.4643 |
| 442 | 0.2632 | 4.4643 |
| 169 | 0.2105 | 3.5714 |
| 279 | 0.2105 | 3.5714 |
| 329 | 0.2105 | 3.5714 |
| 59 | 0.1579 | 2.6786 |
| 71 | 0.1579 | 2.6786 |
| 83 | 0.1579 | 2.6786 |

The metabolite 504.4195 (GTA-504) has the molecular formula C₃₂H₅₆O₄ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 503: 485, 459, 467 and 441. In certain preferred embodiments, nominal parent/daughter mass 503/459 may be used for measuring GTA-504 levels.

### 512.4077 (GTA-512):

512.4
CE: −35 V

| m/z (amu) | intensity (counts) | % intensity |
|---|---|---|
| 315 | 12 | 100 |
| 511 | 8.5 | 70.8333 |
| 151 | 2.3333 | 19.4444 |
| 213 | 1.8333 | 15.2778 |
| 297 | 1.5 | 12.5 |
| 493 | 1.3333 | 11.1111 |
| 195 | 1 | 8.3333 |
| 279 | 1 | 8.3333 |
| 512 | 0.8333 | 6.9444 |
| 512 | 0.6667 | 5.5556 |
| 141 | 0.5 | 4.1667 |
| 171 | 0.5 | 4.1667 |
| 313 | 0.5 | 4.1667 |
| 467 | 0.5 | 4.1667 |
| 169 | 0.3333 | 2.7778 |
| 177 | 0.3333 | 2.7778 |
| 231 | 0.3333 | 2.7778 |
| 251 | 0.3333 | 2.7778 |
| 259 | 0.3333 | 2.7778 |
| 314 | 0.3333 | 2.7778 |

The metabolite 512.4077 (GTA-512) has the molecular formula C₃₀H₅₆O₆ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 511: 493, 315, and 467. In certain preferred embodiments, nominal parent/daughter mass 511/315 may be used for measuring GTA-512 levels.

### 518.3974 (GTA-518):

518.4
CE: −40 V

| m/z (amu) | intensity (counts) | % intensity |
|---|---|---|
| 517 | 0.8182 | 100 |
| 499 | 0.5909 | 72.2222 |
| 115 | 0.4091 | 50 |
| 455 | 0.3636 | 44.4444 |
| 171 | 0.3182 | 38.8889 |
| 171 | 0.3182 | 38.8889 |
| 473 | 0.2727 | 33.3333 |
| 59 | 0.2273 | 27.7778 |
| 401 | 0.2273 | 27.7778 |
| 499 | 0.2273 | 27.7778 |
| 113 | 0.1818 | 22.2222 |
| 389 | 0.1818 | 22.2222 |
| 437 | 0.1818 | 22.2222 |
| 481 | 0.1818 | 22.2222 |
| 71 | 0.1364 | 16.6667 |
| 111 | 0.1364 | 16.6667 |
| 125 | 0.1364 | 16.6667 |
| 203 | 0.1364 | 16.6667 |
| 223 | 0.1364 | 16.6667 |
| 445 | 0.1364 | 16.6667 |

The metabolite 518.3974 (GTA-518) has the molecular formula C₃₂H₅₄O₅ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 517: 499, 473, 499, 481 and 445. In certain preferred embodiments, nominal parent/daughter mass 517/473 may be used for measuring GTA-518 levels.

### 520.4128 (GTA-520):

520.4
CE: −42 V

| m/z (amu) | intensity (counts) | % intensity |
|---|---|---|
| 501 | 2.2353 | 100 |
| 519 | 1.3824 | 61.8421 |
| 457 | 0.8235 | 36.8421 |
| 475 | 0.6176 | 27.6316 |
| 115 | 0.4118 | 18.4211 |
| 59 | 0.3529 | 15.7895 |
| 83 | 0.3529 | 15.7895 |
| 459 | 0.3529 | 15.7895 |
| 502 | 0.3529 | 15.7895 |
| 241 | 0.3235 | 14.4737 |
| 297 | 0.2647 | 11.8421 |
| 71 | 0.2353 | 10.5263 |
| 195 | 0.2353 | 10.5263 |
| 223 | 0.2353 | 10.5263 |
| 279 | 0.2353 | 10.5263 |
| 447 | 0.2353 | 10.5263 |
| 483 | 0.2353 | 10.5263 |
| 97 | 0.2059 | 9.2105 |
| 111 | 0.2059 | 9.2105 |
| 221 | 0.2059 | 9.2105 |

The metabolite 520.4128 (GTA-520) has the molecular formula C₃₂H₅₆O₅ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 519: 501, 457, 475, 459, 447 and 483. In certain preferred embodiments, nominal parent/daughter mass 519/475 may be used for measuring GTA-520 levels.

### 522.4284 (GTA-522):

522.4
CE: −40 V

| m/z (amu) | intensity (counts) | % intensity |
|---|---|---|
| 521 | 1.375 | 100 |
| 503 | 1.2917 | 93.9394 |
| 459 | 0.375 | 27.2727 |
| 241 | 0.3333 | 24.2424 |
| 477 | 0.3333 | 24.2424 |
| 504 | 0.25 | 18.1818 |
| 111 | 0.2083 | 15.1515 |
| 115 | 0.2083 | 15.1515 |
| 171 | 0.2083 | 15.1515 |
| 267 | 0.2083 | 15.1515 |
| 297 | 0.2083 | 15.1515 |
| 441 | 0.2083 | 15.1515 |
| 223 | 0.1667 | 12.1212 |
| 269 | 0.1667 | 12.1212 |
| 271 | 0.1667 | 12.1212 |
| 279 | 0.1667 | 12.1212 |
| 485 | 0.1667 | 12.1212 |
| 522 | 0.1667 | 12.1212 |
| 57 | 0.125 | 9.0909 |
| 59 | 0.125 | 9.0909 |

The metabolite 522.4284 (GTA-522) has the molecular formula C₃₂H₅₈O₅ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 521: 503, 459, 477, 504, 441 and 485. In certain preferred embodiments, nominal parent/daughter mass 521/477 may be used for measuring GTA-522 levels.

### 524.4441 (GTA-524):

524.4
CE: −40 V
m/z (amu)

523
505
487
479
463
461
443
365
337
299
297
281
279
271
269
253
251
243
225
197
171
169
157
155
143
141
139
127
125
123
115
113
111
83

The metabolite 524.4441 (GTA-524) has the molecular formula C₃₂H₆₀O₅ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 523: 505, 461, 479, 506, 443 and 487. In certain preferred embodiments, nominal parent/daughter mass 523/461 may be used for measuring GTA-524 levels.

### 530.4335 (GTA-530):

530.4
CE: −40 V

| m/z (amu) | intensity (counts) | % intensity |
|---|---|---|
| 529 | 1.1563 | 100 |
| 467 | 0.8125 | 70.2703 |
| 511 | 0.8125 | 70.2703 |
| 530 | 0.2188 | 18.9189 |
| 85 | 0.1563 | 13.5135 |
| 485 | 0.1563 | 13.5135 |
| 512 | 0.1563 | 13.5135 |
| 512 | 0.1563 | 13.5135 |
| 75 | 0.125 | 10.8108 |
| 468 | 0.125 | 10.8108 |
| 177 | 0.0938 | 8.1081 |
| 250 | 0.0938 | 8.1081 |
| 251 | 0.0938 | 8.1081 |
| 530 | 0.0938 | 8.1081 |
| 59 | 0.0625 | 5.4054 |
| 97 | 0.0625 | 5.4054 |
| 109 | 0.0625 | 5.4054 |
| 113 | 0.0625 | 5.4054 |
| 195 | 0.0625 | 5.4054 |
| 205 | 0.0625 | 5.4054 |

The metabolite 530.4335 (GTA-530) has the molecular formula C₃₄H₅₈O₄ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 529: 467, 511 and 485. In certain preferred embodiments, nominal parent/daughter mass 529/467 may be used for measuring GTA-530 levels.

### 532.4492 (GTA-532):

532.5
CE: −42 V

| m/z (amu) | intensity (counts) | % intensity |
|---|---|---|
| 513 | 1.375 | 100 |
| 469 | 1.25 | 90.9091 |
| 531 | 0.9375 | 68.1818 |
| 195 | 0.25 | 18.1818 |
| 470 | 0.25 | 18.1818 |
| 470 | 0.25 | 18.1818 |
| 111 | 0.1875 | 13.6364 |
| 181 | 0.1875 | 13.6364 |
| 251 | 0.1875 | 13.6364 |
| 487 | 0.1875 | 13.6364 |
| 514 | 0.1875 | 13.6364 |
| 532 | 0.1875 | 13.6364 |
| 59 | 0.125 | 9.0909 |
| 71 | 0.125 | 9.0909 |
| 97 | 0.125 | 9.0909 |
| 113 | 0.125 | 9.0909 |
| 127 | 0.125 | 9.0909 |
| 495 | 0.125 | 9.0909 |
| 514 | 0.125 | 9.0909 |
| 532 | 0.125 | 9.0909 |

The metabolite 532.4492 (GTA-532) has the molecular formula C₃₄H₆₀O₄ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 531: 513, 469, 487 and 495. In certain preferred embodiments, nominal parent/daughter mass 531/469 may be used for measuring GTA-532 levels.

### 536.4077 (GTA-536):

MS/MS transition 535/473

The metabolite 536.4077 (GTA-536) has the molecular formula C₃₂H₅₆O₆ and can be characterized by the MS/MS transition shown above. In certain embodiments of analysis using N₂ as collision gas and atmospheric pressure chemical ionization (APCI) under negative ionization, it may be preferred to utilize one or more of the observed daughter ions (nominal masses): These include the following daughter ion of parent [M−H] mass 535: 473. In a preferred embodiments, nominal parent/daughter mass 535/573 may be used for measuring GTA-590 levels.

### 538.4233 (GTA-538):

538.4
CE: −40 V

| m/z (amu) | intensity (counts) | % intensity |
|---|---|---|
| 537 | 1.6667 | 100 |
| 519 | 1 | 60 |
| 475 | 0.6667 | 40 |
| 493 | 0.4444 | 26.6667 |
| 59 | 0.3333 | 20 |
| 115 | 0.3333 | 20 |
| 333 | 0.3333 | 20 |
| 501 | 0.3333 | 20 |
| 520 | 0.3333 | 20 |
| 538 | 0.3333 | 20 |
| 101 | 0.2222 | 13.3333 |
| 315 | 0.2222 | 13.3333 |
| 457 | 0.2222 | 13.3333 |
| 538 | 0.2222 | 13.3333 |
| 538 | 0.2222 | 13.3333 |
| 71 | 0.1111 | 6.6667 |
| 143 | 0.1111 | 6.6667 |
| 171 | 0.1111 | 6.6667 |
| 179 | 0.1111 | 6.6667 |
| 221 | 0.1111 | 6.6667 |

The metabolite 538.4233 (GTA-538) has the molecular formula C₃₂H₅₈O₆ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 537: 519, 475, 493, 501 and 457. In certain preferred embodiments, nominal parent/daughter mass 537/475 may be used for measuring GTA-538 levels.

### 540.4389 (GTA-540):

540.5
CE: −35 V

| m/z (amu) | intensity (counts) | % intensity |
|---|---|---|
| 315 | 24.6 | 100 |
| 539 | 15.6 | 63.4146 |
| 223 | 2.4 | 9.7561 |
| 179 | 2.2 | 8.9431 |
| 521 | 1.8 | 7.3171 |
| 297 | 1.2 | 4.878 |
| 495 | 1.2 | 4.878 |
| 477 | 0.8 | 3.252 |
| 540 | 0.8 | 3.252 |
| 241 | 0.6 | 2.439 |
| 259 | 0.6 | 2.439 |
| 316 | 0.6 | 2.439 |
| 540 | 0.6 | 2.439 |
| 125 | 0.4 | 1.626 |
| 171 | 0.4 | 1.626 |
| 225 | 0.4 | 1.626 |
| 257 | 0.4 | 1.626 |
| 279 | 0.4 | 1.626 |
| 313 | 0.4 | 1.626 |
| 314 | 0.4 | 1.626 |

The metabolite 540.4389 (GTA-540) has the molecular formula C₃₂H₆₀O₆ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 539: 315, 521, 495 and 477. In certain preferred embodiments, nominal parent/daughter mass 539/315 may be used for measuring GTA-540 levels.

### 550.4597 (GTA-550):

550.5
CE: −42 V

| m/z (amu) | intensity (counts) | % intensity |
|---|---|---|
| 487 | 1 | 100 |
| 549 | 0.9286 | 92.8571 |
| 531 | 0.7857 | 78.5714 |
| 251 | 0.5714 | 57.1429 |
| 253 | 0.5714 | 57.1429 |
| 111 | 0.4286 | 42.8571 |
| 125 | 0.4286 | 42.8571 |
| 269 | 0.4286 | 42.8571 |
| 271 | 0.4286 | 42.8571 |
| 277 | 0.4286 | 42.8571 |
| 513 | 0.4286 | 42.8571 |
| 71 | 0.3571 | 35.7143 |
| 171 | 0.3571 | 35.7143 |
| 297 | 0.3571 | 35.7143 |
| 469 | 0.3571 | 35.7143 |
| 115 | 0.2857 | 28.5714 |
| 279 | 0.2857 | 28.5714 |
| 295 | 0.2857 | 28.5714 |
| 433 | 0.2857 | 28.5714 |
| 506 | 0.2857 | 28.5714 |

The metabolite 550.4597 (GTA-550) has the molecular formula C₃₄H₆₂O₅ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 549: 487, 531, 251, 253, 513, 469 and 506. In certain preferred embodiments, nominal parent/daughter mass 549/487 may be used for measuring GTA-550 levels.

### 574.4597 (GTA-574):

574.5
CE: −42 V

| m/z (amu) | intensity (counts) | % intensity |
|---|---|---|
| 573.4742 | 1.0571 | 100 |
| 295.2386 | 0.7143 | 67.5676 |
| 555.4666 | 0.5714 | 54.0541 |
| 125.1053 | 0.4857 | 45.9459 |
| 279.2508 | 0.4857 | 45.9459 |
| 171.1051 | 0.4571 | 43.2432 |
| 223.1408 | 0.4286 | 40.5405 |
| 511.4199 | 0.4 | 37.8378 |
| 157.085 | 0.3429 | 32.4324 |
| 493.4546 | 0.3429 | 32.4324 |
| 183.1039 | 0.2857 | 27.027 |
| 277.2282 | 0.2571 | 24.3243 |
| 293.2359 | 0.2571 | 24.3243 |
| 401.3605 | 0.2286 | 21.6216 |
| 113.0966 | 0.2 | 18.9189 |
| 293.2102 | 0.2 | 18.9189 |
| 429.3752 | 0.2 | 18.9189 |
| 249.2203 | 0.1714 | 16.2162 |
| 385.3457 | 0.1714 | 16.2162 |
| 389.3651 | 0.1714 | 16.2162 |

The metabolite 574.4597 (GTA-574) has the molecular formula C₃₆H₆₂O₅ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 573: 295, 223, 555 and 511. In certain preferred embodiments, nominal parent/daughter mass 573/223 may be used for measuring GTA-574 levels.

### 576.4754 (GTA-576):

576.5
CE: −42 V

| m/z (amu) | intensity (counts) | % intensity |
|---|---|---|
| 575 | 2.9048 | 100 |
| 277 | 1.4286 | 49.1803 |
| 297 | 1.4286 | 49.1803 |
| 557 | 1.2381 | 42.623 |
| 513 | 0.9524 | 32.7869 |
| 279 | 0.8095 | 27.8689 |
| 171 | 0.7619 | 26.2295 |
| 183 | 0.5238 | 18.0328 |
| 295 | 0.5238 | 18.0328 |
| 125 | 0.4762 | 16.3934 |
| 403 | 0.4286 | 14.7541 |
| 111 | 0.381 | 13.1148 |
| 495 | 0.381 | 13.1148 |
| 251 | 0.3333 | 11.4754 |
| 293 | 0.3333 | 11.4754 |
| 97 | 0.2857 | 9.8361 |
| 113 | 0.2857 | 9.8361 |
| 205 | 0.2857 | 9.8361 |
| 223 | 0.2857 | 9.8361 |
| 296 | 0.2857 | 9.8361 |

The metabolite 576.4754 (GTA-576) has the molecular formula C₃₆H₆₄O₅ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 575: 277, 297, 557, 513 and 495. In certain preferred embodiments, nominal parent/daughter mass 575/513 may be used for measuring GTA-576 levels.

### 580.5067 (GTA-580):

580.5
CE: −42 V
m/z (amu)
579
561
543
535
517
499
421
407
389
375
299
297
281
281
279
263
253
185
171

The metabolite 580.5067 (GTA-580) has the molecular formula C₃₆H₆₈O₅ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 579: 561, 543, 535, 517 and 499. In certain preferred embodiments, nominal parent/daughter mass 579/517 may be used for measuring GTA-580 levels.

### 590.4546 (GTA-590):

MS/MS transition 589/545

The metabolite 590.4546 (GTA-590) has the molecular formula C₃₆H₆₂O₆ and can be characterized by the MS/MS transition shown above. In certain embodiments of analysis using N₂ as collision gas and atmospheric pressure chemical ionization (APCI) under negative ionization, it may be preferred to utilize one or more of the observed daughter ions (nominal masses): These include the following daughter ion of parent [M−H] mass 589: 545. In a preferred embodiments, nominal parent/daughter mass 589/545 may be used for measuring GTA-590 levels.

### 592.4703 (GTA-592):

592.5
CE: −35 V

| m/z (amu) | intensity (counts) | % intensity |
|---|---|---|
| 555 | | |
| 113 | 16.1667 | 100 |
| 85 | 3.3333 | 20.6186 |
| 103 | 2 | 12.3711 |
| 175 | 2 | 12.3711 |
| 117 | 1.6667 | 10.3093 |
| 59 | 1.3333 | 8.2474 |
| 75 | 1.3333 | 8.2474 |
| 95 | 1.3333 | 8.2474 |
| 99 | 1.3333 | 8.2474 |
| 115 | 1 | 6.1856 |
| 149 | 1 | 6.1856 |
| 87 | 0.8333 | 5.1546 |
| 129 | 0.8333 | 5.1546 |
| 591 | 0.8333 | 5.1546 |
| 157 | 0.6667 | 4.1237 |
| 415 | 0.6667 | 4.1237 |
| 73 | 0.5 | 3.0928 |
| 415 | 0.5 | 3.0928 |
| 71 | 0.3333 | 2.0619 |
| 89 | 0.3333 | 2.0619 |

The metabolite 592.4703 (GTA-592) has the molecular formula C₃₆H₆₄O₆ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 591: 555 and 113. In certain preferred embodiments, nominal parent/daughter mass 591/555 may be used for measuring GTA-592 levels.

### 594.4859 (GTA-594):

594.5
CE: −50 V

| m/z (amu) | intensity (counts) | % intensity |
|---|---|---|
| 371 | 4.2 | 100 |
| 171 | 3.6 | 85.7143 |
| 315 | 3.6 | 85.7143 |
| 575 | 3.6 | 85.7143 |
| 277 | 3.4 | 80.9524 |
| 201 | 3 | 71.4286 |
| 295 | 2.8 | 66.6667 |
| 297 | 2.8 | 66.6667 |
| 593 | 2.8 | 66.6667 |
| 279 | 2.4 | 57.1429 |
| 557 | 2.2 | 52.381 |
| 141 | 1.8 | 42.8571 |
| 313 | 1.6 | 38.0952 |
| 513 | 1.6 | 38.0952 |
| 557 | 1.6 | 38.0952 |
| 125 | 1.4 | 33.3333 |
| 594 | 1.4 | 33.3333 |
| 576 | 1.2 | 28.5714 |
| 113 | 1 | 23.8095 |
| 139 | 1 | 23.8095 |

The metabolite 594.4859 (GTA-594) has the molecular formula C₃₆H₆₆O₆ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 593: 557, 371, 315 and 277. In certain preferred embodiments, nominal parent/daughter mass 593/557 or 593/371 may be used for measuring GTA-594 levels.

### 596.5016 (GTA-596):

596.5
CE: −50 V

| m/z (amu) | intensity (counts) | % intensity |
|---|---|---|
| 279 | 53.6 | 100 |
| 315 | 35.8 | 66.791 |
| 297 | 21.6 | 40.2985 |
| 313 | 9.6 | 17.9104 |
| 577 | 7.4 | 13.806 |
| 281 | 6.8 | 12.6866 |
| 595 | 6.2 | 11.5672 |
| 295 | 3.6 | 6.7164 |
| 171 | 3.4 | 6.3433 |
| 516 | 3.2 | 5.9701 |
| 559 | 2.6 | 4.8507 |
| 125 | 2.4 | 4.4776 |
| 141 | 2 | 3.7313 |
| 127 | 1.8 | 3.3582 |
| 155 | 1.6 | 2.9851 |
| 169 | 1.4 | 2.6119 |
| 185 | 1.4 | 2.6119 |
| 207 | 1.4 | 2.6119 |
| 280 | 1.2 | 2.2388 |
| 373 | 1.2 | 2.2388 |

The metabolite 596.5016 (GTA-596) has the molecular formula C₃₆H₆₈O₆ and can be characterized by the full CID MS/MS fragmentation pattern shown in the above table. In certain embodiments of analysis using N₂ as collision gas and atmospheric pressure chemical ionization (APCI) under negative ionization, it may be preferred to utilize the one or more of the daughter ions (nominal masses) bolded in the table above. These include the following daughter ions of parent [M−H] mass 595: 279, 315, 297, 577 and 559. In certain preferred embodiments, nominal parent/daughter mass 595/559 may be used for measuring GTA-596 levels.

According to the methods described herein, biological samples from a subject may be compared to the same type of sample taken from the normal population to identify differences in the levels of the described GTA biomarkers. The samples can be extracted and analyzed using various analytical platforms including, but not limited to, Fourier transform ion cyclotron resonance mass spectrometry (FTMS) and liquid chromatography mass spectrometry (LC-MS).

The biological samples could originate from anywhere within the body, for example but not limited to, blood (serum/plasma), stool, or biopsy of any solid tissue including tumor, adjacent normal, smooth and skeletal muscle, adipose tissue, liver, skin, hair, brain, kidney, pancreas, lung, colon, stomach, or other. Of particular interest are blood or serum samples. While the term “blood” or “serum” may be used herein, those skilled in the art will recognize that plasma or whole blood or a sub-fraction of whole blood may also be used.

When a blood sample is drawn from a patient there are several ways in which the sample can be processed. The range of processing can be as little as none (i.e. frozen whole blood) or as complex as the isolation of a particular cell type. The most common and routine procedures involve the preparation of either serum or plasma from whole blood. All blood sample processing methods, including spotting of blood samples onto solid-phase supports, such as filter paper or other immobile materials, are also contemplated.

Without wishing to be limiting, the processed blood or plasma sample described above may then be further processed to make it compatible with the methodical analysis technique to be employed in the detection and measurement of the metabolites contained within the processed blood sample. The types of processing can range from as little as no further processing to as complex as differential extraction and chemical derivatization. Extraction methods may include sonication, soxhlet extraction, microwave assisted extraction (MAE), supercritical fluid extraction (SFE), accelerated solvent extraction (ASE), pressurized liquid extraction (PLE), pressurized hot water extraction (PHWE) and/or surfactant assisted extraction (PHWE) in common solvents such as methanol, ethanol, mixtures of alcohols and water, or organic solvents such as ethyl acetate or hexane. A method of particular interest for extracting metabolites for FTMS analysis and for flow injection LC-MS/MS analysis is to perform a liquid/liquid extraction whereby non-polar metabolites dissolve in an organic solvent and polar metabolites dissolve in an aqueous solvent.

The extracted samples may be analyzed using any suitable method including those known in the art. For example, and without wishing to be limiting, extracts of biological samples are amenable to analysis on essentially any mass spectrometry platform, either by direct injection or following chromatographic separation. Typical mass spectrometers are comprised of a source that ionizes molecules within the sample, and a detector for detecting the ionized molecules or fragments of molecules. Non-limiting examples of common sources include electron impact, electrospray ionization (ESI), atmospheric pressure chemical ionization (APCI), atmospheric pressure photo ionization (APPI), matrix assisted laser desorption ionization (MALDI), surface enhanced laser desorption ionization (SELDI), and derivations thereof. Common mass separation and detection systems can include quadrupole, quadrupole ion trap, linear ion trap, time-of-flight (TOF), magnetic sector, ion cyclotron (FTMS), Orbitrap, and derivations and combinations thereof. The advantage of FTMS over other MS-based platforms is its high resolving capability that allows for the separation of metabolites differing by only hundredths of a Dalton, many of which would be missed by lower resolution instruments.

By the term “metabolite”, it is meant specific GTA small molecules, the levels or intensities of which are measured in a sample, and that may be used as markers to diagnose a disease state. These small molecules may also be referred to herein as “metabolite marker”, “metabolite component”, “biomarker”, or “biochemical marker”.

The metabolites are generally characterized by their accurate mass, as measured by mass spectrometry technique. The accurate mass may also be referred to as “accurate neutral mass” or “neutral mass”. The accurate mass of a metabolite is given herein in Daltons (Da), or a mass substantially equivalent thereto. By “substantially equivalent thereto”, it is meant that a +/−5 ppm difference in the accurate mass would indicate the same metabolite. The accurate mass is given as the mass of the neutral metabolite. During the ionization of the metabolites, which occurs during analysis of the sample, the metabolite will cause either a loss or gain of one or more hydrogen atoms and a loss or gain of an electron. This changes the accurate mass to the “ionized mass”, which differs from the accurate mass by the mass of hydrogen atoms and electrons lost or gained during ionization. Unless otherwise specified, the accurate neutral mass will be referred to herein.

Similarly, when a metabolite is described by its molecular formula, the molecular formula of the neutral metabolite will be given. Naturally, the molecular formula of the ionized metabolite will differ from the neutral molecular formula by the number of hydrogen atoms lost or gained during ionization or due to the addition of a non-hydrogen adduct ion.

Data is collected during analysis and quantifying data for one or more than one metabolite is obtained. “Quantifying data” is obtained by measuring the levels or intensities of specific metabolites present in a sample.

The quantifying data is compared to corresponding data from one or more than one reference sample. The “reference sample” is any suitable reference sample for the particular disease state. For example, and without wishing to be limiting in any manner, the reference sample may be a sample from a control individual, i.e., a person not suffering from GI inflammation and/or cancer with or without a family history of GI inflammation and/or cancer (also referred to herein as a “ ‘normal’ counterpart”); the reference sample may also be a sample obtained from a patient clinically diagnosed with GI inflammation and/or cancer. As would be understood by a person of skill in the art, more than one reference sample may be used for comparison to the quantifying data. For example and without wishing to be limiting, the one or more than one reference sample may be a first reference sample obtained from a control individual. In the case of monitoring a subject's change in disease state, the reference sample may include a sample obtained at an earlier time period either pre-therapy or during therapy to compare the change in disease state as a result of therapy.

An “internal control metabolite” refers to an endogenous metabolite naturally present in the patient. Any suitable endogenous metabolite that does not vary over the disease states can be used as the internal control metabolite.

Use of a ratio of the GTA metabolite marker to the internal control metabolite may offer measurement that is more stable and reproducible than measurement of absolute levels of the metabolite marker. As the internal control metabolite is naturally present in all samples and does not appear to vary significantly over disease states, the sample-to-sample variability (due to handling, extraction, etc.) is minimized.

The measurement of GTA metabolite markers according to the methods described herein can in certain embodiments be carried out using assay platforms other than mass spectometric methods. There are multiple types of assay platform options currently available depending on the molecules being detected. These include, but are not limited to, colorimetric chemical assays (UV, or other wavelength), antibody-based enzyme-linked immunosorbant assays (ELISAs), dipstick chemical assays, image analysis such as MRI, petscan, CT scan, and various alternate mass spectrometry-based systems.

In a non-limiting embodiment, a high throughput screening (HTS) assay may be implemented using conventional triple-quadrupole mass spectrometry technology. The HTS assay works by directly injecting a serum extract into the triple-quad mass spectrometer, which then individually isolates each of the parent molecules by single-ion monitoring (SIM). This is followed by the fragmentation of each molecule using an inert gas, such as N₂ (called a collision gas, collectively referred to as collision-induced dissociation or OD). The intensity of a specific fragment from each parent GTA biomarker is then measured and recorded, through a process called multiple-reaction monitoring (MRM). In addition, an internal standard molecule is also added to each sample and subjected to fragmentation as well. This internal standard fragment should have the same intensity in each sample if the method and instrumentation is operating correctly. When all biomarker fragment intensities, as well as the internal standard fragment intensities are collected, a ratio of the biomarker to IS fragment intensity is calculated, and the ratio log-transformed. The values for each subject sample are then compared to a previously determined distribution of disease-positive and controls, to determine the relative likelihood that the person is positive or negative for the disease state.

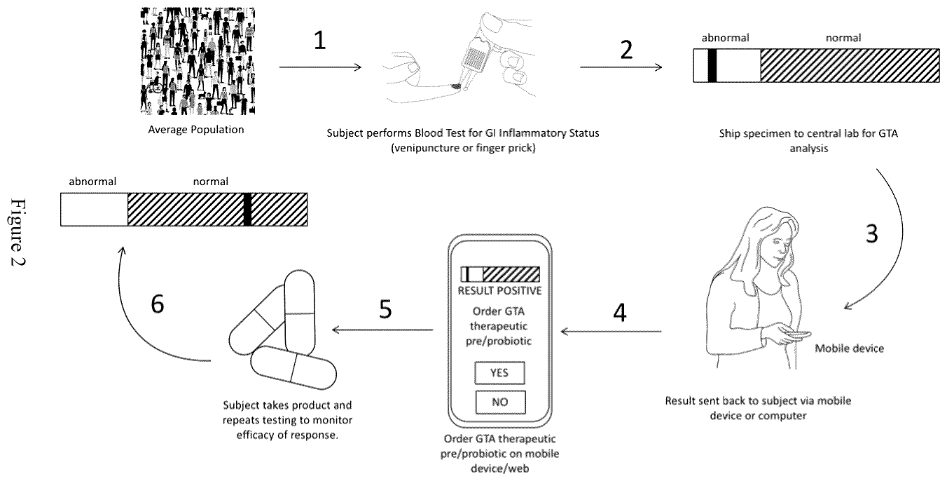

In further embodiments of the present invention, a test kit is provided for a subject to collect a small blood specimen, such as finger-prick dried blood spot or serum sample that can be analyzed by a central processing facility to test GTA metabolite levels as an indicator of GI inflammatory state. The central processing facility then reports the result back to the subject through one of various mechanisms, such as printed report, cloud-based electronic record, or other wireless type of communication. In the case of a positive test result (low GTA level), the subject would have the opportunity to purchase a GTA-augmenting anti-inflammatory prebiotic, probiotic, or synthetic GTA product. FIG. 2 illustrates a schematic diagram of this personalized testing and treatment approach.

A central processing facility can involve numerous options for the deployment of the GTA metabolite test assay. These may include, but are not limited to: 1, the development of MS/MS methods compatible with current laboratory instrumentation and triple-quadrupole mass spectrometers which are readily in place in several labs around the world, and/or 2, the establishment of a testing facility where samples could be shipped and analyzed at one location, and the results sent back to the patient or patient's physician.

Also described herein are therapeutic compositions comprising artificial, natural, or synthetic active agents for increasing endogenous GTA levels within the body.

Such therapeutic compositions may contain probiotic, non-pathogenic bacterial populations effective for increasing GTA levels within the body. These therapeutic compositions may also be useful for the prevention, control, and/or treatment of diseases, disorders and conditions associated with gastrointestinal (GI) inflammation and/or GI-related cancers, including but not limited to colorectal cancer, pancreatic cancer, and ovarian cancer.

In some embodiments, the therapeutic compositions contain prebiotics, e.g., carbohydrates, in conjunction with the microbial populations.

In embodiments of the foregoing methods, kits and compositions, the probiotic, non-pathogenic bacterial populations may comprise one or more bacterial species of the genus Blautia, species Faecalibacterium prausnitzii, genus Bacteroides, family Ruminococcaceae, family Lachnospiraceae, genus Coprococcus, genus Roseburia, genus Oscillospira, species Ruminococcus bromii, genus Ruminococcus, family Costridiaceae, species Dorea formicigenerans, species Bacteroides uniformis, genus Dorea, genus Streptococcus, order Clostridiales, genus Anaerostipes, genus Dialister, species Bifidobacterium adolescentis, family Coriobacteriaceae, genus Faecalibacterium, genus Sutterella, species Bacteroides ovatus, genus Parabacteroides, genus Ruminococcus, species Bacteroides faecis, species Eubacterium biforme, genus Phascolartobacterium, and/or family Enterobacteriaceae.

In further embodiments, the probiotic composition comprises a pharmaceutically acceptable excipient or carrier. In some embodiments, the pharmaceutically acceptable excipient or carrier may be suitable for administration to a mammalian subject by oral or rectal administration.

Non-limiting examples of suitable excipients and carriers include a buffering agent, a preservative, a stabilizer, a binder, a compaction agent, a lubricant, a dispersion enhancer, a disintegration agent, a flavoring agent, a sweetener, and a coloring agent.

Non-limiting examples of suitable buffering agents include sodium citrate, magnesium carbonate, magnesium bicarbonate, calcium carbonate, and calcium bicarbonate.

Non-limiting examples of suitable preservatives include antioxidants, such as alpha-tocopherol and ascorbate, and antimicrobials, such as parabens, chlorobutanol, and phenol.

In cases where a probiotic formulation contains anaerobic bacterial strains, the pharmaceutical formulation and excipients can be selected to prevent exposure of the bacterial strains to oxygen.

Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof

Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

Non-limiting examples of suitable dispersants include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments, the composition comprises a disintegrant. In other embodiments, the disintegrant is a non-effervescent disintegrant. Non-limiting examples of suitable non-effervescent disintegrants include starches such as corn starch, potato starch, pregelatinized and modified starches thereof, sweeteners, clays, such as bentonite, microcrystalline cellulose, alginates, sodium starch glycolate, gums such as agar, guar, locust bean, karaya, pecitin, and tragacanth. In another embodiment, the disintegrant is an effervescent disintegrant. Non-limiting examples of suitable effervescent disintegrants include sodium bicarbonate in combination with citric acid, and sodium bicarbonate in combination with tartaric acid.

Flavoring agents can be chosen from synthetic flavor oils and flavoring aromatics; natural oils; extracts from plants, leaves, flowers, and fruits; and combinations thereof. In some embodiments the flavoring agent is selected from cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

Non-limiting examples of suitable sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

Non-limiting examples of suitable coloring agents include food, drug and cosmetic colors (FD&C), drug and cosmetic colors (D&C), and external drug and cosmetic colors (Ext. D&C).

The weight fraction of the excipient or combination of excipients in the formulation is usually about 99% or less, such as about 95% or less, about 90% or less, about 85% or less, about 80% or less, about 75% or less, about 70% or less, about 65% or less, about 60% or less, about 55% or less, 50% or less, about 45% or less, about 40% or less, about 35% or less, about 30% or less, about 25% or less, about 20% or less, about 15% or less, about 10% or less, about 5% or less, about 2% or less, or about 1% or less of the total weight of the composition.

The compositions disclosed herein can be formulated into a variety of forms and administered by a number of different means. The compositions can be administered orally, or rectally, in formulations containing conventionally acceptable carriers, adjuvants, and vehicles as desired. In an exemplary embodiment, the composition is administered orally.

Solid dosage forms for oral administration include capsules, tablets, caplets, pills, troches, lozenges, powders, and granules. A capsule typically comprises a core material comprising a bacterial composition and a shell wall that encapsulates the core material. In some embodiments, the core material comprises at least one of a solid, a liquid, and an emulsion. In other embodiments, the shell wall material comprises at least one of a soft gelatin, a hard gelatin, and a polymer. Suitable polymers include, but are not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as those formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). In yet other embodiments, at least one polymer functions as taste-masking agents.

Tablets, pills, and the like can be compressed, multiply compressed, multiply layered, and/or coated. The coating can be single or multiple. In one embodiment, the coating material comprises at least one of a saccharide, a polysaccharide, and glycoproteins extracted from at least one of a plant, a fungus, and a microbe. Non-limiting examples include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In some embodiments the coating material comprises a protein. In another embodiment, the coating material comprises at least one of a fat and an oil. In other embodiments, the at least one of a fat and an oil is high temperature melting. In yet another embodiment, the at least one of a fat and an oil is hydrogenated or partially hydrogenated. In one embodiment, the at least one of a fat and an oil is derived from a plant. In other embodiments, the at least one of a fat and an oil comprises at least one of glycerides, free fatty acids, and fatty acid esters. In some embodiments, the coating material comprises at least one edible wax. The edible wax can be derived from animals, insects, or plants. Non-limiting examples include beeswax, lanolin, bayberry wax, carnauba wax, and rice bran wax. Tablets and pills can additionally be prepared with enteric coatings.

Alternatively, powders or granules embodying the bacterial compositions disclosed herein can be incorporated into a food product. In some embodiments, the food product is a drink for oral administration. Non-limiting examples of a suitable drink include fruit juice, a fruit drink, an artificially flavored drink, an artificially sweetened drink, a carbonated beverage, a sports drink, a liquid diary product, a shake, an alcoholic beverage, a caffeinated beverage, infant formula and so forth. Other suitable means for oral administration include aqueous and nonaqueous solutions, emulsions, suspensions and solutions and/or suspensions reconstituted from non-effervescent granules, containing at least one of suitable solvents, preservatives, emulsifying agents, suspending agents, diluents, sweeteners, coloring agents, and flavoring agents.

In some embodiments, the food product can be a solid foodstuff. Suitable examples of a solid foodstuff include without limitation a food bar, a snack bar, a cookie, a brownie, a muffin, a cracker, an ice cream bar, a frozen yogurt bar, and the like.

In other embodiments, the compositions disclosed herein are incorporated into a therapeutic food. In some embodiments, the therapeutic food is a ready-to-use food that optionally contains some or all essential macronutrients and micronutrients. In another embodiment, the compositions disclosed herein are incorporated into a supplementary food that is designed to be blended into an existing meal. In one embodiment, the supplemental food contains some or all essential macronutrients and micronutrients. In another embodiment, the bacterial compositions disclosed herein are blended with or added to an existing food to fortify the food's protein nutrition. Examples include food staples (grain, salt, sugar, cooking oil, margarine), beverages (coffee, tea, soda, beer, liquor, sports drinks), snacks, sweets and other foods.

The microbial compositions, with or without one or more prebiotics, are generally formulated for oral or gastric administration, typically to a mammalian subject. In particular embodiments, the composition is formulated for oral administration as a solid, semi-solid, gel, or liquid form, such as in the form of a pill, tablet, capsule, or lozenge. In some embodiments, such formulations contain or are coated by an enteric coating to protect the bacteria through the stomach and small intestine, although spores are generally resistant to the stomach and small intestines. In other embodiments, the microbial compositions, with or without one or more prebiotics, may be formulated with a germinant to enhance engraftment, or efficacy. In yet other embodiments, the bacterial compositions may be co-formulated or co-administered with prebiotic substances, to enhance engraftment or efficacy. In some embodiments, bacterial compositions may be co-formulated or co-administered with prebiotic substances, to enhance engraftment or efficacy.

The present invention is further defined with reference to the following examples that are not to be construed as limiting.

### EXAMPLES

1. Identification of Gut Microbes Associated with GTA Levels

Methods: High-throughput amplicon sequencing of the microbial V4 variable region of the microbial 16S rRNA gene was performed on total DNA extracted from 405 human colonic mucosa and fecal samples, using an Illumina Miseq instrument. Data from each sample was rarefied to 8,700 total sequences. Operational taxonomic units (OTUs) were filtered by percent contribution to the total, and the top 90% selected for comparison to serum GTA levels. Serum levels of 35 GTAs were determined on the same subjects by flow-injection tandem mass spectrometry. GTA levels were then aligned with OTU-level sequence data, followed by quintile analysis based on GTA level to identify statistically significant different OTUs between the highest and lowest GTA quintile.

Results: Comparison of OTUs between the lowest versus highest serum GTA quintiles across multiple GTAs revealed significant differences (p<E-4) in the relative abundances of several OTUs representing specific microbes, in particular short chain fatty acid-producing bacteria from the genus Blautia and species Faecalibacterium prausnitzii (Tables 1 through 34). A preliminary literature investigation of these microbes revealed roles in colon cancer, fatty acid metabolism and inflammation. Furthermore, we observed that 68% of the lowest GTA quintile comprised ulcerative colitis, Crohn's disease, and cancer, with only 25% healthy controls or non-GI related disease. The highest GTA quintile was comprised primarily of healthy individuals or non-GI related disease individuals, with only 2% of the individuals having Crohn's disease and cancer, and none with ulcerative colitis. These results suggest an involvement of both GTAs and specific microbes in GI-related inflammatory disorders and cancer. As far as we are aware, this is the first report connecting GTA metabolites with Blautia and Faecalibacterium prausnitzii in these processes.

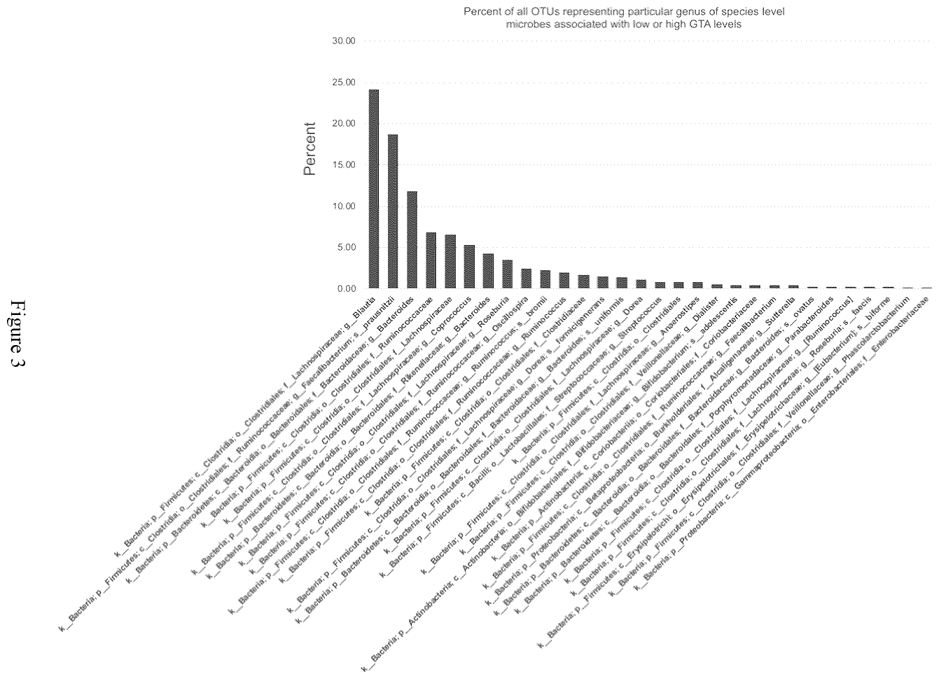

Across all operational taxonomic units (OTUs; groups of organisms based on similarity of RNA sequence), the most frequently associated (p<E-4) gut microbes with GTA production (across all 34 GTAs as shown in Tables 1 through 34) belonged to genus Blautia (24%), species prausnitzii (19%), genus Bacteroides (12%), family Ruminococcaceae (7%), family Lachnospiraceae (7%). The remaining OTU categories and their percent frequencies are shown in FIG. 3.

TABLE 1
Gut microbes corresponding with high and low GTA-446 levels

| OTUs | OTUs | N1 | meanQ1 | N2 | meanQ5 | p value | tstatistic | ratio |
|---|---|---|---|---|---|---|---|---|
| GTA445383 | | 81 | 0.52 | 80 | 2.29 | 2.93E−76 | −34.8 | |
| k_Bacteria; p_Firmicutes; c_Clostridia; o_Clostridiales; f_Lachnospiraceae; | v527 | 81 | 0.37 | 80 | 3.81 | 7.06E−06 | −4.6 | 10.3 |
| g_Blautia | | | | | | | | |
| k_Bacteria; p_Firmicutes; c_Clostridia; o_Clostridiales; f_Ruminococcaceae; | v967 | 81 | 0.15 | 80 | 1.70 | 9.38E−06 | −4.6 | 11.5 |
| g_Faecalibacterium; s_prausnitzii | | | | | | | | |
| k_Bacteria; p_Firmicutes; c_Clostridia; o_Clostridiales; f_Lachnospiraceae; | v5402 | 81 | 0.01 | 80 | 0.25 | 1.40E−05 | −4.5 | 20.2 |
| g_Roseburia | | | | | | | | |
| k_Bacteria; p_Firmicutes; c_Clostridia; o_Clostridiales; f_Lachnospiraceae; | v1007 | 81 | 0.17 | 80 | 1.44 | 1.86E−05 | −4.4 | 8.3 |
| g_Blautia | | | | | | | | |
| k_Bacteria; p_Firmicutes; c_Clostridia; o_Clostridiales; f_Lachnospiraceae; | v5333 | 81 | 0.04 | 80 | 0.30 | 2.72E−05 | −4.3 | 8.1 |
| g_Blautia | | | | | | | | |
| k_Bacteria; p_Firmicutes; c_Clostridia; o_Clostridiales; f_Lachnospiraceae; | v385 | 81 | 0.77 | 80 | 5.2S | 3.69E−05 | −4.2 | 6.9 |
| g_Blautia | | | | | | | | |
| k_Bacteria; p_Firmicutes; c_Clostridia; o_Clostridiales; f_Ruminococcaceae; | v717 | 81 | 0.41 | 80 | 2.38 | 3.93E−05 | −4.2 | 5.8 |
| g_Faecalibacterium; s_prausnitzii | | | | | | | | |
| k_Bacteria; p_Firmicutes; c_Clostridia; o_Clostridiales; f_Lachnospiraceae; | v284 | 81 | 4.04 | 80 | 0.65 | 5.43E−05 | 4.1 | 0.2 |
| g_Blautia | | | | | | | | |
| k_Bacteria; p_Firmicutes; c_Clostridia; o_Clostridiales; f_Ruminococcaceae; | v407 | 81 | 0.48 | 80 | 3.04 | 5.43E−05 | −4.1 | 6.3 |
| g_Faecalibacterium; s_prausnitzii | | | | | | | | |
| k_Bacteria; p_Firmicutes; c_Clostridia; o_Clostridiales; f_Ruminococcaceae | v13337 | 81 | 0.00 | 80 | 0.18 | 6.10E−05 | −4.1 | |
| k_Bacteria; p_Firmicutes; c_Clostridia; o_Clostridiales; f_Lachnospiraceae; | v1068 | 81 | 0.69 | 80 | 0.05 | 1.09E−04 | 4.0 | 0.1 |
| g_Blautia | | | | | | | | |
| k_Bacteria; p_Bacteroidetes; c_Bacteroidia; o_Bacteroidales; | v158 | 81 | 8.16 | 80 | 1.63 | 1.31E−04 | 3.9 | 0.2 |
| f_Bacteroidaceae; g_Bacteroides | | | | | | | | |
| k_Bacteria; p_Bacteroidetes; c_Bacteroidia; o_Bacteroidales; | v333 | 81 | 0.74 | 80 | 5.95 | 1.71E−04 | −3.9 | 8.0 |
| f_Bacteroidaceae; g_Bacteroides | | | | | | | | |
| k_Bacteria; p_Firmicutes; c_Clostridia; o_Clostridiales; f_Lachnospiraceae | v5604 | 81 | 0.04 | 80 | 0.33 | 1.94E−04 | −3.8 | 8.8 |
| k_Bacteria; p_Bacteroidetes; c_Bacteroidia; o_Bacteroidales; | v425 | 81 | 4.48 | 80 | 0.13 | 1.96E−04 | 3.8 | 0.0 |
| f_Bacteroidaceae; g_Bacteroides | | | | | | | | |
| k_Bacteria; p_Firmicutes; c_Clostridia; o_Clostridiales; f_Lachnospiraceae; | v1218 | 81 | 0.20 | 80 | 1.39 | 1.97E−04 | −3.8 | 7.0 |
| g_Blautia | | | | | | | | |
| k_Bacteria; p_Firmicutes; c_Clostridia; o_Clostridiales; f_Lachnospiraceae; | v2737 | 81 | 0.02 | 80 | 0.55 | 2.23E−04 | −3.8 | 22.3 |
| g_Blautia | | | | | | | | |
| k_Bacteria; p_Firmicutes; c_Clostridia; o_Clostridiales; f_Lachnospiraceae; | v5401 | 81 | 0.01 | 80 | 0.35 | 2.25E−04 | −3.8 | 28.3 |
| g_Dorea; s_formicigenerans | | | | | | | | |
| k_Bacteria; p_Firmicutes; c_Clostridia; o_Clostridiales; f_Lachnospiraceae; | v1963 | 81 | 0.07 | 80 | 0.85 | 2.58E−04 | −3.7 | 11.5 |
| g_Blautia | | | | | | | | |
| k_Bacteria; p_Firmicutes; c_Clostridia; o_Clostridiales; f_Ruminococcaceae; | v2878 | 81 | 0.38 | 80 | 0.00 | 2.60E−04 | 3.7 | 0.0 |
| g_Oscillospira | | | | | | | | |
| k_Bacteria; p_Firmicutes; c_Clostridia; o_Clostridiales; f_Lachnospiraceae; | v2169 | 81 | 0.11 | 80 | 0.64 | 2.81E−04 | −3.7 | 5.7 |
| g_Dorea | | | | | | | | |
| k_Bacteria; p_Firmicutes; c_Clostridia; o_Clostridiales | v1156 | 81 | 0.10 | 80 | 1.61 | 3.22E−04 | −3.7 | 16.3 |
| k_Bacteria; p_Firmicutes; c_Clostridia; o_Clostridiales; f_Ruminococcaceae; | v324 | 81 | 1.01 | 80 | 4.10 | 3.40E−04 | −3.7 | 4.0 |
| g_Faecalibacterium; s_prausnitzii | | | | | | | | |
| k_Bacteria; p_Actinobacteria; c_Coriobacteriia; o_Coriobacteriales; | v379 | 81 | 0.23 | 80 | 4.71 | 3.80E−04 | −3.6 | 20.1 |
| f_Coriobacteriaceae | | | | | | | | |
| k_Bacteria; p_Firmicutes; c_Clostridia; o_Clostridiales; f_Lachnospiraceae; | v188 | 81 | 6.64 | 80 | 1.83 | 3.89E−04 | 3.6 | 0.3 |
| g_Blautia | | | | | | | | |
| k_Bacteria; p_Firmicutes; c_Clostridia; o_Clostridiales; f_Clostridiaceae | v4592 | 81 | 0.04 | 80 | 0.59 | 4.01E−04 | −3.6 | 15.9 |
| k_Bacteria; p_Firmicutes; c_Clostridia; o_Clostridiales; f_Ruminococcaceae | v1384 | 81 | 0.15 | 80 | 0.84 | 4.07E−04 | −3.6 | 5.7 |
| k_Bacteria; p_Firmicutes; c_Clostridia; o_Clostridiales; f_Lachnospiraceae; | v323 | 81 | 0.81 | 80 | 4.16 | 4.81E−04 | −3.6 | 5.1 |
| g_Anaerostipes | | | | | | | | |
| k_Bacteria; p_Firmicutes; c_Clostridia; o_Clostridiales; f_Lachnospiraceae; | v513 | 81 | 1.93 | 80 | 0.55 | 4.94E−04 | 3.6 | 0.3 |
| g_Blautia | | | | | | | | |
| k_Bacteria; p_Firmicutes; c_Clostridia; o_Clostridiales; f_Lachnospiraceae; | v696 | 81 | 1.94 | 80 | 0.04 | 5.05E−04 | 3.6 | 0.0 |
| g_[Ruminococcus]; s_gnavus | | | | | | | | |
| k_Bacteria; p_Firmicutes; c_Clostridia; o_Clostridiales; f_Lachnospiraceae; | v282 | 81 | 1.59 | 80 | 5.86 | 5.81E−04 | −3.5 | 3.7 |
| g_Blautia | | | | | | | | |
| k_Bacteria; p_Firmicutes; c_Clostridia; o_Clostridiales; f_Lachnospiraceae; | v1423 | 81 | 0.15 | 80 | 1.06 | 5.88E−04 | −3.5 | 7.2 |
| g_Blautia | | | | | | | | |
| k_Bacteria; p_Firmicutes; c_Clostridia; o_Clostridiales; f_Ruminococcaceae; | v747 | 81 | 0.15 | 80 | 2.41 | 6.59E−04 | −3.5 | 16.3 |
| g_Ruminococcus; s_bromii | | | | | | | | |
| k_Bacteria; p_Firmicutes; c_Clostridia; o_Clostridiales; f_Lachnospiraceae; | v4704 | 81 | 0.04 | 80 | 0.23 | 6.83E−04 | −3.5 | 6.1 |
| g_Roseburia | | | | | | | | |
| k_Bacteria; p_Firmicutes; c_Clostridia; o_Clostridiales; f_Lachnospiraceae; | v1702 | 81 | 0.07 | 80 | 0.88 | 6.86E−04 | −3.5 | 11.8 |
| g_Coprococcus | | | | | | | | |
| k_Bacteria; p_Firmicutes; c_Clostridia; o_Clostridiales; f_Lachnospiraceae; | v979 | 81 | 0.35 | 80 | 1.60 | 6.96E−04 | −3.5 | 4.6 |
| g_Roseburia | | | | | | | | |
| k_Bacteria; p_Firmicutes; c_Clostridia; o_Clostridiales; f_Lachnospiraceae; | v3283 | 81 | 0.11 | 80 | 0.59 | 6.96E−04 | −3.5 | 5.3 |
| g_Dorea; s_formicigenerans | | | | | | | | |
| k_Bacteria; p_Firmicutes; c_Clostridia; o_Clostridiales; f_Lachnospiraceae | v1106 | 81 | 0.30 | 80 | 1.43 | 7.02E−04 | −3.5 | 4.8 |
| k_Bacteria; p_Bacteroidetes; c_Bacteroidia; o_Bacteroidales; | v2977 | 81 | 0.33 | 80 | 0.04 | 7.03E−04 | 3.5 | 0.1 |
| f_Bacteroidaceae; g_Bacteroides | | | | | | | | |
| k_Bacteria; p_Bacteroidetes; c_Bacteroidia; o_Bacteroidales; f_Rikenellaceae; | v5505 | 81 | 0.06 | 80 | 0.31 | 7.26E−04 | −3.4 | 5.1 |
| g_Bacteroides | | | | | | | | |
| k_Bacteria; p_Bacteroidetes; ac_Bacteroidia; o_Bacteroidales; | v529 | 81 | 0.15 | 80 | 1.36 | 7.28E−04 | −3.4 | 9.2 |
| f_Bacteroidaceae; g_Bacteroides | | | | | | | | |
| k_Bacteria; p_Firmicutes; c_Clostridia; o_Clostridiales; f_Lachnospiraceae; | v6 | 81 | 42.17 | 80 | 96.50 | 7.46E−04 | −3.4 | 2.3 |
| g_Roseburia | | | | | | | | |
| k_Bacteria; p_Firmicutes; c_Clostridia; o_Clostridiales; f_Lachnospiraceae; | v2461 | 81 | 0.15 | 80 | 0.68 | 7.93E−04 | −3.4 | 4.6 |
| g_Coprococcus | | | | | | | | |
| k_Bacteria; p_Firmicutes; c_Clostridia; o_Clostridiales; f_Lachnospiraceae; | v782 | 81 | 1.38 | 80 | 0.29 | 8.34E−04 | 3.4 | 0.2 |
| g_Coprococcus | | | | | | | | |
| k_Bacteria; p_Bacteroidetes; c_Bacteroidia; o_Bacteroidales; | v1449 | 81 | 0.09 | 80 | 0.64 | 8.75E−04 | −3.4 | 7.4 |
| f_Bacteroidaceae; g_Bacteroides; s_ovatus | | | | | | | | |
| k_Bacteria; p_Bacteroidetes; c_Bacteroidia; o_Bacteroidales; | v14672 | 81 | 0.15 | 80 | 0.00 | 8.82E−04 | 3.4 | 0.0 |
| f_Bacteroidaceae; g_Bacteroides | | | | | | | | |
| k_Bacteria; p_Firmicutes; c_Clostridia; o_Clostridiales; f_Lachnospiraceae; | v1678 | 81 | 0.17 | 80 | 0.59 | 9.48E−04 | −3.4 | 3.4 |
| g_Roseburia | | | | | | | | |
| k_Bacteria; p_Firmicutes; c_Clostridia; o_Clostridiales; f_Ruminococcaceae | v3864 | 81 | 0.01 | 80 | 0.28 | 9.52E−04 | −3.4 | 22.3 |

TABLE 2
Gut microbes corresponding with high and low GTA-448 levels

| OTUs | OTUs | N1 | meanQ1 | N2 | meanQ5 | pscore | tstatistic | ratio |
|---|---|---|---|---|---|---|---|---|
| GTA447385 | | 81 | 0.68 | 81 | 2.61 | 1.1E−76 | −34.9 | |
| k_Bacteria; p_Firmicutes; c_Clostridia; o_Clostridiales; f_Lachnospiraceae; | v1007 | 81 | 0.15 | 81 | 1.62 | 7.6E−06 | −4.6 | 10.92 |
| g_Blautia | | | | | | | | |
| k_Bacteria; p_Firmicutes; c_Clostridia; o_Clostridiales; f_Lachnospiraceae; | v527 | 81 | 0.46 | 81 | 3.65 | 2.7E−05 | −4.3 | 8.00 |
| g_Blautia | | | | | | | | |
| k_Bacteria; p_Bacteroidetes; c_Bacteroidia; o_Bacteroidales; f_Bacteroidaceae; | v158 | 81 | 7.89 | 81 | 1.74 | 6.0E−05 | 4.1 | 0.22 |
| g_Bacteroides | | | | | | | | |
| k_Bacteria; p_Firmicutes; c_Clostridia; o_Clostridiales; f_Lachnospiraceae; | v385 | 81 | 0.94 | 81 | 5.22 | 8.8E−05 | −4.0 | 5.57 |
| g_Blautia | | | | | | | | |
| k_Bacteria; p_Bacteroidetes; c_Bacteroidia; o_Bacteroidales; f_Bacteroidaceae; | v2977 | 81 | 0.33 | 81 | 0.04 | 1.3E−04 | 3.9 | 0.11 |
| g_Bacteroides | | | | | | | | |
| k_Bacteria; p_Firmicutes; c_Clostridia; o_Clostridiales; f_Ruminococcaceae; | v407 | 81 | 0.68 | 81 | 3.32 | 1.3E−04 | −3.9 | 4.89 |
| g_Faecalibacterium; s_prausnitzii | | | | | | | | |
| k_Bacteria; p_Firmicutes; c_Clostridia; o_Clostridiales; f_Lachnospiraceae; | v492 | 81 | 1.74 | 81 | 0.38 | 1.4E−04 | 3.9 | 0.22 |
| g_Coprococcus | | | | | | | | |
| k_Bacteria; p_Firmicutes; c_Clostridia; o_Clostridiales; f_Lachnospiraceae; | v782 | 81 | 1.12 | 81 | 0.22 | 1.6E−04 | 3.9 | 0.20 |
| g_Coprococcus | | | | | | | | |
| k_Bacteria; p_Firmicutes; c_Clostridia; o_Clostridiales; f_Lachnospiraceae; | v1423 | 81 | 0.15 | 81 | 1.19 | 1.7E−04 | −3.8 | 8.00 |
| g_Blautia | | | | | | | | |
| k_Bacteria; p_Bacteroidetes; c_Bacteroidia; o_Bacteroidales; f_Bacteroidaceae; | v396 | 81 | 2.78 | 81 | 1.01 | 2.8E−04 | 3.7 | 0.36 |
| g_Bacteroides | | | | | | | | |
| k_Bacteria; p_Firmicutes; c_Clostridia; o_Clostridiales; f_Ruminococcaceae | v8216 | 81 | 0.00 | 81 | 0.19 | 2.9E−04 | −3.7 | |
| k_Bacteria; p_Firmicutes; c_Clostridia; o_Clostridiales; f_Ruminococcaceae; | v7784 | 81 | 0.21 | 81 | 0.00 | 3.6E−04 | 3.6 | 0.00 |
| g_Faecalibacterium; s_prausnitzii | | | | | | | | |
| k_Bacteria; p_Firmicutes; c_Clostridia; o_Clostridiales; f_Ruminococcaceae | v13337 | 81 | 0.01 | 81 | 0.17 | 3.6E−04 | −3.6 | 14.00 |
| k_Bacteria; p_Firmicutes; c_Clostridia; o_Clostridiales; f_Lachnospiraceae; | v284 | 81 | 3.38 | 81 | 0.84 | 4.4E−04 | 3.6 | 0.25 |
| g_Blautia | | | | | | | | |
| k_Bacteria; p_Firmicutes; c_Clostridia; o_Clostridiales; f_Lachnospiraceae; | v2737 | 81 | 0.07 | 81 | 0.59 | 4.4E−04 | −3.6 | 8.00 |
| g_Blautia | | | | | | | | |
| k_Bacteria; p_Bacteroidetes; c_Bacteroidia; o_Bacteroidales; f_Bacteroidaceae; | v14672 | 81 | 0.19 | 81 | 0.01 | 4.8E−04 | 3.6 | 0.07 |
| g_Bacteroides | | | | | | | | |
| k_Bacteria; p_Firmicutes; c_Clostridia; o_Clostridiales; f_Ruminococcaceae; | v324 | 81 | 1.32 | 81 | 4.62 | 5.0E−04 | −3.6 | 3.50 |
| g_Faecalibacterium; s_prausnitzii | | | | | | | | |
| k_Bacteria; p_Firmicutes; c_Clostridia; o_Clostridiales; f_Lachnospiraceae; | v188 | 81 | 5.63 | 81 | 1.88 | 5.1E−04 | 3.5 | 0.33 |
| g_Blautia | | | | | | | | |
| k_Bacteria; p_Firmicutes; c_Clostridia; o_Clostridiales; f_Lachnospiraceae; | v1702 | 81 | 0.10 | 81 | 0.90 | 6.2E−04 | −3.5 | 9.13 |
| g_Coprococcus | | | | | | | | |
| k_Bacteria; p_Firmicutes; c_Clostridia; o_Clostridiales; f_Lachnospiraceae; | v5333 | 81 | 0.05 | 81 | 0.25 | 6.4E−04 | −3.5 | 5.00 |
| g_Blautia | | | | | | | | |
| k_Bacteria; p_Firmicutes; c_Clostridia; o_Clostridiales; f_Lachnospiraceae; | v5401 | 81 | 0.01 | 81 | 0.31 | 7.6E−04 | −3.4 | 25.00 |
| g_Dorea; s_formicigenerans | | | | | | | | |
| k_Bacteria; p_Firmicutes; c_Clostridia; o_Clostridiales; f_Ruminococcaceae; | v967 | 81 | 0.35 | 81 | 1.54 | 7.9E−04 | −3.4 | 4.46 |
| g_Faecalibacterium; s_prausnitzii | | | | | | | | |
| k_Bacteria; p_Firmicutes; c_Clostridia; o_Clostridiales; f_Ruminococcaceae | v1384 | 81 | 0.15 | 81 | 0.79 | 3.6E−04 | −3.4 | 5.33 |
| k_Bacteria; p_Bacteroidetes; c_Bacteroidia; o_Bacteroidales; f_Bacteroidaceae; | v2930 | 81 | 0.04 | 81 | 0.49 | 9.5E−04 | −3.4 | 13.33 |
| g_Bacteroides | | | | | | | | |
| k_Bacteria; p_Bacteroidetes; c_Bacteroidia; o_Bacteroidales; f_Bacteroidaceae; | v333 | 81 | 1.27 | 81 | 4.59 | 9.5E−04 | −3.4 | 3.61 |
| g_Bacteroides | | | | | | | | |

TABLE 3
Gut microbes corresponding with high and low GTA-450 levels

| OTUs | OTUs | N1 | meanQ1 | N2 | meanQ5 | pscore | tstatistic | ratio |
|---|---|---|---|---|---|---|---|---|
| GTA445405 | | 81 | 2.89 | 80 | 11.66 | 4.85E−70 | −31.4 | |
| k_Bacteria; p_Firmicutes; c_Clostridia; o_Clostridiales; f_Lachnospiraceae; | v1007 | 81 | 0.16 | 80 | 1.69 | 2.31E−05 | −4.4 | 10.51 |
| g_Blautia | | | | | | | | |
| k_Bacteria; p_Firmicutes; c_Clostridia; o_Clostridiales; f_Lachnospiraceae; | v5402 | 81 | 0.01 | 80 | 0.24 | 2.87E−05 | −4.3 | 19.24 |
| g_Roseburia | | | | | | | | |
| k_Bacteria; p_Firmicutes; c_Clostridia; o_Clostridiales; f_Lachnospiraceae; | v385 | 81 | 0.94 | 80 | 5.04 | 9.32E−05 | −4.0 | 5.37 |
| g_Blautia | | | | | | | | |
| k_Bacteria; p_Firmicutes; c_Clostridia; o_Clostridiales; f_Lachnospiraceae; | v527 | 81 | 0.48 | 80 | 3.58 | 1.49E−04 | −3.9 | 7.43 |
| g_Blautia | | | | | | | | |
| k_Bacteria; p_Firmicutes; c_Clostridia; o_Clostridiales; f_Lachnospiraceae; | v323 | 81 | 1.19 | 80 | 5.33 | 1.92E−04 | −3.8 | 4.49 |
| g_Anaerostipes | | | | | | | | |
| k_Bacteria; p_Firmicutes; c_Clostridia; o_Clostridiales; f_Lachnospiraceae; | v284 | 81 | 4.23 | 80 | 0.99 | 1.95E−04 | 3.8 | 0.23 |
| g_Blautia | | | | | | | | |
| k_Bacteria; p_Firmicutes; c_Clostridia; o_Clostridiales; f_Ruminococcaceae | v8216 | 81 | 0.00 | 80 | 0.18 | 2.03E−04 | −3.8 | |
| k_Bacteria; p_Bacteroidetes; c_Bacteroidia; o_Bacteroidales; f_Bacteroidaceae; | v158 | 81 | 7.05 | 80 | 2.15 | 3.24E−04 | 3.7 | 0.30 |
| g_Bacteroides | | | | | | | | |
| k_Bacteria; p_Firmicutes; c_Clostridia; o_Clostridiales; f_Lachnospiraceae; | v188 | 81 | 7.17 | 80 | 2.00 | 3.63E−04 | 3.6 | 0.28 |
| g_Blautia | | | | | | | | |
| k_Bacteria; p_Firmicutes; c_Clostridia; o_Clostridiales; f_Lachnospiraceae; | v492 | 81 | 2.44 | 80 | 0.40 | 4.45E−04 | 3.6 | 0.16 |
| g_Coprococcus | | | | | | | | |
| k_Bacteria; p_Firmicutes; c_Clostridia; o_Clostridiales; f_Lachnospiraceae; | v12957 | 81 | 0.16 | 80 | 0.00 | 4.68E−04 | 3.6 | 0.00 |
| g_Coprococcus | | | | | | | | |
| k_Bacteria; p_Firmicutes; c_Clostridia; o_Clostridiales; f_Lachnospiraceae; | v1423 | 81 | 0.16 | 80 | 1.29 | 5.09E−04 | −3.5 | 8.02 |
| g_Blautia | | | | | | | | |
| k_Bacteria; p_Firmicutes; c_Clostridia; o_Clostridiales; f_Ruminococcaceae; | v2874 | 81 | 0.47 | 80 | 0.06 | 5.41E−04 | 3.5 | 0.13 |
| g_Faecalibacterium; s_prausnitzii | | | | | | | | |
| k_Bacteria; p_Firmicutes; c_Clostridia; o_Clostridiales; f_Lachnospiraceae; | v5333 | 81 | 0.07 | 80 | 0.30 | 5.62E−04 | −3.5 | 4.05 |
| g_Blautia | | | | | | | | |
| k_Bacteria; p_Bacteroidetes; c_Bacteroidia; o_Bacteroidales; f_Bacteroidaceae; | v333 | 81 | 1.22 | 80 | 4.81 | 5.97E−04 | −3.5 | 3.94 |
| g_Bacteroides | | | | | | | | |
| k_Bacteria; p_Firmicutes; c_Clostridia; o_Clostridiales; f_Ruminococcaceae | v1384 | 81 | 0.19 | 80 | 0.86 | 6.05E−04 | −3.5 | 4.66 |
| k_Bacteria; p_Firmicutes; c_Clostridia; o_Clostridiales; f_Lachnospiraceae | v4916 | 81 | 0.02 | 80 | 0.36 | 6.56E−04 | −3.5 | 14.68 |
| k_Bacteria; p_Firmicutes; c_Clostridia; o_Clostridiales; f_Lachnospiraceae; | v8885 | 81 | 0.00 | 80 | 0.15 | 7.55E−04 | −3.4 | |
| g_Roseburia | | | | | | | | |
| k_Bacteria; p_Bacteroidetes; c_Bacteroidia; o_Bacteroidales; f_Bacteroidaceae; | v490 | 81 | 3.67 | 80 | 0.40 | 8.08E−04 | 3.4 | 0.11 |
| g_Bacteroides | | | | | | | | |
| k_Bacteria; p_Firmicutes; c_Clostridia; o_Clostridiales; f_Lachnospiraceae; | v5168 | 81 | 0.02 | 80 | 0.26 | 8.73E−04 | −3.4 | 10.63 |
| g_Roseburia | | | | | | | | |

TABLE 4
Gut microbes corresponding with high and low GTA-452 levels.

| OTUs | OTUs | N1 | meanQ1 | N2 | meanQ5 | p value | tstatistic | ratio |
|---|---|---|---|---|---|---|---|---|
| GTA451407 | | 81 | 0.92 | 80 | 3.44 | 4E−72 | −32.49 | |
| k_Bacteria; p_Firmicutes; c_Clostrida; o_Clostridiales; f_Lachnospiraceae; | v1007 | 81 | 0.15 | 80 | 1.66 | 8E−06 | −4.62 | 11.22 |
| g_Blautia | | | | | | | | |
| k_Bacteria; p_Firmicutes; c_Clostrida; o_Clostridiales; f_Ruminococcaceae; | v407 | 81 | 0.51 | 80 | 3.41 | 2E−05 | −4.38 | 6.74 |
| g_Faecalibacterium; s_prausnitzii | | | | | | | | |
| k_Bacteria; p_Firmicutes; c_Clostrida; o_Clostridiales; f_Lachnospiraceae; | v284 | 81 | 4.83 | 80 | 0.86 | 2E−05 | 4.37 | 0.18 |
| g_Blautia | | | | | | | | |
| k_Bacteria; p_Firmicutes; c_Clostrida; o_Clostridiales; f_Lachnospiraceae; | v188 | 81 | 7.96 | 80 | 1.84 | 2E−05 | 4.37 | 0.23 |
| g_Blautia | | | | | | | | |
| k_Bacteria; p_Firmicutes; c_Clostrida; o_Clostridiales; f_Ruminococcaceae; | v967 | 81 | 0.15 | 80 | 1.54 | 4E−05 | −4.25 | 10.38 |
| g_Faecalibacterium; s_prausnitzii | | | | | | | | |
| k_Bacteria; p_Bacteroidetes; c_Bacteroidia; o_Bacteroidales; f_Bacteroidaceae; | v158 | 81 | 8.01 | 80 | 2.06 | 4E−05 | 4.22 | 0.26 |
| g_Bacteroides | | | | | | | | |
| k_Bacteria; p_Firmicutes; c_Clostrida; o_Clostridiales; f_Lachnospiraceae; | v2737 | 81 | 0.02 | 80 | 0.64 | 5E−05 | −4.17 | 25.32 |
| g_Blautia | | | | | | | | |
| k_Bacteria; p_Firmicutes; c_Clostrida; o_Clostridiales; f_Lachnospiraceae; | v527 | 81 | 0.44 | 80 | 3.20 | 6E−05 | −4.12 | 7.20 |
| g_Blautia | | | | | | | | |
| k_Bacteria; p_Firmicutes; c_Clostrida; o_Clostridiales; f_Lachnospiraceae; | v385 | 81 | 0.86 | 80 | 4.61 | 7E−05 | −4.07 | 5.34 |
| g_Blautia | | | | | | | | |
| k_Bacteria; p_Firmicutes; c_Clostrida; o_Clostridiales; f_Ruminococcaceae; | v324 | 81 | 1.04 | 80 | 4.41 | 9E−05 | −4.03 | 4.25 |
| g_Faecalibacterium; s_prausnitzii | | | | | | | | |
| k_Bacteria; p_Firmicutes; c_Clostrida; o_Clostridiales; f_Lachnospiraceae; | v1423 | 81 | 0.15 | 80 | 1.16 | 2E−04 | −3.79 | 7.85 |
| g_Blautia | | | | | | | | |
| k_Bacteria; p_Bacteroidetes; c_Bacteroidia; o_Bacteroidales; f_Bacteroidaceae; | v490 | 81 | 4.37 | 80 | 0.44 | 2E−04 | 3.78 | 0.10 |
| g_Bacteroides | | | | | | | | |
| k_Bacteria; p_Bacteroidetes; c_Bacteroidia; o_Bacteroidales; f_Bacteroidaceae; | v396 | 81 | 2.93 | 80 | 1.14 | 3E−04 | 3.72 | 0.39 |
| g_Bacteroides | | | | | | | | |
| k_Bacteria; p_Firmicutes; c_Clostrida; o_Clostridiales; f_Ruminococcaceae | v1998 | 81 | 0.12 | 80 | 0.96 | 3E−04 | −3.72 | 7.80 |
| k_Bacteria; p_Firmicutes; c_Clostrida; o_Clostridiales; f_Ruminococcaceae | v1384 | 81 | 0.15 | 80 | 1.15 | 3E−04 | −3.70 | 7.76 |
| k_Bacteria; p_Firmicutes; c_Clostrida; o_Clostridiales; f_Lachnospiraceae; | v4400 | 81 | 0.30 | 80 | 0.06 | 3E−04 | 3.70 | 0.21 |
| g_Blautia | | | | | | | | |
| k_Bacteria; p_Firmicutes; c_Clostrida; o_Clostridiales; f_Lachnospiraceae; | v1963 | 81 | 0.12 | 80 | 0.91 | 3E−04 | −3.67 | 7.39 |
| g_Blautia | | | | | | | | |
| k_Bacteria; p_Firmicutes; c_Clostrida; o_Clostridiales; f_Lachnospiraceae | v1884 | 81 | 0.47 | 80 | 0.01 | 3E−04 | 3.67 | 0.03 |
| k_Bacteria; p_Firmicutes; c_Clostrida; o_Clostridiales; f_Lachnospiraceae; | v1702 | 81 | 0.09 | 80 | 1.08 | 4E−04 | −3.64 | 12.44 |
| g_Coprococcus | | | | | | | | |
| k_Bacteria; p_Firmicutes; c_Clostrida; o_Clostridiales; f_Ruminococcaceae; | v717 | 81 | 0.38 | 80 | 1.99 | 4E−04 | −3.62 | 5.19 |
| g_Faecalibacterium; s_prausnitzii | | | | | | | | |
| k_Bacteria; p_Firmicutes; c_Clostrida; o_Clostridiales; f_Ruminococcaceae; | v2170 | 81 | 0.04 | 80 | 0.91 | 4E−04 | −3.61 | 24.64 |
| g_Ruminococcus | | | | | | | | |
| k_Bacteria; p_Bacteroidetes; c_Bacteroidia; o_Bacteroidales; f_Bacteroidaceae; | v2977 | 81 | 0.41 | 80 | 0.06 | 5E−04 | 3.55 | 0.15 |
| g_Bacteroides | | | | | | | | |
| k_Bacteria; p_Firmicutes; c_Clostrida; o_Clostridiales; f_Lachnospiraceae; | v2739 | 81 | 0.06 | 80 | 0.48 | 5E−04 | −3.55 | 7.69 |
| g_[Ruminococcus]; s_torques | | | | | | | | |
| k_Bacteria; p_Firmicutes; c_Clostrida; o_Clostridiales; f_Ruminococcaceae; | v4745 | 81 | 0.04 | 80 | 0.38 | 6E−04 | −3.50 | 10.12 |
| g_Oscillospira | | | | | | | | |
| k_Bacteria; p_Bacteroidetes; c_Bacteroidia; o_Bacteroidales; f_Bacteroidaceae; | v3380 | 81 | 0.41 | 80 | 0.06 | 6E−04 | 3.50 | 0.15 |
| g_Bacteroides; s_ovatus | | | | | | | | |
| k_Bacteria; p_Firmicutes; c_Clostrida; o_Clostridiales; f_Lachnospiraceae; | v1218 | 81 | 0.31 | 80 | 1.44 | 6E−04 | −3.49 | 4.66 |
| g_Blautia | | | | | | | | |
| k_Bacteria; p_Bacteroidetes; c_Bacteroidia; o_Bacteroidales; f_Bacteroidaceae; | v1332 | 81 | 1.54 | 80 | 0.09 | 7E−04 | 3.44 | 0.06 |
| g_Bacteroides; s_uniformis | | | | | | | | |
| k_Bacteria; p_Firmicutes; c_Clostrida; o_Clostridiales; f_Ruminococcaceae; | v3094 | 81 | 0.05 | 80 | 0.58 | 8E−04 | −3.42 | 11.64 |
| g_Ruminococcus | | | | | | | | |
| k_Bacteria; p_Firmicutes; c_Clostrida; o_Clostridiales; f_Ruminococcaceae | v8216 | 81 | 0.01 | 80 | 0.18 | 8E−04 | −3.41 | 14.17 |
| k_Bacteria; p_Firmicutes; c_Clostrida; o_Clostridiales; f_Lachnospiraceae; | v1459 | 81 | 0.14 | 80 | 0.88 | 8E−04 | −3.41 | 6.44 |
| g_Blautia | | | | | | | | |
| k_Bacteria; p_Bacteroidetes; c_Bacteroidia; o_Bacteroidales; f_Bacteroidaceae; | v333 | 81 | 1.01 | 80 | 4.25 | 8E−04 | −3.41 | 4.20 |
| g_Bacteroides | | | | | | | | |
| k_Bacteria; p_Firmicutes; c_Clostrida; o_Clostridiales; f_Lachnospiraceae; | v513 | 81 | 2.36 | 80 | 0.79 | 9E−04 | 3.40 | 0.33 |
| g_Blautia | | | | | | | | |
| k_Bacteria; p_Bacteroidetes; c_Bacteroidia; o_Bacteroidales; f_Bacteroidaceae; | v13964 | 81 | 0.15 | 80 | 0.00 | 9E−04 | 3.39 | 0.00 |
| g_Bacteroides | | | | | | | | |

TABLE 5
Gut microbes corresponding with high and low GTA-464 levels:

| OTUs | QTUs | N1 | meanQ1 | N2 | meanQ5 | p value | tstatistic | ratio |
|---|---|---|---|---|---|---|---|---|
| GTA463419 | | 81 | 2.22 | 81 | 8.07 | 2.4E−68 | −30.39 | |
| k_Bacteria; p_Firmicutes; c_Clostrida; o_Clostridiales; f_Lachnospiraceae; | v1007 | 81 | 0.16 | 81 | 1.78 | 7.9E−06 | −4.62 | 11.08 |
| g_Blautia | | | | | | | | |
| k_Bacteria; p_Firmicutes; c_Clostrida; o_Clostridiales; f_Lachnospiraceae; | v284 | 81 | 4.51 | 81 | 0.79 | 1.5E−05 | 4.47 | 0.18 |
| g_Blautia | | | | | | | | |
| k_Bacteria; p_Firmicutes; c_Clostrida; o_Clostridiales; f_Lachnospiraceae; | v385 | 81 | 0.81 | 81 | 5.21 | 2.7E−05 | −4.32 | 6.39 |
| g_Blautia | | | | | | | | |
| k_Bacteria; p_Firmicutes; c_Clostrida; o_Clostridiales; f_Ruminococcaceae; | v967 | 81 | 0.17 | 81 | 1.59 | 3.2E−05 | −4.28 | 9.21 |
| g_Faecalibacterium; s_prausnitzii | | | | | | | | |
| k_Bacteria; p_Firmicutes; c_Clostrida; o_Clostridiales; f_Lachnospiraceae; | v527 | 81 | 0.44 | 81 | 3.81 | 3.4E−05 | −4.27 | 8.58 |
| g_Blautia | | | | | | | | |
| k_Bacteria; p_Firmicutes; c_Clostrida; o_Clostridiales; f_Lachnospiraceae; | v1068 | 81 | 0.79 | 81 | 0.09 | 4.0E−05 | 4.22 | 0.11 |
| g_Blautia | | | | | | | | |
| k_Bacteria; p_Firmicutes; c_Clostrida; o_Clostridiales; f_Lachnospiraceae; | v5402 | 81 | 0.01 | 81 | 0.22 | 6.6E−05 | −4.10 | 18.000 |
| g_Roseburia | | | | | | | | |
| k_Bacteria; p_Firmicutes; c_Clostrida; o_Clostridiales; f_Ruminococcaceae; | v407 | 81 | 0.53 | 81 | 3.16 | 8.8E−05 | −4.02 | 5.95 |
| g_Faecalibacterium; s_prausnitzii | | | | | | | | |
| k_Bacteria; p_Firmicutes; c_Clostrida; o_Clostridiales; f_Lachnospiraceae; | v188 | 81 | 7.48 | 81 | 1.93 | 1.1E−04 | 3.97 | 0.26 |
| g_Blautia | | | | | | | | |
| k_Bacteria; p_Bacteroidetes; c_Bacteroidia; o_Bacteroidales; | v490 | 81 | 4.33 | 81 | 0.42 | 1.9E−04 | 3.81 | 0.10 |
| f_Bacteroidaceae; g_Bacteroides | | | | | | | | |
| k_Bacteria; p_Bacteroidetes; c_Bacteroidia; o_Bacteroidales; | v333 | 81 | 1.02 | 81 | 4.84 | 2.1E−04 | −3.79 | 4.72 |
| f_Bacteroidaceae; g_Bacteroides | | | | | | | | |
| k_Bacteria; p_Firmicutes; c_Clostrida; o_Clostridiales; f_Lachnospiraceae; | v1423 | 81 | 0.15 | 81 | 1.36 | 2.2E−04 | −3.78 | 9.17 |
| g_Blautia | | | | | | | | |
| k_Bacteria; p_Firmicutes; c_Clostrida; o_Clostridiales; f_Lachnospiraceae; | v1702 | 81 | 0.07 | 81 | 0.94 | 2.6E−04 | −3.74 | 12.67 |
| g_Coprococcus | | | | | | | | |
| k_Bacteria; p_Firmicutes; c_Clostrida; o_Clostridiales; f_Ruminococcaceae; | v717 | 81 | 0.40 | 81 | 2.06 | 2.7E−04 | −3.72 | 5.22 |
| g_Faecalibacterium; s_prausnitzii | | | | | | | | |
| k_Bacteria; p_Bacteroidetes; c_Bacteroidia; o_Bacteroidales; | v158 | 81 | 6.94 | 81 | 1.93 | 2.8E−04 | 3.71 | 0.28 |
| f_Bacteroidaceae; g_Bacteroides | | | | | | | | |
| k_Bacteria; p_Firmicutes; c_Clostrida; o_Clostridiales; f_Lachnospiraceae; | v1691 | 81 | 0.20 | 81 | 0.65 | 3.0E−04 | −3.69 | 3.31 |
| g_Roseburia | | | | | | | | |
| k_Bacteria; p_Firmicutes; c_Clostrida; o_Clostridiales; f_Lachnospiraceae; | v6 | 81 | 41.49 | 81 | 115.75 | 3.1E−04 | −3.68 | 2.79 |
| g_Roseburia | | | | | | | | |
| k_Bacteria; p_Firmicutes; c_Clostrida; o_Clostridiales; f_Lachnospiraceae; | v5168 | 81 | 0.01 | 81 | 0.27 | 3.8E−04 | −3.63 | 22.00 |
| g_Roseburia | | | | | | | | |
| k_Bacteria; p_Firmicutes; c_Clostrida; o_Clostridiales; f_Lachnospiraceae; | v5401 | 81 | 0.01 | 81 | 0.33 | 4.0E−04 | −3.62 | 27.00 |
| g_Dorea; s_formicigenerans | | | | | | | | |
| k_Bacteria; p_Firmicutes; c_Clostrida; o_Clostridiales; f_Lachnospiraceae; | v1938 | 81 | 0.72 | 81 | 0.11 | 4.2E−04 | 3.60 | 0.16 |
| g_Coprococcus | | | | | | | | |
| k_Bacteria; p_Firmicutes; c_Clostrida; o_Clostridiales; f_Ruminococcaceae; | v4745 | 81 | 0.04 | 81 | 0.38 | 4.4E−04 | −3.59 | 10.33 |
| g_Oscillospira | | | | | | | | |
| k_Bacteria; p_Firmicutes; c_Clostrida; o_Clostridiales; f_Ruminococcaceae; | v324 | 81 | 1.09 | 81 | 4.36 | 4.5E−04 | −3.58 | 4.01 |
| g_Faecalibacterium; s_prausnitzii | | | | | | | | |
| k_Bacteria; p_Firmicutes; c_Clostrida; o_Clostridiales; f_Lachnospiraceae; | v4201 | 81 | 0.25 | 81 | 0.01 | 4.8E−04 | 3.57 | 0.05 |
| g_Blautia | | | | | | | | |
| k_Bacteria; p_Bacteroidetes; c_Bacteroidia; o_Bacteroidales; | v3024 | 81 | 0.06 | 81 | 0.36 | 6.7E−04 | −3.47 | 5.80 |
| f_Bacteroidaceae; g_Bacteroides | | | | | | | | |
| k_Bacteria; p_Firmicutes; c_Clostrida; o_Clostridiales; f_Ruminococcaceae | v13337 | 81 | 0.01 | 81 | 0.16 | 7.0E−04 | −3.46 | 13.00 |
| k_Bacteria; p_Firmicutes; c_Clostrida; o_Clostridiales; f_Lachnospiraceae | v4916 | 81 | 0.02 | 81 | 0.36 | 7.2E−04 | −3.45 | 14.50 |
| k_Bacteria; p_Firmicutes; c_Clostrida; o_Clostridiales; f_Ruminococcaceae | v1384 | 81 | 0.17 | 81 | 0.83 | 7.6E−04 | −3.43 | 4.79 |
| k_Bacteria; p_Firmicutes; c_Clostrida; o_Clostridiales; f_Lachnospiraceae; | v2737 | 81 | 0.07 | 81 | 0.57 | 8.1E−04 | −3.42 | 7.67 |
| g_Blautia | | | | | | | | |
| k_Bacteria; p_Firmicutes; c_Clostrida; o_Clostridiales; f_Lachnospiraceae | v494 | 81 | 0.43 | 81 | 2.88 | 8.3E−04 | −3.41 | 6.66 |
| k_Bacteria; p_Firmicutes; c_Clostrida; o_Clostridiales; f_Lachnospiraceae; | v597 | 81 | 0.70 | 81 | 2.00 | 8.8E−04 | −3.39 | 2.84 |
| g_Roseburia | | | | | | | | |
| k_Bacteria; p_Firmicutes; c_Clostrida; o_Clostridiales; f_Ruminococcaceae; | v2874 | 81 | 0.49 | 81 | 0.09 | 9.4E−04 | 3.37 | 0.17 |
| g_Faecalibacterium; s_prausnitzii | | | | | | | | |

TABLE 6
Gut microbes corresponding with high and low GTA-466 levels:

| OTUs | OTUs | N1 | meanQ1 | N2 | meanQ5 | p value | tstatistic | ratio |
|---|---|---|---|---|---|---|---|---|
| GTA465403 | | 82 | 0.54 | 81 | 2.06 | 2.83E−62 | −27.29 | |
| k_Bacteria; p_Bacteroidetes; c_Bacteroidia; o_Bacteroidales; f_Bacteroidaceae; | v158 | 82 | 8.71 | 81 | 1.74 | 9.72E−06 | 4.57 | 0.20 |
| g_Bacteroides | | | | | | | | |
| k_Bacteria; p_Firmicutes; c_Clostrida; o_Clostridiales; f_Lachnospiraceae; | v385 | 82 | 0.78 | 81 | 5.88 | 1.14E−05 | −4.53 | 7.53 |
| g_Blautia | | | | | | | | |
| k_Bacteria; p_Firmicutes; c_Clostrida; o_Clostridiales; f_Ruminococcaceae; | v407 | 82 | 0.48 | 81 | 3.51 | 1.74E−05 | −4.43 | 7.37 |
| g_Faecalibacterium; s_prausnitzii | | | | | | | | |
| k_Bacteria; p_Firmicutes; c_Clostrida; o_Clostridiales; f_Lachnospiraceae; | v1007 | 82 | 0.28 | 81 | 1.86 | 2.32E−05 | −4.36 | 6.65 |
| g_Blautia | | | | | | | | |
| k_Bacteria; p_Firmicutes; c_Clostrida; o_Clostridiales; f_Lachnospiraceae; | v527 | 82 | 0.43 | 81 | 4.11 | 2.43E−05 | −4.35 | 9.63 |
| g_Blautia | | | | | | | | |
| k_Bacteria; p_Firmicutes; c_Clostrida; o_Clostridiales; f_Lachnospiraceae; | v284 | 82 | 4.16 | 81 | 0.80 | 4.36E−05 | 4.20 | 0.19 |
| g_Blautia | | | | | | | | |
| k_Bacteria; p_Firmicutes; c_Clostrida; o_Clostridiales; f_Lachnospiraceae; | v1063 | 82 | 0.65 | 81 | 0.06 | 4.50E−05 | 4.19 | 0.10 |
| g_Blautia | | | | | | | | |
| k_Bacteria; p_Firmicutes; c_Clostrida; o_Clostridiales; f_Ruminococcaceae; | v324 | 82 | 1.02 | 81 | 4.49 | 5.34E−05 | −4.15 | 4.39 |
| g_Faecalibacterium; s_prausnitzii | | | | | | | | |
| k_Bacteria; p_Firmicutes; c_Clostrida; o_Clostridiales; f_Lachnospiraceae; | v5333 | 82 | 0.04 | 81 | 0.28 | 5.47E−05 | −4.15 | 7.76 |
| g_Blautia | | | | | | | | |
| k_Bacteria; p_Bacteroidetes; c_Bacteroidia; o_Bacteroidales; f_Bacteroidaceae; | v396 | 82 | 3.04 | 81 | 1.04 | 7.07E−05 | 4.08 | 0.34 |
| g_Bacteroides | | | | | | | | |
| k_Bacteria; p_Firmicutes; c_Clostrida; o_Clostridiales; f_Lachnospiraceae; | v9422 | 82 | 0.00 | 81 | 0.22 | 8.76E−05 | −4.02 | |
| g_Roseburia | | | | | | | | |
| k_Bacteria; p_Bacteroidetes; c_Bacteroidia; o_Bacteroidales; f_Bacteroidaceae; | v2977 | 82 | 0.41 | 81 | 0.04 | 1.01E−04 | 3.99 | 0.09 |
| g_Bacteroides | | | | | | | | |
| k_Bacteria; p_Firmicutes; c_Clostrida; o_Clostridiales; f_Lachnospiraceae; | v2737 | 82 | 0.02 | 81 | 0.58 | 1.15E−04 | −3.95 | 23.79 |
| g_Blautia | | | | | | | | |
| k_Bacteria; p_Firmicutes; c_Clostrida; o_Clostridiales; f_Lachnospiraceae; | v1423 | 82 | 0.23 | 81 | 1.48 | 1.84E−04 | −3.83 | 6.39 |
| g_Blautia | | | | | | | | |
| k_Bacteria; p_Firmicutes; c_Clostrida; o_Clostridiales; f_Lachnospiraceae; | v1691 | 82 | 0.21 | 81 | 0.73 | 2.31E−04 | −3.77 | 3.51 |
| g_Roseburia | | | | | | | | |
| k_Bacteria; p_Firmicutes; c_Clostrida; o_Clostridiales; f_Ruminococcaceae | v13337 | 82 | 0.00 | 81 | 0.15 | 2.43E−04 | −3.75 | |
| k_Bacteria; p_Firmicutes; c_Clostrida; o_Clostridiales; f_Lachnospiraceae; | v188 | 82 | 6.82 | 81 | 1.95 | 3.39E−04 | 3.66 | 0.29 |
| g_Blautia | | | | | | | | |
| k_Bacteria; p_Firmicutes; c_Clostrida; o_Clostridiales; f_Lachnospiraceae; | v5168 | 82 | 0.01 | 81 | 0.27 | 3.45E−04 | −3.66 | 22.27 |
| g_Roseburia | | | | | | | | |
| k_Bacteria; p_Firmicutes; c_Clostrida; o_Clostridiales; f_Ruminococcaceae; | v967 | 82 | 0.29 | 81 | 1.51 | 4.01E−04 | −3.62 | 5.15 |
| g_Faecalibacterium; s_prausnitzii | | | | | | | | |
| k_Bacteria; p_Firmicutes; c_Clostrida; o_Clostridiales; f_Ruminococcaceae | v360 | 82 | 1.00 | 81 | 3.77 | 4.06E−04 | −3.61 | 3.77 |
| k_Bacteria; p_Firmicutes; c_Clostrida; o_Clostridiales; f_Ruminococcaceae | v942 | 82 | 0.27 | 81 | 1.48 | 4.13E−04 | −3.61 | 5.52 |
| k_Bacteria; p_Bacteroidetes; c_Bacteroidia; o_Bacteroidales; f_Bacteroidaceae; | v425 | 82 | 4.62 | 81 | 0.48 | 4.19E−04 | 3.60 | 0.10 |
| g_Bacteroides | | | | | | | | |
| k_Bacteria; p_Firmicutes; c_Clostrida; o_Clostridiales; f_Ruminococcaceae | v8216 | 82 | 0.00 | 81 | 0.17 | 5.11E−04 | −3.55 | |
| k_Bacteria; p_Firmicutes; c_Clostrida; o_Clostridiales; f_Lachnospiraceae; | v8145 | 82 | 0.00 | 81 | 0.19 | 5.74E−04 | −3.51 | |
| g_Roseburia | | | | | | | | |
| k_Bacteria; p_Firmicutes; c_Clostrida; o_Clostridiales; f_Lachnospiraceae; | v1702 | 82 | 0.13 | 81 | 0.95 | 5.85E−04 | −3.51 | 7.09 |
| g_Coprococcus | | | | | | | | |
| k_Bacteria; p_Firmicutes; c_Clostrida; o_Clostridiales; f_Ruminococcaceae | v294 | 82 | 1.23 | 81 | 4.67 | 6.62E−04 | −3.47 | 3.79 |
| k_Bacteria; p_Firmicutes; c_Clostrida; o_Clostridiales; f_Lachnospiraceae | v4916 | 82 | 0.02 | 81 | 0.36 | 6.63E−04 | −3.47 | 14.68 |
| k_Bacteria; p_Firmicutes; c_Clostrida; o_Clostridiales; f_Lachnospiraceae; | v1218 | 82 | 0.33 | 81 | 1.42 | 7.58E−04 | −3.43 | 4.31 |
| g_Blautia | | | | | | | | |
| k_Bacteria; p_Firmicutes; c_Clostrida; o_Clostridiales | v1156 | 82 | 0.10 | 81 | 1.49 | 7.68E−04 | −3.43 | 15.31 |
| k_Bacteria; p_Firmicutes; c_Clostrida; o_Clostridiales; f_Ruminococcaceae; | v4745 | 82 | 0.05 | 81 | 0.38 | 8.23E−04 | −3.41 | 7.85 |
| g_Oscillospira | | | | | | | | |
| k_Bacteria; p_Bacteroidetes; c_Bacteroidia; o_Bacteroidales; f_Bacteroidaceae; | v2930 | 82 | 0.04 | 81 | 0.49 | 8.70E−04 | −3.39 | 13.50 |
| g_Bacteroides | | | | | | | | |
| k_Bacteria; p_Bacteroidetes; c_Bacteroidia; o_Bacteroidales; f_Bacteroidaceae; | v13964 | 82 | 0.15 | 81 | 0.00 | 8.87E−04 | 3.39 | 0.00 |
| g_Bacteroides | | | | | | | | |
| k_Bacteria; p_Firmicutes; c_Clostrida; o_Clostridiales; f_Lachnospiraceae | v957 | 82 | 0.33 | 81 | 1.30 | 8.90E−04 | −3.39 | 3.94 |
| k_Bacteria; p_Bacteroidetes; c_Bacteroidia; o_Bacteroidales; | v1695 | 82 | 0.18 | 81 | 0.79 | 9.59E−04 | −3.36 | 4.32 |
| f_Porphyromonadaceae; g_Parabacteroides | | | | | | | | |
| k_Bacteria; p_Bacteroidetes; c_Bacteroidia; o_Bacteroidales; f_Bacteroidaceae; | v490 | 82 | 3.93 | 81 | 0.47 | 9.59E−04 | 3.36 | 0.12 |
| g_Bacteroides | | | | | | | | |

TABLE 7
Gut microbes corresponding with high and low GTA-468 levels:

| OTUs | OTUs | N1 | meanQ1 | N2 | meanQ5 | p value | tstatistit | ratio |
|---|---|---|---|---|---|---|---|---|
| GTA467423 | | 81 | 1.23 | 80 | 5.30 | 2.52E−68 | −30.50 | |
| k_Bacteria; p_Firmicutes; c_Clostrida; o_Clostridiales; f_Lachnospiraceae; | v1007 | 81 | 0.12 | 80 | 1.68 | 2.71E−06 | −4.87 | 13.57 |
| g_Blautia | | | | | | | | |
| k_Bacteria; p_Firmicutes; c_Clostrida; o_Clostridiales; f_Ruminococcaceae; | v967 | 81 | 0.12 | 80 | 1.65 | 7.27E−06 | −4.64 | 13.37 |
| g_Faecalibacterium; s_prausnitzii | | | | | | | | |
| k_Bacteria; p_Firmicutes; c_Clostrida; o_Clostridiales; f_Lachnospiraceae; | v284 | 81 | 5.57 | 80 | 0.73 | 1.09E−05 | 4.54 | 0.13 |
| g_Blautia | | | | | | | | |
| k_Bacteria; p_Firmicutes; c_Clostrida; o_Clostridiales; f_Lachnospiraceae; | v527 | 81 | 0.38 | 80 | 3.73 | 1.18E−05 | −4.52 | 9.73 |
| g_Blautia | | | | | | | | |
| k_Bacteria; p_Firmicutes; c_Clostrida; o_Clostridiales; f_Lachnospiraceae; | v385 | 81 | 0.70 | 80 | 5.30 | 2.26E−05 | −4.37 | 7.53 |
| g_Blautia | | | | | | | | |
| k_Bacteria; p_Firmicutes; c_Clostrida; o_Clostridiales; f_Lachnospiraceae; | v2737 | 81 | 0.01 | 80 | 0.63 | 2.56E−05 | −4.34 | 50.62 |
| g_Blautia | | | | | | | | |
| k_Bacteria; p_Firmicutes; c_Clostrida; o_Clostridiales; f_Ruminococcaceae; | v324 | 81 | 0.91 | 80 | 4.51 | 2.64E−05 | −4.33 | 4.94 |
| g_Faecalibacterium; s_prausnitzii | | | | | | | | |
| k_Bacteria; p_Firmicutes; c_Clostrida; o_Clostridiales; f_Lachnospiraceae; | v188 | 81 | 8.79 | 80 | 1.79 | 2.63E−05 | 4.33 | 0.20 |
| g_Blautia | | | | | | | | |
| k_Bacteria; p_Firmicutes; c_Clostrida; o_Clostridiales; f_Ruminococcaceae; | v407 | 81 | 0.47 | 80 | 3.31 | 2.71E−05 | −4.32 | 7.06 |
| g_Faecalibacterium; s_prausnitzii | | | | | | | | |
| k_Bacteria; p_Firmicutes; c_Clostrida; o_Clostridiales; f_Ruminococcaceae; | v717 | 81 | 0.32 | 80 | 2.15 | 6.26E−05 | −4.11 | 6.70 |
| g_Faecalibacterium; s_prausnitzii | | | | | | | | |
| k_Bacteria; p_Firmicutes; c_Clostrida; o_Clostridiales; f_Lachnospiraceae; | v1068 | 81 | 0.72 | 80 | 0.08 | 9.53E−05 | 4.00 | 0.10 |
| g_Blautia | | | | | | | | |
| k_Bacteria; p_Bacteroidetes; c_Bacteroidia; o_Bacteroidales; f_Rikenellaceae; | v5504 | 81 | 0.00 | 80 | 0.19 | 1.04E−04 | −3.98 | |
| g_Bacteroides | | | | | | | | |
| k_Bacteria; p_Bacteroidetes; c_Bacteroidia; o_Bacteroidales; f_Bacteroidaceae; | v158 | 81 | 6.78 | 80 | 1.48 | 1.12E−04 | 3.96 | 0.22 |
| g_Bacteroides | | | | | | | | |
| k_Bacteria; p_Bacteroidetes; c_Bacteroidia; o_Bacteroidales; f_Bacteroidaceae; | v333 | 81 | 0.72 | 80 | 4.31 | 1.80E−04 | −3.84 | 6.02 |
| g_Bacteroides | | | | | | | | |
| k_Bacteria; p_Firmicutes; c_Clostrida; o_Clostridiales; f_Lachnospiraceae; | v1423 | 81 | 0.14 | 80 | 1.15 | 1.93E−04 | −3.82 | 8.47 |
| g_Blautia | | | | | | | | |
| k_Bacteria; p_Firmicutes; c_Clostrida; o_Clostridiales; f_Lachnospiraceae; | v2461 | 81 | 0.11 | 80 | 0.68 | 2.34E−04 | −3.77 | 6.08 |
| g_Coprococcus | | | | | | | | |
| k_Bacteria; p_Firmicutes; c_Clostrida; o_Clostridiales; f_Lachnospiraceae; | v1459 | 81 | 0.14 | 80 | 0.98 | 2.63E−04 | −3.73 | 7.18 |
| g_Blautia | | | | | | | | |
| k_Bacteria; p_Firmicutes; c_Clostrida; o_Clostridiales; f_Ruminococcaceae; | v3094 | 81 | 0.04 | 80 | 0.51 | 2.82E−04 | −3.71 | 13.84 |
| g_Ruminococcus | | | | | | | | |
| k_Bacteria; p_Firmicutes; c_Clostrida; o_Clostridiales; f_Lachnospiraceae; | v5333 | 81 | 0.05 | 80 | 0.28 | 2.93E−04 | −3.70 | 5.57 |
| g_Blautia | | | | | | | | |
| k_Bacteria; p_Firmicutes; c_Clostrida; o_Clostridiales; f_Lachnospiraceae | v4916 | 81 | 0.00 | 80 | 0.34 | 3.21E−04 | −3.68 | |
| k_Bacteria; p_Firmicutes; c_Clostrida; o_Clostridiales; f_Ruminococcaceae | v1384 | 81 | 0.10 | 80 | 0.96 | 3.28E−04 | −3.67 | 9.75 |
| k_Bacteria; p_Firmicutes; c_Clostrida; o_Clostridiales; f_Ruminococcaceae | v1998 | 81 | 0.07 | 80 | 0.83 | 3.40E−04 | −3.66 | 11.14 |
| k_Bacteria; p_Firmicutes; c_Clostrida; o_Clostridiales; f_Ruminococcaceae | v13337 | 81 | 0.00 | 80 | 0.14 | 4.71E−04 | −3.57 | |
| k_Bacteria; p_Firmicutes; c_Clostrida; o_Clostridiales; f_Clostridiaceae | v4592 | 81 | 0.04 | 80 | 0.59 | 4.75E−04 | −3.57 | 15.86 |
| k_Bacteria; p_Bacteroidetes; c_Bacteroidia; o_Bacteroidales; f_Bacteroidaceae; | v1332 | 81 | 1.58 | 80 | 0.08 | 4.77E−04 | 3.57 | 0.05 |
| g_Bacteroides; s_uniformis | | | | | | | | |
| k_Bacteria; p_Firmicutes; c_Clostrida; o_Clostridiales; f_Lachnospiraceae; | v1702 | 81 | 0.09 | 80 | 0.90 | 5.60E−04 | −3.52 | 10.41 |
| g_Coprococcus | | | | | | | | |
| k_Bacteria; p_Bacteroidetes; c_Bacteroidia; o_Bacteroidales; f_Bacteroidaceae; | v3024 | 81 | 0.04 | 80 | 0.30 | 5.61E−04 | −3.52 | 8.10 |
| g_Bacteroides | | | | | | | | |
| k_Bacteria; p_Firmicutes; c_Clostrida; o_Clostridiales; f_Lachnospiraceae | v5604 | 81 | 0.05 | 80 | 0.33 | 5.74E−04 | −3.51 | 6.58 |
| k_Bacteria; p_Firmicutes; c_Clostrida; o_Clostridiales; f_Ruminococcaceae; | v2170 | 81 | 0.01 | 80 | 0.74 | 5.74E−04 | −3.51 | 59.74 |
| g_Ruminococcus | | | | | | | | |
| k_Bacteria; p_Actinobacteria; c_Coriobacteriia; o_Coriobacteriales; | v2334 | 81 | 0.10 | 80 | 0.55 | 6.26E−04 | −3.49 | 5.57 |
| f_Coriobacteriaceae; g_Collinsella; s_aerofaciens | | | | | | | | |
| k_Bacteria; p_Firmicutes; c_Clostrida; o_Clostridiales; f_Lachnospiraceae; | v323 | 81 | 1.05 | 80 | 4.85 | 6.45E−04 | −3.48 | 4.62 |
| g_Anaerostipes | | | | | | | | |
| k_Bacteria; p_Firmicutes; c_Clostrida; o_Clostridiales; f_Lachnospiraceae | v1540 | 81 | 0.11 | 80 | 0.74 | 6.50E−04 | −3.48 | 6.64 |
| k_Bacteria; p_Firmicutes; c_Clostrida; o_Clostridiales; f_Ruminococcaceae; | v4745 | 81 | 0.02 | 80 | 0.35 | 6.63E−04 | −3.47 | 14.17 |
| g_Oscillospira | | | | | | | | |
| k_Bacteria; p_Firmicutes; c_Clostrida; o_Clostridiales; f_Lachnospiraceae; | v3283 | 81 | 0.07 | 80 | 0.48 | 6.74E−04 | −3.47 | 6.41 |
| g_Dorea; s_formicigenerans | | | | | | | | |
| k_Bacteria; p_Firmicutes; c_Clostrida; o_Clostridiales; f_Lachnospiraceae; | v782 | 81 | 1.37 | 80 | 0.24 | 6.78E−04 | 3.47 | 0.17 |
| g_Coprococcus | | | | | | | | |
| k_Bacteria; p_Firmicutes; c_Clostrida; o_Clostridiales; f_Lachnospiraceae; | v5401 | 81 | 0.01 | 80 | 0.31 | 6.95E−04 | −3.46 | 25.31 |
| g_Dorea; s_formicigenerans | | | | | | | | |
| k_Bacteria; p_Bacteroidetes; c_Bacteroidia; o_Bacteroidales; f_Bacteroidaceae; | v490 | 81 | 5.20 | 80 | 0.31 | 7.01E−04 | 3.46 | 0.06 |
| g_Bacteroides | | | | | | | | |
| k_Bacteria; p_Firmicutes; c_Clostrida; o_Clostridiales; f_Lachnospiraceae; | v513 | 81 | 2.48 | 80 | 0.75 | 7.65E−04 | 3.43 | 0.30 |
| g_Blautia | | | | | | | | |
| k_Bacteria; p_Bacteroidetes; c_Bacteroidia; o_Bacteroidales; f_Bacteroidaceae; | v731 | 81 | 0.06 | 80 | 2.60 | 8.02E−04 | −3.42 | 42.12 |
| g_Bacteroides | | | | | | | | |
| k_Bacteria; p_Firmicutes; c_Clostrida; o_Clostridiales; f_Ruminococcaceae | v360 | 81 | 0.86 | 80 | 3.29 | 8.15E−04 | −3.41 | 3.30 |
| k_Bacteria; p_Bacteroidetes; c_Bacteroidia; o_Bacteroidales; f_Bacteroidaceae; | v10047 | 81 | 0.01 | 80 | 0.18 | 8.16E−04 | −3.41 | 14.17 |
| g_Bacteroides | | | | | | | | |
| k_Bacteria; p_Firmicutes; c_Clostrida; o_Clostridiales; f_Lachnospiraceae; | v1963 | 81 | 0.12 | 80 | 0.84 | 8.42E−04 | −3.40 | 6.78 |
| g_Blautia | | | | | | | | |
| k_Bacteria; p_Bacteroidetes; c_Bacteroidia; o_Bacteroidales; f_Bacteroidaceae; | v2977 | 81 | 0.30 | 80 | 0.03 | 8.63E−04 | 3.40 | 0.08 |
| g_Bacteroides | | | | | | | | |
| k_Bacteria; p_Firmicutes; c_Clostrida; o_Clostridiales; f_Ruminococcaceae; | v2874 | 81 | 0.52 | 80 | 0.10 | 8.76E−04 | 3.39 | 0.19 |
| g_Faecalibacterium; s_prausnitzii | | | | | | | | |
| k_Bacteria; p_Bacteroidetes; c_Bacteroidia; o_Bacteroidales; f_Bacteroidaceae; | v396 | 81 | 2.47 | 80 | 0.93 | 9.60E−04 | 3.36 | 0.37 |
| g_Bacteroides | | | | | | | | |

TABLE 8
Gut microbes corresponding with high and low GTA-474 levels:

| OTUs | OTUs | N1 | meanQ1 | N2 | meanQ5 | p value | tstatistic | ratio |
|---|---|---|---|---|---|---|---|---|
| GTA473429 | | 83 | 0.51 | 80 | 1.75 | 4.4E−68 | −30.14 | |
| k_Bacteria; p_Firmicutes; c_Clostrida; o_Clostridiales; f_Lachnospiraceae; | v527 | 83 | 0.41 | 80 | 4.11 | 3.1E−06 | −4.83 | 10.04 |
| g_Blautia | | | | | | | | |
| k_Bacteria; p_Firmicutes; c_Clostrida; o_Clostridiales; f_Lachnospiraceae; | v385 | 83 | 0.80 | 80 | 5.59 | 1.4E−05 | −4.49 | 7.03 |
| g_Blautia | | | | | | | | |
| k_Bacteria; p_Firmicutes; c_Clostrida; o_Clostridiales; f_Lachnospiraceae; | v1007 | 83 | 0.16 | 80 | 1.31 | 5.1E−05 | −4.16 | 8.38 |
| g_Blautia | | | | | | | | |
| k_Bacteria; p_Firmicutes; c_Clostrida; o_Clostridiales; f_Ruminococcaceae; | v407 | 83 | 0.63 | 80 | 3.21 | 7.3E−05 | −4.07 | 5.13 |
| g_Faecalibacterium; s_prausnitzii | | | | | | | | |
| k_Bacteria; p_Firmicutes; c_Clostrida; o_Clostridiales; f_Ruminococcaceae | v8216 | 83 | 0.00 | 80 | 0.20 | 1.2E−04 | −3.95 | |
| k_Bacteria; p_Firmicutes; c_Clostrida; o_Clostridiales; f_Ruminococcaceae; | v324 | 83 | 1.10 | 80 | 4.41 | 2.5E−04 | −3.74 | 4.02 |
| g_Faecalibacterium; s_prausnitzii | | | | | | | | |
| k_Bacteria; p_Firmicutes; c_Clostrida; o_Clostridiales; f_Lachnospiraceae | v2563 | 83 | 0.05 | 80 | 0.71 | 4.1E−04 | −3.61 | 14.78 |
| k_Bacteria; p_Firmicutes; c_Clostrida; o_Clostridiales | v1156 | 83 | 0.17 | 80 | 1.65 | 4.2E−04 | −3.60 | 9.78 |
| k_Bacteria; p_Firmicutes; c_Clostrida; o_Clostridiales; f_Lachnospiraceae; | v1702 | 83 | 0.14 | 80 | 1.08 | 4.9E−04 | −3.56 | 7.44 |
| g_Coprococcus | | | | | | | | |
| k_Bacteria; p_Firmicutes; c_Clostrida; o_Clostridiales; f_Ruminococcaceae | v13337 | 83 | 0.01 | 80 | 0.16 | 5.3E−04 | −3.54 | 13.49 |
| k_Bacteria; p_Firmicutes; c_Clostrida; o_Clostridiales; f_Lachnospiraceae | v4916 | 83 | 0.02 | 80 | 0.38 | 5.4E−04 | −3.53 | 15.56 |
| k_Bacteria; p_Firmicutes; c_Clostrida; o_Clostridiales; f_Veillonellaceae; | v8010 | 83 | 0.16 | 80 | 0.00 | 5.6E−04 | 3.52 | 0.00 |
| g_Dialister | | | | | | | | |
| k_Bacteria; p_Firmicutes; c_Clostrida; o_Clostridiales; f_Lachnospiraceae; | v1423 | 83 | 0.13 | 80 | 1.03 | 5.9E−04 | −3.50 | 7.73 |
| g_Blautia | | | | | | | | |
| k_Bacteria; p_Firmicutes; c_Clostrida; o_Clostridiales; f_Lachnospiraceae; | v5333 | 83 | 0.07 | 80 | 0.29 | 7.9E−04 | −3.42 | 3.98 |
| g_Blautia | | | | | | | | |

TABLE 9
Gut microbes corresponding with high and low GTA-476 levels:

| OTUs | OTUs | N1 | meanQ1 | N2 | meanQ5 | p value | tstatistit | ratio |
|---|---|---|---|---|---|---|---|---|
| GTA475431 | | 82 | 0.66 | 79 | 2.43 | 5.5E−80 | −36.97 | |
| k_Bacteria; p_Firmicutes; c_Clostrida; o_Clostridiales; f_Lachnospiraceae; | v1007 | 82 | 0.12 | 79 | 1.63 | 5.3E−06 | −4.71 | 13.39 |
| g_Blautia | | | | | | | | |
| k_Bacteria; p_Firmicutes; c_Clostrida; o_Clostridiales; f_Lachnospiraceae; | v527 | 82 | 0.46 | 79 | 4.01 | 1.0E−05 | −4.55 | 8.66 |
| g_Blautia | | | | | | | | |
| k_Bacteria; p_Firmicutes; c_Clostrida; o_Clostridiales; f_Lachnospiraceae; | v335 | 82 | 0.89 | 79 | 5.77 | 1.2E−05 | −4.52 | 6.48 |
| g_Blautia | | | | | | | | |
| k_Bacteria; p_Firmicutes; c_Clostrida; o_Clostridiales; f_Ruminococcaceae; | v407 | 82 | 0.68 | 79 | 3.65 | 3.4E−05 | −4.27 | 5.34 |
| g_Faecalibacterium; s_prausnitzii | | | | | | | | |
| k_Bacteria; p_Firmicutes; c_Clostrida; o_Clostridiales; f_Ruminococcaceae; | v324 | 82 | 1.26 | 79 | 4.99 | 7.1E−05 | −4.08 | 3.97 |
| g_Faecalibacterium; s_prausnitzii | | | | | | | | |
| k_Bacteria; p_Firmicutes; c_Clostrida; o_Clostridiales; f_Lachnospiraceae; | v782 | 82 | 1.56 | 79 | 0.19 | 8.6E−05 | 4.03 | 0.12 |
| g_Coprococcus | | | | | | | | |
| k_Bacteria; p_Firmicutes; c_Clostrida; o_Clostridiales; f_Lachnospiraceae; | v1423 | 82 | 0.15 | 79 | 1.25 | 1.0E−04 | −3.99 | 8.56 |
| g_Blautia | | | | | | | | |
| k_Bacteria; p_Firmicutes; c_Clostrida; o_Clostridiales; f_Lachnospiraceae; | v284 | 82 | 4.00 | 79 | 0.94 | 1.9E−04 | 3.82 | 0.23 |
| g_Blautia | | | | | | | | |
| k_Bacteria; p_Firmicutes; c_Clostrida; o_Clostridiales; f_Ruminococcaceae; | v4393 | 82 | 0.00 | 79 | 0.20 | 2.6E−04 | −3.74 | |
| g_Oscillospira | | | | | | | | |
| k_Bacteria; p_Firmicutes; c_Clostrida; o_Clostridiales; f_Lachnospiraceae; | v1702 | 82 | 0.11 | 79 | 1.08 | 2.8E−04 | −3.71 | 9.80 |
| g_Coprococcus | | | | | | | | |
| k_Bacteria; p_Firmicutes; c_Clostrida; o_Clostridiales; f_Lachnospiraceae; | v5402 | 82 | 0.02 | 79 | 0.22 | 3.3E−04 | −3.67 | 8.82 |
| g_Roseburia | | | | | | | | |
| k_Bacteria; p_Firmicutes; c_Clostrida; o_Clostridiales; f_Lachnospiraceae | v2563 | 82 | 0.05 | 79 | 0.82 | 3.7E−04 | −3.64 | 16.87 |
| k_Bacteria; p_Firmicutes; c_Clostrida; o_Clostridiales; f_Lachnospiraceae | v957 | 82 | 0.35 | 79 | 1.76 | 4.1E−04 | −3.61 | 4.98 |
| k_Bacteria; p_Firmicutes; c_Clostrida; o_Clostridiales; f_Lachnospiraceae; | v492 | 82 | 2.33 | 79 | 0.42 | 4.3E−04 | 3.60 | 0.18 |
| g_Coprococcus | | | | | | | | |
| k_Bacteria; p_Bacteroidetes; c_Bacteroidia; o_Bacteroidales; f_Bacteroidaceae; | v158 | 82 | 7.39 | 79 | 2.29 | 4.7E−04 | 3.57 | 0.31 |
| g_Bacteroides | | | | | | | | |
| k_Bacteria; p_Firmicutes; c_Clostrida; o_Clostridiales; f_Lachnospiraceae; | v1391 | 82 | 0.12 | 79 | 0.96 | 5.6E−04 | −3.52 | 7.89 |
| g_Roseburia; s_faecis | | | | | | | | |
| k_Bacteria; p_Firmicutes; c_Clostrida; o_Clostridiales; f_Lachnospiraceae; | v323 | 82 | 1.44 | 79 | 5.37 | 5.6E−04 | −3.52 | 3.73 |
| g_Anaerostipes | | | | | | | | |
| k_Bacteria; p_Firmicutes; c_Clostrida; o_Clostridiales; f_Lachnospiraceae | v1540 | 82 | 0.21 | 79 | 1.14 | 5.7E−04 | −3.52 | 5.50 |
| k_Bacteria; p_Firmicutes; c_Clostrida; o_Clostridiales; f_Lachnospiraceae | v1106 | 82 | 0.50 | 79 | 1.80 | 6.0E−04 | −3.50 | 3.59 |
| k_Bacteria; p_Firmicutes; c_Clostrida; o_Clostridiales; f_Ruminococcaceae; | v2170 | 82 | 0.05 | 79 | 0.80 | 6.5E−04 | −3.48 | 16.35 |
| g_Ruminococcus | | | | | | | | |
| k_Bacteria; p_Firmicutes; c_Clostrida; o_Clostridiales; f_Lachnospiraceae; | v1938 | 82 | 0.60 | 79 | 0.11 | 7.2E−04 | 3.45 | 0.19 |
| g_Coprococcus | | | | | | | | |
| k_Bacteria; p_Firmicutes; c_Clostrida; o_Clostridiales; f_Lachnospiraceae | v4916 | 82 | 0.02 | 79 | 0.43 | 7.5E−04 | −3.44 | 17.65 |
| k_Bacteria; p_Firmicutes; c_Clostrida; o_Clostridiales; f_Lachnospiraceae; | v188 | 82 | 6.68 | 79 | 2.09 | 7.9E−04 | 3.42 | 0.31 |
| g_Blautia | | | | | | | | |
| k_Bacteria; p_Bacteroidetes; c_Bacteroidia; o_Bacteroidales; f_Bacteroidaceae; | v490 | 82 | 3.76 | 79 | 0.46 | 8.5E−04 | 3.40 | 0.12 |
| g_Bacteroides | | | | | | | | |
| k_Bacteria; p_Bacteroidetes; c_Bacteroidia; o_Bacteroidales; f_Bacteroidaceae; | v2930 | 82 | 0.04 | 79 | 0.49 | 9.0E−04 | −3.39 | 13.49 |
| g_Bacteroides | | | | | | | | |
| k_Bacteria; p_Firmicutes; c_Clostrida; o_Clostridiales; f_Lachnospiraceae; | v1691 | 82 | 0.18 | 79 | 0.65 | 9.3E−04 | −3.37 | 3.53 |
| g_Roseburia | | | | | | | | |

TABLE 10
Gut microbes corresponding with high and low GTA-478 levels:

| OTUs | OTUs | N1 | meanQ1 | N2 | meanQ5 | p value | tstatistic | ratio |
|---|---|---|---|---|---|---|---|---|
| GTA477433 | | 81 | 0.57 | 81 | 2.18 | 1.0E−75 | −34.34 | |
| k_Bacteria; p_Firmicutes; c_Clostrida; o_Clostridiales; f_Lachnospiraceae; | v782 | 81 | 1.30 | 81 | 0.20 | 3.1E−05 | 4.29 | 0.15 |
| g_Coprococcus | | | | | | | | |
| k_Bacteria; p_Firmicutes; c_Clostrida; o_Clostridiales; f_Lachnospiraceae; | v527 | 81 | 0.46 | 81 | 3.38 | 5.1E−05 | −4.16 | 7.41 |
| g_Blautia | | | | | | | | |
| k_Bacteria; p_Firmicutes; c_Clostrida; o_Clostridiales; f_Lachnospiraceae; | v284 | 81 | 4.28 | 81 | 0.95 | 5.6E−05 | 4.14 | 0.22 |
| g_Blautia | | | | | | | | |
| k_Bacteria; p_Firmicutes; c_Clostrida; o_Clostridiales; f_Lachnospiraceae; | v492 | 81 | 1.75 | 81 | 0.38 | 8.5E−05 | 4.03 | 0.22 |
| g_Coprococcus | | | | | | | | |
| k_Bacteria; p_Firmicutes; c_Clostrida; o_Clostridiales; f_Lachnospiraceae; | v385 | 81 | 0.93 | 81 | 4.70 | 9.5E−05 | −4.00 | 5.08 |
| g_Blautia | | | | | | | | |
| k_Bacteria; p_Firmicutes; c_Clostrida; o_Clostridiales; f_Lachnospiraceae; | v188 | 81 | 7.20 | 81 | 1.95 | 1.5E−04 | 3.89 | 0.27 |
| g_Blautia | | | | | | | | |
| k_Bacteria; p_Actinobacteria; c_Actinobacteria; o_Bifidobacteriales; | v949 | 81 | 0.10 | 81 | 0.90 | 1.9E−04 | −3.82 | 9.13 |
| f_Bifidobacteriaceae; g_Bifidobacterium; s_adolescentis | | | | | | | | |
| k_Bacteria; p_Firmicutes; c_Clostrida; o_Clostridiales; f_Lachnospiraceae; | v1007 | 81 | 0.22 | 81 | 1.46 | 2.2E−04 | −3.78 | 6.56 |
| g_Blautia | | | | | | | | |
| k_Bacteria; p_Firmicutes; c_Clostrida; o_Clostridiales; f_Ruminococcaceae; | v407 | 81 | 0.72 | 81 | 3.02 | 2.6E−04 | −3.74 | 4.22 |
| g_Faecalibacterium; s_prausnitzii | | | | | | | | |
| k_Bacteria; p_Firmicutes; c_Clostrida; o_Clostridiales; f_Ruminococcaceae | v13337 | 81 | 0.00 | 81 | 0.15 | 2.7E−04 | −3.73 | |
| k_Bacteria; p_Bacteroidetes; c_Bacteroidia; o_Bacteroidales; f_Bacteroidaceae; | v490 | 81 | 4.17 | 81 | 0.40 | 3.4E−04 | 3.66 | 0.09 |
| g_Bacteroides | | | | | | | | |
| k_Bacteria; p_Bacteroidetes; c_Bacteroidia; o_Bacteroidales; f_Bacteroidaceae; | v2930 | 81 | 0.00 | 81 | 0.48 | 3.4E−04 | −3.66 | |
| g_Bacteroides | | | | | | | | |
| k_Bacteria; p_Firmicutes; c_Clostrida; o_Clostridiales; f_Lachnospiraceae | v4916 | 81 | 0.02 | 81 | 0.38 | 4.7E−04 | −3.57 | 15.50 |
| k_Bacteria; p_Firmicutes; c_Clostrida; o_Clostridiales; f_Ruminococcaceae | v8216 | 81 | 0.01 | 81 | 0.19 | 4.8E−04 | −3.57 | 15.00 |
| k_Bacteria; p_Bacteroidetes; c_Bacteroidia; o_Bacteroidales; f_Bacteroidaceae; | v529 | 81 | 0.14 | 81 | 1.41 | 7.2E−04 | −3.45 | 10.36 |
| g_Bacteroides | | | | | | | | |
| k_Bacteria; p_Firmicutes; c_Clostrida; o_Clostridiales; f_Ruminococcaceae; | v324 | 81 | 1.26 | 81 | 4.44 | 7.2E−04 | −3.45 | 3.53 |
| g_Faecalibacterium; s_prausnitzii | | | | | | | | |
| k_Bacteria; p_Firmicutes; c_Clostrida; o_Clostridiales; f_Lachnospiraceae | v1884 | 81 | 0.42 | 81 | 0.01 | 7.3E−04 | 3.44 | 0.03 |
| k_Bacteria; p_Firmicutes; c_Clostrida; o_Clostridiales; f_Ruminococcaceae | v942 | 81 | 0.30 | 81 | 1.56 | 7.8E−04 | −3.43 | 5.25 |
| k_Bacteria; p_Bacteroidetes; c_Bacteroidia; o_Bacteroidales; f_Bacteroidaceae; | v158 | 81 | 7.56 | 81 | 2.75 | 8.2E−04 | 3.41 | 0.36 |
| g_Bacteroides | | | | | | | | |
| k_Bacteria; p_Firmicutes; c_Clostrida; o_Clostridiales; f_Lachnospiraceae; | v1702 | 81 | 0.12 | 81 | 1.01 | 8.4E−04 | −3.40 | 8.20 |
| g_Coprococcus | | | | | | | | |
| k_Bacteria; p_Actinobacteria; c_Coriobacteriia; o_Coriobacteriales; | v1305 | 81 | 0.11 | 81 | 1.04 | 8.5E−04 | −3.40 | 9.33 |
| f_Coriobacteriaceae; g_Collinsella; s_aerofaciens | | | | | | | | |
| k_Bacteria; p_Bacteroidetes; c_Bacteroidia; o_Bacteroidales; f_Bacteroidaceae; | v396 | 81 | 2.85 | 81 | 1.22 | 8.8E−04 | 3.39 | 0.43 |
| g_Bacteroides | | | | | | | | |
| k_Bacteria; p_Firmicutes; c_Clostrida; o_Clostridiales; f_Ruminococcaceae; | v7546 | 81 | 0.17 | 81 | 0.01 | 9.0E−04 | 3.38 | 0.07 |
| g_Faecalibacterium; s_prausnitzii | | | | | | | | |
| k_Bacteria; p_Firmicutes; c_Clostrida; o_Clostridiales; f_Lachnospiraceae; | v1218 | 81 | 0.31 | 81 | 1.37 | 9.3E−04 | −3.37 | 4.44 |
| g_Blautia | | | | | | | | |

TABLE 11
Gut microbes corresponding with high and low GTA-484 levels:

| OTUs | OTUs | N1 | meanQ1 | N2 | meanQ5 | p value | tstatistic | ratio |
|---|---|---|---|---|---|---|---|---|
| GTA483315 | | 82 | 0.21 | 81 | 1.18 | 1.31E−52 | −22.94 | |
| k_Bacteria; p_Firmicutes; c_Clostrida; o_Clostridiales; f_Lachnospiraceae; | v5333 | 82 | 0.04 | 81 | 0.26 | 1.01E−04 | −3.99 | 7.09 |
| g_Blautia | | | | | | | | |
| k_Bacteria; p_Bacteroidetes; c_Bacteroidia; o_Bacteroidales; f_Bacteroidaceae; | v490 | 82 | 4.33 | 81 | 0.23 | 1.09E−04 | 3.97 | 0.05 |
| g_Bacteroides | | | | | | | | |
| k_Bacteria; p_Firmicutes; c_Clostrida; o_Clostridiales; f_Lachnospiraceae; | v385 | 82 | 0.82 | 81 | 4.21 | 2.83E−04 | −3.71 | 5.15 |
| g_Blautia | | | | | | | | |
| k_Bacteria; p_Firmicutes; c_Clostrida; o_Clostridiales; f_Lachnospiraceae; | v527 | 82 | 0.48 | 81 | 2.90 | 3.20E−04 | −3.68 | 6.10 |
| g_Blautia | | | | | | | | |
| k_Bacteria; p_Firmicutes; c_Clostrida; o_Clostridiales; f_Lachnospiraceae; | v1007 | 82 | 0.29 | 81 | 1.51 | 3.67E−04 | −3.64 | 5.15 |
| g_Blautia | | | | | | | | |
| k_Bacteria; p_Firmicutes; c_Clostrida; o_Clostridiales; f_Ruminococcaceae; | v4393 | 82 | 0.01 | 81 | 0.25 | 4.39E−04 | −3.59 | 20.25 |
| g_Oscillospira | | | | | | | | |
| k_Bacteria; p_Firmicutes; c_Clostrida; o_Clostridiales; f_Lachnospiraceae; | v284 | 82 | 4.23 | 81 | 1.28 | 5.74E−04 | 3.51 | 0.30 |
| g_Blautia | | | | | | | | |
| k_Bacteria; p_Firmicutes; c_Clostrida; o_Clostridiales; f_Lachnospiraceae; | v1068 | 82 | 0.96 | 81 | 0.22 | 6.36E−04 | 3.48 | 0.23 |
| g_Blautia | | | | | | | | |
| k_Bacteria; p_Bacteroidetes; c_Bacteroidia; o_Bacteroidales; f_Bacteroidaceae; | v14672 | 82 | 0.15 | 81 | 0.00 | 3.87E−04 | 3.39 | 0.00 |
| g_Bacteroides | | | | | | | | |
| k_Bacteria; p_Firmicutes; c_Clostrida; o_Clostridiales; f_Ruminococcaceae; | v2874 | 82 | 0.41 | 81 | 0.06 | 9.90E−04 | 3.35 | 0.15 |
| g_Faecalibacterium; s_prausnitzii | | | | | | | | |

TABLE 12
Gut microbes corresponding with high and low GTA-490 levels:

| OTUs | OTUs | N1 | meanQ1 | N2 | meanQ5 | p value | tstatistic | ratio |
|---|---|---|---|---|---|---|---|---|
| GTA489445 | | 81 | 0.51 | 80 | 1.92 | 6.7E−66 | −29.26 | |
| k_Bacteria; p_Firmicutes; c_Clostrida; o_Clostridiales; f_Lachnospiraceae; | v1007 | 81 | 0.22 | 80 | 1.71 | 2.6E−05 | −4.33 | 7.71 |
| g_Blautia | | | | | | | | |
| k_Bacteria; p_Firmicutes; c_Clostrida; o_Clostridiales; f_Lachnospiraceae; | v527 | 81 | 0.49 | 80 | 4.10 | 3.1E−05 | −4.29 | 8.30 |
| g_Blautia | | | | | | | | |
| k_Bacteria; p_Firmicutes; c_Clostrida; o_Clostridiales; f_Ruminococcaceae; | v407 | 81 | 0.72 | 80 | 3.51 | 6.1E−05 | −4.12 | 4.91 |
| g_Faecalibacterium; s_prausnitzii | | | | | | | | |
| k_Bacteria; p_Firmicutes; c_Clostrida; o_Clostridiales; f_Lachnospiraceae; | v385 | 81 | 0.94 | 80 | 5.51 | 6.9E−05 | −4.09 | 5.88 |
| g_Blautia | | | | | | | | |
| k_Bacteria; p_Firmicutes; c_Clostrida; o_Clostridiales; f_Lachnospiraceae; | v284 | 81 | 4.09 | 80 | 0.85 | 1.1E−04 | 3.98 | 0.21 |
| g_Blautia | | | | | | | | |
| k_Bacteria; p_Firmicutes; c_Clostrida; o_Clostridiales; f_Ruminococcaceae | v13337 | 81 | 0.01 | 80 | 0.19 | 1.6E−04 | −3.86 | 15.19 |
| k_Bacteria; p_Firmicutes; c_Clostrida; o_Clostridiales; f_Lachnospiraceae; | v1068 | 81 | 0.70 | 80 | 0.09 | 2.0E−04 | 3.81 | 0.12 |
| g_Blautia | | | | | | | | |
| k_Bacteria; p_Firmicutes; c_Clostrida; o_Clostridiales | v1156 | 81 | 0.06 | 80 | 1.61 | 2.2E−04 | −3.78 | 26.12 |
| k_Bacteria; p_Bacteroidetes; c_Bacteroidia; o_Bacteroidales; f_Bacteroidaceae; | v9826 | 81 | 0.22 | 80 | 0.00 | 3.8E−04 | 3.63 | 0.00 |
| g_Bacteroides; s_uniformis | | | | | | | | |
| k_Bacteria; p_Firmicutes; c_Clostrida; o_Clostridiales; f_Lachnospiraceae; | v3283 | 81 | 0.11 | 80 | 0.61 | 4.9E−04 | −3.56 | 5.51 |
| g_Dorea; s_formicigenerans | | | | | | | | |
| k_Bacteria; p_Firmicutes; c_Clostrida; o_Clostridiales; f_Lachnospiraceae; | v1702 | 81 | 0.11 | 80 | 0.93 | 5.0E−04 | −3.55 | 8.33 |
| g_Coprococcus | | | | | | | | |
| k_Bacteria; p_Firmicutes; c_Clostrida; o_Clostridiales; f_Lachnospiraceae; | v1423 | 81 | 0.21 | 80 | 1.31 | 6.2E−04 | −3.49 | 6.25 |
| g_Blautia | | | | | | | | |
| k_Bacteria; p_Firmicutes; c_Clostrida; o_Clostridiales; f_Ruminococcaceae; | v324 | 81 | 1.47 | 80 | 4.74 | 6.4E−04 | −3.48 | 3.22 |
| g_Faecalibacterium; s_prausnitzii | | | | | | | | |
| k_Bacteria; p_Firmicutes; c_Clostrida; o_Clostridiales; f_Lachnospiraceae | v4916 | 81 | 0.02 | 80 | 0.36 | 6.6E−04 | −3.48 | 14.68 |
| k_Bacteria; p_Bacteroidetes; c_Bacteroidia; o_Bacteroidales; f_Rikenellaceae; | v5505 | 81 | 0.05 | 80 | 0.29 | 6.8E−04 | −3.47 | 5.82 |
| g_Bacteroides | | | | | | | | |
| k_Bacteria; p_Bacteroidetes; c_Bacteroidia; o_Bacteroidales; f_Bacteroidaceae; | v158 | 81 | 7.33 | 80 | 2.26 | 7.0E−04 | 3.46 | 0.31 |
| g_Bacteroides | | | | | | | | |
| k_Bacteria; p_Firmicutes; c_Clostrida; o_Clostridiales; f_Lachnospiraceae; | v5333 | 81 | 0.06 | 80 | 0.28 | 7.5E−04 | −3.44 | 4.45 |
| g_Blautia | | | | | | | | |
| k_Bacteria; p_Bacteroidetes; c_Bacteroidia; o_Bacteroidales; f_Bacteroidaceae; | v184 | 81 | 11.21 | 80 | 2.55 | 8.1E−04 | 3.41 | 0.23 |
| g_Bacteroides | | | | | | | | |
| k_Bacteria; p_Firmicutes; c_Clostrida; o_Clostridiales; f_Lachnospiraceae; | v188 | 81 | 6.64 | 80 | 2.10 | 8.4E−04 | 3.41 | 0.32 |
| g_Blautia | | | | | | | | |
| k_Bacteria; p_Bacteroidetes; c_Bacteroidia; o_Bacteroidales; f_Bacteroidaceae; | v490 | 81 | 5.31 | 80 | 0.46 | 8.4E−04 | 3.41 | 0.09 |
| g_Bacteroides | | | | | | | | |
| k_Bacteria; p_Firmicutes; c_Clostrida; o_Clostridiales; f_Ruminococcaceae | v8216 | 81 | 0.01 | 80 | 0.19 | 9.1E−04 | −3.38 | 15.19 |
| k_Bacteria; p_Firmicutes; c_Clostrida; o_Clostridiales; f_Clostridiaceae; | v8086 | 81 | 0.00 | 80 | 0.16 | 9.5E−04 | −3.37 | |
| g_SMB53 | | | | | | | | |
| k_Bacteria; p_Firmicutes; c_Clostrida; o_Clostridiales; f_Ruminococcaceae | v3864 | 81 | 0.01 | 80 | 0.28 | 9.5E−04 | −3.37 | 22.27 |

TABLE 13
Gut microbes corresponding with high and low GTA-492 levels:

| OTUs | OTUs | N1 | meanQ1 | N2 | meanQ5 | p value | tstatistic | ratio |
|---|---|---|---|---|---|---|---|---|
| GTA491241 | | 81 | 0.02 | 79 | 0.75 | 1.01E−62 | −27.80 | |
| k_Bacteria; p_Bacteroidetes; c_Bacteroidia; o_Bacteroidales; | v158 | 81 | 10.64 | 79 | 1.89 | 3.78E−07 | 5.12 | 0.18 |
| f_Bacteroidaceae; g_Bacteroides | | | | | | | | |
| k_Bacteria; p_Firmicutes; c_Clostrida; o_Clostridiales; f_Ruminococcaceae; | v407 | 81 | 0.46 | 79 | 4.14 | 2.60E−06 | −4.88 | 9.06 |
| g_Faecalibacterium; s_prausnitzii | | | | | | | | |
| k_Bacteria; p_Firmicutes; c_Clostrida; o_Clostridiales; f_Lachnospiraceae; | v527 | 81 | 0.38 | 79 | 3.97 | 2.98E−06 | −4.85 | 10.39 |
| g_Blautia | | | | | | | | |
| k_Bacteria; p_Firmicutes; c_Clostrida; o_Clostridiales; f_Ruminococcaceae; | v967 | 81 | 0.21 | 79 | 2.01 | 7.62E−06 | −4.63 | 9.59 |
| g_Faecalibacterium; s_prausnitzii | | | | | | | | |
| k_Bacteria; p_Firmicutes; c_Clostrida; o_Clostridiales; f_Lachnospiraceae; | v1007 | 81 | 0.19 | 79 | 1.57 | 7.67E−06 | −4.63 | 8.48 |
| g_Blautia | | | | | | | | |
| k_Bacteria; p_Firmicutes; c_Clostrida; o_Clostridiales; f_Ruminococcaceae; | v324 | 81 | 0.99 | 79 | 4.99 | 9.21E−06 | −4.58 | 5.05 |
| g_Faecalibacterium; s_prausnitzii | | | | | | | | |
| k_Bacteria; p_Firmicutes; c_Clostrida; o_Clostridiales; f_Lachnospiraceae; | v3283 | 81 | 0.05 | 79 | 0.66 | 9.30E−06 | −4.53 | 13.33 |
| g_Dorea; s_formicigenerans | | | | | | | | |
| k_Bacteria; p_Firmicutes; c_Clostrida; o_Clostridiales; f_Lachnospiraceae; | v1068 | 81 | 0.83 | 79 | 0.06 | 1.19E−05 | 4.52 | 0.08 |
| g_Blautia | | | | | | | | |
| k_Bacteria; p_Firmicutes; c_Clostrida; o_Clostridiales; f_Lachnospiraceae; | v284 | 81 | 3.57 | 79 | 0.48 | 1.41E−05 | 4.48 | 0.13 |
| g_Blautia | | | | | | | | |
| k_Bacteria; p_Bacteroidetes; c_Bacteroidia; o_Bacteroidales; | v396 | 81 | 3.42 | 79 | 1.15 | 1.79E−05 | 4.42 | 0.34 |
| f_Bacteroidaceae; g_Bacteroides | | | | | | | | |
| k_Bacteria; p_Firmicutes; c_Clostrida; o_Clostridiales; f_Lachnospiraceae; | v385 | 81 | 0.60 | 79 | 5.10 | 2.05E−05 | −4.39 | 8.43 |
| g_Blautia | | | | | | | | |
| k_Bacteria; p_Firmicutes; c_Clostrida; o_Clostridiales; f_Ruminococcaceae; | v717 | 81 | 0.47 | 79 | 2.77 | 5.37E−05 | −4.15 | 5.91 |
| g_Faecalibacterium; s_prausnitzii | | | | | | | | |
| k_Bacteria; p_Firmicutes; c_Clostrida; o_Clostridiales; f_Ruminococcaceae; | v125 | 81 | 2.52 | 79 | 10.48 | 6.00E−05 | −4.12 | 4.16 |
| g_Oscillospira | | | | | | | | |
| k_Bacteria; p_Bacteroidetes; c_Bacteroidia; o_Bacteroidales; | v2977 | 81 | 0.43 | 79 | 0.04 | 9.02E−05 | 4.02 | 0.09 |
| f_Bacteroidaceae; g_Bacteroides | | | | | | | | |
| k_Bacteria; p_Firmicutes; c_Clostrida; o_Clostridiales; f_Lachnospiraceae; | v2169 | 81 | 0.09 | 79 | 0.65 | 9.11E−05 | −4.02 | 7.47 |
| g_Dorea | | | | | | | | |
| k_Bacteria; p_Firmicutes; c_Clostrida; o_Clostridiales; f_Lachnospiraceae; | v282 | 81 | 1.53 | 79 | 6.44 | 9.60E−05 | −4.00 | 4.21 |
| g_Blautia | | | | | | | | |
| k_Bacteria; p_Firmicutes; c_Clostrida; o_Clostridiales; f_Clostridaceae | v4592 | 81 | 0.01 | 79 | 0.61 | 1.19E−04 | −3.95 | 49.22 |
| k_Bacteria; p_Firmicutes; c_Clostrida; o_Clostridiales; f_Lachnospiraceae; | v5333 | 81 | 0.05 | 79 | 0.30 | 1.28E−04 | −3.93 | 6.15 |
| g_Blautia | | | | | | | | |
| k_Bacteria; p_Firmicutes; c_Clostrida; o_Clostridiales; f_Lachnospiraceae; | v5401 | 81 | 0.04 | 79 | 0.41 | 1.75E−04 | −3.84 | 10.94 |
| g_Dorea; s_formicigenerans | | | | | | | | |
| k_Bacteria; p_Firmicutes; c_Clostrida; o_Clostridiales; f_Lachnospiraceae; | v2737 | 81 | 0.05 | 79 | 0.58 | 1.92E−04 | −3.82 | 11.79 |
| g_Blautia | | | | | | | | |
| k_Bacteria; p_Firmicutes; c_Clostrida; o_Clostridiales; f_Lachnospiraceae | v1679 | 81 | 0.17 | 79 | 1.01 | 2.15E−04 | −3.79 | 5.86 |
| k_Bacteria; p_Firmicutes; c_Clostrida; o_Clostridiales; f_Lachnospiraceae; | v1423 | 81 | 0.16 | 79 | 1.15 | 2.46E−04 | −3.75 | 7.18 |
| g_Blautia | | | | | | | | |
| k_Bacteria; p_Firmicutes; c_Clostrida; o_Clostridiales; f_Ruminococcaceae | v111 | 81 | 4.33 | 79 | 15.13 | 2.48E−04 | −3.75 | 3.49 |
| k_Bacteria; p_Firmicutes; c_Clostrida; o_Clostridiales; f_Lachnospiraceae; | v1218 | 81 | 0.22 | 79 | 1.30 | 2.58E−04 | −3.74 | 5.87 |
| g_Blautia | | | | | | | | |
| k_Bacteria; p_Firmicutes; c_Clostrida; o_Clostridiales | v183 | 81 | 1.95 | 79 | 6.38 | 2.83E−04 | −3.71 | 3.27 |
| k_Bacteria; p_Bacteroidetes; c_Bacteroidia; o_Bacteroidales; | v2002 | 81 | 0.69 | 79 | 0.19 | 2.86E−04 | 3.71 | 0.27 |
| f_Bacteroidaceae; g_Bacteroides | | | | | | | | |
| k_Bacteria; p_Firmicutes; c_Clostrida; o_Clostridiales; f_Lachnospiraceae | v1106 | 81 | 0.27 | 79 | 1.72 | 3.53E−04 | −3.65 | 6.34 |
| k_Bacteria; p_Bacteroidetes; c_Bacteroidia; o_Bacteroidales; | v2503 | 81 | 0.49 | 79 | 0.14 | 3.59E−04 | 3.65 | 0.28 |
| f_Bacteroidaceae; g_Bacteroides | | | | | | | | |
| k_Bacteria; p_Bacteroidetes; c_Bacteroidia; o_Bacteroidales; | v3564 | 81 | 0.05 | 79 | 0.59 | 3.84E−04 | −3.63 | 12.05 |
| f_Porphyromonadaceae; g_Parabacteroides; s_distasonis | | | | | | | | |
| k_Bacteria; p_Firmicutes; c_Clostrida; o_Clostridiales; f_Ruminococcaceae | v1998 | 81 | 0.16 | 79 | 0.95 | 3.85E−04 | −3.63 | 5.92 |
| k_Bacteria; p_Firmicutes; c_Clostrida; o_Clostridiales; f_Lachnospiraceae; | v952 | 81 | 0.85 | 79 | 0.23 | 4.29E−04 | 3.60 | 0.27 |
| g_Blautia | | | | | | | | |
| k_Bacteria; p_Firmicutes; c_Clostrida; o_Clostridiales; f_Ruminococcaceae; | v871 | 81 | 0.33 | 79 | 2.20 | 4.71E−04 | −3.57 | 6.61 |
| g_Faecalibacterium; s_prausnitzii | | | | | | | | |
| k_Bacteria; p_Bacteroidetes; c_Bacteroidia; o_Bacteroidales; | v7569 | 81 | 0.40 | 79 | 0.00 | 4.72E−04 | 3.57 | 0.00 |
| f_Bacteroidaceae; g_Bacteroides | | | | | | | | |
| k_Bacteria; p_Firmicutes; c_Clostrida; o_Clostridiales; f_Lachnospiraceae; | v188 | 81 | 5.72 | 79 | 1.56 | 4.98E−04 | 3.56 | 0.27 |
| g_Blautia | | | | | | | | |
| k_Bacteria; p_Bacteroidetes; c_Bacteroidia; o_Bacteroidales; | v425 | 81 | 3.62 | 79 | 0.14 | 6.21E−04 | 3.49 | 0.04 |
| f_Bacteroidaceae; g_Bacteroides | | | | | | | | |
| k_Bacteria; p_Firmicutes; c_Clostrida; o_Clostridiales; f_Lachnospiraceae | v4916 | 81 | 0.01 | 79 | 0.32 | 6.34E−04 | −3.49 | 25.63 |
| k_Bacteria; p_Firmicutes; c_Clostrida; o_Clostridiales; f_Lachnospiraceae; | v979 | 81 | 0.42 | 79 | 1.71 | 6.62E−04 | −3.47 | 4.07 |
| g_Roseburia | | | | | | | | |
| k_Bacteria; p_Firmicutes; c_Clostrida; o_Clostridiales; f_Ruminococcaceae; | v11393 | 81 | 0.00 | 79 | 0.15 | 6.94E−04 | −3.46 | |
| g_Faecalibacterium; s_prausnitzii | | | | | | | | |
| k_Bacteria; p_Bacteroidetes; c_Bacteroidia; o_Bacteroidales; | v19274 | 81 | 0.00 | 79 | 0.15 | 6.94E−04 | −3.46 | |
| f_Rikenellaceae; g_Bacteroides | | | | | | | | |
| k_Bacteria; p_Bacteroidetes; c_Bacteroidia; o_Bacteroidales; | v4067 | 81 | 0.06 | 79 | 0.42 | 7.59E−04 | −3.43 | 6.77 |
| f_Bacteroidaceae; g_Bacteroides | | | | | | | | |
| k_Bacteria; p_Firmicutes; c_Clostrida; o_Clostridiales; f_Ruminococcaceae | v360 | 81 | 1.05 | 79 | 3.23 | 7.64E−04 | −3.43 | 3.08 |
| k_Bacteria; p_Firmicutes; c_Clostrida; o_Clostridiales; | v535 | 81 | 0.83 | 79 | 2.72 | 7.94E−04 | −3.42 | 3.29 |
| f_[Mogibacteriaceae] | | | | | | | | |
| k_Bacteria; p_Firmicutes; c_Clostrida; o_Clostridiales; f_Ruminococcaceae; | v3353 | 81 | 0.01 | 79 | 0.46 | 8.14E−04 | −3.41 | 36.91 |
| g_Oscillospira | | | | | | | | |
| k_Bacteria; p_Firmicutes; c_Clostrida; o_Clostridiales; f_Ruminococcaceae; | v12412 | 81 | 0.01 | 79 | 0.19 | 8.28E−04 | −3.41 | 15.38 |
| g_Oscillospira | | | | | | | | |
| k_Bacteria; p_Proteobacteria; c_Betaproteobacteria; o_Burkholderiales, | v880 | 81 | 1.53 | 79 | 0.22 | 8.39E−04 | 3.40 | 0.14 |
| f_Alcaligenaceae; g_Sutterella | | | | | | | | |
| k_Bacteria; p_Firmicutes; c_Clostrida; o_Clostridiales; f_Lachnospiraceae | v8584 | 81 | 0.01 | 79 | 0.20 | 8.47E−04 | −3.40 | 16.41 |
| k_Bacteria; p_Firmicutes; c_Clostrida; o_Clostridiales; f_Veillonellaceae; | v479 | 81 | 0.05 | 79 | 6.11 | 8.56E−04 | −3.40 | 123.81 |
| g_Phascolarctobacterium | | | | | | | | |
| k_Bacteria; p_Firmicutes; c_Erysipelotrichi; o_Erysipelotrichales; | v14817 | 81 | 0.00 | 79 | 0.16 | 8.73E−04 | −3.39 | |
| f_Erysipelotrichaceae; g_[Eubacterium]; s_biforme | | | | | | | | |
| k_Bacteria; p_Proteobacteria; c_Betaproteobacteria; o_Burkholderiales, | v2949 | 81 | 0.56 | 79 | 0.03 | 9.28E−04 | 3.38 | 0.05 |
| f_Alcaligenaceae; g_Sutterella | | | | | | | | |
| k_Bacteria; p_Firmicutes; c_Clostrida; o_Clostridiales; f_Clostridiaceae | v12858 | 81 | 0.00 | 79 | 0.20 | 9.30E−04 | −3.37 | |
| k_Bacteria; p_Bacteroidetes; c_Bacteroidia; o_Bacteroidales; | v333 | 81 | 1.25 | 79 | 5.86 | 9.58E−04 | −3.37 | 4.70 |
| f_Bacteroidaceae; g_Bacteroides | | | | | | | | |
| k_Bacteria; p_Firmicutes; c_Clostrida; o_Clostridiales; f_Lachnospiraceae; | v2461 | 81 | 0.14 | 79 | 0.63 | 9.59E−04 | −3.37 | 4.66 |
| g_Coprococcus | | | | | | | | |
| k_Bacteria; p_Proteobacteria; c_Betaproteobacteria; o_Burkholderiales, | v457 | 81 | 2.95 | 79 | 0.66 | 9.81E−04 | 3.36 | 0.22 |
| f_Alcaligenaceae; g_Sutterella | | | | | | | | |
| k_Bacteria; p_Firmicutes; c_Clostrida; o_Clostridiales; f_Lachnospiraceae; | v1702 | 81 | 0.14 | 79 | 0.92 | 9.98E−04 | −3.35 | 6.80 |
| g_Coprococcus | | | | | | | | |

TABLE 14
Gut microbes corresponding with high and low GTA-494 levels:

| OTUs | OTUs | N1 | meanQ1 | N2 | meanQ5 | p value | tstatistic | ratio |
|---|---|---|---|---|---|---|---|---|
| GTA493449 | | 81 | 0.76 | 81 | 2.87 | 3.7E−66 | −29.28 | |
| k_Bacteria; p_Firmicutes; c_Clostrida; o_Clostridiales; f_Ruminococcaceae; | v2874 | 81 | 0.58 | 81 | 0.06 | 8.1E−05 | 4.05 | 0.11 |
| g_Faecalibacterium; s_prausnitzii | | | | | | | | |
| k_Bacteria; p_Firmicutes; c_Clostrida; o_Clostridiales; f_Lachnospiraceae; | v527 | 81 | 0.38 | 81 | 2.91 | 1.1E−04 | −3.97 | 7.61 |
| g_Blautia | | | | | | | | |
| k_Bacteria; p_Firmicutes; c_Clostrida; o_Clostridiales; f_Lachnospiraceae; | v385 | 81 | 0.70 | 81 | 4.17 | 1.3E−04 | −3.91 | 5.93 |
| g_Blautia | | | | | | | | |
| k_Bacteria; p_Firmicutes; c_Clostrida; o_Clostridiales; f_Ruminococcaceae; | v407 | 81 | 0.46 | 81 | 3.21 | 1.3E−04 | −3.91 | 7.03 |
| g_Faecalibacterium; s_prausnitzii | | | | | | | | |
| k_Bacteria; p_Firmicutes; c_Clostrida; o_Clostridiales; f_Lachnospiraceae; | v1007 | 81 | 0.16 | 81 | 1.35 | 1.4E−04 | −3.90 | 8.38 |
| g_Blautia | | | | | | | | |
| k_Bacteria; p_Firmicutes; c_Clostrida; o_Clostridiales; f_Lachnospiraceae; | v5333 | 81 | 0.05 | 81 | 0.30 | 1.7E−04 | −3.85 | 6.00 |
| g_Blautia | | | | | | | | |
| k_Bacteria; p_Firmicutes; c_Clostrida; o_Clostridiales; f_Lachnospiraceae; | v284 | 81 | 4.28 | 81 | 1.05 | 2.0E−04 | 3.81 | 0.24 |
| g_Blautia | | | | | | | | |
| k_Bacteria; p_Firmicutes; c_Clostrida; o_Clostridiales; f_Ruminococcaceae; | v967 | 81 | 0.15 | 81 | 1.54 | 2.1E−04 | −3.80 | 10.42 |
| g_Faecalibacterium; s_prausnitzii | | | | | | | | |
| k_Bacteria; p_Firmicutes; c_Clostrida; o_Clostridiales; f_Ruminococcaceae; | v324 | 81 | 0.93 | 81 | 4.21 | 2.2E−04 | −3.78 | 4.55 |
| g_Faecalibacterium; s_prausnitzii | | | | | | | | |
| k_Bacteria; p_Firmicutes; c_Clostrida; o_Clostridiales; f_Ruminococcaceae | v8216 | 81 | 0.00 | 81 | 0.17 | 2.2E−04 | −3.78 | |
| k_Bacteria; p_Firmicutes; c_Clostrida; o_Clostridiales; f_Lachnospiraceae; | v188 | 81 | 7.37 | 81 | 2.14 | 2.8E−04 | 3.71 | 0.29 |
| g_Blautia | | | | | | | | |
| k_Bacteria; p_Firmicutes; c_Clostrida; o_Clostridiales; f_Lachnospiraceae | v2563 | 81 | 0.04 | 81 | 0.65 | 3.8E−04 | −3.63 | 17.67 |
| k_Bacteria; p_Bacteroidetes; c_Bacteroidia; o_Bacteroidales; f_Bacteroidaceae; | v14672 | 81 | 0.19 | 81 | 0.01 | 4.8E−04 | 3.57 | 0.07 |
| g_Bacteroides | | | | | | | | |
| k_Bacteria; p_Firmicutes; c_Clostrida; o_Clostridiales; f_Ruminococcaceae; | v717 | 81 | 0.38 | 81 | 2.27 | 4.9E−04 | −3.56 | 5.94 |
| g_Faecalibacterium; s_prausnitzii | | | | | | | | |
| k_Bacteria; p_Bacteroidetes; c_Bacteroidia; o_Bacteroidales; f_Rikenellaceae; | v5505 | 81 | 0.06 | 81 | 0.31 | 5.7E−04 | −3.51 | 5.00 |
| g_Bacteroides | | | | | | | | |
| k_Bacteria; p_Firmicutes; c_Clostrida; o_Clostridiales; f_Ruminococcaceae | v1384 | 81 | 0.15 | 81 | 0.80 | 6.8E−04 | −3.47 | 5.42 |
| k_Bacteria; p_Bacteroidetes; c_Bacteroidia; o_Bacteroidales; f_Bacteroidaceae; | v333 | 81 | 0.90 | 81 | 4.17 | 7.0E−04 | −3.46 | 4.63 |
| g_Bacteroides | | | | | | | | |
| k_Bacteria; p_Firmicutes; c_Clostrida; o_Clostridiales; f_Ruminococcaceae; | v13634 | 81 | 0.00 | 81 | 0.15 | 8.2E−04 | −3.41 | |
| g_Faecalibacterium; s_prausnitzii | | | | | | | | |
| k_Bacteria; p_Firmicutes; c_Clostrida; o_Clostridiales; f_Lachnospiraceae | v4916 | 81 | 0.02 | 81 | 0.35 | 9.0E−04 | −3.38 | 14.00 |
| k_Bacteria; p_Bacteroidetes; c_Bacteroidia; o_Bacteroidales; f_Bacteroidaceae; | v2930 | 81 | 0.04 | 81 | 0.49 | 9.5E−04 | −3.37 | 13.33 |
| g_Bacteroides | | | | | | | | |
| k_Bacteria; p_Firmicutes; c_Clostrida; o_Clostridiales; f_Lachnospiraceae | v1884 | 81 | 0.37 | 81 | 0.04 | 9.7E−04 | 3.36 | 0.10 |
| k_Bacteria; p_Bacteroidetes; c_Bacteroidia; o_Bacteroidales; f_Bacteroidaceae; | v15255 | 81 | 0.12 | 81 | 0.00 | 9.9E−04 | 3.36 | 0.00 |
| g_Bacteroides; s_uniformis | | | | | | | | |

TABLE 15
Gut microbes corresponding with high and low GTA-502 levels:

| OTUs | OTUs | N1 | meanQ1 | N2 | meanQ5 | p value | tstatistic | ratio | inverse |
|---|---|---|---|---|---|---|---|---|---|
| GTA501457 | | 81 | 0.19 | 80 | 0.89 | 5.2E−55 | −24.12 | | |
| k_Bacteria; p_Firmicutes; c_Clostrida; o_Clostridiales; | v8216 | 81 | 0.00 | 80 | 0.20 | 1.4E−04 | −3.90 | | [symbol] #DIV/0. |
| f_Ruminococcaceae | | | | | | | | | |
| k_Bacteria; p_Bacteroidetes; c_Bacteroidia; o_Bacteroidales; | v12263 | 81 | 0.00 | 80 | 0.16 | 9.5E−04 | −3.37 | | [symbol] #DIV/0. |
| f_Rikenellaceae; g_Bacteroides | | | | | | | | | |
| k_Bacteria; p_Bacteroidetes; c_Bacteroidia; o_Bacteroidales; | v4516 | 81 | 0.01 | 80 | 0.41 | 9.5E−04 | −3.37 | 33.41 | −0.02993 |
| f_Rikenellaceae; g_Bacteroides | | | | | | | | | |
| k_Bacteria; p_Firmicutes; c_Clostrida; o_Clostridiales; | v527 | 81 | 0.43 | 80 | 3.05 | 3.8E−04 | −3.63 | 7.06 | −0.14167 |
| f_Lachnospiraceae; g_Blautia | | | | | | | | | |
| k_Bacteria; p_Firmicutes; c_Clostrida; o_Clostridiales; | v806 | 81 | 1.65 | 80 | 0.65 | 9.1E−04 | 3.38 | 0.39 | −2.54511 |
| f_Lachnospiraceae | | | | | | | | | |
| k_Bacteria; p_Firmicutes; c_Clostrida; o_Clostridiales; | v2371 | 81 | 0.59 | 80 | 0.21 | 1.8E−04 | 3.83 | 0.36 | −2.78867 |
| f_Lachnospiraceae | | | | | | | | | |
| k_Bacteria; p_Firmicutes; c_Clostrida; o_Clostridiales; | v1791 | 81 | 0.90 | 80 | 0.25 | 3.2E−04 | 3.68 | 0.28 | −3.60494 |
| f_Lachnospiraceae; g_Dorea | | | | | | | | | |
| k_Bacteria; p_Firmicutes; c_Clostrida; o_Clostridiales; | v404 | 81 | 3.80 | 80 | 1.00 | 6.5E−04 | 3.48 | 0.26 | −3.80247 |
| f_Lachnospiraceae; g_Coprococcus | | | | | | | | | |
| k_Bacteria; p_Bacteroidetes; c_Bacteroidia; o_Bacteroidales; | v4941 | 81 | 0.37 | 80 | 0.06 | 2.7E−04 | 3.73 | 0.17 | −5.92593 |
| f_Bacteroidaceae; g_Bacteroides | | | | | | | | | |
| k_Bacteria; p_Firmicutes; c_Clostrida; o_Clostridiales; | v4835 | 81 | 0.41 | 80 | 0.01 | 8.6E−04 | 3.40 | 0.03 | −32.5926 |
| f_Lachnospiraceae; g_Coprococcus | | | | | | | | | |

TABLE 16
Gut microbes corresponding with high and low GTA-504 levels:

| OTUs | OTUs | N1 | meanQ1 | N2 | meanQ5 | p value | tstatistic | ratio |
|---|---|---|---|---|---|---|---|---|
| GTA503459 | | 81 | 0.17 | 80 | 0.77 | 8.4E−80 | −36.87 | |
| k_Bacteria; p_Bacteroidetes; c_Bacteroidia; o_Bacteroidales; f_Rikenellaceae; | v3729 | 81 | 0.02 | 80 | 0.53 | 6.9E−05 | −4.09 | 21.26 |
| g_Bacteroides | | | | | | | | |
| k_Bacteria; p_Firmicutes; c_Clostrida; o_Clostridiales; f_Lachnospiraceae; | v3710 | 81 | 0.04 | 80 | 0.35 | 1.4E−04 | −3.91 | 9.45 |
| g_Blautia | | | | | | | | |
| k_Bacteria; p_Bacteroidetes; c_Bacteroidia; o_Bacteroidales; f_Bacteroidaceae; | v9826 | 81 | 0.17 | 80 | 0.00 | 2.5E−04 | 3.75 | 0.00 |
| g_Bacteroides; s_uniformis | | | | | | | | |
| k_Bacteria; p_Firmicutes; c_Clostrida; o_Clostridiales; f_Ruminococcaceae; | v2237 | 81 | 0.63 | 80 | 0.16 | 2.6E−04 | 3.74 | 0.26 |
| g_Faecalibacterium; s_prausnitzii | | | | | | | | |
| k_Bacteria; p_Firmicutes; c_Clostrida; o_Clostridiales; f_Lachnospiraceae; | v1007 | 81 | 0.12 | 80 | 1.20 | 3.3E−04 | −3.67 | 9.72 |
| g_Blautia | | | | | | | | |
| k_Bacteria; p_Bacteroidetes; c_Bacteroidia; o_Bacteroidales; f_Rikenellaceae; | v2034 | 81 | 0.15 | 80 | 0.89 | 3.5E−04 | −3.65 | 5.99 |
| g_Bacteroides | | | | | | | | |
| k_Bacteria; p_Bacteroidetes; c_Bacteroidia; o_Bacteroidales; f_Bacteroidaceae; | v7386 | 81 | 0.27 | 80 | 0.01 | 4.1E−04 | 3.61 | 0.05 |
| g_Bacteroides; s_uniformis | | | | | | | | |
| k_Bacteria; p_Bacteroidetes; c_Bacteroidia; o_Bacteroidales; f_Bacteroidaceae; | v2930 | 81 | 0.00 | 80 | 0.48 | 4.4E−04 | −3.59 | |
| g_Bacteroides | | | | | | | | |
| k_Bacteria; p_Bacteroidetes; c_Bacteroidia; o_Bacteroidales; f_Rikenellaceae; | v47 | 81 | 11.05 | 80 | 52.38 | 5.0E−04 | −3.55 | 4.74 |
| g_Bacteroides | | | | | | | | |
| k_Bacteria; p_Firmicutes; c_Clostrida; o_Clostridiales; f_Ruminococcaceae; | v2874 | 81 | 0.48 | 80 | 0.06 | 5.0E−04 | 3.55 | 0.13 |
| g_Faecalibacterium; s_prausnitzii | | | | | | | | |
| k_Bacteria; p_Firmicutes; c_Clostrida; o_Clostridiales; f_Ruminococcaceae; | v215 | 81 | 7.07 | 80 | 1.69 | 5.0E−04 | 3.55 | 0.24 |
| g_Faecalibacterium; s_prausnitzii | | | | | | | | |
| k_Bacteria; p_Bacteroidetes; c_Bacteroidia; o_Bacteroidales; f_Rikenellaceae; | v12263 | 81 | 0.00 | 80 | 0.19 | 5.7E−04 | −3.52 | |
| g_Bacteroides | | | | | | | | |
| k_Bacteria; p_Bacteroidetes; c_Bacteroidia; o_Bacteroidales; f_Bacteroidaceae; | v12 | 81 | 90.41 | 80 | 25.61 | 5.7E−04 | 3.52 | 0.28 |
| g_Bacteroides; s_uniformis | | | | | | | | |
| k_Bacteria; p_Firmicutes; c_Clostrida; o_Clostridiales; f_Ruminococcaceae; | v7546 | 81 | 0.23 | 80 | 0.01 | 5.7E−04 | 3.52 | 0.05 |
| g_Faecalibacterium; s_prausnitzii | | | | | | | | |
| k_Bacteria; p_Firmicutes; c_Clostrida; o_Clostridiales; f_Ruminococcaceae; | v1058 | 81 | 0.25 | 80 | 1.41 | 6.2E−04 | −3.49 | 5.72 |
| g_Ruminococcus; s_bromii | | | | | | | | |
| k_Bacteria; p_Firmicutes; c_Clostrida; o_Clostridiales; f_Lachnospiraceae; | v527 | 81 | 0.42 | 80 | 2.53 | 7.3E−04 | −3.45 | 6.02 |
| g_Blautia | | | | | | | | |
| k_Bacteria; p_Firmicutes; c_Clostrida; o_Clostridiales; f_Lachnospiraceae; | v3447 | 81 | 0.44 | 80 | 0.04 | 8.1E−04 | 3.41 | 0.08 |
| g_Blautia | | | | | | | | |
| k_Bacteria; p_Firmicutes; c_Clostrida; o_Clostridiales; f_Ruminococcaceae; | v723 | 81 | 1.77 | 80 | 0.49 | 8.8E−04 | 3.39 | 0.23 |
| g_Faecalibacterium; s_prausnitzii | | | | | | | | |
| k_Bacteria; p_Firmicutes; c_Clostrida; o_Clostridiales; f_Ruminococcaceae; | v8111 | 81 | 0.01 | 80 | 0.20 | 9.3E−04 | −3.37 | 16.20 |
| g_Ruminococcus; s_bromii | | | | | | | | |

TABLE 17
Gut microbes corresponding with high and low GTA-512 levels:

| OTUs | OTUs | N1 | meanQ1 | N2 | meanQ5 | p value | tstatistic | ratio |
|---|---|---|---|---|---|---|---|---|
| GTA511315 | | 82 | 0.31 | 80 | 2.81 | 1.1E−50 | −22.19 | |
| k_Bacteria; p_Firmicutes; c_Clostrida; o_Clostridiales; f_Lachnospiraceae | v957 | 82 | 0.16 | 80 | 1.30 | 1.8E−05 | −4.42 | 8.20 |
| k_Bacteria; p_Firmicutes; c_Clostrida; o_Clostridiales; f_Lachnospiraceae; | v527 | 82 | 0.39 | 80 | 3.33 | 2.9E−05 | −4.30 | 8.52 |
| g_Blautia | | | | | | | | |
| k_Bacteria; p_Firmicutes; c_Clostrida; o_Clostridiales; f_Lachnospiraceae; | V1007 | 82 | 0.20 | 80 | 1.64 | 3.1E−05 | −4.29 | 8.39 |
| g_Blautia | | | | | | | | |
| k_Bacteria; p_Firmicutes; c_Clostrida; o_Clostridiales; f_Ruminococcaceae; | v967 | 82 | 0.12 | 80 | 1.50 | 3.7E−05 | −4.25 | 12.30 |
| g_Faecalibacterium; s_prausnitzii | | | | | | | | |
| k_Bacteria; p_Firmicutes; c_Clostrida; o_Clostridiales; f_Lachnospiraceae; | v385 | 82 | 0.73 | 80 | 4.61 | 4.3E−05 | −4.20 | 6.30 |
| g_Blautia | | | | | | | | |
| k_Bacteria; p_Firmicutes; c_Clostrida; o_Clostridiales; f_Ruminococcaceae; | v407 | 82 | 0.32 | 80 | 2.71 | 5.9E−05 | −4.13 | 8.55 |
| g_Faecalibacterium; s_prausnitzii | | | | | | | | |
| k_Bacteria; p_Firmicutes; c_Clostrida; o_Clostridiales; f_Lachnospiraceae; | v782 | 82 | 1.59 | 80 | 0.19 | 6.3E−05 | 4.11 | 0.12 |
| g_Coprococcus | | | | | | | | |
| k_Bacteria; p_Firmicutes; c_Clostrida; o_Clostridiales; f_Lachnospiraceae; | v188 | 82 | 9.22 | 80 | 2.58 | 7.1E−05 | 4.08 | 0.23 |
| g_Blautia | | | | | | | | |
| k_Bacteria; p_Firmicutes; c_Clostrida; o_Clostridiales; f_Lachnospiraceae | v1540 | 82 | 0.05 | 80 | 0.85 | 8.1E−05 | −4.05 | 17.42 |
| k_Bacteria; p_Firmicutes; c_Clostrida; o_Clostridiales; f_Lachnospiraceae; | v284 | 82 | 5.67 | 80 | 1.34 | 9.2E−05 | 4.01 | 0.24 |
| g_Blautia | | | | | | | | |
| k_Bacteria; p_Firmicutes; c_Clostrida; o_Clostridiales; f_Ruminococcaceae; | v324 | 82 | 0.70 | 80 | 3.66 | 1.3E−04 | −3.92 | 5.27 |
| g_Faecalibacterium; s_prausnitzii | | | | | | | | |
| k_Bacteria; p_Firmicutes; c_Clostrida; o_Clostridiales; f_Ruminococcaceae; | v717 | 82 | 0.29 | 80 | 1.99 | 1.5E−04 | −3.88 | 6.79 |
| g_Faecalibacterium; s_prausnitzii | | | | | | | | |
| k_Bacteria; p_Firmicutes; c_Clostrida; o_Clostridiales; f_Lachnospiraceae; | v2737 | 82 | 0.01 | 80 | 0.54 | 2.2E−04 | −3.78 | 44.08 |
| g_Blautia | | | | | | | | |
| k_Bacteria; p_Firmicutes; c_Clostrida; o_Clostridiales; f_Lachnospiraceae; | v1423 | 82 | 0.13 | 80 | 1.16 | 2.3E−04 | −3.77 | 8.67 |
| g_Blautia | | | | | | | | |
| k_Bacteria; p_Firmicutes; c_Clostrida; o_Clostridiales; f_Ruminococcaceae | v942 | 82 | 0.26 | 80 | 1.60 | 2.4E−04 | −3.76 | 6.25 |
| k_Bacteria; p_Firmicutes; c_Clostrida; o_Clostridiales; f_Lachnospiraceae; | v1068 | 82 | 0.93 | 80 | 0.25 | 3.3E−04 | 3.67 | 0.27 |
| g_Blautia | | | | | | | | |
| k_Bacteria; p_Firmicutes; c_Clostrida; o_Clostridiales; f_Ruminococcaceae; | v516 | 82 | 0.46 | 80 | 2.50 | 3.5E−04 | −3.65 | 5.39 |
| g_Faecalibacterium; s_prausnitzii | | | | | | | | |
| k_Bacteria; p_Firmicutes; c_Clostrida; o_Clostridiales; f_Ruminococcaceae; | v2874 | 82 | 0.50 | 80 | 0.06 | 3.6E−04 | 3.65 | 0.13 |
| g_Faecalibacterium; s_prausnitzii | | | | | | | | |
| k_Bacteria; p_Bacteroidetes; c_Bacteroidia; o_Bacteroidales; | v490 | 82 | 4.16 | 80 | 0.41 | 3.9E−04 | 3.62 | 0.10 |
| f_Bacteroidaceae; g_Bacteroides | | | | | | | | |
| k_Bacteria; p_Firmicutes; c_Clostrida; o_Clostridiales; f_Ruminococcaceae; | v10175 | 82 | 0.01 | 80 | 0.19 | 3.9E−04 | −3.62 | 15.38 |
| g_Faecalibacterium; s_prausnitzii | | | | | | | | |
| k_Bacteria; p_Firmicutes; c_Clostrida; o_Clostridiales; f_Lachnospiraceae; | v3283 | 82 | 0.05 | 80 | 0.38 | 3.9E−04 | −3.62 | 7.69 |
| g_Dorea; s_formicigenerans | | | | | | | | |
| k_Bacteria; p_Firmicutes; c_Clostrida; o_Clostridiales; f_Ruminococcaceae | v294 | 82 | 1.18 | 80 | 5.08 | 4.3E−04 | −3.60 | 4.29 |
| k_Bacteria; p_Firmicutes; c_Clostrida; o_Clostridiales; f_Ruminococcaceae | v13337 | 82 | 0.00 | 80 | 0.14 | 4.3E−04 | −3.59 | |
| k_Bacteria; p_Firmicutes; c_Clostrida; o_Clostridiales; f_Lachnospiraceae; | v1218 | 82 | 0.23 | 80 | 1.44 | 5.3E−04 | −3.54 | 6.20 |
| g_Blautia | | | | | | | | |
| k_Bacteria; p_Firmicutes; c_Clostrida; o_Clostridiales; f_Lachnospiraceae; | v492 | 82 | 2.48 | 80 | 0.38 | 5.3E−04 | 3.54 | 0.15 |
| g_Coprococcus | | | | | | | | |
| k_Bacteria; p_Firmicutes; c_Clostrida; o_Clostridiales; f_Lachnospiraceae | v4916 | 82 | 0.00 | 80 | 0.40 | 6.3E−04 | −3.49 | |
| k_Bacteria; p_Firmicutes; c_Clostrida; o_Clostridiales; f_Lachnospiraceae | v688 | 82 | 0.35 | 80 | 2.03 | 6.3E−04 | −3.49 | 5.73 |
| k_Bacteria; p_Firmicutes; c_Clostrida; o_Clostridiales; f_Veillonellaceae; | v783 | 82 | 1.93 | 80 | 0.43 | 6.5E−04 | 3.48 | 0.22 |
| g_Dialister | | | | | | | | |
| k_Bacteria; p_Firmicutes; c_Clostrida; o_Clostridiales; f_Ruminococcaceae | v360 | 82 | 1.05 | 80 | 3.84 | 6.6E−04 | −3.47 | 3.66 |
| k_Bacteria; p_Bacteroidetes; c_Bacteroidia; o_Bacteroidales; | v731 | 82 | 0.06 | 80 | 2.11 | 6.8E−04 | −3.47 | 34.64 |
| f_Bacteroidaceae; g_Bacteroides | | | | | | | | |
| k_Bacteria; p_Firmicutes; c_Clostrida; o_Clostridiales; f_Lachnospiraceae; | v1702 | 82 | 0.11 | 80 | 0.90 | 7.3E−04 | −3.44 | 8.20 |
| g_Coprococcus | | | | | | | | |
| k_Bacteria; p_Firmicutes; c_Clostrida; o_Clostridiales; f_Ruminococcaceae; | v1123 | 82 | 0.21 | 80 | 1.40 | 7.5E−04 | −3.44 | 6.75 |
| f_Facalibecterium | | | | | | | | |
| k_Bacteria; p_Firmicutes; c_Clostrida; o_Clostridiales; f_Veillonellaceae; | v617 | 82 | 2.13 | 80 | 0.63 | 7.9E−04 | 3.42 | 0.29 |
| g_Dialister | | | | | | | | |
| k_Bacteria; p_Firmicutes; c_Clostrida; o_Clostridiales; f_Veillonellaceae; | v7341 | 82 | 0.24 | 80 | 0.00 | 8.2E−04 | 3.41 | 0.00 |
| g_Dialister | | | | | | | | |
| k_Bacteria; p_Bacteroidetes; c_Bacteroidia; o_Bacteroidales; f_Rikenellacaea; | v21051 | 82 | 0.00 | 80 | 0.13 | 8.5E−04 | −3.40 | |
| g_Bacteroides | | | | | | | | |
| k_Bacteria; p_Firmicutes; c_Clostrida; o_Clostridiales; f_Lachnospiraceae; | v5168 | 82 | 0.04 | 80 | 0.29 | 8.5E−04 | −3.40 | 7.86 |
| g_Roseburia | | | | | | | | |
| k_Bacteria; p_Bacteroidetes; c_Bacteroidia; o_Bacteroidales; | v7465 | 82 | 0.24 | 80 | 0.04 | 8.8E−04 | 3.39 | 0.15 |
| f_Bacteroidaceae; g_Bacteroides | | | | | | | | |
| k_Bacteria; p_Firmicutes; c_Clostrida; o_Clostridiales; f_Lachnospiraceae; | v1963 | 82 | 0.13 | 80 | 0.88 | 9.3E−04 | −3.37 | 6.52 |
| g_Blautia | | | | | | | | |

TABLE 18
Gut microbes corresponding with high and low GTA-518 levels:

| OTUs | OTUs | N1 | meanQ1 | N2 | meanQ5 | p value | tstatistic | ratio |
|---|---|---|---|---|---|---|---|---|
| GTA517473 | | 82 | 0.17 | 81 | 0.85 | 1.9E−66 | −29.32 | |
| k_Bacteria; p_Firmicutes; c_Clostrida; o_Clostridiales; f_Lachnospiraceae; | v9916 | 82 | 0.00 | 81 | 0.21 | 9.4E−04 | −3.37 | |
| g_Blautia | | | | | | | | |
| k_Bacteria; p_Firmicutes; c_Clostrida; o_Clostridiales; f_Ruminococcaceae | v8216 | 82 | 0.01 | 81 | 0.20 | 5.0E−04 | −3.56 | 16.20 |
| k_Bacteria; p_Firmicutes; c_Clostrida; o_Clostridiales; f_Ruminococcaceae | v13337 | 82 | 0.01 | 81 | 0.17 | 3.3E−04 | −3.67 | 14.17 |
| k_Bacteria; p_Firmicutes; c_Clostrida; o_Clostridiales; f_Lachnospiraceae | v2563 | 82 | 0.06 | 81 | 0.73 | 4.1E−04 | −3.61 | 11.95 |
| k_Bacteria; p_Bacteroidetes; c_Bacteroidia; o_Bacteroidales; f_Bacteroidaceae; | v529 | 82 | 0.21 | 81 | 1.63 | 6.1E−04 | −3.50 | 7.86 |
| g_Bacteroides | | | | | | | | |
| k_Bacteria; p_Firmicutes; c_Clostrida; o_Clostridiales; f_Lachnospiraceae; | v527 | 82 | 0.50 | 81 | 3.79 | 2.2E−05 | −4.37 | 7.58 |
| g_Blautia | | | | | | | | |
| k_Bacteria; p_Firmicutes; c_Clostrida; o_Clostridiales; f_Lachnospiraceae; | v1007 | 82 | 0.21 | 81 | 1.35 | 2.5E−04 | −3.75 | 6.49 |
| g_Blautia | | | | | | | | |
| k_Bacteria; p_Firmicutes; c_Clostrida; o_Clostridiales; f_Lachnospiraceae; | v1702 | 82 | 0.16 | 81 | 1.02 | 9.3E−04 | −3.37 | 6.46 |
| g_Coprococcus | | | | | | | | |
| k_Bacteria; p_Firmicutes; c_Clostrida; o_Clostridiales; f_Lachnospiraceae; | v5333 | 82 | 0.05 | 81 | 0.28 | 1.6E−04 | −3.87 | 5.82 |
| g_Blautia | | | | | | | | |
| k_Bacteria; p_Firmicutes; c_Clostrida; o_Clostridiales; f_Ruminococcaceae; | v407 | 82 | 0.63 | 81 | 3.60 | 1.8E−05 | −4.43 | 5.68 |
| g_Faecalibacterium; s_prausnitzii | | | | | | | | |
| k_Bacteria; p_Firmicutes; c_Clostrida; o_Clostridiales; f_Lachnospiraceae; | v385 | 82 | 0.90 | 81 | 5.12 | 7.8E−05 | −4.05 | 5.68 |
| g_Blautia | | | | | | | | |
| k_Bacteria; p_Firmicutes; c_Clostrida; o_Clostridiales; f_Ruminococcaceae; | v324 | 82 | 1.32 | 81 | 4.99 | 1.9E−04 | −3.82 | 3.79 |
| g_Faecalibacterium; s_prausnitzii | | | | | | | | |

TABLE 19
Gut microbes corresponding with high and low GTA-520 levels:

| OTUs | OTUs | N1 | meanQ1 | N2 | meanQ5 | p value | tstatistic | ratio |
|---|---|---|---|---|---|---|---|---|
| GTA519475 | | 81 | 0.30 | 79 | 1.21 | 7.8E−74 | −33.59 | |
| k_Bacteria; p_Firmicutes; c_Clostridia; o_Clostridiales; f_Lachnospiraceae | v4916 | 81 | 0.02 | 79 | 0.42 | 2.0E−04 | −3.81 | 16.92 |
| k_Bacteria; p_Firmicutes; c_Clostridia; o_Clostridiales; f_Ruminococcaceae | v8216 | 81 | 0.01 | 79 | 0.18 | 7.4E−04 | −3.44 | 14.35 |
| k_Bacteria; p_Firmicutes; c_Clostridia; o_Clostridiales; f_Lachnospiraceae | v2563 | 81 | 0.06 | 79 | 0.78 | 2.0E−04 | −3.81 | 12.71 |
| k_Bacteria; p_Firmicutes; c_Clostridia; o_Clostridiales; f_Lachnospiraceae; | v1007 | 81 | 0.22 | 79 | 1.47 | 1.6E−04 | −3.86 | 6.61 |
| g_Blautia | | | | | | | | |
| k_Bacteria; p_Firmicutes; c_Clostridia; o_Clostridiales; f_Lachnospiraceae; | v527 | 81 | 0.48 | 79 | 3.15 | 1.5E−04 | −3.89 | 6.55 |
| g_Blautia | | | | | | | | |
| k_Bacteria; p_Firmicutes; c_Clostridia; o_Clostridiales; f_Lachnospiraceae; | v1702 | 81 | 0.16 | 79 | 1.05 | 8.9E−04 | −3.39 | 6.55 |
| g_Coprococcus | | | | | | | | |
| k_Bacteria; p_Firmicutes; c_Clostridia; o_Clostridiales; f_Lachnospiraceae; | v385 | 81 | 0.88 | 79 | 4.63 | 4.1E−05 | −1.22 | 5.29 |
| g_Blautia | | | | | | | | |
| k_Bacteria; p_Firmicutes; c_Clostridia; o_Clostridiales; f_Ruminococcaceae; | v407 | 81 | 0.59 | 79 | 3.08 | 1.4E−04 | −3.90 | 5.19 |
| g_Faecalibacterium; s_prausnitzii | | | | | | | | |
| k_Bacteria; p_Bacteroidetes; c_Bacteroidia; o_Bacteroidales; f_Bacteroidaceae; | v333 | 81 | 1.14 | 79 | 4.73 | 5.5E−04 | −3.52 | 4.17 |
| g_Bacteroides | | | | | | | | |
| k_Bacteria; p_Firmicutes; c_Clostridia; o_Clostridiales; f_Lachnospiraceae; | v5333 | 81 | 0.07 | 79 | 0.29 | 8.7E−04 | −3.39 | 3.93 |
| g_Blautia | | | | | | | | |
| k_Bacteria; p_Firmicutes; c_Clostridia; o_Clostridiales; f_Ruminococcaceae; | v324 | 81 | 1.33 | 79 | 4.22 | 7.2E−04 | −3.45 | 3.16 |
| g_Faecalibacterium; s_prausnitzii | | | | | | | | |
| k_Bacteria; p_Firmicutes; c_Clostridia; o_Clostridiales; f_Lachnospiraceae; | v782 | 81 | 1.12 | 79 | 0.20 | 5.0E−04 | 3.56 | 0.18 |
| g_Coprococcus | | | | | | | | |

TABLE 20
Gut microbes corresponding with high and low GTA-522 levels:

| OTUs | OTUs | N1 | meanQ1 | N2 | meanQ5 | p value | tstatistic | ratio |
|---|---|---|---|---|---|---|---|---|
| GTA521477 | | 81 | 0.15 | 81 | 0.77 | 4.8E−70 | −31.27 | |
| k_Bacteria; p_Firmicutes; c_Clostridia; o_Clostridiales; f_Clostridiaceae | v5030 | 81 | 0.02 | 81 | 0.30 | 7.9E−04 | −3.42 | 12.00 |
| k_Bacteria; p_Firmicutes; c_Clostridia; o_Clostridiales; f_Lachnospiraceae | v2563 | 81 | 0.07 | 81 | 0.65 | 3.3E−04 | −3.67 | 8.83 |
| k_Bacteria; p_Firmicutes; c_Clostridia; o_Clostridiales; f_Ruminococcaceae; | v10175 | 81 | 0.02 | 81 | 0.20 | 8.6E−04 | −3.40 | 8.00 |
| g_Faecalibacterium; s_prausnitzii | | | | | | | | |
| k_Bacteria; p_Firmicutes; c_Clostridia; o_Clostridiales; f_Lachnospiraceae; | v527 | 81 | 0.60 | 81 | 2.44 | 5.3E−04 | −3.53 | 4.04 |
| g_Blautia | | | | | | | | |
| k_Bacteria; p_Firmicutes; c_Clostridia; o_Clostridiales; f_Lachnospiraceae; | v385 | 81 | 1.04 | 81 | 3.77 | 5.9E−04 | −3.51 | 3.63 |
| g_Blautia | | | | | | | | |

TABLE 21
Gut microbes corresponding with high and low GTA-524 levels:

| OTUs | OTUs | N1 | meanQ1 | N2 | meanQ5 | p value | tstatistic | ratio |
|---|---|---|---|---|---|---|---|---|
| GTA523461 | | 83 | 0.06 | 79 | 0.54 | 3.6E−78 | −35.74 | |
| k_Bacteria; p_Firmicutes; c_Clostridia; o_Clostridiales; f_Ruminococcaceae; | v9350 | 83 | 0.00 | 79 | 0.23 | 6.7E−04 | −3.47 | |
| g_Ruminococcus | | | | | | | | |
| k_Bacteria; p_Bacteroidetes; c_Bacteroidia; o_Bacteroidales; f_Rikenellaceae; | v12263 | 83 | 0.00 | 79 | 0.18 | 3.3E−04 | −3.41 | |
| g_Bacteroides | | | | | | | | |
| k_Bacteria; p_Firmicutes; c_Clostridia; o_Clostridiales; f_Ruminococcaceae; | v1902 | 83 | 0.05 | 79 | 0.68 | 4.1E−04 | −3.61 | 14.18 |
| g_Ruminococcus | | | | | | | | |
| k_Bacteria; p_Firmicutes; c_Clostridia; o_Clostridiales; f_Lachnospiraceae; | v1391 | 83 | 0.08 | 79 | 0.94 | 5.0E−04 | −3.55 | 11.11 |
| g_Roseburia; s_faecis | | | | | | | | |
| k_Bacteria; p_Firmicutes; c_Clostridia; o_Clostridiales; f_Lachnospiraceae | v2563 | 83 | 0.07 | 79 | 0.71 | 7.8E−04 | −3.42 | 9.81 |
| k_Bacteria; p_Firmicutes; c_Clostridia; o_Clostridiales; f_Ruminococcaceae; | v6775 | 83 | 0.02 | 79 | 0.23 | 9.8E−04 | −3.36 | 9.46 |
| g_Oscillospira | | | | | | | | |
| k_Bacteria; p_Firmicutes; c_Clostridia; o_Clostridiales; f_Ruminococcaceae; | v1754 | 83 | 0.12 | 79 | 0.90 | 6.2E−04 | −3.49 | 7.46 |
| g_Ruminococcus | | | | | | | | |
| k_Bacteria; p_Bacteroidetes; c_Bacteroidia; o_Bacteroidales; f_Rikenellaceae; | v2034 | 83 | 0.10 | 79 | 0.68 | 4.9E−04 | −3.56 | 7.09 |
| g_Bacteroides | | | | | | | | |
| k_Bacteria; p_Firmicutes; c_Clostridia; o_Clostridiales; f_Lachnospiraceae; | v1007 | 83 | 0.22 | 79 | 1.51 | 8.9E−05 | −4.02 | 6.95 |
| g_Blautia | | | | | | | | |
| k_Bacteria; p_Firmicutes; c_Clostridia; o_Clostridiales; f_Lachnospiraceae; | v1423 | 83 | 0.17 | 79 | 1.11 | 5.0E−04 | −3.56 | 6.60 |
| g_Blautia | | | | | | | | |
| k_Bacteria; p_Firmicutes; c_Clostridia; o_Clostridiales; f_Lachnospiraceae; | v3710 | 83 | 0.06 | 79 | 0.33 | 6.0E−04 | −3.50 | 5.46 |
| g_Blautia | | | | | | | | |
| k_Bacteria; p_Firmicutes; c_Clostridia; o_Clostridiales; f_Lachnospiraceae; | v527 | 83 | 0.66 | 79 | 3.27 | 3.0E−04 | −3.70 | 4.93 |
| g_Blautia | | | | | | | | |
| k_Bacteria; p_Firmicutes; c_Clostridia; o_Clostridiales; f_Lachnospiraceae; | v385 | 83 | 1.10 | 79 | 4.82 | 1.4E−04 | −3.90 | 4.40 |
| g_Blautia | | | | | | | | |
| k_Bacteria; p_Bacteroidetes; c_Bacteroidia; o_Bacteroidales; f_Bacteroidaceae; | v333 | 83 | 1.17 | 79 | 4.38 | 9.0E−04 | −3.38 | 3.75 |
| g_Bacteroides | | | | | | | | |
| k_Bacteria; p_Firmicutes; c_Clostridia; o_Clostridiales; f_Lachnospiraceae; | v1286 | 83 | 0.29 | 79 | 0.97 | 3.0E−04 | −3.69 | 3.37 |
| g_Blautia | | | | | | | | |
| k_Bacteria; p_Firmicutes; c_Bacilli; o_Lactobacillales; f_Streptococcaceae; | v92 | 83 | 21.10 | 79 | 1.77 | 6.2E−04 | 3.49 | 0.08 |
| g_Streptococcus | | | | | | | | |
| k_Bacteria; p_Firmicutes; c_Clostridia; o_Clostridiales; f_Ruminococcaceae; | v2874 | 83 | 0.48 | 79 | 0.04 | 1.6E−04 | 3.86 | 0.08 |
| g_Faecalibacterium; s_prausnitzii | | | | | | | | |

TABLE 22
Gut microbes corresponding with high and low GTA-530 levels:

| OTUs | OTUs | N1 | meanQ1 | N2 | meanQ5 | p value | tstatistic | ratio |
|---|---|---|---|---|---|---|---|---|
| GTA523467 | | 82 | 0.06 | 81 | 0.50 | 8.9E−87 | −10.78 | |
| k_Bacteria; p_Firmicutes; c_Clostridia; o_Clostridiales; f_Ruminococcaceae; | v5983 | 82 | 0.00 | 81 | 0.28 | 8.4E−05 | −1.04 | |
| g_Ruminococcus | | | | | | | | |
| k_Bacteria; p_Firmicutes; c_Clostridia; o_Clostridiales; f_Ruminococcaceae | v8216 | 82 | 0.00 | 81 | 0.17 | 5.1E−04 | −3.55 | |
| k_Bacteria; p_Firmicutes; c_Clostridia; o_Clostridiales; f_Ruminococcaceae; | v20488 | 82 | 0.00 | 81 | 0.12 | 9.2E−04 | −3.38 | |
| g_Ruminococcus | | | | | | | | |
| k_Bacteria; p_Bacteroidetes; c_Bacteroidia; o_Bacteroidales; f_Rikenellaceae; | v3729 | 82 | 0.02 | 81 | 0.47 | 2.8E−04 | −3.71 | 19.23 |
| g_Bacteroides | | | | | | | | |
| k_Bacteria; p_Firmicutes; c_Clostridia; o_Clostridiales; f_Lachnospiraceae; | v3710 | 82 | 0.02 | 81 | 0.36 | 3.7E−05 | −4.24 | 14.68 |
| g_Blautia | | | | | | | | |
| k_Bacteria; p_Bacteroidetes; c_Bacteroidia; o_Bacteroidales; f_Rikenellaceae; | v5504 | 82 | 0.02 | 81 | 0.23 | 7.4E−04 | −3.44 | 9.62 |
| g_Bacteroides | | | | | | | | |
| k_Bacteria; p_Bacteroidetes; c_Bacteroidia; o_Bacteroidales; f_Rikenellaceae; | v2034 | 82 | 0.09 | 81 | 0.75 | 1.7E−04 | −3.85 | 8.82 |
| g_Bacteroides | | | | | | | | |
| k_Bacteria; p_Bacteroidetes; c_Bacteroidia; o_Bacteroidales; f_Rikenellaceae; | v47 | 82 | 8.85 | 81 | 48.07 | 4.1E−04 | −3.61 | 5.43 |
| g_Bacteroides | | | | | | | | |
| k_Bacteria; p_Firmicutes; c_Clostridia; o_Clostridiales; f_Lachnospiraceae; | v1286 | 82 | 0.30 | 81 | 1.12 | 2.5E−04 | −3.74 | 3.68 |
| g_Blautia | | | | | | | | |

TABLE 23
Gut microbes corresponding with high and low GTA-532 levels:

| OTUs | OTUs | N1 | meanQ1 | N2 | meanQ5 | p value | tstatistic | ratio |
|---|---|---|---|---|---|---|---|---|
| GTA531469 | | 81 | 0.11 | 81 | 0.61 | 1.6E−81 | −37.72 | |
| k_Bacteria; p_Firmicutes; c_Clostridia; o_Clostridiales; f_Ruminococcaceae; | v439 | 81 | 0.09 | 81 | 3.41 | 8.5E−04 | −3.40 | 39.43 |
| g_Oscillospira | | | | | | | | |
| k_Bacteria; p_Bacteroidetes; c_Bacteroidia; o_Bacteroidales; f_Rikenellaceae; | v3729 | 81 | 0.04 | 81 | 0.62 | 7.8E−04 | −3.42 | 16.67 |
| g_Bacteroides | | | | | | | | |
| k_Bacteria; p_Firmicutes; c_Clostridia; o_Clostridiales; f_Ruminococcaceae; | v5983 | 81 | 0.02 | 81 | 0.27 | 8.2E−04 | −3.41 | 11.00 |
| g_Ruminococcus | | | | | | | | |
| k_Bacteria; p_Firmicutes; c_Clostridia; o_Clostridiales; f_Lachnospiraceae; | v3710 | 81 | 0.04 | 81 | 0.36 | 9.3E−05 | −1.01 | 9.67 |
| g_Blautia | | | | | | | | |
| k_Bacteria; p_Firmicutes; c_Clostridia; o_Clostridiales; f_Ruminococcaceae; | v1819 | 81 | 0.14 | 81 | 0.72 | 8.8E−04 | −3.39 | 5.27 |
| g_Ruminococcus; s_bromii | | | | | | | | |
| k_Bacteria; p_Firmicutes; c_Clostridia; o_Clostridiales; f_Lachnospiraceae; | v1286 | 81 | 0.30 | 81 | 1.07 | 4.8E−04 | −3.56 | 3.63 |
| g_Blautia | | | | | | | | |
| k_Bacteria; p_Firmicutes; c_Clostridia; o_Clostridiales; f_Lachnospiraceae; | v1791 | 81 | 0.88 | 81 | 0.28 | 0.1E−04 | 3.38 | 0.32 |
| g_Dorea | | | | | | | | |
| k_Bacteria; p_Firmicutes; c_Clostridia; o_Clostridiales; f_Ruminococcaceae; | v1783 | 81 | 0.65 | 81 | 0.20 | 7.8E−04 | 3.43 | 0.30 |
| g_Faecalibacterium; s_prausnitzii | | | | | | | | |
| k_Bacteria; p_Firmicutes; c_Clostridia; o_Clostridiales; f_Ruminococcaceae; | v5891 | 81 | 0.23 | 81 | 0.01 | 5.2E−04 | 3.54 | 0.05 |
| g_Faecalibacterium; s_prausnitzii | | | | | | | | |

TABLE 24
Gut microbes corresponding with high and low GTA-536 levels:

| OTUs | OTUs | N1 | meanQ1 | N2 | meanQ5 | p value | tstatistic | ratio |
|---|---|---|---|---|---|---|---|---|
| GTA535473 | | 81 | 0.15 | 80 | 0.75 | 1.1E−73 | −33.35 | |
| k_Bacteria; p_Firmicutes; c_Clostridia; o_Clostridiales; f_Lachnospiraceae | v4916 | 81 | 0.02 | 80 | 0.40 | 2.8E−04 | −3.72 | 16.20 |
| k_Bacteria; p_Firmicutes; c_Clostridia; o_Clostridiales; f_Ruminococcaceae; | v1902 | 81 | 0.06 | 80 | 0.68 | 7.2E−04 | −3.45 | 10.93 |
| g_Ruminococcus | | | | | | | | |
| k_Bacteria; p_Firmicutes; c_Clostridia; o_Clostridiales; f_Lachnospiraceae | v2563 | 81 | 0.06 | 80 | 0.66 | 7.7E−04 | −3.43 | 10.73 |
| k_Bacteria; p_Firmicutes; c_Clostridia; o_Clostridiales; f_Lachnospiraceae; | v527 | 81 | 0.51 | 80 | 2.95 | 4.3E−04 | −3.56 | 5.83 |
| g_Blautia | | | | | | | | |
| k_Bacteria; p_Firmicutes; c_Clostridia; o_Clostridiales; f_Ruminococcaceae; | v407 | 81 | 0.62 | 80 | 2.81 | 2.9E−04 | −3.71 | 4.56 |
| g_Faecalibacterium; s_prausnitzii | | | | | | | | |
| k_Bacteria; p_Firmicutes; c_Clostridia; o_Clostridiales; f_Lachnospiraceae; | v385 | 81 | 0.98 | 80 | 4.18 | 5.9E−04 | −3.50 | 4.28 |
| g_Blautia | | | | | | | | |
| k_Bacteria; p_Bacteroidetes; c_Bacteroidia; o_Bacteroidales; f_Bacteroidaceae; | v10464 | 81 | 0.15 | 80 | 0.00 | 8.8E−04 | 3.39 | 0.00 |
| g_Bacteroides; s_uniformis | | | | | | | | |
| k_Bacteria; p_Firmicutes; c_Clostridia; o_Clostridiales; f_Lachnospiraceae; | v782 | 81 | 1.21 | 80 | 0.18 | 3.1E−04 | 3.69 | 0.14 |
| g_Coprococcus | | | | | | | | |
| k_Bacteria; p_Firmicutes; c_Clostridia; o_Clostridiales; f_Lachnospiraceae; | v3066 | 81 | 0.27 | 80 | 0.04 | 6.4E−04 | 3.48 | 0.14 |
| g_Coprococcus | | | | | | | | |
| k_Bacteria; p_Firmicutes; c_Clostridia; o_Clostridiales; f_Ruminococcaceae; | v2874 | 81 | 0.37 | 80 | 0.04 | 8.9E−04 | 3.39 | 0.10 |
| g_Faecalibacterium; s_prausnitzii | | | | | | | | |
| k_Bacteria; p_Bacteroidetes; c_Bacteroidia; o_Bacteroidales; f_Bacteroidaceae; | v6523 | 81 | 0.27 | 80 | 0.03 | 9.0E−04 | 3.38 | 0.09 |
| g_Bacteroides; s_uniformis | | | | | | | | |
| k_Bacteria; p_Bacteroidetes; c_Bacteroidia; o_Bacteroidales; f_Bacteroidaceae; | v9826 | 81 | 0.17 | 80 | 0.01 | 9.7E−04 | 3.36 | 0.07 |
| g_Bacteroides; s_uniformis | | | | | | | | |

TABLE 25
Gut microbes corresponding with high and low GTA-538 levels:

| OTUs | OTUs | N1 | meanQ1 | N2 | meanQ5 | p value | tstatistic | ratio |
|---|---|---|---|---|---|---|---|---|
| GTA537475 | | 81 | 0.71 | 80 | 2.83 | 9.2E−61 | −26.76 | |
| k_Bacteria; p_Firmicutes; c_Clostridia; o_Clostridiales; | v8216 | 81 | 0.00 | 80 | 0.18 | 2.0E−04 | −3.80 | [symbol] #DIV/0. |
| f_Ruminococcaceae | | | | | | | | |
| k_Bacteria; p_Firmicutes; c_Clostridia; o_Clostridiales; f_Lachnospiraceae; | v5431 | 81 | 0.01 | 80 | 0.23 | 5.0E−04 | −3.55 | 18.22 |
| g_Roseburia | | | | | | | | |
| k_Bacteria; p_Bacteroidetes; c_Bacteroidia; o_Bacteroidales; | v5504 | 81 | 0.01 | 80 | 0.20 | 9.3E−04 | −3.37 | 16.20 |
| f_Rikenellaceae; g_Bacteroides | | | | | | | | |
| k_Bacteria; p_Firmicutes; c_Clostridia; o_Clostridiales; f_Lachnospiraceae | v4916 | 81 | 0.02 | 80 | 0.38 | 4.3E−04 | −3.60 | 15.19 |
| k_Bacteria; p_Firmicutes; c_Clostridia; o_Clostridiales; f_Lachnospiraceae | v2563 | 81 | 0.05 | 80 | 0.66 | 4.6E−04 | −3.58 | 13.42 |
| k_Bacteria; p_Firmicutes; c_Clostridia; o_Clostridiales; f_Lachnospiraceae; | v5168 | 81 | 0.02 | 80 | 0.25 | 6.7E−04 | −3.47 | 10.12 |
| g_Roseburia | | | | | | | | |
| k_Bacteria; p_Firmicutes; c_Clostridia; o_Clostridiales; f_Lachnospiraceae; | v527 | 81 | 0.41 | 80 | 2.88 | 1.6E−04 | −3.86 | 7.06 |
| g_Blautia | | | | | | | | |
| k_Bacteria; p_Firmicutes; c_Clostridia; o_Clostridiales; | v967 | 81 | 0.26 | 80 | 1.58 | 5.9E−04 | −3.51 | 6.07 |
| f_Ruminococcaceae; g_Faecalibacterium; s_prausnitzii | | | | | | | | |
| k_Bacteria; p_Firmicutes; c_Clostridia; o_Clostridiales; f_Lachnospiraceae; | v385 | 81 | 0.70 | 80 | 4.14 | 1.0E−04 | −3.98 | 5.88 |
| g_Blautia | | | | | | | | |
| k_Bacteria; p_Firmicutes; c_Clostridia; o_Clostridiales; | v1998 | 81 | 0.14 | 80 | 0.76 | 9.6E−04 | −3.36 | 5.61 |
| f_Ruminococcaceae | | | | | | | | |
| k_Bacteria; p_Bacteroidetes; c_Bacteroidia; o_Bacteroidales; | v333 | 81 | 0.90 | 80 | 4.86 | 1.2E−04 | −3.94 | 5.40 |
| f_Bacteroidaceae; g_Bacteroides | | | | | | | | |
| k_Bacteria; p_Bacteroidetes; c_Bacteroidia; o_Bacteroidales; | v3024 | 81 | 0.07 | 80 | 0.38 | 8.7E−04 | −3.39 | 5.06 |
| f_Bacteroidaceae; g_Bacteroides | | | | | | | | |
| k_Bacteria; p_Firmicutes; c_Clostridia; o_Clostridiales; f_Lachnospiraceae; | v1007 | 81 | 0.25 | 80 | 1.24 | 7.3E−04 | −3.44 | 5.01 |
| g_Blautia | | | | | | | | |
| k_Bacteria; p_Bacteroidetes; c_Bacteroidia; o_Bacteroidales; | v158 | 81 | 8.31 | 80 | 2.55 | 8.7E−04 | 3.39 | 0.31 |
| f_Bacteroidaceae; g_Bacteroides | | | | | | | | |
| k_Bacteria; p_Firmicutes; c_Clostridia; o_Clostridiales; f_Lachnospiraceae; | v513 | 81 | 1.96 | 80 | 0.54 | 4.2E−04 | 3.60 | 0.27 |
| g_Blautia | | | | | | | | |
| k_Bacteria; p_Firmicutes; c_Clostridia; o_Clostridiales; f_Lachnospiraceae; | v188 | 81 | 7.17 | 80 | 1.70 | 9.5E−05 | 4.00 | 0.24 |
| g_Blautia | | | | | | | | |
| k_Bacteria; p_Firmicutes; c_Clostridia; o_Clostridiales; f_Lachnospiraceae; | v284 | 81 | 4.35 | 80 | 0.86 | 8.7E−05 | 4.03 | 0.20 |
| g_Blautia | | | | | | | | |
| k_Bacteria; p_Bacteroidetes; c_Bacteroidia; o_Bacteroidales; | v14672 | 81 | 0.17 | 80 | 0.01 | 9.7E−04 | 3.36 | 0.07 |
| f_Bacteroidaceae; g_Bacteroides | | | | | | | | |

TABLE 26
Gut microbes corresponding with high and low GTA-540 levels:

| OTUs | OTUs | N1 | meanQ1 | N2 | meanQ5 | p value | tstatistic | ratio |
|---|---|---|---|---|---|---|---|---|
| GTA539315 | | 82 | 1.36 | 81 | 7.95 | 2.0E−52 | −22.86 | |
| k_Bacteria; p_Firmicutes; c_Clostridia; o_Clostridiales; f_Lachnospiraceae | v4916 | 82 | 0.00 | 81 | 0.31 | 3.7E−04 | −3.64 | |
| k_Bacteria; p_Proteobacteria; c_Gammaproteobacteria; o_Enterobacteriales; | v13141 | 82 | 0.00 | 81 | 0.17 | 5.1E−04 | −3.55 | |
| f_Enterobacteriaceae | | | | | | | | |
| k_Bacteria; p_Firmicutes; c_Clostridia; o_Clostridiales; f_Lachnospiraceae; | v2737 | 82 | 0.02 | 81 | 0.49 | 8.2E−04 | −3.41 | 20.25 |
| g_Blautia | | | | | | | | |
| k_Bacteria; p_Firmicutes; c_Clostridia; o_Clostridiales; f_Lachnospiraceae; | v5168 | 82 | 0.01 | 81 | 0.25 | 6.7E−04 | −3.47 | 20.25 |
| g_Roseburia | | | | | | | | |
| k_Bacteria; p_Firmicutes; c_Clostridia; o_Clostridiales; f_Ruminococcaceae; | v6941 | 82 | 0.01 | 81 | 0.21 | 9.0E−04 | −3.38 | 17.21 |
| g_Faecalibacterium; s_prausnitzii | | | | | | | | |
| k_Bacteria; p_Firmicutes; c_Clostridia; o_Clostridiales; f_Lachnospiraceae | v2563 | 82 | 0.04 | 81 | 0.60 | 8.7E−04 | −3.39 | 16.53 |
| k_Bacteria; p_Firmicutes; c_Clostridia; o_Clostridiales; f_Lachnospiraceae; | v6547 | 82 | 0.02 | 81 | 0.36 | 9.6E−04 | −3.36 | 14.68 |
| g_Anaerostipes | | | | | | | | |
| k_Bacteria; p_Bacteroidetes; c_Bacteroidia; o_Bacteroidales; f_Bacteroidaceae; | v3093 | 82 | 0.05 | 81 | 0.46 | 7.2E−04 | −3.45 | 9.36 |
| g_Bacteroides | | | | | | | | |
| k_Bacteria; p_Firmicutes; c_Clostridia; o_Clostridiales; f_Ruminococcaceae; | v967 | 82 | 0.17 | 81 | 1.51 | 2.4E−04 | −3.75 | 8.82 |
| g_Faecalibacterium; s_prausnitzii | | | | | | | | |
| k_Bacteria; p_Firmicutes; c_Clostridia; o_Clostridiales; f_Lachnospiraceae; | v323 | 82 | 0.90 | 81 | 6.63 | 7.0E−04 | −3.46 | 7.35 |
| g_Anaerostipes | | | | | | | | |
| k_Bacteria; p_Firmicutes; c_Clostridia; o_Clostridiales; f_Lachnospiraceae; | v527 | 82 | 0.41 | 81 | 2.77 | 3.2E−04 | −3.68 | 6.67 |
| g_Blautia | | | | | | | | |
| k_Bacteria; p_Firmicutes; c_Clostridia; o_Clostridiales; f_Lachnospiraceae; | v3283 | 82 | 0.06 | 81 | 0.38 | 9.4E−04 | −3.37 | 6.28 |
| g_Dorea; s_formicigenerans | | | | | | | | |
| k_Bacteria; p_Firmicutes; c_Clostridia; o_Clostridiales; f_Ruminococcaceae; | v717 | 82 | 0.41 | 81 | 2.32 | 4.6E−04 | −3.58 | 5.60 |
| g_Faecalibibacterium; s_prausnitzii | | | | | | | | |
| k_Bacteria; p_Firmicutes; c_Clostridia; o_Clostridiales; f_Lachnospiraceae; | v385 | 82 | 0.74 | 81 | 4.16 | 2.5E−04 | −3.75 | 5.59 |
| g_Blautis | | | | | | | | |
| k_Bacteria; p_Firmicutes; c_Clostridia; o_Clostridiales; f_Lachnospiraceae; | v1007 | 82 | 0.23 | 81 | 1.21 | 9.7E−04 | −3.36 | 5.22 |
| g_Blautis | | | | | | | | |
| k_Bacteria; p_Bacteroidetes; c_Bacteroidia; o_Bacteroidales; f_Bacteroidaceae; | v333 | 82 | 0.99 | 81 | 4.32 | 7.4E−04 | −3.44 | 4.37 |
| g_Bacteroides | | | | | | | | |
| k_Bacteria; p_Firmicutes; c_Clostridia; o_Clostridiales; f_Lachnospiraceae; | v1691 | 82 | 0.18 | 81 | 0.67 | 2.6E−04 | −3.73 | 3.64 |
| g_Roseburia | | | | | | | | |
| k_Bacteria; p_Firmicutes; c_Clostridia; o_Clostridiales; f_Lachnospiraceae; | v12957 | 82 | 0.15 | 81 | 0.00 | 8.9E−04 | 3.39 | 0.00 |
| g_Coprococcus | | | | | | | | |
| k_Bacteria; p_Bacteroidetes; c_Bacteroidia; o_Bacteroidales; f_Bacteroidaceae; | v13964 | 82 | 0.16 | 81 | 0.00 | 4.7E−04 | 3.57 | 0.00 |
| g_Bacteroides | | | | | | | | |
| k_Bacteria; p_Bacteroidetes; c_Bacteroidia; o_Bacteroidales; f_Bacteroidaceae; | v158 | 82 | 8.95 | 81 | 3.44 | 6.1E−04 | 3.50 | 0.38 |
| g_Bacteroides | | | | | | | | |
| k_Bacteria; p_Firmicutes; c_Clostridia; o_Clostridiales; f_Lachnospiraceae; | v4400 | 82 | 0.29 | 81 | 0.06 | 7.6E−04 | 3.43 | 0.21 |
| g_Blautis | | | | | | | | |

TABLE 27
Gut microbes corresponding with high and low GTA-550 levels:

| OTUs | OTUs | N1 | meanQ1 | N2 | meanQ5 | p value | tstatistic | ratio |
|---|---|---|---|---|---|---|---|---|
| GTA549487 | | 81 | 0.00 | 81 | 0.43 | 1.1E−70 | −31.61 | |
| k_Bacteria; p_Firmicutes; c_Clostridia; o_Clostridiales; f_Ruminococcaceae; | v439 | 81 | 0.17 | 81 | 3.57 | 5.4E−04 | −3.53 | 20.64 |
| g_Oscillospira | | | | | | | | |
| k_Bacteria; p_Bacteroidetes; c_Bacteroidia; o_Bacteroidales; f_Rikenellaceae; | v3729 | 81 | 0.02 | 81 | 0.38 | 8.0E−04 | −3.42 | 15.50 |
| g_Bacteroides | | | | | | | | |
| k_Bacteria; p_Firmicutes; c_Clostridia; o_Clostridiales; f_Clostridiaceae | v5030 | 81 | 0.02 | 81 | 0.27 | 8.2E−04 | −3.41 | 11.00 |
| k_Bacteria; p_Firmicutes; c_Clostridia; o_Clostridiales; f_Lachnospiraceae | v2563 | 81 | 0.09 | 81 | 0.78 | 3.5E−04 | −3.65 | 9.00 |
| k_Bacteria; p_Bacteroidetes; c_Bacteroidia; o_Bacteroidales; f_Rikenellaceae; | v2034 | 81 | 0.10 | 81 | 0.68 | 9.1E−04 | −3.38 | 6.88 |
| g_Bacteroides | | | | | | | | |
| k_Bacteria; p_Bacteroidetes; c_Bacteroidia; o_Bacteroidales; f_Rikenellaceae; | v47 | 81 | 7.74 | 81 | 43.90 | 8.1E−04 | −3.41 | 5.67 |
| g_Bacteroides | | | | | | | | |
| k_Bacteria; p_Firmicutes; c_Clostridia; o_Clostridiales; f_Ruminococcaceae; | v2 | 81 | 283.12 | 81 | 133.02 | 5.7E−04 | 3.52 | 0.47 |
| g_Faecalibacterium; s_prausnitzii | | | | | | | | |
| k_Bacteria; p_Firmicutes; c_Clostridia; o_Clostridiales; f_Ruminococcaceae; | v1392 | 81 | 0.94 | 81 | 0.32 | 8.7E−04 | 3.39 | 0.34 |
| g_Faecalibacterium; s_prausnitzii | | | | | | | | |
| k_Bacteria; p_Firmicutes; c_Clostridia; o_Clostridiales; f_Ruminococcaceae; | v723 | 81 | 1.77 | 81 | 0.54 | 7.8E−04 | 3.43 | 0.31 |
| g_Faecalibacterium; s_prausnitzii | | | | | | | | |
| k_Bacteria; p_Firmicutes; c_Clostridia; o_Clostridiales; f_Ruminococcaceae; | v2464 | 81 | 0.44 | 81 | 0.14 | 6.9E−04 | 3.46 | 0.31 |
| g_Faecalibacterium; s_prausnitzii | | | | | | | | |
| k_Bacteria; p_Firmicutes; c_Clostridia; o_Clostridiales; f_Ruminococcaceae; | v1783 | 81 | 0.69 | 81 | 0.19 | 3.0E−04 | 3.70 | 0.27 |
| g_Faecalibacterium; s_prausnitzii | | | | | | | | |
| k_Bacteria; p_Bacteroidetes; c_Bacteroidia; o_Bacteroidales; f_Bacteroidaceae; | v2505 | 81 | 0.49 | 81 | 0.11 | 8.6E−04 | 3.40 | 0.22 |
| g_Bacteroides | | | | | | | | |
| k_Bacteria; p_Firmicutes; c_Clostridia; o_Clostridiales; f_Lachnospiraceae; | v2917 | 81 | 0.47 | 81 | 0.07 | 5.7E−04 | 3.52 | 0.16 |
| g_Coprococcus | | | | | | | | |
| k_Bacteria; p_Firmicutes; c_Clostridia; o_Clostridiales; f_Lachnospiraceae; | v3447 | 81 | 0.47 | 81 | 0.06 | 9.5E−04 | 3.37 | 0.13 |
| g_Blautia | | | | | | | | |
| k_Bacteria; p_Firmicutes; c_Clostridia; o_Clostridiales; f_Ruminococcaceae; | v5762 | 81 | 0.17 | 81 | 0.01 | 9.0E−04 | 3.38 | 0.07 |
| g_Faecalibacterium; s_prausnitzii | | | | | | | | |
| k_Bacteria; p_Firmicutes; c Bacilli; o_Lactobacillales; f_Streptococcaceae; | v8681 | 81 | 0.31 | 81 | 0.01 | 7.6E−04 | 3.43 | 0.04 |
| g_Streptococcus | | | | | | | | |

Table 28: Gut microbes corresponding with high and low GTA-574 levels:

TABLE 29
Gut microbes corresponding with high and low GTA-576 levels:

| OTUs | OTUs | N1 | meanQ1 | N2 | meanQ5 | p value | tstatistic | ratio |
|---|---|---|---|---|---|---|---|---|
| GTA575513 | | 82 | 0.08 | 81 | 0.78 | 1.3E−68 | −30.41 | |
| k_Bacteria; p_Firmicutes; c_Clostridia; o_Clostridiales; f_Clostridiaceae | 5030 | 82 | 0.02 | 81 | 0.30 | 7.3E−04 | −3.45 | 12.15 |
| k_Bacteria; p_Firmicutes; c_Bacilli; o_Lactobacillales; f_Streptococcaceae; | 11309 | 82 | 0.15 | 81 | 0.00 | 8.9E−04 | 3.39 | 0.00 |
| g_Streptococcus | | | | | | | | |

TABLE 30
Gut microbes corresponding with high and low GTA-580 levels:

| OTUs | OTUs | N1 | meanQ1 | N2 | meanQ5 | p value | tstatistic | ratio |
|---|---|---|---|---|---|---|---|---|
| GTA579517 | | 81 | −0.05 | 81 | 0.32 | 2.2E−72 | −32.51 | |
| k_Bacteria; p_Firmicutes; c_Clostridia; o_Clostridiales; f_Ruminococcaceae; | v1058 | 81 | 0.25 | 81 | 1.79 | 2.3E−05 | −4.36 | 7.25 |
| g_Ruminococcus; s_bromii | | | | | | | | |
| k_Bacteria; p_Firmicutes; c_Clostridia; o_Clostridiales; f_Ruminococcaceae; | v125 | 81 | 3.83 | 81 | 12.06 | 8.8E−05 | −4.03 | 3.15 |
| g_Oscillospira | | | | | | | | |
| k_Bacteria; p_Firmicutes; c_Clostridia; o_Clostridiales; f_Ruminococcaceae; | v21 | 81 | 23.67 | 81 | 90.96 | 1.3E−04 | −3.91 | 3.84 |
| g_Ruminococcus; s_bromii | | | | | | | | |
| k_Bacteria; p_Firmicutes; c_Clostridia; o_Clostridiales; f_Ruminococcaceae; | v1783 | 81 | 0.69 | 81 | 0.15 | 1.4E−04 | 3.90 | 0.21 |
| g_Faecalibacterium; s_prausnitzii | | | | | | | | |
| k_Bacteria; p_Firmicutes; c_Clostridia; o_Clostridiales; f_Ruminococcaceae; | v1583 | 81 | 0.25 | 81 | 0.98 | 1.8E−04 | −3.84 | 3.95 |
| g_Ruminococcus; s_bromii | | | | | | | | |
| k_Bacteria; p_Firmicutes; c_Clostridia; o_Clostridiales; f_Ruminococcaceae; | v1819 | 81 | 0.21 | 81 | 1.17 | 1.9E−04 | −3.82 | 5.59 |
| g_Ruminococcus; s_bromii | | | | | | | | |
| k_Bacteria; p_Firmicutes; c_Clostridia; o_Clostridiales; f_Ruminococcaceae; | v3887 | 81 | 0.05 | 81 | 0.33 | 1.9E−04 | −3.82 | 6.75 |
| g_Ruminococcus; s_bromii | | | | | | | | |
| k_Bacteria; p_Firmicutes; c_Clostridia; o_Clostridiales; f_Ruminococcaceae; | v773 | 81 | 0.51 | 81 | 2.01 | 1.9E−04 | −3.82 | 3.98 |
| g_Ruminococcus; s_bromii | | | | | | | | |
| k_Bacteria; p_Bacteroidetes; c_Bacteroidia; o_Bacteroidales; f_Rikenellaceae; | v1211 | 81 | 0.11 | 81 | 1.28 | 2.2E−04 | −3.78 | 11.56 |
| g_Bacteroides | | | | | | | | |
| k_Bacteria; p_Firmicutes; c_Clostridia; o_Clostridiales; f_Ruminococcaceae; | v439 | 81 | 0.11 | 81 | 6.14 | 2.3E−04 | −3.77 | 55.22 |
| g_Oscillospira | | | | | | | | |
| k_Bacteria; p_Firmicutes; c_Clostridia; o_Clostridiales; f_Ruminococcaceae; | v2 | 81 | 268.67 | 81 | 109.30 | 2.3E−04 | 3.77 | 0.41 |
| g_Faecalibacterium; s_prausnitzii | | | | | | | | |
| k_Bacteria; p_Firmicutes; c_Clostridia; o_Clostridiales; f_Ruminococcaceae; | v1465 | 81 | 0.89 | 81 | 0.16 | 2.4E−04 | 3.76 | 0.18 |
| g_Faecalibacterium; s_prausnitzii | | | | | | | | |
| k_Bacteria; p_Bacteroidetes; c_Bacteroidia; o_Bacteroidales; f_Rikenellaceae; | v2034 | 81 | 0.06 | 81 | 0.84 | 2.9E−04 | −3.70 | 13.60 |
| g_Bacteroides | | | | | | | | |
| k_Bacteria; p_Firmicutes; c_Clostridia; o_Clostridiales; f_Ruminococcaceae; | v723 | 81 | 1.67 | 81 | 0.42 | 3.2E−04 | 3.68 | 0.25 |
| g_Faecalibacterium; s_prausnitzii | | | | | | | | |
| k_Bacteria; p_Firmicutes; c_Clostridia; o_Clostridiales; f_Ruminococcaceae; | v1154 | 81 | 0.27 | 81 | 2.16 | 3.6E−04 | −3.65 | 7.95 |
| g_Ruminococcus; s_bromii | | | | | | | | |
| k_Bacteria; p_Bacteroidetes; c_Bacteroidia; o_Bacteroidales; f_Rikenellaceae; | v47 | 81 | 5.67 | 81 | 54.98 | 4.1E−04 | −3.61 | 9.70 |
| g_Bacteroides | | | | | | | | |
| k_Bacteria; p_Firmicutes; c_Clostridia; o_Clostridiales; f_Ruminococcaceae; | v1392 | 81 | 0.86 | 81 | 0.25 | 4.2E−04 | 3.60 | 0.29 |
| g_Faecalibacterium; s_prausnitzii | | | | | | | | |
| k_Bacteria; p_Firmicutes; c_Clostridia; o_Clostridiales; f_Ruminococcaceae; | v2591 | 81 | 0.09 | 81 | 0.65 | 4.9E−04 | −3.56 | 7.57 |
| g_Ruminococcus; s_bromii | | | | | | | | |
| k_Bacteria; p_Firmicutes; c_Clostridia; o_Clostridiales; f_Ruminococcaceae; | v4663 | 81 | 0.05 | 81 | 0.33 | 5.3E−04 | −3.54 | 6.75 |
| g_Ruminococcus | | | | | | | | |
| k_Bacteria; p_Firmicutes; c_Clostridia; o_Clostridiales; f_Ruminococcaceae; | v4509 | 81 | 0.11 | 81 | 0.51 | 5.9E−04 | −3.51 | 4.56 |
| g_Ruminococcus; s_bromii | | | | | | | | |
| k_Bacteria; p_Firmicutes; c_Clostridia; o_Clostridiales; f_Ruminococcaceae; | v7176 | 81 | 0.01 | 81 | 0.31 | 5.9E−04 | −3.51 | 25.00 |
| g_Ruminococcus; s_bromii | | | | | | | | |
| k_Bacteria; p_Firmicutes; c_Clostridia; o_Clostridiales; f_Ruminococcaceae; | v2464 | 81 | 0.43 | 81 | 0.11 | 7.0E−04 | 3.46 | 0.26 |
| g_Faecalibacterium; s_prausnitzii | | | | | | | | |
| k_Bacteria; p_Firmicutes; c_Clostridia; o_Clostridiales; f_Ruminococcaceae; | v545 | 81 | 0.79 | 81 | 2.80 | 7.1E−04 | −3.45 | 3.55 |
| g_Ruminococcus; s_bromii | | | | | | | | |
| k_Bacteria; p_Firmicutes; c_Clostridia; o_Clostridiales; f_Ruminococcaceae; | v1356 | 81 | 0.93 | 81 | 0.27 | 8.0E−04 | 3.42 | 0.29 |
| g_Faecalibacterium; s_prausnitzii | | | | | | | | |
| k_Bacteria; p_Firmicutes; c_Clostridia; o_Clostridiales; f_Ruminococcaceae; | v950 | 81 | 0.00 | 81 | 2.12 | 8.6E−04 | −3.40 | |
| g_Ostillospira | | | | | | | | |

TABLE 31
Gut microbes corresponding with high and low GTA-590 levels:

| OTUs | OTUs | N1 | meanQ1 | N2 | meanQ5 | p value | tstatistic | ratio |
|---|---|---|---|---|---|---|---|---|
| GTA589545 | | 83 | −0.16 | 78 | 0.09 | 6.3E−125 | −73.80 | |
| k_Bacteria; p_Firmicutes; c_Clostridia; o_Clostridiales; | v1356 | 83 | 1.04 | 78 | 0.15 | 3.8E−06 | 4.79 | 0.15 |
| f_Ruminococcaceae; g_Faecalibacterium; s_prausnitzii | | | | | | | | |
| k_Bacteria; p_Firmicutes; c_Clostridia; o_Clostridiales; | v2340 | 83 | 0.59 | 78 | 0.09 | 7.3E−06 | 4.64 | 0.15 |
| f_Ruminococcaceae; g_Faecalibacterium; s_prausnitzii | | | | | | | | |
| k_Bacteria; p_Firmicutes; c_Clostridia; o_Clostridiales; | v215 | 83 | 10.13 | 78 | 2.58 | 1.0E−05 | 4.56 | 0.25 |
| f_Ruminococcaceae; g_Faecalibacterium; s_prausnitzii | | | | | | | | |
| k_Bacteria; p_Firmicutes; c_Clostridia; o_Clostridiales; | v2 | 83 | 312.16 | 78 | 102.50 | 1.1E−05 | 4.54 | 0.33 |
| f_Ruminococcaceae; g_Faecalibacterium; s_prausnitzii | | | | | | | | |
| k_Bacteria; p_Firmicutes; c_Clostridia; o_Clostridiales; | v723 | 83 | 2.57 | 78 | 0.64 | 1.8E−05 | 4.42 | 0.25 |
| f_Ruminococcaceae; g_Faecalibacterium; s_prausnitzii | | | | | | | | |
| k_Bacteria; p_Firmicutes; c_Clostridia; o_Clostridiales; | v2206 | 83 | 0.63 | 78 | 0.06 | 1.9E−05 | 4.41 | 0.10 |
| f_Ruminococcaceae; g_Faecalibacterium; s_prausnitzii | | | | | | | | |
| k_Bacteria; p_Firmicutes; c_Clostridia; o_Clostridiales; | v2558 | 83 | 0.65 | 78 | 0.06 | 2.0E−05 | 4.40 | 0.10 |
| f_Ruminococcaceae; g_Faecalibacterium; s_prausnitzii | | | | | | | | |
| k_Bacteria; p_Firmicutes; c_Clostridia; o_Clostridiales; | v2044 | 83 | 0.58 | 78 | 0.12 | 4.7E−05 | 4.19 | 0.20 |
| f_Ruminococcaceae; g_Faecalibacterium; s_prausnitzii | | | | | | | | |
| k_Bacteria; p_Firmicutes; c_Clostridia; o_Clostridiales; | v1783 | 83 | 0.78 | 78 | 0.14 | 5.0E−05 | 4.17 | 0.18 |
| f_Ruminococcaceae; g_Faecalibacterium; s_prausnitzii | | | | | | | | |
| k_Bacteria; p_Firmicutes; c_Clostridia; o_Clostridiales; | v1465 | 83 | 1.06 | 78 | 0.13 | 5.0E−05 | 4.17 | 0.12 |
| f_Ruminococcaceae; g_Faecalibacterium; s_prausnitzii | | | | | | | | |
| k_Bacteria; p_Firmicutes; c_Clostridia; o_Clostridiales; | v1791 | 83 | 0.75 | 78 | 0.15 | 5.8E−05 | 4.13 | 0.21 |
| f_Lachnospiraceae; g_Dorea | | | | | | | | |
| k_Bacteria; p_Firmicutes; c_Clostridia; o_Clostridiales; | v56 | 83 | 32.48 | 78 | 9.08 | 6.1E−05 | 4.12 | 0.28 |
| f_Lachnospiraceae; g_Dorea | | | | | | | | |
| k_Bacteria; p_Firmicutes; c_Clostridia; o_Clostridiales; | v2874 | 83 | 0.60 | 78 | 0.08 | 6.8E−05 | 4.09 | 0.13 |
| f_Ruminococcaceae; g_Faecalibacterium; s_prausnitzii | | | | | | | | |
| k_Bacteria; p_Firmicutes; c_Clostridia; o_Clostridiales; | v1978 | 83 | 0.67 | 78 | 0.15 | 7.2E−05 | 4.08 | 0.23 |
| f_Ruminococcaceae; g_Faecalibacterium; s_prausnitzii | | | | | | | | |
| k_Bacteria; p_Firmicutes; c_Clostridia; o_Clostridiales; | v891 | 83 | 1.42 | 78 | 0.24 | 7.5E−05 | 4.07 | 0.17 |
| f_Lachnospiraceae; g_Dorea | | | | | | | | |
| k_Bacteria; p_Firmicutes; c_Clostridia; o_Clostridiales; | v2282 | 83 | 0.58 | 78 | 0.13 | 8.0E−05 | 4.05 | 0.22 |
| f_Ruminococcaceae; g_Faecalibacterium; s_prausnitzii | | | | | | | | |
| k_Bacteria; p_Bacteroidetes; c_Bacteroidia; o_Bacteroidales; | v10462 | 83 | 0.00 | 78 | 0.17 | 8.0E−05 | −4.05 | [symbol] #DIV/0. |
| f_Rikenellaceae; g_Bacteroides | | | | | | | | |
| k_Bacteria; p_Firmicutes; c_Clostridia; o_Clostridiales; | v1566 | 83 | 1.06 | 78 | 0.17 | 8.5E−05 | 4.04 | 0.16 |
| f_Ruminococcaceae; g_Faecalibacterium; s_prausnitzii | | | | | | | | |
| k_Bacteria; p_Firmicutes; c_Clostridia; o_Clostridiales; | v2157 | 83 | 0.58 | 78 | 0.12 | 8.5E−05 | 4.03 | 0.20 |
| f_Ruminococcaceae; g_Faecalibacterium; s_prausnitzii | | | | | | | | |
| k_Bacteria; p_Firmicutes; c_Clostridia; o_Clostridiales; | v1392 | 83 | 0.88 | 78 | 0.22 | 8.5E−05 | 4.03 | 0.25 |
| f_Ruminococcaceae; g_Faecalibacterium; s_prausnitzii | | | | | | | | |
| k_Bacteria; p_Firmicutes; c_Clostridia; o_Clostridiales; | v473 | 83 | 3.33 | 78 | 0.49 | 1.4E−04 | 3.90 | 0.15 |
| f_Ruminococcaceae | | | | | | | | |
| k_Bacteria; p_Firmicutes; c_Clostridia; o_Clostridiales; | v4334 | 83 | 0.33 | 78 | 0.04 | 1.6E−04 | 3.87 | 0.12 |
| f_Ruminococcaceae; g_Faecalibacterium; s_prausnitzii | | | | | | | | |
| k_Bacteria; p_Firmicutes; c_Clostridia; o_Clostridiales; | v3447 | 83 | 0.55 | 78 | 0.04 | 1.8E−04 | 3.84 | 0.07 |
| f_Lachnospiraceae; g_Blautia | | | | | | | | |
| k_Bacteria; p_Firmicutes; c_Clostridia; o_Clostridiales; | v608 | 83 | 1.72 | 78 | 0.56 | 1.8E−04 | 3.83 | 0.33 |
| f_Ruminococcaceae; g_Faecalibacterium; s_prausnitzii | | | | | | | | |
| k_Bacteria; p_Firmicutes; c_Clostridia; o_Clostridiales; | v1673 | 83 | 0.71 | 78 | 0.19 | 1.8E−04 | 3.83 | 0.27 |
| f_Ruminococcaceae; g_Faecalibacterium; s_prausnitzii | | | | | | | | |
| k_Bacteria; p_Firmicutes; c_Clostridia; o_Clostridiales; | v1549 | 83 | 0.72 | 78 | 0.19 | 1.8E−04 | 3.83 | 0.27 |
| f_Ruminococcaceae; g_Faecalibacterium; s_prausnitzii | | | | | | | | |
| k_Bacteria; p_Bacteroidetes; c_Clostridia; o_Bacteroidales; | v1211 | 83 | 0.04 | 78 | 0.90 | 2.0E−04 | −3.80 | 24.83 |
| f_Rikenellaceae; g_Bacteroides | | | | | | | | |
| k_Bacteria; p_Firmicutes; c_Clostridia; o_Clostridiales; | v927 | 83 | 1.84 | 78 | 0.49 | 2.1E−04 | 3.80 | 0.26 |
| f_Ruminococcaceae; g_Faecalibacterium; s_prausnitzii | | | | | | | | |
| k_Bacteria; p_Firmicutes; c_Clostridia; o_Clostridiales; | v3526 | 83 | 0.42 | 78 | 0.08 | 2.4E−04 | 3.76 | 0.18 |
| f_Ruminococcaceae; g_Faecalibacterium; s_prausnitzii | | | | | | | | |
| k_Bacteria; p_Firmicutes; c_Clostridia; o_Clostridiales; | v3579 | 83 | 0.43 | 78 | 0.05 | 2.4E−04 | 3.76 | 0.12 |
| f_Ruminococcaceae; g_Faecalibacterium; s_prausnitzii | | | | | | | | |
| k_Bacteria; p_Firmicutes; c_Clostridia; o_Clostridiales; | v567 | 83 | 2.20 | 78 | 0.47 | 2.4E−04 | 3.76 | 0.22 |
| f_Lachnospiraceae; g_Dorea | | | | | | | | |
| k_Bacteria; p_Firmicutes; c_Clostridia; o_Clostridiales; | v2298 | 83 | 0.39 | 78 | 0.05 | 2.7E−04 | 3.73 | 0.13 |
| f_Ruminococcaceae; g_Faecalibacterium; s_prausnitzii | | | | | | | | |
| k_Bacteria; p_Firmicutes; c_Clostridia; o_Clostridiales; | v8162 | 83 | 0.25 | 78 | 0.01 | 2.8E−04 | 3.72 | 0.05 |
| f_Lachnospiraceae; g_Blautia | | | | | | | | |
| k_Bacteria; p_Firmicutes; c_Clostridia; o_Clostridiales; | v6347 | 83 | 0.24 | 78 | 0.01 | 2.9E−04 | 3.71 | 0.05 |
| f_Ruminococcaceae; g_Faecalibacterium; s_prausnitzii | | | | | | | | |
| k_Bacteria; p_Firmicutes; c_Clostridia; o_Clostridiales; | v1377 | 83 | 0.83 | 78 | 0.24 | 3.3E−04 | 3.67 | 0.29 |
| f_Ruminococcaceae; g_Faecalibacterium; s_prausnitzii | | | | | | | | |
| k_Bacteria; p_Firmicutes; c_Clostridia; o_Clostridiales; | v352 | 83 | 4.48 | 78 | 0.69 | 3.5E−04 | 3.66 | 0.15 |
| f_Ruminococcaceae; g_Faecalibacterium | | | | | | | | |
| k_Bacteria; p_Firmicutes; c_Clostridia; o_Clostridiales; | v1580 | 83 | 0.92 | 78 | 0.13 | 3.5E−04 | 3.65 | 0.14 |
| f_Ruminococcaceae; g_Faecalibacterium | | | | | | | | |
| k_Bacteria; p_Firmicutes; c_Clostridia; o_Clostridiales; | v2135 | 83 | 0.64 | 78 | 0.13 | 3.7E−04 | 3.64 | 0.20 |
| f_Ruminococcaceae; g_Faecalibacterium; s_prausnitzii | | | | | | | | |
| k_Bacteria; p_Firmicutes; c_Clostridia; o_Clostridiales; | v3672 | 83 | 0.37 | 78 | 0.04 | 3.7E−04 | 3.64 | 0.10 |
| f_Ruminococcaceae; g_Faecalibacterium; s_prausnitzii | | | | | | | | |
| k_Bacteria; p_Firmicutes; c_Clostridia; o_Clostridiales; | v1372 | 83 | 1.13 | 78 | 0.06 | 3.8E−04 | 3.63 | 0.06 |
| f_Ruminococcaceae; g_Faecalibacterium; s_prausnitzii | | | | | | | | |
| k_Bacteria; p_Firmicutes; c_Clostridia; o_Clostridiales; | v2696 | 83 | 0.51 | 78 | 0.10 | 4.1E−04 | 3.61 | 0.20 |
| f_Ruminococcaceae; g_Faecalibacterium; s_prausnitzii | | | | | | | | |
| k_Bacteria; p_Firmicutes; c_Clostridia; o_Clostridiales; | v1822 | 83 | 0.78 | 78 | 0.22 | 4.2E−04 | 3.60 | 0.28 |
| f_Ruminococcaceae; g_Faecalibacterium; s_prausnitzii | | | | | | | | |
| k_Bacteria; p_Bacteroidetes; c_Clostridia; o_Bacteroidales; | v3729 | 83 | 0.01 | 78 | 0.45 | 4.3E−04 | −3.60 | 37.24 |
| f_Rikenellaceae; g_Bacteroides | | | | | | | | |
| k_Bacteria; p_Firmicutes; c_Clostridia; o_Clostridiales; | v2186 | 83 | 0.61 | 78 | 0.12 | 4.4E−04 | 3.59 | 0.19 |
| f_Ruminococcaceae; g_Faecalibacterium; s_prausnitzii | | | | | | | | |
| k_Bacteria; p_Firmicutes; c_Clostridia; o_Clostridiales; | v364 | 83 | 0.80 | 78 | 4.01 | 5.0E−04 | −3.55 | 5.05 |
| f_Ruminococcaceae | | | | | | | | |
| k_Bacteria; p_Firmicutes; c_Clostridia; o_Clostridiales; | v1812 | 83 | 0.80 | 78 | 0.23 | 5.0E−04 | 3.55 | 0.29 |
| f_Ruminococcaceae; g_Faecalibacterium; s_prausnitzii | | | | | | | | |
| k_Bacteria; p_Firmicutes; c_Clostridia; o_Clostridiales; | v2237 | 83 | 0.70 | 78 | 0.14 | 5.3E−04 | 3.54 | 0.20 |
| f_Ruminococcaceae; g_Faecalibacterium; s_prausnitzii | | | | | | | | |
| k_Bacteria; p_Bacteroidetes; c_Bacteroidia; o_Bacteroidales; | v47 | 83 | 3.63 | 78 | 44.60 | 5.4E−04 | −3.53 | 12.30 |
| f_Rikenellaceae; g_Bacteroides | | | | | | | | |
| k_Bacteria; p_Firmicutes; c_Clostridia; o_Clostridiales; | v1730 | 83 | 0.76 | 78 | 0.22 | 5.5E−04 | 3.53 | 0.29 |
| f_Ruminococcaceae; g_Faecalibacterium; s_prausnitzii | | | | | | | | |
| k_Bacteria; p_Firmicutes; c_Clostridia; o_Clostridiales; | v438 | 83 | 3.25 | 78 | 0.27 | 5.5E−04 | 3.52 | 0.08 |
| f_Ruminococcaceae; g_Oscillospira | | | | | | | | |
| k_Bacteria; p_Firmicutes; c_Clostridia; o_Clostridiales; | v652 | 83 | 2.31 | 78 | 0.51 | 5.9E−04 | 3.51 | 0.22 |
| f_Ruminococcaceae; g_Faecalibacterium; s_prausnitzii | | | | | | | | |
| k_Bacteria; p_Firmicutes; c_Clostridia; o_Clostridiales; | v556 | 83 | 2.72 | 78 | 0.35 | 6.2E−04 | 3.49 | 0.13 |
| f_Ruminococcaceae; g_Faecalibacterium; s_prausnitzii | | | | | | | | |
| k_Bacteria; p_Firmicutes; c_Clostridia; o_Clostridiales; | v1739 | 83 | 0.66 | 78 | 0.17 | 6.3E−04 | 3.49 | 0.25 |
| f_Ruminococcaceae; g_Faecalibacterium; s_prausnitzii | | | | | | | | |
| k_Bacteria; p_Firmicutes; c_Clostridia; o_Clostridiales; | v2788 | 83 | 0.51 | 78 | 0.13 | 6.5E−04 | 3.48 | 0.25 |
| f_Ruminococcaceae; g_Faecalibacterium; s_prausnitzii | | | | | | | | |
| k_Bacteria; p_Firmicutes; c_Clostridia; o_Clostridiales; | v6416 | 83 | 0.24 | 78 | 0.01 | 7.5E−04 | 3.44 | 0.05 |
| f_Ruminococcaceae; g_Faecalibacterium; s_prausnitzii | | | | | | | | |
| k_Bacteria; p_Firmicutes; c_Clostridia; o_Clostridiales; | v4689 | 83 | 0.29 | 78 | 0.05 | 7.8E−04 | 3.42 | 0.18 |
| f_Ruminococcaceae; g_Faecalibacterium; s_prausnitzii | | | | | | | | |
| k_Bacteria; p_Firmicutes; c_Clostridia; o_Clostridiales; | v2138 | 83 | 0.67 | 78 | 0.15 | 8.0E−04 | 3.42 | 0.23 |
| f_Ruminococcaceae; g_Faecalibacterium; s_prausnitzii | | | | | | | | |
| k_Bacteria; p_Bacteroidetes; c_Bacteroidia; o_Bacteroidales; | v2013 | 83 | 0.64 | 78 | 0.17 | 8.1E−04 | 3.42 | 0.26 |
| f_Bacteroidaceae; g_Bacteroides | | | | | | | | |
| k_Bacteria; p_Firmicutes; c_Clostridia; o_Clostridiales; | v13610 | 83 | 0.17 | 78 | 0.00 | 8.2E−04 | 3.41 | 0.00 |
| f_Ruminococcaceae; g_Faecalibacterium; s_prausnitzii | | | | | | | | |
| k_Bacteria; p_Firmicutes; c_Clostridia; o_Clostridiales; | v439 | 83 | 0.53 | 78 | 5.06 | 8.7E−04 | −3.39 | 9.55 |
| f_Ruminococcaceae; g_Oscillospira | | | | | | | | |
| k_Bacteria; p_Firmicutes; c_Clostridia; o_Clostridiales | v3796 | 83 | 0.18 | 78 | 0.00 | 9.2E−04 | 3.38 | 0.00 |
| k_Bacteria; p_Firmicutes; c_Clostridia; o_Clostridiales; | v1534 | 83 | 0.89 | 78 | 0.23 | 9.4E−04 | 3.37 | 0.26 |
| f_Ruminococcaceae; g_Faecalibacterium; s_prausnitzii | | | | | | | | |
| k_Bacteria; p_Firmicutes; c_Clostridia; o_Clostridiales; | v1583 | 83 | 0.10 | 78 | 0.64 | 9.5E−04 | −3.37 | 6.65 |
| f_Ruminococcaceae; g_Ruminococcus; s_bromii | | | | | | | | |
| k_Bacteria; p_Firmicutes; c_Clostridia; o_Clostridiales; | v2464 | 83 | 0.45 | 78 | 0.13 | 9.9E−04 | 3.36 | 0.29 |
| f_Ruminococcaceae; g_Faecalibacterium; s_prausnitzii | | | | | | | | |

TABLE 32
Gut microbes corresponding with high and low GTA-592 levels:

| OTUs | OTUs | N1 | meanQ1 | N2 | meanQ5 | p value | tstatistic | ratio |
|---|---|---|---|---|---|---|---|---|
| GTA591555 | | 81 | −0.02 | 78 | 0.60 | 1.3E−48 | −21.49 | |
| k_Bacteria; p_Firmicutes; c_Clostridia; o_Clostridiales; f_Ruminococcaceae | v364 | 81 | 0.74 | 78 | 4.49 | 3.5E−04 | −3.66 | 6.06 |
| k_Bacteria; p_Firmicutes; c_Clostridia; o_Clostridiales; f_Ruminococcaceae; | v2874 | 81 | 0.43 | 78 | 0.05 | 3.2E−04 | 3.41 | 0.12 |
| g_Faecalibacterium; s_prausnitzii | | | | | | | | |

TABLE 33
Gut microbes corresponding with high and low GTA-594 levels:

| OTUs | OTUs | N1 | meanQ1 | N2 | meanQ5 | p value | tstatistic | ratio |
|---|---|---|---|---|---|---|---|---|
| GTA593557 | | 81 | 0.37 | 81 | 2.08 | 8.1E−52 | −22.67 | |
| k_Bacteria; p_Firmicutes; c_Clostridia; o_Clostridiales; f_Ruminococcaceae; | v15704 | 81 | 0.00 | 81 | 0.15 | 8.2E−04 | −3.41 | |
| g_Faecalibacterium; s_prausnitzii | | | | | | | | |
| k_Bacteria; p_Firmicutes; c_Clostridia; o_Clostridiales; f_Clostridiaceae | v5030 | 81 | 0.01 | 81 | 0.31 | 2.4E−04 | −3.76 | 25.00 |
| k_Bacteria; p_Bacteroidetes; c_Bacteroidis; o_Bacteroidales; f_Rikenellaceae; | v7275 | 81 | 0.02 | 81 | 0.22 | 8.7E−04 | −3.39 | 9.00 |
| g_Bacteroides | | | | | | | | |
| k_Bacteria; p_Firmicutes; c_Clostridia; o_Clostridiales; f_Ruminococcacese; | v3121 | 81 | 0.21 | 81 | 0.00 | 6.2E−04 | 3.49 | 0.00 |
| g_Ruminococcus | | | | | | | | |
| k_Bacteria; p_Firmicutes; c_Bacilli; o_Lactobacillales; f_Streptococcacese; | v10586 | 81 | 0.20 | 81 | 0.00 | 6.4E−04 | 3.48 | 0.00 |
| g_Streptococcus | | | | | | | | |
| k_Bacteria; p_Bacteroidetes; c_Bacteroidia; o_Bacteroidales; f_Bacteroidaceae; | v3514 | 81 | 0.27 | 81 | 0.06 | 5.2E−04 | 3.54 | 0.23 |
| g_Bacteroides | | | | | | | | |
| k_Bacteria; p_Firmicutes; c_Bacilli; o_Lactobacillales; f_Streptococcaceae; | v3501 | 81 | 0.63 | 81 | 0.10 | 7.4E−04 | 3.44 | 0.16 |
| g_Streptoccoccus | | | | | | | | |
| k_Bacteria; p_Firmicutes; c_Clostridia; o_Clostridiales; f_Lachnospiraceae; | v4161 | 81 | 0.21 | 81 | 0.02 | 8.9E−04 | 3.39 | 0.12 |
| g_Blautia | | | | | | | | |

TABLE 34
Gut microbes corresponding with high and low GTA-596 levels:

| OTUs | OTUs | N1 | meanQ1 | N2 | meanQ5 | p value | tstatistic | ratio |
|---|---|---|---|---|---|---|---|---|
| GTA595559 | | 82 | 0.19 | 81 | 1.61 | 3.1E−34 | −15.63 | |
| k_Bacteria; p_Firmicutes; c_Clostridia; o_Clostridiales; f_Lachnospiraceae | v2563 | 82 | 0.06 | 81 | 0.56 | 7.1E−04 | −3.45 | 9.11 |
| k_Bacteria; p_Firmicutes; c_Clostridia; o_Clostridiales; f_Clostridiaceae | v5030 | 82 | 0.04 | 81 | 0.33 | 7.5E−04 | −3.44 | 9.11 |
| k_Bacteria; p_Bacteroidetes; c_Bacteroidia; o_Bacteroidales; f_Bacteroidaceae; | v2467 | 82 | 0.44 | 81 | 0.12 | 8.9E−04 | 3.39 | 0.28 |
| g_Bacteroides | | | | | | | | |
| k_Bacteria; p_Firmicutes; c_Bacilli; o_Lactobacillales; f_Streptococcaceae; | v3501 | 82 | 0.70 | 81 | 0.10 | 3.2E−04 | 3.68 | 0.14 |
| g_Streptococcus | | | | | | | | |

### 2. Measuring GTA Levels in Gut Microbe Samples

Three human, one dog and one pig fecal samples were incubated in brain heart infusion (BHI) media under aerobic and anaerobic conditions for 24, 48, 72 and 96 hours. Cell pellets were extracted by mechanical lysis and sonication in organic solvents, followed by the analysis of the solvents by tandem mass spectrometry to determine GTA levels.

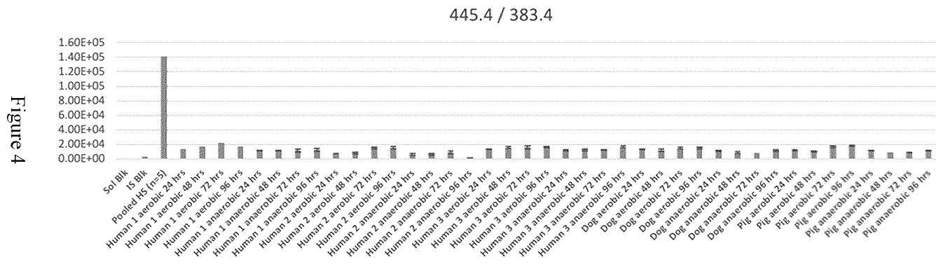

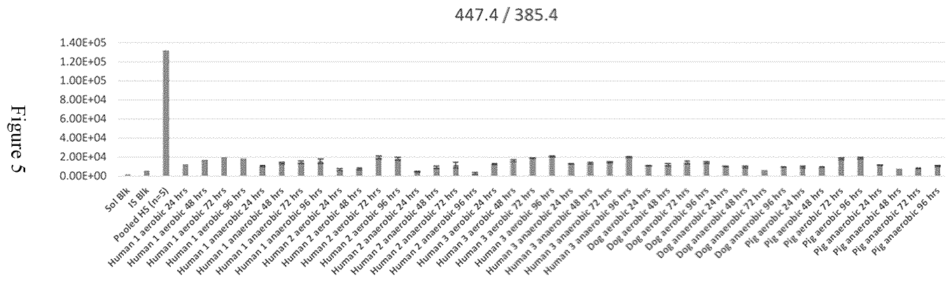

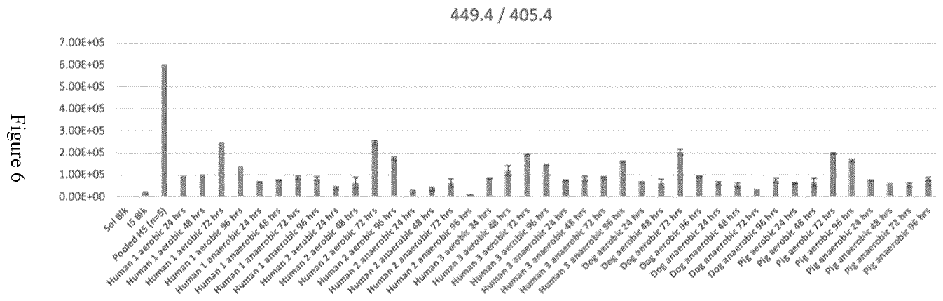

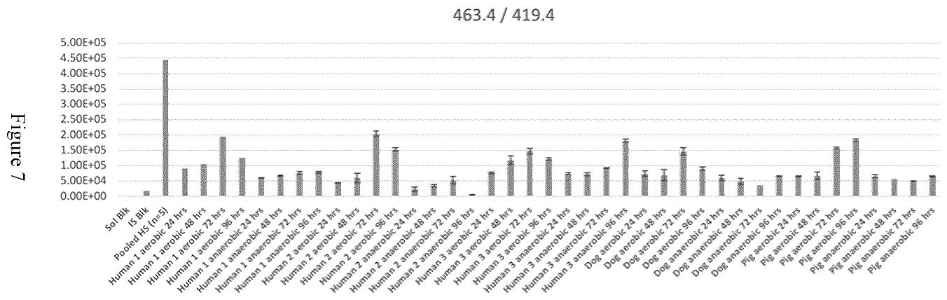

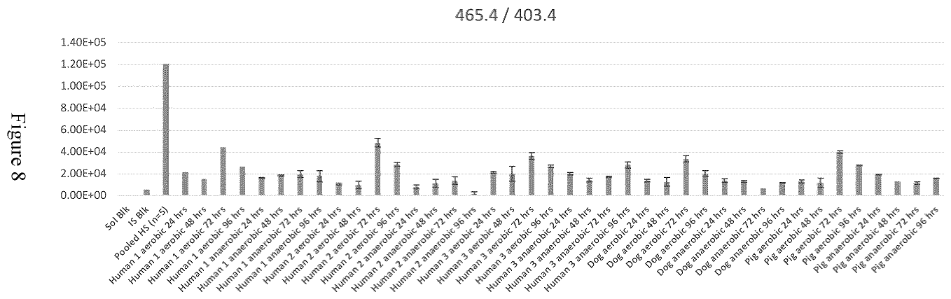

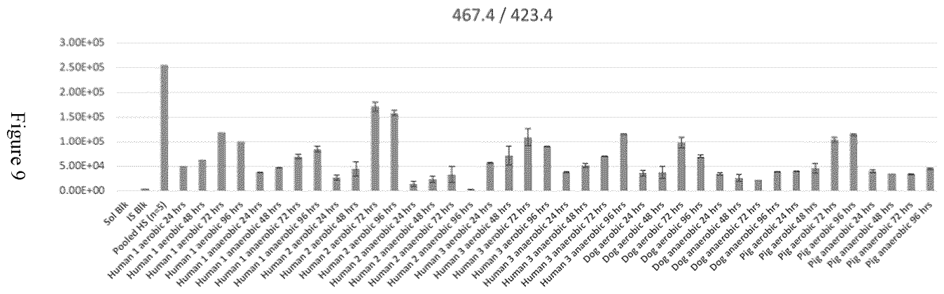

Selected GTAs were detected in most samples above background levels. For example, GTA-445.4/383.4 and GTA 447.4/385.4 were detected at relatively low levels compared to a human serum sample, but still above background levels. GTAs 449.4/405.4, 463.4/419.4, 465.4 /403.4 were all detected at levels well above background and approaching 50% of a human serum sample, particularly at 72 hours across all conditions analyzed. These results are shown in FIGS. 4-9.

The results provide the first evidence that GTAs appear to be the products of gut microbes. The present invention therefore provides for the use of microbial sources to produce GTAs or to augment GTA levels in subjects by providing probiotics containing combinations of GTA-producing microbes. This can include the commercial production of GTAs using industrial fermentation systems, methods of isolating, selecting and/or enriching for microbial strains involved in GTA production.

One or more currently preferred embodiments have been described by way of example. It will be apparent to persons skilled in the art that a number of variations and modifications can be made without departing from the scope of the invention as defined in the claims.

### REFERENCES

Ben-Neriah, Y. & M. Karin (2011) Inflammation meets cancer, with NF-kappaB as the matchmaker. Nat Immunol, 12, 715-23.

Chapkin, R., D. McMurray & J. Lupton (2007) Colon cancer, fatty acids and anti-inflammatory compounds. Curr Opin Gastroenterol, 23, 48-54.

Demaria, S., E. Pikarsky, M. Karin, L. Coussens, Y. Chen, E. El-Omar, G. Trinchieri, S. Dubinett, J. Mao, E. Szabo, A. Krieg, G. Weiner, B. Fox, G. Coukos, E. Wang, R. Abraham, M. Carbone & M. Lotze (2010) Cancer and inflammation: promise for biologic therapy. J Immunother, 33, 335-351.

Freitas, R. & C. A. M. Fraga (2018) NF-kappaB-IKKbeta pathway as a target for drug development: realities, challenges and perspectives. Curr Drug Targets.

Hussain, S. P. & C. C. Harris (2007) Inflammation and cancer: an ancient link with novel potentials. Int J Cancer, 121, 2373-80.

Itzkowitz, S. & X. Yio (2004) Inflammation and cancer IV. Colorectal cancer in inflammatory bowel disease: the role of inflammation. Am J Physiol Gastrointest Liver Physiol, 287, G7-17.

Maccio, A. & C. Madeddu (2012) Inflammation and ovarian cancer. Cytokine, 58, 133-47.

Marusawa, H. & B. J. Jenkins (2014) Inflammation and gastrointestinal cancer: an overview. Cancer Lett, 345, 153-6.

Ritchie, S., P. Ahiahonu, D. Jayasinghe, D. Heath, J. Liu, Y. Lu, W. Jin, A. Kavianpour, Y. Yamazaki, A. Khan, M. Hossain, K. Su-Myat, P. Wood, K. Krenitsky, I. Takemasa, M. Miyake, M. Sekimoto, M. Monden, H. Matsubara, F. Nomura & D. Goodenowe (2010a) Reduced levels of hydroxylated, polyunsaturated ultra long-chain fatty acids in the serum of colorectal cancer patients: implications for early screening and detection. BMC medicine, 8.

Ritchie, S., D. Heath, Y. Yamazaki, B. Grimmalt, A. Kavianpour, K. Krenitsky, H. Elshoni, I. Takemasa, M. Miyake, M. Sekimoto, M. Monden, T. Tomonaga, H. Matsubara, K. Sogawa, K. Matsushita, F. Nomura & D. Goodenowe (2010b) Reduction of novel circulating long-chain fatty acids in colorectal cancer patients is independent of tumor burden and correlates with age. BMC gastroenterology, 10.

Ritchie, S. A., H. Akita, I. Takemasa, H. Eguchi, E. Pastural, H. Nagano, M. Monden, Y. Doki, M. Mori, W. Jin, T. T. Sajobi, D. Jayasinghe, B. Chitou, Y. Yamazaki, T. White & D. B. Goodenowe (2013a) Metabolic system alterations in pancreatic cancer patient serum: potential for early detection. BMC Cancer, 13, 416.

Ritchie, S. A., B. Chitou, Q. Zheng, D. Jayasinghe, W. Jin, A. Mochizuki & D. B. Goodenowe (2015) Pancreatic cancer serum biomarker PC-594: Diagnostic performance and comparison to CA19-9. World J Gastroenterol, 21, 6604-12.

Ritchie, S. A., D. Heath, Y. Yamazaki, B. Grimmalt, A. Kavianpour, K. Krenitsky, H. Elshoni, I.

Takemasa, M. Miyake, M. Sekimoto, M. Monden, T. Tomonaga, H. Matsubara, K. Sogawa, K. Matsushita, F. Nomura & D. B. Goodenowe (2010c) Reduction of novel circulating long-chain fatty acids in colorectal cancer patients is independent of tumor burden and correlates with age. BMC Gastroenterol, 10, 140.

Ritchie, S. A., D. Jayasinghe, G. F. Davies, P. Ahiahonu, H. Ma & D. B. Goodenowe (2011) Human serum-derived hydroxy long-chain fatty acids exhibit anti-inflammatory and anti-proliferative activity. J Exp Clin Cancer Res, 30, 59.

Ritchie, S. A., J. Tonita, R. Alvi, D. Lehotay, H. Elshoni, S. Myat, J. McHattie & D. B. Goodenowe (2013b) Low-serum GTA-446 anti-inflammatory fatty acid levels as a new risk factor for colon cancer. Int J Cancer, 132, 355-62.

Schwartsburd, P. (2004) Age-promoted creation of a pro-cancer microenvironment by inflammation: pathogenesis of dyscoordinated feedback control. Mech Ageing Dev, 125, 581-590.

Spehlmann, M. & L. Eckmann (2009) Nuclear factor-kappa B in intestinal protection and destruction. Curr Opin Gastroenterol, 25, 92-99.

Surh, Y., K. Chun, H. Cha, S. Han, Y. Keum, K. Park & S. Lee (2001) Molecular mechanisms underlying chemopreventive activities of anti-inflammatory phytochemicals: down-regulation of COX-2 and iNOS through suppression of NF-kappa B activation. Mutat Res, 480-481, 243-268.

Terzic, J., S. Grivennikov, E. Karin & M. Karin (2010) Inflammation and colon cancer. Gastroenterology, 138, 2101-2114 e5.

Wu, Y., S. Antony, J. L. Meitzler & J. H. Doroshow (2014) Molecular mechanisms underlying chronic inflammation-associated cancers. Cancer Lett, 345, 164-73.

Xu, C., G. Shen, C. Chen, C. Gelinas & A. Kong (2005) Suppression of NF-kappaB and NF-kappaB-regulated gene expression by sulforaphane and PEITC through IkappaBalpha, IKK pathway in human prostate cancer PC-3 cells. Oncogene, 24, 4486-4495.

The above listed publications are each incorporated herein by reference in their entirety.

What is claimed is:

1. A method for increasing gastric tract acid (GTA) production in a mammalian subject, comprising administering a therapeutically-effective amount of a composition comprising at least one live or attenuated culture containing a microbial species selected from the genus Blautia, species Faecalibacterium prausnitzii, genus Bacteroides, family Ruminococcaceae, family Lachnospiraceae, genus Coprococcus, genus Roseburia, genus Oscillospira, species Ruminococcus bromii, genus Ruminococcus, family Costridiaceae, species Dorea formicigenerans, species Bacteroides uniformis, genus Dorea, genus Streptococcus, order Clostridiales, genus Anaerostipes, genus Dialister, species Bifidobacterium adolescentis, family Coriobacteriaceae, genus Faecalibacterium, genus Sutterella, species Bacteroides ovatus, genus Parabacteroides, genus Ruminococcus, species Bacteroides faecis, species Eubacterium biforme, genus Phascolartobacterium, and family Enterobacteriaceae; or a prebiotic composition which increases growth and/or viability of said microbial species in the gut; wherein the composition increases the synthesis of at least one GTA dicarboxylic fatty acid metabolite in said subject.

2. The method of claim 1, wherein the method further comprises a step of measuring circulating levels of one or more GTA dicarboxylic fatty acid metabolite in a subject, and administering said composition if the levels of said one or more GTA dicarboxylic fatty acid metabolite in said subject are lower than a predetermined control level, an earlier test value for said subject, or a normal level for healthy subjects.

3. The method of claim 2, wherein the control comprises a predetermined threshold value for said at least one GTA dicarboxylic fatty acid metabolite.

4. The method of claim 1, wherein said composition comprises a live or attenuated culture of a microbial species from the genus Blautia, a live or attenuated culture of Faecalibacterium prausnitzii, or a combination thereof, within a pharmaceutically-acceptable carrier suitable for administration to the gastrointestinal tract of said subject.

5. The method of claim 1, wherein said GTA dicarboxylic fatty acid metabolite is a dicarboxylic fatty acid between 28 and 36 carbons comprised of a dimeric fatty acid structure of two shorter chains ranging between 14 and 18 carbons in length joined by a single or double bond.

6. The method of claim 1, wherein said GTA dicarboxylic fatty acid metabolite is selected from the group consisting of: GTA-446, GTA-448, GTA-450, GTA-452, GTA-464, GTA-466, GTA-468, GTA-474, GTA-476, GTA-478, GTA-484, GTA-490, GTA-492, GTA-494, GTA-502, GTA-504, GTA-512, GTA-518, GTA-520, GTA-522, GTA-524, GTA-530, GTA-532, GTA-536, GTA-538, GTA-540, GTA-550, GTA-574, GTA-576, GTA-580, GTA-590, GTA-592, GTA-594, and GTA-596.

7. The method of claim 1, wherein said GTA dicarboxylic fatty acid metabolite has an accurate neutral mass within 1 PPM Dalton mass accuracy of 446.3396 (GTA-446), 448.3553 (GTA-448), 450.3709 (GTA-450), 452.3866 (GTA-452), 464.3522 (GTA-464), 466.3661 (GTA-466), 468.3814 (GTA-468), 474.3736 (GTA-474), 476.3866 GTA-476, 478.4022 (GTA-478), 484.3764 (GTA-484), 490.3658 (GTA-490), 492.3815 (GTA-492), 494.3971 (GTA-494), 502.4022 (GTA-502), 504.4195 (GTA-504), 512.4077 (GTA-512), 518.3974 (GTA-518), 520.4128 (GTA-520), 522.4284 (GTA-522), 524.4441 (GTA-524), 530.4335 (GTA-530), 532.4492 (GTA-532), 536.4077 (GTA-536), 538.4233 (GTA-538), 540.4389 (GTA-540), 550.4597 (GTA-550), 574.4597 (GTA-574), 576.4754 (GTA-576), 580.5067 (GTA-580), 590.4546 (GTA-590), 592.4703 (GTA-592), 594.4859 (GTA-594), or 596.5016 (GTA-596).

8. The method of claim 1, wherein said GTA dicarboxylic fatty acid metabolite has a molecular formula of C₂₈H₄₆O₄ (GTA-446), C₂₈H₄₈O₄ (GTA-448), C₂₈H₅₀O₄ (GTA-450), C₂₈H₅₂O₄ (GTA-452), 464.3522 (GTA-464), 466.3661 (GTA-466), C₂₈H₅₂O(GTA-468), C₃₀H₅₀O₄ (GTA-474), C₃₀H₅₂O₄ GTA-476, C₃₀H₅₄O₄ (GTA-478), C₂₈H₅₂O₆ (GTA-484), C₃₀H₅₀O₅ (GTA-490), C₃₀H₅₂O₅ (GTA-492), C₃₀H₅₄O₅ (GTA-494), C₃₂H₅₄O₄ (GTA-502), C₃₂H₅₆O₄ (GTA-504), C₃₀H₅₆O₆ (GTA-512), C₃₂H₅₄O₅ (GTA-518), C₃₂H₅₆O₅ (GTA-520), C₃₂H₅₈O₅ (GTA-522), C₃₂H₆₀O₅ GTA-524, C₃₄H₅₈O₄ (GTA-530), C₃₄H₆₀O₄ (GTA-532), C₃₂H₅₆O₆ (GTA-536), C₃₂H₅₈O₆ (GTA-538), C₃₂H₆₀O₆ (GTA-540), C₃₄H₆₂O₅ (GTA-550), C₃₆H₆₂O₅ (GTA-574), C₃₆H₆₄O₅ (GTA-576), C₃₆H₆₈O₅ (GTA-580), C₃₆H₆₂O₆ (GTA-590), C₃₆H₆₄O₆ (GTA-592), C₃₆H₆₆O₆ (GTA-594), or C₃₆H₆₈O₆ (GTA-596).

9. The method of claim 2, wherein said GTA dicarboxylic fatty acid metabolite is measured using collision induced dissociation (CID) tandem mass spectrometry and is selected from one or more of the GTA dicarboxylic fatty acid metabolites listed below:
GTA-446, having an accurate neutral mass within 1 PPM Dalton mass accuracy of 446.3396, the molecular formula of C₂₈H₄₆O₄, and characterized by a OD MS/MS fragmentation pattern using N₂ as collision gas and analyzed in atmospheric pressure chemical ionization (APCI) under negative ionization comprising daughter ions of parent [M−H] mass 445: 427, 409, 401, and 383,
GTA-448, having an accurate neutral mass within 1 PPM Dalton mass accuracy of 448.3553, the molecular formula of C₂₈H₄₈O₄, and characterized by a OD MS/MS fragmentation pattern using N₂ as collision gas and analyzed in atmospheric pressure chemical ionization (APCI) under negative ionization comprising daughter ions of parent [M−H] mass 447: 429, 411, 403, and 385,
GTA-450, having an accurate neutral mass within 1 PPM Dalton mass accuracy of 450.3709, the molecular formula of C₂₈H₅₀O₄, and characterized by a OD MS/MS fragmentation pattern using N₂ as collision gas and analyzed in atmospheric pressure chemical ionization (APCI) under negative ionization comprising daughter ions of parent [M−H] mass 449: 431, 413, 405, and 387,
GTA-452, having an accurate neutral mass within 1 PPM Dalton mass accuracy of 452.3866, the molecular formula of C₂₈H₅₂O₄, and characterized by a OD MS/MS fragmentation pattern using N₂ as collision gas and analyzed in atmospheric pressure chemical ionization (APCI) under negative ionization comprising daughter ions of parent [M−H] mass 451: 433, 407, and 389,
GTA-464, having an accurate neutral mass within 1 PPM Dalton mass accuracy of 464.3522, the molecular formula of C₂₈H₄₈O₅, and characterized by a OD MS/MS fragmentation pattern using N₂ as collision gas and analyzed in atmospheric pressure chemical ionization (APCI) under negative ionization comprising daughter ions of parent [M−H] mass 463: 445, 419, 401, and 383,
GTA-466, having an accurate neutral mass within 1 PPM Dalton mass accuracy of 466.3661, the molecular formula of C₂₈H₅₀O₅, and characterized by a OD MS/MS fragmentation pattern using N₂ as collision gas and analyzed in atmospheric pressure chemical ionization (APCI) under negative ionization comprising daughter ions of parent [M−H] mass 465: 447, 421, and 403,
GTA-468, having an accurate neutral mass within 1 PPM Dalton mass accuracy of 468.3814, having the molecular formula of C₂₈H₅₂O₅, and characterized by a OD MS/MS fragmentation pattern using N₂ as collision gas and analyzed in atmospheric pressure chemical ionization (APCI) under negative ionization comprising daughter ions of parent [M−H] mass 467: 449, 423, and 405,
GTA-474, having an accurate neutral mass within 1 PPM Dalton mass accuracy of 474.3736, having the molecular formula of C₃₀H₅₀O₄, and characterized by a OD MS/MS fragmentation pattern using N₂ as collision gas and analyzed in atmospheric pressure chemical ionization (APCI) under negative ionization comprising daughter ions of parent [M−H] mass 473: 455, 429, and 411,
GTA-476, having an accurate neutral mass within 1 PPM Dalton mass accuracy of 476.3866, having the molecular formula of C₃₀H₅₂O₄, and characterized by a OD MS/MS fragmentation pattern using N₂ as collision gas and analyzed in atmospheric pressure chemical ionization (APCI) under negative ionization comprising daughter ions of parent [M−H] mass 475: 457, 431, 439 and 413,
GTA-478, having an accurate neutral mass within 1 PPM Dalton mass accuracy of 478.4022, having the molecular formula of C₃₀H₅₄O₄, and characterized by a OD MS/MS fragmentation pattern using N₂ as collision gas and analyzed in atmospheric pressure chemical ionization (APCI) under negative ionization comprising daughter ions of parent [M−H] mass 477: 459, 433, 441 and 415,
GTA-484, having an accurate neutral mass within 1 PPM Dalton mass accuracy of 484.3764, having the molecular formula of C₂₈H₅₂O₆, and characterized by a OD MS/MS fragmentation pattern using N₂ as collision gas and analyzed in atmospheric pressure chemical ionization (APCI) under negative ionization comprising daughter ions of parent [M−H] mass 483: 465, 315, 439 483, 421, and 447,
GTA-490, having an accurate neutral mass within 1 PPM Dalton mass accuracy of 490.3658, having the molecular formula of C₃₀H₅₀O₅, and characterized by a OD MS/MS fragmentation pattern using N₂ as collision gas and analyzed in atmospheric pressure chemical ionization (APCI) under negative ionization comprising daughter ions of parent [M−H] mass 489: 445, 471, 427 and 319,
GTA-492, having an accurate neutral mass within 1 PPM Dalton mass accuracy of 492.3815, having the molecular formula of C₃₀H₅₂O₅, and characterized by a OD MS/MS fragmentation pattern using N₂ as collision gas and analyzed in atmospheric pressure chemical ionization (APCI) under negative ionization comprising daughter ions of parent [M−H] mass 491: 241, 249, 267, 473, and 447,
GTA-494, having an accurate neutral mass within 1 PPM Dalton mass accuracy of 494.3971, having the molecular formula of C₃₀H₅₄O₅, and characterized by a OD MS/MS fragmentation pattern using N₂ as collision gas and analyzed in atmospheric pressure chemical ionization (APCI) under negative ionization comprising daughter ions of parent [M−H] mass 493: 475, 215, and 449,
GTA-502, having an accurate neutral mass within 1 PPM Dalton mass accuracy of 502.4022, having the molecular formula of C₃₂H₅₄O₄, and characterized by a OD MS/MS fragmentation pattern using N₂ as collision gas and analyzed in atmospheric pressure chemical ionization (APCI) under negative ionization comprising daughter ions of parent [M−H] mass 501: 483, 457, 465 and 439,
GTA-504, having an accurate neutral mass within 1 PPM Dalton mass accuracy of 504.4195, having the molecular formula of C₃₂H₅₆O₄, and characterized by a OD MS/MS fragmentation pattern using N₂ as collision gas and analyzed in atmospheric pressure chemical ionization (APCI) under negative ionization comprising daughter ions of parent [M−H] mass 503: 485, 459, 467 and 441,
GTA-512, having an accurate neutral mass within 1 PPM Dalton mass accuracy of 512.4077, having the molecular formula of C₃₀H₅₆O₆, and characterized by a OD MS/MS fragmentation pattern using N₂ as collision gas and analyzed in atmospheric pressure chemical ionization (APCI) under negative ionization comprising daughter ions of parent [M−H] mass 511: 493, 315, and 467,
GTA-518, having an accurate neutral mass within 1 PPM Dalton mass accuracy of 518.3974, having the molecular formula of C₃₂H₅₄O₅, and characterized by a OD MS/MS fragmentation pattern using N₂ as collision gas and analyzed in atmospheric pressure chemical ionization (APCI) under negative ionization comprising daughter ions of parent [M−H] mass 517: 499, 473, 499, 481 and 445,
GTA-520, having an accurate neutral mass within 1 PPM Dalton mass accuracy of 520.4128, having the molecular formula of C₃₂H₅₆O₅, and characterized by a OD MS/MS fragmentation pattern using N₂ as collision gas and analyzed in atmospheric pressure chemical ionization (APCI) under negative ionization comprising daughter ions of parent [M−H] mass 519: 501, 457, 475, 459, 447 and 483,
GTA-522, having an accurate neutral mass within 1 PPM Dalton mass accuracy of 522.4284, having the molecular formula of C₃₂H₅₈O₅, and characterized by a OD MS/MS fragmentation pattern using N₂ as collision gas and analyzed in atmospheric pressure chemical ionization (APCI) under negative ionization comprising daughter ions of parent [M−H] mass 521: 503, 459, 477, 504, 441 and 485,
GTA-524, having an accurate neutral mass within 1 PPM Dalton mass accuracy of 524.4441, having the molecular formula of C₃₂H₆₀O₅, and characterized by a OD MS/MS fragmentation pattern using N₂ as collision gas and analyzed in atmospheric pressure chemical ionization (APCI) under negative ionization comprising daughter ions of parent [M−H] mass 523: 505, 461, 479, 506, 443 and 487,
GTA-530, having an accurate neutral mass within 1 PPM Dalton mass accuracy of 530.4335, having the molecular formula of C₃₄H₅₈O₄, and characterized by a OD MS/MS fragmentation pattern using N₂ as collision gas and analyzed in atmospheric pressure chemical ionization (APCI) under negative ionization comprising daughter ions of parent [M−H] mass 529: 467, 511 and 485,
GTA-532, having an accurate neutral mass within 1 PPM Dalton mass accuracy of 532.4492, having the molecular formula of C₃₄H₆₀O₄, and characterized by a OD MS/MS fragmentation pattern using N₂ as collision gas and analyzed in atmospheric pressure chemical ionization (APCI) under negative ionization comprising daughter ions of parent [M−H] mass 531: 513, 469, 487 and 495,
GTA-536, having an accurate neutral mass within 1 PPM Dalton mass accuracy of 536.4077, having the molecular formula of C₃₂H₅₆O₆, and characterized by a OD MS/MS fragmentation pattern using N₂ as collision gas and analyzed in atmospheric pressure chemical ionization (APCI) under negative ionization comprising daughter ion of parent [M−H] mass 535: 473,
GTA-538, having an accurate neutral mass within 1 PPM Dalton mass accuracy of 538.4233, having the molecular formula of C₃₂H₅₈O₆, and characterized by a OD MS/MS fragmentation pattern using N₂ as collision gas and analyzed in atmospheric pressure chemical ionization (APCI) under negative ionization comprising daughter ions of parent [M−H] mass 537: 519, 475, 493, 501 and 457,
GTA-540, having an accurate neutral mass within 1 PPM Dalton mass accuracy of 540.4389, having the molecular formula of C₃₂H₆₀O₆, and characterized by a OD MS/MS fragmentation pattern using N₂ as collision gas and analyzed in atmospheric pressure chemical ionization (APCI) under negative ionization comprising daughter ions of parent [M−H] mass 539: 315, 521, 495 and 477,
GTA-550, having an accurate neutral mass within 1 PPM Dalton mass accuracy of 550.4597, having the molecular formula of C₃₄H₆₂O₅, and characterized by a OD MS/MS fragmentation pattern using N₂ as collision gas and analyzed in atmospheric pressure chemical ionization (APCI) under negative ionization comprising daughter ions of parent [M−H] mass 549: 487, 531, 251, 253, 513, 469 and 506,
GTA-574, having an accurate neutral mass within 1 PPM Dalton mass accuracy of 574.4597, having the molecular formula of C₃₆H₆₂O₅, and characterized by a OD MS/MS fragmentation pattern using N₂ as collision gas and analyzed in atmospheric pressure chemical ionization (APCI) under negative ionization comprising daughter ions of parent [M−H] mass 573:
295, 223, 555 and 511, GTA-576, having an accurate neutral mass within 1 PPM Dalton mass accuracy of 576.4754, having the molecular formula of C₃₆H₆₄O₅, and characterized by a OD MS/MS fragmentation pattern using N₂ as collision gas and analyzed in atmospheric pressure chemical ionization (APCI) under negative ionization comprising daughter ions of parent [M−H] mass 575: 277, 297, 557, 513 and 495,
GTA-580, having an accurate neutral mass within 1 PPM Dalton mass accuracy of 580.5067, having the molecular formula of C₃₆H₆₈O₅, and characterized by a OD MS/MS fragmentation pattern using N₂ as collision gas and analyzed in atmospheric pressure chemical ionization (APCI) under negative ionization comprising daughter ions of parent [M−H] mass 579: 561, 543, 535, 517 and 499,
GTA-590, having an accurate neutral mass within 1 PPM Dalton mass accuracy of 590.4546, having the molecular formula of C₃₆H₆₂O₆, and characterized by a OD MS/MS fragmentation pattern using N₂ as collision gas and analyzed in atmospheric pressure chemical ionization (APCI) under negative ionization comprising daughter ion of parent [M−H] mass 589: 545,
GTA-592, having an accurate neutral mass within 1 PPM Dalton mass accuracy of 592.4703, having the molecular formula of C₃₆H₆₄O₆, and characterized by a OD MS/MS fragmentation pattern using N₂ as collision gas and analyzed in atmospheric pressure chemical ionization (APCI) under negative ionization comprising daughter ions of parent [M−H] mass 591: 555 and 113,
GTA-594, having an accurate neutral mass within 1 PPM Dalton mass accuracy of 594.4859, having the molecular formula of C₃₆H₆₆O₆, and characterized by a OD MS/MS fragmentation pattern using N₂ as collision gas and analyzed in atmospheric pressure chemical ionization (APCI) under negative ionization comprising daughter ions of parent [M−H] mass 593: 557 371, 315 and 277, and
GTA-596, having an accurate neutral mass within 1 PPM Dalton mass accuracy of 596.5016, having the molecular formula of C₃₆H₆₈O₆, and characterized by a OD MS/MS fragmentation pattern using N₂ as collision gas and analyzed in atmospheric pressure chemical ionization (APCI) under negative ionization comprising daughter ions of parent [M−H] mass 595: 279, 315, 297, 577 and 559.

10. The method of claim 1, wherein said GTA dicarboxylic fatty acid metabolite is GTA-446, having the formula C₂₈H₄₆O₄ and the structure:

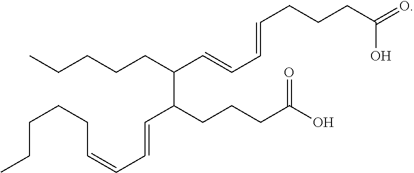 [SMILES: CCCCC/C=C\C=C\C(CCCC(=O)O)C(/C=C/C=C/CCCC(=O)O)CCCCC]

11. A method for determining gastrointestinal inflammation status within the body by measuring circulating levels of one or more GTA dicarboxylic fatty acid metabolite, wherein said GTA dicarboxylic fatty acid metabolite is a dicarboxylic fatty acid between 28 and 36 carbons comprised of a dimeric fatty acid structure of two shorter chains ranging between 14 and 18 carbons in length joined by a single or double bond,
wherein if a level one or more of said GTA dicarboxylic fatty acid metabolites in said subject are lower than a predetermined control level, an earlier test value for said subject, or a normal level for healthy subjects, the subject is assessed as having or being at risk for gastrointestinal inflammation.

12. The method of claim 11, wherein said GTA dicarboxylic fatty acid metabolite is selected from the group consisting of: GTA-446, GTA-448, GTA-450, GTA-452, GTA-464, GTA-466, GTA-468, GTA-474, GTA-476, GTA-478, GTA-484, GTA-490, GTA-492, GTA-494, GTA-502, GTA-504, GTA-512, GTA-518, GTA-520, GTA-522, GTA-524, GTA-530, GTA-532, GTA-536, GTA-538, GTA-540, GTA-550, GTA-574, GTA-576, GTA-580, GTA-590, GTA-592, GTA-594, and GTA-596.

13. The method of claim 12, wherein said GTA dicarboxylic fatty acid metabolite has an accurate neutral mass within 1 PPM Dalton mass accuracy of 446.3396 (GTA-446), 448.3553 (GTA-448), 450.3709 (GTA-450), 452.3866 (GTA-452), 464.3522 (GTA-464), 466.3661 (GTA-466), 468.3814 (GTA-468), 474.3736 (GTA-474), 476.3866 GTA-476, 478.4022 (GTA-478), 484.3764 (GTA-484), 490.3658 (GTA-490), 492.3815 (GTA-492), 494.3971 (GTA-494), 502.4022 (GTA-502), 504.4195 (GTA-504), 512.4077 (GTA-512), 518.3974 (GTA-518), 520.4128 (GTA-520), 522.4284 (GTA-522), 524.4441 (GTA-524), 530.4335 (GTA-530), 532.4492 (GTA-532), 536.4077 (GTA-536), 538.4233 (GTA-538), 540.4389 (GTA-540), 550.4597 (GTA-550), 574.4597 (GTA-574), 576.4754 (GTA-576), 580.5067 (GTA-580), 590.4546 (GTA-590), 592.4703 (GTA-592), 594.4859 (GTA-594), or 596.5016 (GTA-596).

14. The method of claim 12, wherein said GTA dicarboxylic fatty acid metabolite has a molecular formula of C₂₈H₄₆O₄ (GTA-446), C₂₈H₄₈O₄ (GTA-448), C₂₈H₅₀O₄ (GTA-450), C₂₈H₅₂O₄ (GTA-452), 464.3522 (GTA-464), 466.3661 (GTA-466), C₂₈H₅₂O(GTA-468), C₃₀H₅₀O₄ (GTA-474), C₃₀H₅₂O₄ GTA-476, C₃₀H₅₄O₄ (GTA-478), C₂₈H₅₂O₆ (GTA-484), C₃₀H₅₀O₅ (GTA-490), C₃₀H₅₂O₅ (GTA-492), C₃₀H₅₄O₅ (GTA-494), C₃₂H₅₄O₄ (GTA-502), C₃₂H₅₆O₄ (GTA-504), C₃₀H₅₆O₆ (GTA-512), C₃₂H₅₄O₅ (GTA-518), C₃₂H₅₆O₅ (GTA-520), C₃₂H₅₈O₅ (GTA-522), C₃₂H₆₀O₅ GTA-524, C₃₄H₅₈O₄ (GTA-530), C₃₄H₆₀O₄ (GTA-532), C₃₂H₅₆O₆ (GTA-536), C₃₂H₅₈O₆ (GTA-538), C₃₂H₆₀O₆ (GTA-540), C₃₄H₆₂O₅ (GTA-550), C₃₆H₆₂O₅ (GTA-574), C₃₆H₆₄O₅ (GTA-576), C₃₆H₆₈O₅ (GTA-580), C₃₆H₆₂O₆ (GTA-590), C₃₆H₆₄O₆ (GTA-592), C₃₆H₆₆O₆ (GTA-594), or C₃₆H₆₈O₆ (GTA-596).

15. The method of claim 11, wherein said GTA dicarboxylic fatty acid metabolite is measured using collision induced dissociation (CID) tandem mass spectrometry and is selected from one or more of the GTA dicarboxylic fatty acid metabolites listed below:
GTA-446, having an accurate neutral mass within 1 PPM Dalton mass accuracy of 446.3396, the molecular formula of C₂₈H₄₆O₄, and characterized by a OD MS/MS fragmentation pattern using N₂ as collision gas and analyzed in atmospheric pressure chemical ionization (APCI) under negative ionization comprising daughter ions of parent [M−H] mass 445: 427, 409, 401, and 383,
GTA-448, having an accurate neutral mass within 1 PPM Dalton mass accuracy of 448.3553, the molecular formula of C₂₈H₄₈O₄, and characterized by a OD MS/MS fragmentation pattern using N₂ as collision gas and analyzed in atmospheric pressure chemical ionization (APCI) under negative ionization comprising daughter ions of parent [M−H] mass 447: 429, 411, 403, and 385,
GTA-450, having an accurate neutral mass within 1 PPM Dalton mass accuracy of 450.3709, the molecular formula of C₂₈H₅₀O₄, and characterized by a OD MS/MS fragmentation pattern using N₂ as collision gas and analyzed in atmospheric pressure chemical ionization (APCI) under negative ionization comprising daughter ions of parent [M−H] mass 449: 431, 413, 405, and 387,
GTA-452, having an accurate neutral mass within 1 PPM Dalton mass accuracy of 452.3866, the molecular formula of C₂₈H₅₂O₄, and characterized by a OD MS/MS fragmentation pattern using N₂ as collision gas and analyzed in atmospheric pressure chemical ionization (APCI) under negative ionization comprising daughter ions of parent [M−H] mass 451: 433, 407, and 389,
GTA-464, having an accurate neutral mass within 1 PPM Dalton mass accuracy of 464.3522, the molecular formula of C₂₈H₄₈O₅, and characterized by a OD MS/MS fragmentation pattern using N₂ as collision gas and analyzed in atmospheric pressure chemical ionization (APCI) under negative ionization comprising daughter ions of parent [M−H] mass 463: 445, 419, 401, and 383,
GTA-466, having an accurate neutral mass within 1 PPM Dalton mass accuracy of 466.3661, the molecular formula of C₂₈H₅₀O₅, and characterized by a OD MS/MS fragmentation pattern using N₂ as collision gas and analyzed in atmospheric pressure chemical ionization (APCI) under negative ionization comprising daughter ions of parent [M−H] mass 465: 447, 421, and 403,
GTA-468, having an accurate neutral mass within 1 PPM Dalton mass accuracy of 468.3814, having the molecular formula of C₂₈H₅₂O, and characterized by a OD MS/MS fragmentation pattern using N₂ as collision gas and analyzed in atmospheric pressure chemical ionization (APCI) under negative ionization comprising daughter ions of parent [M−H] mass 467: 449, 423, and 405,
GTA-474, having an accurate neutral mass within 1 PPM Dalton mass accuracy of 474.3736, having the molecular formula of C₃₀H₅₀O₄, and characterized by a OD MS/MS fragmentation pattern using N₂ as collision gas and analyzed in atmospheric pressure chemical ionization (APCI) under negative ionization comprising daughter ions of parent [M−H] mass 473: 455, 429, and 411,
GTA-476, having an accurate neutral mass within 1 PPM Dalton mass accuracy of 476.3866, having the molecular formula of C₃₀H₅₂O₄, and characterized by a OD MS/MS fragmentation pattern using N₂ as collision gas and analyzed in atmospheric pressure chemical ionization (APCI) under negative ionization comprising daughter ions of parent [M−H] mass 475: 457, 431, 439 and 413,
GTA-478, having an accurate neutral mass within 1 PPM Dalton mass accuracy of 478.4022, having the molecular formula of C₃₀H₅₄O₄, and characterized by a OD MS/MS fragmentation pattern using N₂ as collision gas and analyzed in atmospheric pressure chemical ionization (APCI) under negative ionization comprising daughter ions of parent [M−H] mass 477: 459, 433, 441 and 415,
GTA-484, having an accurate neutral mass within 1 PPM Dalton mass accuracy of 484.3764, having the molecular formula of C₂₈H₅₂O₆, and characterized by a OD MS/MS fragmentation pattern using N₂ as collision gas and analyzed in atmospheric pressure chemical ionization (APCI) under negative ionization comprising daughter ions of parent [M−H] mass 483: 465, 315, 439 483, 421, and 447,
GTA-490, having an accurate neutral mass within 1 PPM Dalton mass accuracy of 490.3658, having the molecular formula of C₃₀H₅₀O₅, and characterized by a OD MS/MS fragmentation pattern using N₂ as collision gas and analyzed in atmospheric pressure chemical ionization (APCI) under negative ionization comprising daughter ions of parent [M−H] mass 489: 445, 471, 427 and 319,
GTA-492, having an accurate neutral mass within 1 PPM Dalton mass accuracy of 492.3815, having the molecular formula of C₃₀H₅₂O₅, and characterized by a OD MS/MS fragmentation pattern using N₂ as collision gas and analyzed in atmospheric pressure chemical ionization (APCI) under negative ionization comprising daughter ions of parent [M−H] mass 491: 241, 249, 267, 473, and 447,
GTA-494, having an accurate neutral mass within 1 PPM Dalton mass accuracy of 494.3971, having the molecular formula of C₃₀H₅₄O₅, and characterized by a OD MS/MS fragmentation pattern using N₂ as collision gas and analyzed in atmospheric pressure chemical ionization (APCI) under negative ionization comprising daughter ions of parent [M−H] mass 493: 475, 215, and 449,
GTA-502, having an accurate neutral mass within 1 PPM Dalton mass accuracy of 502.4022, having the molecular formula of C₃₂H₅₄O₄, and characterized by a OD MS/MS fragmentation pattern using N₂ as collision gas and analyzed in atmospheric pressure chemical ionization (APCI) under negative ionization comprising daughter ions of parent [M−H] mass 501: 483, 457, 465 and 439,
GTA-504, having an accurate neutral mass within 1 PPM Dalton mass accuracy of 504.4195, having the molecular formula of C₃₂H₅₆O₄, and characterized by a OD MS/MS fragmentation pattern using N₂ as collision gas and analyzed in atmospheric pressure chemical ionization (APCI) under negative ionization comprising daughter ions of parent [M−H] mass 503: 485, 459, 467 and 441,
GTA-512, having an accurate neutral mass within 1 PPM Dalton mass accuracy of 512.4077, having the molecular formula of C₃₀H₅₆O₆, and characterized by a OD MS/MS fragmentation pattern using N₂ as collision gas and analyzed in atmospheric pressure chemical ionization (APCI) under negative ionization comprising daughter ions of parent [M−H] mass 511: 493, 315, and 467,
GTA-518, having an accurate neutral mass within 1 PPM Dalton mass accuracy of 518.3974, having the molecular formula of C₃₂H₅₄O₅, and characterized by a OD MS/MS fragmentation pattern using N₂ as collision gas and analyzed in atmospheric pressure chemical ionization (APCI) under negative ionization comprising daughter ions of parent [M−H] mass 517: 499, 473, 499, 481 and 445,
GTA-520, having an accurate neutral mass within 1 PPM Dalton mass accuracy of 520.4128, having the molecular formula of C₃₂H₅₆O₅, and characterized by a OD MS/MS fragmentation pattern using N₂ as collision gas and analyzed in atmospheric pressure chemical ionization (APCI) under negative ionization comprising daughter ions of parent [M−H] mass 519: 501, 457, 475, 459, 447 and 483,
GTA-522, having an accurate neutral mass within 1 PPM Dalton mass accuracy of 522.4284, having the molecular formula of C₃₂H₅₈O₅, and characterized by a OD MS/MS fragmentation pattern using N₂ as collision gas and analyzed in atmospheric pressure chemical ionization (APCI) under negative ionization comprising daughter ions of parent [M−H] mass 521: 503, 459, 477, 504, 441 and 485,
GTA-524, having an accurate neutral mass within 1 PPM Dalton mass accuracy of 524.4441, having the molecular formula of C₃₂H₆₀O₅, and characterized by a OD MS/MS fragmentation pattern using N₂ as collision gas and analyzed in atmospheric pressure chemical ionization (APCI) under negative ionization comprising daughter ions of parent [M−H] mass 523: 505, 461, 479, 506, 443 and 487,
GTA-530, having an accurate neutral mass within 1 PPM Dalton mass accuracy of 530.4335, having the molecular formula of C₃₄H₅₈O₄, and characterized by a OD MS/MS fragmentation pattern using N₂ as collision gas and analyzed in atmospheric pressure chemical ionization (APCI) under negative ionization comprising daughter ions of parent [M−H] mass 529: 467, 511 and 485,
GTA-532, having an accurate neutral mass within 1 PPM Dalton mass accuracy of 532.4492, having the molecular formula of C₃₄H₆₀O₄, and characterized by a OD MS/MS fragmentation pattern using N₂ as collision gas and analyzed in atmospheric pressure chemical ionization (APCI) under negative ionization comprising daughter ions of parent [M−H] mass 531: 513, 469, 487 and 495,
GTA-536, having an accurate neutral mass within 1 PPM Dalton mass accuracy of 536.4077, having the molecular formula of C₃₂H₅₆O₆, and characterized by a OD MS/MS fragmentation pattern using N₂ as collision gas and analyzed in atmospheric pressure chemical ionization (APCI) under negative ionization comprising daughter ion of parent [M−H] mass 535: 473,
GTA-538, having an accurate neutral mass within 1 PPM Dalton mass accuracy of 538.4233, having the molecular formula of C₃₂H₅₈O₆, and characterized by a OD MS/MS fragmentation pattern using N₂ as collision gas and analyzed in atmospheric pressure chemical ionization (APCI) under negative ionization comprising daughter ions of parent [M−H] mass 537: 519, 475, 493, 501 and 457,
GTA-540, having an accurate neutral mass within 1 PPM Dalton mass accuracy of 540.4389, having the molecular formula of C₃₂H₆₀O₆, and characterized by a OD MS/MS fragmentation pattern using N₂ as collision gas and analyzed in atmospheric pressure chemical ionization (APCI) under negative ionization comprising daughter ions of parent [M−H] mass 539: 315, 521, 495 and 477,
GTA-550, having an accurate neutral mass within 1 PPM Dalton mass accuracy of 550.4597, having the molecular formula of C₃₄H₆₂O₅, and characterized by a OD MS/MS fragmentation pattern using N₂ as collision gas and analyzed in atmospheric pressure chemical ionization (APCI) under negative ionization comprising daughter ions of parent [M−H] mass 549: 487, 531, 251, 253, 513, 469 and 506,
GTA-574, having an accurate neutral mass within 1 PPM Dalton mass accuracy of 574.4597, having the molecular formula of C₃₆H₆₂O₅, and characterized by a OD MS/MS fragmentation pattern using N₂ as collision gas and analyzed in atmospheric pressure chemical ionization (APCI) under negative ionization comprising daughter ions of parent [M−H] mass 573: 295, 223, 555 and 511,
GTA-576, having an accurate neutral mass within 1 PPM Dalton mass accuracy of 576.4754, having the molecular formula of C₃₆H₆₄O₅, and characterized by a OD MS/MS fragmentation pattern using N₂ as collision gas and analyzed in atmospheric pressure chemical ionization (APCI) under negative ionization comprising daughter ions of parent [M−H] mass 575: 277, 297, 557, 513 and 495,
GTA-580, having an accurate neutral mass within 1 PPM Dalton mass accuracy of 580.5067, having the molecular formula of C₃₆H₆₈O₅, and characterized by a OD MS/MS fragmentation pattern using N₂ as collision gas and analyzed in atmospheric pressure chemical ionization (APCI) under negative ionization comprising daughter ions of parent [M−H] mass 579: 561, 543, 535, 517 and 499,
GTA-590, having an accurate neutral mass within 1 PPM Dalton mass accuracy of 590.4546, having the molecular formula of C₃₆H₆₂O₆, and characterized by a OD MS/MS fragmentation pattern using N₂ as collision gas and analyzed in atmospheric pressure chemical ionization (APCI) under negative ionization comprising daughter ion of parent [M−H] mass 589: 545,
GTA-592, having an accurate neutral mass within 1 PPM Dalton mass accuracy of 592.4703, having the molecular formula of C₃₆H₆₄O₆, and characterized by a OD MS/MS fragmentation pattern using N₂ as collision gas and analyzed in atmospheric pressure chemical ionization (APCI) under negative ionization comprising daughter ions of parent [M−H] mass 591: 555 and 113,
GTA-594, having an accurate neutral mass within 1 PPM Dalton mass accuracy of 594.4859, having the molecular formula of C₃₆H₆₆O₆, and characterized by a OD MS/MS fragmentation pattern using N₂ as collision gas and analyzed in atmospheric pressure chemical ionization (APCI) under negative ionization comprising daughter ions of parent [M−H] mass 593: 557 371, 315 and 277, and
GTA-596, having an accurate neutral mass within 1 PPM Dalton mass accuracy of 596.5016, having the molecular formula of C₃₆H₆₈O₆, and characterized by a OD MS/MS fragmentation pattern using N₂ as collision gas and analyzed in atmospheric pressure chemical ionization (APCI) under negative ionization comprising daughter ions of parent [M−H] mass 595: 279, 315, 297, 577 and 559.

16. The method of claim 11, wherein said GTA dicarboxylic fatty acid metabolite is GTA-446, having the formula C₂₈H₄₆O₄ and the structure:

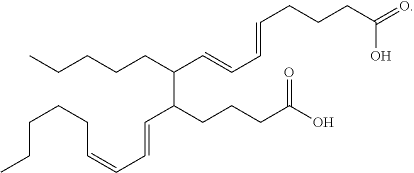 [SMILES: CCCCC/C=C\C=C\C(CCCC(=O)O)C(/C=C/C=C/CCCC(=O)O)CCCCC]

17. A kit for detecting and treating a gastric tract acid (GTA) insufficiency in a mammalian subject, comprising:
a blood specimen collection device for collecting a blood sample from said mammalian subject,
packaging and instructions for submitting the blood sample to a central processing facility to test levels in said blood sample of one or more GTA dicarboxylic fatty acid metabolite, wherein said GTA dicarboxylic fatty acid metabolite is a dicarboxylic fatty acid between 28 and 36 carbons comprised of a dimeric fatty acid structure of two shorter chains ranging between 14 and 18 carbons in length joined by a single or double bond; and
instructions for obtaining the results of testing said blood sample from the central processing facility, wherein in the case of a positive test result comprising a detected low GTA level, a GTA-augmenting anti-inflammatory prebiotic, probiotic, or synthetic GTA product is provided.

18. The kit of claim 17, wherein the GTA-augmenting anti-inflammatory prebiotic, probiotic, or synthetic GTA product is a composition comprising at least one live or attenuated culture containing a microbial species selected from the genus Blautia, species Faecalibacterium prausnitzii, genus Bacteroides, family Ruminococcaceae, family Lachnospiraceae, genus Coprococcus, genus Roseburia, genus Oscillospira, species Ruminococcus bromii, genus Ruminococcus, family Costridiaceae, species Dorea formicigenerans, species Bacteroides uniformis, genus Dorea, genus Streptococcus, order Clostridiales, genus Anaerostipes, genus Dialister, species Bifidobacterium adolescentis, family Coriobacteriaceae, genus Faecalibacterium, genus Sutterella, species Bacteroides ovatus, genus Parabacteroides, genus Ruminococcus, species Bacteroides faecis, species Eubacterium biforme, genus Phascolartobacterium, and family Enterobacteriaceae; or a prebiotic composition which increases growth and/or viability of said microbial species in the gut; wherein the composition increases the synthesis of at least one GTA dicarboxylic fatty acid metabolite in said subject.

19. The kit of claim 17, wherein said composition is provided if the levels of said one or more GTA dicarboxylic fatty acid metabolite in said subject are lower than a predetermined control level, an earlier test value for said subject, or a normal level for healthy subjects.

20. The kit of claim 19, wherein the control comprises a predetermined threshold value for said at least one GTA dicarboxylic fatty acid metabolite.

21. The kit of claim 17, wherein said composition comprises a live or attenuated culture of a microbial species from the genus Blautia, a live or attenuated culture of Faecalibacterium prausnitzii, or a combination thereof, within a pharmaceutically-acceptable carrier suitable for administration to the gastrointestinal tract of said subject.

22. The kit of claim 17, wherein said GTA dicarboxylic fatty acid metabolite is selected from the group consisting of: GTA-446, GTA-448, GTA-450, GTA-452, GTA-464, GTA-466, GTA-468, GTA-474, GTA-476, GTA-478, GTA-484, GTA-490, GTA-492, GTA-494, GTA-502, GTA-504, GTA-512, GTA-518, GTA-520, GTA-522, GTA-524, GTA-530, GTA-532, GTA-536, GTA-538, GTA-540, GTA-550, GTA-574, GTA-576, GTA-580, GTA-590, GTA-592, GTA-594, and GTA-596.

23. The kit of claim 22, wherein said GTA dicarboxylic fatty acid metabolite has an accurate neutral mass within 1 PPM Dalton mass accuracy of 446.3396 (GTA-446), 448.3553 (GTA-448), 450.3709 (GTA-450), 452.3866 (GTA-452), 464.3522 (GTA-464), 466.3661 (GTA-466), 468.3814 (GTA-468), 474.3736 (GTA-474), 476.3866 GTA-476, 478.4022 (GTA-478), 484.3764 (GTA-484), 490.3658 (GTA-490), 492.3815 (GTA-492), 494.3971 (GTA-494), 502.4022 (GTA-502), 504.4195 (GTA-504), 512.4077 (GTA-512), 518.3974 (GTA-518), 520.4128 (GTA-520), 522.4284 (GTA-522), 524.4441 (GTA-524), 530.4335 (GTA-530), 532.4492 (GTA-532), 536.4077 (GTA-536), 538.4233 (GTA-538), 540.4389 (GTA-540), 550.4597 (GTA-550), 574.4597 (GTA-574), 576.4754 (GTA-576), 580.5067 (GTA-580), 590.4546 (GTA-590), 592.4703 (GTA-592), 594.4859 (GTA-594), or 596.5016 (GTA-596).

24. The kit of claim 22, wherein said GTA dicarboxylic fatty acid metabolite has a molecular formula of C₂₈H₄₆O₄ (GTA-446), C₂₈H₄₈O₄ (GTA-448), C₂₈H₅₀O₄ (GTA-450), C₂₈H₅₂O₄ (GTA-452), 464.3522 (GTA-464), 466.3661 (GTA-466), C₂₈H₅₂O(GTA-468), C₃₀H₅₀O₄ (GTA-474), C₃₀H₅₂O₄ GTA-476, C₃₀H₅₄O₄ (GTA-478), C₂₈H₅₂O₆ (GTA-484), C₃₀H₅₀O₅ (GTA-490), C₃₀H₅₂O(GTA-492), C₃₀H₅₄O₅ (GTA-494), C₃₂H₅₄O₄ (GTA-502), C₃₂H₅₆O₄ (GTA-504), C₃₀H₅₆O₆ (GTA-512), C₃₂H₅₄O₅ (GTA-518), C₃₂H₅₆O₅ (GTA-520), C₃₂H₅₈O₅ (GTA-522), C₃₂H₆₀O₅ GTA-524, C₃₄H₅₈O₄ (GTA-530), C₃₄H₆₀O₄ (GTA-532), C₃₂H₅₆O₆ (GTA-536), C₃₂H₅₈O₆ (GTA-538), C₃₂H₆₀O₆ (GTA-540), C₃₄H₆₂O₅ (GTA-550), C₃₆H₆₂O₅ (GTA-574), C₃₆H₆₄O₅ (GTA-576), C₃₆H₆₈O₅(GTA-580), C₃₆H₆₂O₆(GTA-590), C₃₆H₆₄O₆ (GTA-592), C₃₆H₆₆O₆ (GTA-594), or C₃₆H₆₈O₆ (GTA-596).

25. The kit of claim 17, wherein said GTA dicarboxylic fatty acid metabolite is measured using collision induced dissociation (CID) tandem mass spectrometry and is selected from one or more of the GTA dicarboxylic fatty acid metabolites listed below:
GTA-446, having an accurate neutral mass within 1 PPM Dalton mass accuracy of 446.3396, the molecular formula of C₂₈H₄₆O₄, and characterized by a OD MS/MS fragmentation pattern using N₂ as collision gas and analyzed in atmospheric pressure chemical ionization (APCI) under negative ionization comprising daughter ions of parent [M−H] mass 445: 427, 409, 401, and 383,
GTA-448, having an accurate neutral mass within 1 PPM Dalton mass accuracy of 448.3553, the molecular formula of C₂₈H₄₈O₄, and characterized by a OD MS/MS fragmentation pattern using N₂ as collision gas and analyzed in atmospheric pressure chemical ionization (APCI) under negative ionization comprising daughter ions of parent [M−H] mass 447: 429, 411, 403, and 385,
GTA-450, having an accurate neutral mass within 1 PPM Dalton mass accuracy of 450.3709, the molecular formula of C₂₈H₅₀O₄, and characterized by a OD MS/MS fragmentation pattern using N₂ as collision gas and analyzed in atmospheric pressure chemical ionization (APCI) under negative ionization comprising daughter ions of parent [M−H] mass 449: 431, 413, 405, and 387,
GTA-452, having an accurate neutral mass within 1 PPM Dalton mass accuracy of 452.3866, the molecular formula of C₂₈H₅₂O₄, and characterized by a OD MS/MS fragmentation pattern using N₂ as collision gas and analyzed in atmospheric pressure chemical ionization (APCI) under negative ionization comprising daughter ions of parent [M−H] mass 451: 433, 407, and 389,
GTA-464, having an accurate neutral mass within 1 PPM Dalton mass accuracy of 464.3522, the molecular formula of C₂₈H₄₈O₅, and characterized by a OD MS/MS fragmentation pattern using N₂ as collision gas and analyzed in atmospheric pressure chemical ionization (APCI) under negative ionization comprising daughter ions of parent [M−H] mass 463: 445, 419, 401, and 383,
GTA-466, having an accurate neutral mass within 1 PPM Dalton mass accuracy of 466.3661, the molecular formula of C₂₈H₅₀O₅, and characterized by a OD MS/MS fragmentation pattern using N₂ as collision gas and analyzed in atmospheric pressure chemical ionization (APCI) under negative ionization comprising daughter ions of parent [M−H] mass 465: 447, 421, and 403,
GTA-468, having an accurate neutral mass within 1 PPM Dalton mass accuracy of 468.3814, having the molecular formula of C₂₈H₅₂O, and characterized by a OD MS/MS fragmentation pattern using N₂ as collision gas and analyzed in atmospheric pressure chemical ionization (APCI) under negative ionization comprising daughter ions of parent [M−H] mass 467: 449, 423, and 405,
GTA-474, having an accurate neutral mass within 1 PPM Dalton mass accuracy of 474.3736, having the molecular formula of C₃₀H₅₀O₄, and characterized by a OD MS/MS fragmentation pattern using N₂ as collision gas and analyzed in atmospheric pressure chemical ionization (APCI) under negative ionization comprising daughter ions of parent [M−H] mass 473: 455, 429, and 411,
GTA-476, having an accurate neutral mass within 1 PPM Dalton mass accuracy of 476.3866, having the molecular formula of C₃₀H₅₂O₄, and characterized by a OD MS/MS fragmentation pattern using N₂ as collision gas and analyzed in atmospheric pressure chemical ionization (APCI) under negative ionization comprising daughter ions of parent [M−H] mass 475: 457, 431, 439 and 413,
GTA-478, having an accurate neutral mass within 1 PPM Dalton mass accuracy of 478.4022, having the molecular formula of C₃₀H₅₄O₄, and characterized by a OD MS/MS fragmentation pattern using N₂ as collision gas and analyzed in atmospheric pressure chemical ionization (APCI) under negative ionization comprising daughter ions of parent [M−H] mass 477: 459, 433, 441 and 415,
GTA-484, having an accurate neutral mass within 1 PPM Dalton mass accuracy of 484.3764, having the molecular formula of C₂₈H₅₂O₆, and characterized by a OD MS/MS fragmentation pattern using N₂ as collision gas and analyzed in atmospheric pressure chemical ionization (APCI) under negative ionization comprising daughter ions of parent [M−H] mass 483: 465, 315, 439 483, 421, and 447,
GTA-490, having an accurate neutral mass within 1 PPM Dalton mass accuracy of 490.3658, having the molecular formula of C₃₀H₅₀O₅, and characterized by a OD MS/MS fragmentation pattern using N₂ as collision gas and analyzed in atmospheric pressure chemical ionization (APCI) under negative ionization comprising daughter ions of parent [M−H] mass 489: 445, 471, 427 and 319,
GTA-492, having an accurate neutral mass within 1 PPM Dalton mass accuracy of 492.3815, having the molecular formula of C₃₀H₅₂O₅, and characterized by a OD MS/MS fragmentation pattern using N₂ as collision gas and analyzed in atmospheric pressure chemical ionization (APCI) under negative ionization comprising daughter ions of parent [M−H] mass 491: 241, 249, 267, 473, and 447,
GTA-494, having an accurate neutral mass within 1 PPM Dalton mass accuracy of 494.3971, having the molecular formula of C₃₀H₅₄O₅, and characterized by a OD MS/MS fragmentation pattern using N₂ as collision gas and analyzed in atmospheric pressure chemical ionization (APCI) under negative ionization comprising daughter ions of parent [M−H] mass 493: 475, 215, and 449,
GTA-502, having an accurate neutral mass within 1 PPM Dalton mass accuracy of 502.4022, having the molecular formula of C₃₂H₅₄O₄, and characterized by a OD MS/MS fragmentation pattern using N₂ as collision gas and analyzed in atmospheric pressure chemical ionization (APCI) under negative ionization comprising daughter ions of parent [M−H] mass 501: 483, 457, 465 and 439,
GTA-504, having an accurate neutral mass within 1 PPM Dalton mass accuracy of 504.4195, having the molecular formula of C₃₂H₅₆O₄, and characterized by a OD MS/MS fragmentation pattern using N₂ as collision gas and analyzed in atmospheric pressure chemical ionization (APCI) under negative ionization comprising daughter ions of parent [M−H] mass 503: 485, 459, 467 and 441,
GTA-512, having an accurate neutral mass within 1 PPM Dalton mass accuracy of 512.4077, having the molecular formula of C₃₀H₅₆O₆, and characterized by a OD MS/MS fragmentation pattern using N₂ as collision gas and analyzed in atmospheric pressure chemical ionization (APCI) under negative ionization comprising daughter ions of parent [M−H] mass 511: 493, 315, and 467,
GTA-518, having an accurate neutral mass within 1 PPM Dalton mass accuracy of 518.3974, having the molecular formula of C₃₂H₅₄O₅, and characterized by a OD MS/MS fragmentation pattern using N₂ as collision gas and analyzed in atmospheric pressure chemical ionization (APCI) under negative ionization comprising daughter ions of parent [M−H] mass 517: 499, 473, 499, 481 and 445,
GTA-520, having an accurate neutral mass within 1 PPM Dalton mass accuracy of 520.4128, having the molecular formula of C₃₂H₅₆O₅, and characterized by a OD MS/MS fragmentation pattern using N₂ as collision gas and analyzed in atmospheric pressure chemical ionization (APCI) under negative ionization comprising daughter ions of parent [M−H] mass 519: 501, 457, 475, 459, 447 and 483,
GTA-522, having an accurate neutral mass within 1 PPM Dalton mass accuracy of 522.4284, having the molecular formula of C₃₂H₅₈O₅, and characterized by a OD MS/MS fragmentation pattern using N₂ as collision gas and analyzed in atmospheric pressure chemical ionization (APCI) under negative ionization comprising daughter ions of parent [M−H] mass 521: 503, 459, 477, 504, 441 and 485,
GTA-524, having an accurate neutral mass within 1 PPM Dalton mass accuracy of 524.4441, having the molecular formula of C₃₂H₆₀O₅, and characterized by a OD MS/MS fragmentation pattern using N₂ as collision gas and analyzed in atmospheric pressure chemical ionization (APCI) under negative ionization comprising daughter ions of parent [M−H] mass 523: 505, 461, 479, 506, 443 and 487,
GTA-530, having an accurate neutral mass within 1 PPM Dalton mass accuracy of 530.4335, having the molecular formula of C₃₄H₅₈O₄, and characterized by a OD MS/MS fragmentation pattern using N₂ as collision gas and analyzed in atmospheric pressure chemical ionization (APCI) under negative ionization comprising daughter ions of parent [M−H] mass 529: 467, 511 and 485,
GTA-532, having an accurate neutral mass within 1 PPM Dalton mass accuracy of 532.4492, having the molecular formula of C₃₄H₆₀O₄, and characterized by a OD MS/MS fragmentation pattern using N₂ as collision gas and analyzed in atmospheric pressure chemical ionization (APCI) under negative ionization comprising daughter ions of parent [M−H] mass 531: 513, 469, 487 and 495,
GTA-536, having an accurate neutral mass within 1 PPM Dalton mass accuracy of 536.4077, having the molecular formula of C₃₂H₅₆O₆, and characterized by a OD MS/MS fragmentation pattern using N₂ as collision gas and analyzed in atmospheric pressure chemical ionization (APCI) under negative ionization comprising daughter ion of parent [M−H] mass 535: 473,
GTA-538, having an accurate neutral mass within 1 PPM Dalton mass accuracy of 538.4233, having the molecular formula of C₃₂H₅₈O₆, and characterized by a OD MS/MS fragmentation pattern using N₂ as collision gas and analyzed in atmospheric pressure chemical ionization (APCI) under negative ionization comprising daughter ions of parent [M−H] mass 537: 519, 475, 493, 501 and 457,
GTA-540, having an accurate neutral mass within 1 PPM Dalton mass accuracy of 540.4389, having the molecular formula of C₃₂H₆₀O₆, and characterized by a OD MS/MS fragmentation pattern using N₂ as collision gas and analyzed in atmospheric pressure chemical ionization (APCI) under negative ionization comprising daughter ions of parent [M−H] mass 539: 315, 521, 495 and 477,
GTA-550, having an accurate neutral mass within 1 PPM Dalton mass accuracy of 550.4597, having the molecular formula of C₃₄H₆₂O₅, and characterized by a OD MS/MS fragmentation pattern using N₂ as collision gas and analyzed in atmospheric pressure chemical ionization (APCI) under negative ionization comprising daughter ions of parent [M−H] mass 549: 487, 531, 251, 253, 513, 469 and 506,
GTA-574, having an accurate neutral mass within 1 PPM Dalton mass accuracy of 574.4597, having the molecular formula of C₃₆H₆₂O₅, and characterized by a OD MS/MS fragmentation pattern using N₂ as collision gas and analyzed in atmospheric pressure chemical ionization (APCI) under negative ionization comprising daughter ions of parent [M−H] mass 573: 295, 223, 555 and 511,
GTA-576, having an accurate neutral mass within 1 PPM Dalton mass accuracy of 576.4754, having the molecular formula of C₃₆H₆₄O₅, and characterized by a OD MS/MS fragmentation pattern using N₂ as collision gas and analyzed in atmospheric pressure chemical ionization (APCI) under negative ionization comprising daughter ions of parent [M−H] mass 575: 277, 297, 557, 513 and 495,
GTA-580, having an accurate neutral mass within 1 PPM Dalton mass accuracy of 580.5067, having the molecular formula of C₃₆H₆₈O₅, and characterized by a OD MS/MS fragmentation pattern using N₂ as collision gas and analyzed in atmospheric pressure chemical ionization (APCI) under negative ionization comprising daughter ions of parent [M−H] mass 579: 561, 543, 535, 517 and 499,
GTA-590, having an accurate neutral mass within 1 PPM Dalton mass accuracy of 590.4546, having the molecular formula of C₃₆H₆₂O₆, and characterized by a OD MS/MS fragmentation pattern using N₂ as collision gas and analyzed in atmospheric pressure chemical ionization (APCI) under negative ionization comprising daughter ion of parent [M−H] mass 589: 545,
GTA-592, having an accurate neutral mass within 1 PPM Dalton mass accuracy of 592.4703, having the molecular formula of C₃₆H₆₄O₆, and characterized by a OD MS/MS fragmentation pattern using N₂ as collision gas and analyzed in atmospheric pressure chemical ionization (APCI) under negative ionization comprising daughter ions of parent [M−H] mass 591: 555 and 113,
GTA-594, having an accurate neutral mass within 1 PPM Dalton mass accuracy of 594.4859, having the molecular formula of C₃₆H₆₆O₆, and characterized by a OD MS/MS fragmentation pattern using N₂ as collision gas and analyzed in atmospheric pressure chemical ionization (APCI) under negative ionization comprising daughter ions of parent [M−H] mass 593: 557 371, 315 and 277, and
GTA-596, having an accurate neutral mass within 1 PPM Dalton mass accuracy of 596.5016, having the molecular formula of C₃₆H₆₈O₆, and characterized by a OD MS/MS fragmentation pattern using N₂ as collision gas and analyzed in atmospheric pressure chemical ionization (APCI) under negative ionization comprising daughter ions of parent [M−H] mass 595: 279, 315, 297, 577 and 559.

26. The kit of claim 17, wherein said GTA dicarboxylic fatty acid metabolite is GTA-446, having the formula C₂₈H₄₆O₄ and the structure:

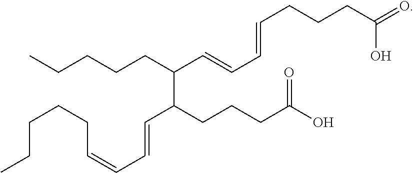 [SMILES: CCCCC/C=C\C=C\C(CCCC(=O)O)C(/C=C/C=C/CCCC(=O)O)CCCCC]

27. The kit of claim 17, wherein said gastric tract acid (GTA) insufficiency is an indicator of a gastrointestinal (GI) inflammatory state.
